(19) United States
(12) Patent Application Publication
(10) Pub. No.: US 2018/0312473 A1
(43) Pub. Date: Nov. 1, 2018

(54) PHENAZINE DERIVATIVES AS ANTIMICROBIAL AGENTS

(71) Applicant: Robert William Huigens, III, (US)

(72) Inventor: Robert William Huigens, III, (US)

(21) Appl. No.: 15/744,319

(22) PCT Filed: Jul. 15, 2016
(86) PCT No.: PCT/US16/42439

§ 371 Date: Jan. 12, 2018

Related U.S. Application Data
(60) Provisional application No. 62/193,045, filed on Jul. 15, 2015.
(60) Provisional application No. 62/243,658, filed on Oct. 19, 2015.
(60) Provisional application No. 62/313,665, filed on Mar. 25, 2016.

(51) Int. Cl. C07D 241/46; C07D 241/38; A61P 31/04

(52) U.S. Cl. CPC C07D 241/46; A61P 31/04; C07D 241/38

(57) ABSTRACT

The present invention provides novel phenazine derivatives, such as compounds of Formula (I′) (e.g., Formula (I)), (II), and (III), and pharmaceutically acceptable salts thereof. The compounds of the invention are expected to be antimicrobial agents and may act by a microbial warfare strategy (e.g., a reactive oxygen species (ROS)-based competition strategy). The present invention also provides pharmaceutical compositions, kits, uses, and methods that involve the compounds of the invention and may be useful in preventing or treating a microbial infection (e.g., a bacterial infection or mycobacterial infection) in a subject, inhibiting the mycobacterium), inhibiting the formation and/or growth of a biofilm, reducing or clearing a biofilm, and/or disinfecting a surface.

### RELATED APPLICATIONS

The present invention claims priority under 35 U.S.C. § 119 (e) to U.S. provisional patent applications, U.S. Ser. No. 62/193,045, filed Jul. 15, 2015, U.S. Ser. No. 62/243,658, filed Oct. 19, 2015, and U.S. Ser. No. 62/313,665, filed Mar. 25, 2016, each of which is incorporated herein by reference.

### BACKGROUND OF THE INVENTION

The emergence of multidrug resistant microbial infections (e.g., bacterial infections) has led to a serious global crisis. Almost every class of antibiotic that has been introduced into the clinic has been met with the development of drug resistant bacteria (A. E. Clatworthy, E. Pierson, and D. T. Hung, Nat. Chem. Biol., 2007, 3, 541-548; K. Lewis, Nat. Rev. Drug Discov., 2013, 12, 371-387). Despite the growing need for new antimicrobial agents, many pharmaceutical companies have abandoned their antimicrobial discovery programs as the anticipated success with target-based, high-throughput screening (HTS) campaigns has yet to be realized (K. Lewis, Nat. Rev. Drug Discov., 2013, 12, 371-387; S. J. Projan, Curr. Opin. Microbiol., 2003, 6, 427-430; E. D. Brown and G. D. Wright, Chem. Rev., 2005, 105, 759-774; D. J. Payne, M. N. Gwynn, D. J. Holmes, and D. L. Pompliano, Nat. Rev. Drug Discov., 2007, 6, 29-40). The health care emergency that has resulted from drug resistant microbial infections has been gaining momentum over the past four decades as only two new classes of antibiotics have been introduced into the clinic (K. Lewis, Nat. Rev. Drug Discov., 2013, 12, 371-387; E. D. Brown and G. D. Wright, Chem. Rev., 2005, 105, 759-774).

A wide range of microorganisms produce potent antibiotics as agents of microbial warfare and competition. As a result, the large majority of the antibiotic arsenal is based on such natural products discovered in the antibiotic golden era between the 1940s and 1960s (e.g., penicillin, streptomycin, erythromycin, tetracycline, vancomycin) or their synthetic derivatives (Lewis, Nat. Rev. Drug Discov., 2013, 12, 371-387). In fact, very few clinically useful treatment options for microbial infections have been developed from purely synthetic origins (e.g., sulfonamides, quinolones, oxazolidinones).

In addition to infections resulting from planktonic bacteria, biofilms also play a key role in pathogenesis. The NIH has stated that bacterial biofilms are associated with up to 80% of all bacterial infections. Biofilms are notorious for their resistance to conventional antibiotic treatments. Currently, there is a desperate need for clinically useful anti-biofilm agents as there are no FDA-approved drugs that effectively target biofilm machinery. Innovative antimicrobial strategies are needed to meet the biomedical challenges of microbial infections, especially those resulting from multidrug resistant microbial infections and pathogenic bacterial biofilms.

### SUMMARY OF THE INVENTION

The present invention provides novel halogenated phenazine derivatives (HPs, HP analogues), such as compounds of Formulae (I′) (e.g., Formula (I)), (II), and (III), and salts, hydrates, solvates, polymorphs, co-crystals, tautomers, stereoisomers, isotopically labeled derivatives, prodrugs, thereof:

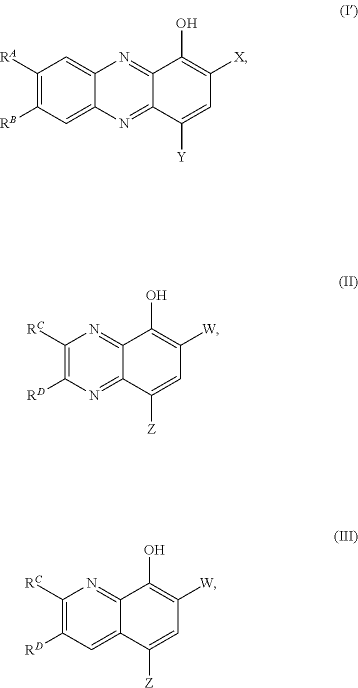

wherein X, Y, R^{A}, R^{B}, W, Z, R^{C}, and R^{D} are as described herein.

Exemplary compounds of the invention include, but are not limited to:

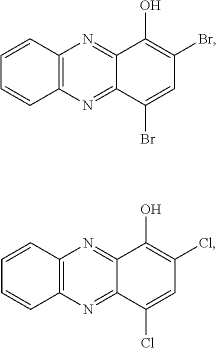 [SMILES: Oc1c(Br)cc(Br)c2nc3ccccc3nc12 ; Oc1c(Cl)cc(Cl)c2nc3ccccc3nc12]

and pharmaceutically acceptable salts, solvates, hydrates, polymorphs, co-crystals, tautomers, stereoisomers, isotopically labeled derivatives, and prodrugs thereof.

Exemplary compounds of the invention also include, but are not limited to:

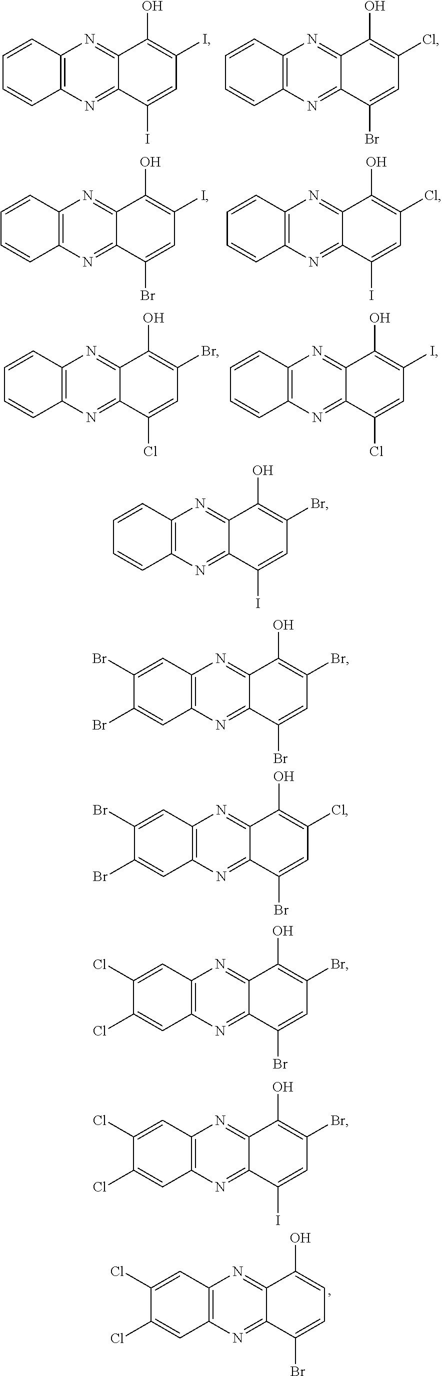

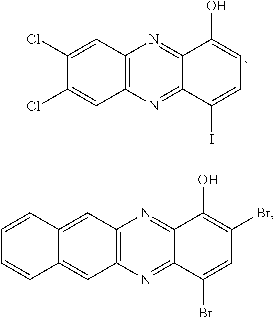

and pharmaceutically acceptable salts, solvates, hydrates, polymorphs, co-crystals, tautomers, stereoisomers, isotopically labeled derivatives, and prodrugs thereof.

Exemplary compounds of the invention also include, but are not limited to:

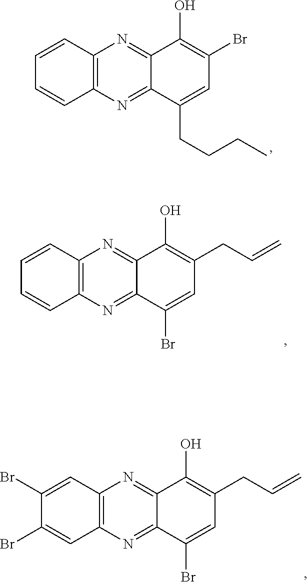 [SMILES: CCCCc1cc(Br)c(O)c2nc3ccccc3nc12 ; C=CCc1cc(Br)c2nc3ccccc3nc2c1O ; C=CCc1cc(Br)c2nc3cc(Br)c(Br)cc3nc2c1O]

and pharmaceutically acceptable salts, solvates, hydrates, polymorphs, co-crystals, tautomers, stereoisomers, isotopically labeled derivatives, and prodrugs thereof.

Exemplary compounds of the invention also include, but are not limited to:

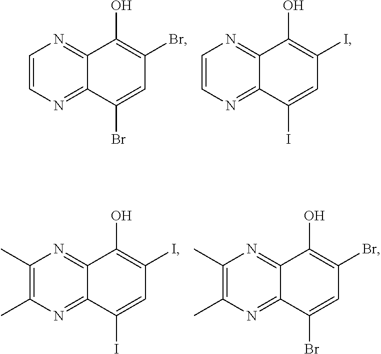

and pharmaceutically acceptable salts, solvates, hydrates, polymorphs, co-crystals, tautomers, stereoisomers, isotopically labeled derivatives, and prodrugs thereof.

Exemplary compounds of the invention also include, but are not limited to:

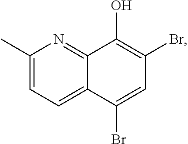

and pharmaceutically acceptable salts, solvates, hydrates, polymorphs, co-crystals, tautomers, stereoisomers, isotopically labeled derivatives, and prodrugs thereof.

The compounds of the invention may exhibit antimicrobial activity (e.g., antibacterial activity, such as antibacterial activity against strains of Staphylococcus aureus (e.g., methicillin-resistant strains of Staphylococcus aureus), strains of Staphylococcus epidermidis (e.g., a methicillin-resistant strain of Staphylococcus epidermidis (MRSE)), and strains of Enterococcus faecium (e.g., vancomycin-resistant strains of Enterococcus faecium)). Without wishing to be bound by any particular theory, it is thought that the compounds of the invention may act by a microbial warfare strategy (e.g., a reactive oxygen species (ROS)-based competition strategy) similar to the one employed by Pseudomonas aeruginosa (P. aeruginosa). The inventive compounds may generate ROS in, near, or around a microorganism (e.g., bacterium, mycobacterium, archaeon, protist, fungus, or parasite), which may be toxic to the microorganism. Moreover, the inventive compounds may be able to reduce, inhibit, and/or remove biofilms (e.g., Staphylococcus aureus biofilms (e.g., MRSA biofilms) and/or Staphylococcus epidermidis biofilms (e.g., MRSE biofilms)). The inventive compounds preferably have minimal or no adverse side effects. In certain embodiments, the inventive compounds have low cytotoxicity with respect to mammalian cells and/or demonstrate low hemolysis activity.

In another aspect, the present invention provides compositions including a compound of the invention and optionally an excipient. In certain embodiments, the composition includes an effective amount of the compound for disinfecting a surface. In certain embodiments, the composition is a pharmaceutical composition including a compound of the invention and optionally a pharmaceutically acceptable excipient. In certain embodiments, a pharmaceutical composition of the invention includes an effective amount of a compound of the invention for administration to a subject. In certain embodiments, the pharmaceutical composition is useful in a method of the invention (e.g., a method of treating a microbial infection, preventing a microbial infection, inhibiting the growth of a microorganism, inhibiting the reproduction of a microorganism, killing a microorganism, inhibiting the formation and/or growth of a biofilm, reducing or removing a biofilm, or disinfecting a surface). In certain embodiments, the microorganism is a microorganism described herein. In certain embodiments, the microorganism is a bacterium. In certain embodiments, the bacterium is a Gram-positive bacterium (e.g., a Staphylococcus species or Enterococcus species). In certain embodiments, the bacterium is a Gram-negative bacterium (e.g., an Acinetobacter species). In certain embodiments, the microorganism is a mycobacterium (e.g., a strain of Mycobacterium tuberculosis).

Another aspect of the present invention relates to methods of treating and/or preventing a microbial infection in a subject in need thereof, the method including administering to the subject a therapeutically or prophylactically effective amount of a compound or pharmaceutical composition of the invention. In certain embodiments, the microbial infection is treated and/or prevented by the inventive methods. The microbial infections that may be treated and/or prevented by the inventive methods include, but are not limited to, microbial respiratory tract infections, microbial gastrointestinal tract infections, microbial urogenital tract infections, microbial bloodstream infections, microbial ear infections, microbial skin infections, microbial oral infections, microbial dental infections, microbial wound or surgical site infections, microbial infections associated with cystic fibrosis, and microbial infections associated with implanted devices. In certain embodiments, the microbial infection described herein is a bacterial infection. In certain embodiments, the bacterium causing the bacterial infections is a Gram-positive bacterium (e.g., a Staphylococcus species or Enterococcus species). In certain embodiments, the bacterium causing the bacterial infections is a Gram-negative bacterium (e.g., an Acinetobacter species). In certain embodiments, the microbial infection described herein is a mycobacterial infection (e.g., an infection caused by Mycobacterium tuberculosis). In certain embodiments, the subject is a human. In certain embodiments, the subject is a human with cystic fibrosis. In certain embodiments, the subject is a non-human animal.

In another aspect, the present invention provides methods of inhibiting the growth of a microorganism (e.g., a bacterium, mycobacterium, archaeon, protist, fungus, or parasite) in vitro or in vivo.

In yet another aspect, the present invention provides methods of inhibiting the reproduction of a microorganism (e.g., a bacterium, mycobacterium, archaeon, protist, fungus, or parasite) in vitro or in vivo.

In yet another aspect, the present invention provides methods of killing a microorganism (e.g., a bacterium, mycobacterium, archaeon, protist, fungus, or parasite) in intro or in vivo.

In certain embodiments, an inventive method includes contacting a microorganism (e.g., bacterium, mycobacterium, archaeon, protist, fungus, or parasite) with a compound or pharmaceutical composition of the invention in an amount effective at inhibiting the growth and/or reproduction of or killing the microorganism.

Another aspect of the invention relates to methods of inhibiting the formation and/or growth of, reducing, or removing a biofilm, the method including contacting the biofilm with an effective amount of a compound or pharmaceutical composition of the invention. In certain embodiments, the biofilm includes a microorganism (e.g., a bacterium, mycobacterium, archaeon, protist, fungus, or parasite). In certain embodiments, the biofilm includes bacteria. The biofilm may include one or more species of bacteria and/or other microorganisms.

Another aspect of the present invention relates to methods of disinfecting a surface, the methods including contacting the surface with an effective amount of a compound or composition of the invention. In certain embodiments, the surface is a biological surface (e.g., skin). In certain embodiments, the surface is a non-biological surface.

Another aspect of the present invention relates to kits comprising a container with a compound or composition (e.g., pharmaceutical composition) of the invention. The kits of the invention may include a single dose or multiple doses of the compound or pharmaceutical composition thereof. The provided kits may be useful in a method of the invention (e.g., a method of treating a microbial infection, preventing a microbial infection, inhibiting the growth of a microorganism (e.g., bacterium, mycobacterium, archaeon, protist, fungus, or parasite), inhibiting the reproduction of a microorganism, killing a microorganism, inhibiting the formation and/or growth of a biofilm, reducing or removing a biofilm, or disinfecting a surface). A kit of the invention may further include instructions for using the kit (e.g., instructions for using the compound or composition (e.g., pharmaceutical composition) included in the kit).

In another aspect, the present invention provides uses of the compounds and pharmaceutical compositions of the invention for manufacturing a medicament for treating and/or preventing a microbial infection.

In another aspect, the present invention provides the compounds and pharmaceutical compositions of the invention for use in methods of preventing and/or treating a microbial infection.

In another aspect, the present invention provides the compounds and pharmaceutical compositions of the invention for treating and/or preventing a microbial infection.

The present application refers to various issued patent, published patent applications, journal articles, and other publications, all of which are incorporated herein by reference. The details of one or more embodiments of the invention are set forth herein. Other features, objects, and advantages of the invention will be apparent from the Detailed Description, Examples, and Claims.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

When a range of values is listed, it is intended to encompass each value and subrange within the range. For example “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

The term “aliphatic” refers to alkyl, alkenyl, alkynyl, and carbocyclic groups. Likewise, the term “heteroaliphatic” as used herein, refers to heteroalkyl, heteroalkenyl, heteroalkynyl, and heterocyclic groups.

“Alkyl” refers to a radical of a straight-chain or branched saturated hydrocarbon group having from 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents. In certain embodiments, the alkyl group is unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is substituted C₁₋₁₀ alkyl (such as substituted C₁₋₆ alkyl, e.g., —CH₂F, —CHF₂, —CF₃, Bn).

“Alkenyl” refers to a radical of a straight-chain or branched hydrocarbon group having from 2 to 20 carbon atoms, one or more carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl. In an alkenyl group, a C═C double bond for which the stereochemistry is not specified (e.g., —CH═CHCH₃ or

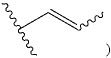

may be an (E)- or (Z)-double bond

“Alkynyl” refers to a radical of a straight-chain or branched hydrocarbon group having from 2 to 20 carbon atoms, one or more carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclic ring, as defined above, is fused with one or more carbocyclyl groups wherein the point of attachment is either on the carbocyclyl or heterocyclic ring, or ring systems wherein the heterocyclic ring, as defined above, is fused with one or more aryl or heteroaryl groups, wherein the point of attachment is on the heterocyclic ring, and in such instances, the number of ring members continue to designate the number of ring members in the heterocyclic ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, and thiiranyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing three heteroatoms include, without limitation, triazinyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” refers to a radical of a monocyclic or polycyclic (e.g., bicyclic or tricyclic) 4n+2 aromatic ring system (e.g., having 6, 10, or 14 p electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Aralkyl” is a subset of alkyl and aryl and refers to an optionally substituted alkyl group substituted by an optionally substituted aryl group. In certain embodiments, the aralkyl is optionally substituted benzyl. In certain embodiments, the aralkyl is benzyl. In certain embodiments, the aralkyl is optionally substituted phenethyl. In certain embodiments, the aralkyl is phenethyl.

“Heteroaryl” refers to a radical of a 5-10 membered monocyclic or bicyclic 4n+2 aromatic ring system (e.g., having 6 or 10 p electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl, and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Heteroaralkyl” is a subset of alkyl and heteroaryl and refers to an optionally substituted alkyl group substituted by an optionally substituted heteroaryl group.

“Partially unsaturated” refers to a group that includes at least one double or triple bond. A “partially unsaturated” ring system is further intended to encompass rings having multiple sites of unsaturation, but is not intended to include aromatic groups (e.g., aryl or heteroaryl groups) as herein defined. Likewise, “saturated” refers to a group that does not contain a double or triple bond, i.e., contains all single bonds.

Alkyl, alkenyl, alkynyl, carbocyclyl, heterocyclyl, aryl, and heteroaryl groups, which are divalent bridging groups are further referred to using the suffix -ene, e.g., alkylene, alkenylene, alkynylene, carbocyclylene, heterocyclylene, arylene, and heteroarylene.

The term “optionally substituted” refers to substituted or unsubstituted.

Alkyl, alkenyl, alkynyl, carbocyclyl, heterocyclyl, aryl, and heteroaryl groups are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group) if not otherwise provided explicitly. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb} C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X is a counterion;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;

each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

In certain embodiments, the carbon atom substituents are independently halogen, substituted or unsubstituted C₁₋₆ alkyl, or —OR^{aa}.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

“Halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

“Acyl” refers to a moiety selected from the group consisting of —C(═O)R^{aa}, —CHO, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —C(═O)NR^{bb} SO₂R^{aa}, —C(═S)N(R^{bb})₂, —C(═O)SR^{aa}, or —C(═S)SR^{aa}, wherein R^{aa} and R^{bb} are as defined herein.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on a nitrogen atom is a nitrogen protecting group (also referred to as an amino protecting group). Nitrogen protecting groups include, but are not limited to, —OH, —OR^{aa}, —N(R^{cc})₂, —C(═O)R^{aa}, C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methyl sulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenyl ethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethyl sulfonamide, and phenacylsulfonamide.

Other nitrogen protecting groups include, but are not limited to, phenothiazinyl-(10)-acyl derivative, N′-p-toluenesulfonylaminoacyl derivative, N-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb}) 2)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), tert-butoxycarbonyl, methylthiomethyl (MTM), t-butylthiomethyl, (phenyldimethyl silyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethyl silyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethyl silylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S, S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate, alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb}) 2)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (I′) (e.g., Formula (I)), (II), or (III) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R.x H₂O, wherein R is the compound and wherein x is a number greater than 0. A given compound may form more than one type of hydrate, including, e.g., monohydrates (x is 1), lower hydrates (x is a number greater than 0 and smaller than 1, e.g., hemihydrates (R.0.5H₂O)), and polyhydrates (x is a number greater than 1, e.g., dihydrates (R.2H₂O) and hexahydrates (R.6H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

Tautomeric forms may be relevant to the attainment of the optimal chemical reactivity and biological activity of a compound of interest.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

“Polymorph” refers to a particular polymorphic variant of a given compound. Polymorphism is the ability of a solid substance of a given chemical composition to exist in more than one form or crystalline structure. Polymorphism can exist as a result of differences in crystal packing (packing polymorphism), conformational differences (conformational polymorphism), or changes due to co-crystalization with other chemical entities (pseudopolymorphism). Polymorphism is an important aspect of pharmaceutical development, in which case drugs typically receive regulatory approval for only a single form. Distinct polymorphic forms frequently vary considerably in terms of their physical properties. Altered dissolution rates, thermal stability, and hygroscopicity are frequently observed.

The term “prodrugs” refers to compounds that have cleavable groups and become by solvolysis or under physiological conditions the compounds of Formula (I′) (e.g., Formula (I)), (II), or (III), which are pharmaceutically active in vivo. Such examples include, but are not limited to, ester derivatives and the like. Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds of this invention are particular prodrugs. In some cases it is desirable to prepare double ester type prodrugs such as (acyloxy)alkyl esters or ((alkoxycarbonyl)oxy)alkylesters. C₁ to C₈ alkyl, C₂-C₈ alkenyl, C₂-C₈ alkynyl, aryl, C₇-C₁₂ substituted aryl, and C₇-C₁₂ arylalkyl esters of the compounds of Formula (I′) (e.g., Formula (I)), (II), or (III) may be preferred.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. A “patient” refers to a human subject in need of treatment of a disease. The subject may also be a plant. In certain embodiments, the plant is a land plant. In certain embodiments, the plant is a non-vascular land plant. In certain embodiments, the plant is a vascular land plant. In certain embodiments, the plant is a seed plant. In certain embodiments, the plant is a cultivated plant. In certain embodiments, the plant is a dicot. In certain embodiments, the plant is a monocot. In certain embodiments, the plant is a flowering plant. In some embodiments, the plant is a cereal plant, e.g., maize, corn, wheat, rice, oat, barley, rye, or millet. In some embodiments, the plant is a legume, e.g., a bean plant, e.g., soybean plant. In some embodiments, the plant is a tree or shrub.

The terms “administer,” “administering,” or “administration,” refers to implanting, absorbing, ingesting, injecting, inhaling, or otherwise introducing an inventive compound, or a pharmaceutical composition thereof.

The terms “treatment,” “treat,” and “treating” refer to reversing, alleviating, delaying the onset of, or inhibiting the progress of a microbial infection (e.g., a bacterial infection or mycobacterial infection). In some embodiments, treatment may be administered after one or more signs or symptoms have developed or have been observed. In other embodiments, treatment may be administered in the absence of signs or symptoms of the disease or condition. For example, treatment may be administered to a susceptible individual prior to the onset of symptoms (e.g., in light of exposure to microorganisms, in light of a history of symptoms, and/or in light of genetic or other susceptibility factors). Treatment may also be continued after symptoms have resolved, for example, to delay and/or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, an effective amount is an amount effective for inhibiting the growth of a microorganism, for inhibiting the reproduction of a microorganism, or for killing a microorganism. In certain embodiments, an effective amount is an amount effective for inhibiting the formation of a biofilm, for inhibiting the growth of a biofilm, for reducing a biofilm, or for clearing a biofilm. In certain embodiments, an effective amount is an amount effective for disinfecting a surface (e.g., killing at least 80%, at least 90%, at least 99%, at least 99.9%, or at least 99.99% of the microorganisms on the surface). In certain embodiments, an effective amount is an amount effective for killing a persister cell.

A “therapeutically effective amount” of a compound of Formula (I′) (e.g., Formula (I)), (II), or (III) is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent. In certain embodiments, a therapeutically effective amount is effective for treating a microbial infection (e.g., a bacterial infection or mycobacterial infection) in a subject, for inhibiting the growth and/or reproduction of a microorganism (e.g., a bacterium), for killing a microorganism (e.g., a bacterium), for inhibiting the formation and/or growth of a biofilm, for reducing or clearing a biofilm, and/or for disinfecting a surface.

A “prophylactically effective amount” of a compound of Formula (I′) (e.g., Formula (I)), (II), or (III) is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is effective for preventing a microbial infection (e.g., a bacterial infection or mycobacterial infection) in a subject, for inhibiting the growth and/or reproduction of a microorganism (e.g., a bacterium), for killing a microorganism (e.g., a bacterium), for inhibiting the formation and/or growth of a biofilm, for reducing or clearing a biofilm, and/or for disinfecting a surface.

The term “inhibition”, “inhibiting”, “inhibit,” “inhibitory,” or “inhibitor” refers to the ability of a compound to reduce, slow, halt, or prevent activity of a particular biological process (e.g., the growth or reproduction) of a microorganism (e.g., a bacterium, mycobacterium, archaeon, protist, fungus, or parasite) relative to vehicle.

The term “minimum inhibitory concentration” or “MIC” refers to the lowest concentration of a compound that will inhibit the visible growth of a microorganism (e.g., a bacterium, mycobacterium, archaeon, protist, fungus, or parasite) after overnight (e.g., about 16 to about 20 hours, or about 16 to about 18 hours) incubation of the microorganism with the compound at about 37° C.

The term “half maximal inhibitory concentration” or “IC₅₀” of a compound refers to the concentration of the compound that inhibits the growth of half of an inoculum of a microorganism (e.g., a bacterium, mycobacterium, archaeon, protist, fungus, or parasite).

The term “microorganism” refers to a microscopic organism, which may be a single-cell or multicellular organism. In certain embodiments, the microorganism is a bacterium, mycobacterium, archaeon, protist (e.g., protozoon, alga), fungus (e.g., yeast, mold), or parasite. In certain embodiments, the microorganism is a bacterium. In certain embodiments, the length or diameter of a microorganism is at most about 10 cm, at most about 1 cm, at most about 1 mm, at most about 100 μm, at most about 10 μm, at most about 1 μm, at most about 100 nm, or at most about 10 nm. In certain embodiments, the length or diameter of a microorganism is at most about 10 μm.

The term “biofilm” refers to a group of microorganisms (e.g., bacteria) in which cells of the microorganisms stick to each other on a surface. These adherent cells are frequently embedded within a self-produced matrix of extracellular polymeric substance (EPS). The EPS is a polymeric conglomeration generally composed of extracellular DNA, proteins, and polysaccharides. Biofilms may form on living or non-living surfaces and can be prevalent in natural, industrial, and hospital settings. The cells growing in a biofilm are physiologically distinct from planktonic cells of the same microorganism, which are single-cells that may float or swim in a liquid medium. Biofilms have been found to be involved in a wide variety of microbial infections. Biofilms are formed by numerous Gram-negative and Gram-positive bacterial species. Non-limiting examples include Bacillus spp, Staphylococcus spp, Pseudomonas spp, and Acinetobacter spp.

The term “microbial warfare” refers to a first microorganism producing a substance (e.g., an antibiotic) that is toxic to a second microorganism but is not toxic or less toxic, compared to the second microorganism, to the first microorganism. When a second microorganism in close proximity to the first microorganism contacts the substance, the growth and/or reproduction of the second microorganism may be inhibited, or the second microorganism may be killed. As a result, the first microorganism may gain a competitive advantage over the second microorganism in close proximity to the first microorganism in terms of survival, growth, and/or reproduction.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucus, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. Biological samples also include those biological samples that are transgenic, such as transgenic oocyte, sperm cell, blastocyst, embryo, fetus, donor cell, or cell nucleus.

The term “planktonic” refers to any of the group of passively floating, drifting, or somewhat motile organisms occurring in a liquid medium (e.g., an aqueous solution). This group includes, but is not limited to, microscopic bacteria, algae, or protozoa.

### BRIEF DESCRIPTION OF THE DRAWINGS

The accompanying drawings, which constitute a part of this specification, illustrate several embodiments of the invention and together with the description, serve to explain the principles of the invention.

FIG. 1 shows marine phenazine antibiotic 201 and an MRSA biofilm-eradicating agent, halogenated phenazine 202, as unique starting points to target persistent bacteria.

FIG. 2A shows exemplary results of Calgary Biofilm Device assays of 214 and vancomycin (“Vanco.”) against MRSA-2. FIG. 2B shows Live/Dead staining of MRSA-2 biofilms following treatment with 214. FIG. 2C shows Life/Dead staining of MRSA-2 biofilms following treatment with 213.

FIG. 3 shows eradication of MRSA-2 persister cells in a non-biofilm (stationary) culture. Compound 214 demonstrated effective persister cell killing compared to lead MRSA antibiotics and QAC-10. “Vanco” denotes vancomycin. “Dapto” denotes daptomycin.

FIG. 4 shows a flowchart illustrating a Calgary Biofilm Device (CBD) assay.

FIG. 5A shows MRSA-2 persister cell kill kinetics (killing of stationary cultures) of select compounds. FIG. 5B shows additional MRSA-2 persister cell kill kinetics (killing of stationary cultures) of select compounds. “Vanco” denotes vancomycin.

FIGS. 6A to 6E show halogenated phenazine cytotoxicity results (Triton-X=100% cell death; medium only: 0% cell death). FIG. 6A: compound 202. FIG. 6B: compound 209. FIG. 6C: compound 210. FIG. 6D: compound 213. FIG. 6E: compound 214.

FIG. 7 shows synthetic analogues of marine phenazine antibiotic (1) that were evaluated during these investigations.

FIGS. 8A to 8D shows Schemes 3A to 3D: (FIG. 8A (Scheme 3A)) mono- and di-halogenation routes to HP analogues 2-21, (FIG. 8B (Scheme 3B)) Wohl-Aue synthesis of HP 18, (FIG. 8C (Scheme 3C)) Suzuki Route to 4-butyl HP 22, and (FIG. 8D (Scheme 3D))O-allylation/Clainsen Rearrangement leading to HPs 23-24. Reagents and conditions of Schemes 3A to 3D: (a) NBS, NCS, or NIS, CH₂Cl₂, 19-96%; (b) KI, NaIO₄, NaCl, AcOH:H₂O (9:1), 86%; (c) BBr₃, CH₂Cl₂, —78° C. to r.t., 65-99%; (d) (i) KOH, PhMe, reflux, 2%; (ii) BBr₃, CH₂Cl₂, −78° C. to r.t., 99%; (iii) NBS, CH₂Cl₂, 46%; (e) (i) allyl bromide, K₂CO₃, acetone, reflux, 85-88%; (ii) microwave irradiation, EtOH, 99%; (iii) NBS, CH₂Cl₂, 39-99%; (f) (i) n-butylboronic acid pinacol ester, 20 mol % Pd(PPh₃)₄, NaOH, PhMe:H₂O (2:1), 36%; (ii) BBr₃, CH₂Cl₂, 90%; (iii) NBS, CH₂Cl₂, 53%.

FIG. 9 shows Calgary Biofilm Device assays to quantify planktonic (MBC) and biofilm (MBEC) killing efficiencies against MRSA-2, MRSE and VRE.

FIG. 10 shows biofilm cell killing (CFU/mL) for HP 14 obtained by colony counts from Calgary Biofilm Device pegs.

FIG. 11 shows live/dead staining of established MRSE 35984 biofilms treated with HP 14.

FIG. 12 shows structure-activity relationships (MIC/MBEC against MRSA-2) of all 29 HP analogues investigated during these studies. Diverse sub-classes of HP analogues with antibacterial and biofilm eradication activities against MRSA-2.

FIG. 13 shows detailed structure-activity relationships and antibacterial profiles of select halogenated phenazine analogues against MRSA, MRSE, VRE and MtB.

FIGS. 14A to 14D show UV-Vis analysis of metal chelation with 2 and 29. FIG. 14A shows HP 2 binding copper(II) resulting in a loss of absorbance due to complex precipitation (insoluble). FIG. 14B shows halogenated quinoline 29 binding copper(II) results in a shift in absorbance, which remains soluble. FIG. 14C shows HP 2 binds iron(II) resulting in a shift in absorbance corresponding to a soluble HP-iron(II) complex (λmax=550 nm). FIG. 14D shows 29 binding iron(II) resulting in a shift in absorbance.

FIG. 15 shows HPs 2 and 29 complex formation with Cu(II) and Fe(II) determined by spectrophotometrically quantifying the concentration of free phenazine in solution following incubation with varying equivalents of CuSO₄

FIG. 16 shows HPs 2 and 29 complex formation with Cu(II) and Fe(II). The concentrations of free HP 2 in solution were determined from a calibration curve of serial dilutions of HP 2 in dimethyl sulfoxide.

FIG. 17 shows biofilm cell killing (CFU/mL) for HP 14 and 16 obtained by colony counts from Calgary Biofilm Device pegs.

FIGS. 18A to 18D show the kill kinetics of exponential growth cultures of S. aureus (FIG. 18A), MRSA-2 (FIG. 18B), S. epidermidis (FIG. 18C), and E. faecium (FIG. 18D) (rapidly-dividing bacteria) of select compounds.

FIG. 19 shows halogenated phenazine cytotoxicity results (Triton-X=100% cell death; Medium Only: 0% cell death).

FIGS. 20A to 20C show spectrophotometric determination of dissociation constants for HP 2. FIG. 20A shows pH-dependent spectra scan results of HP 2. FIG. 20B shows absorbance vs. pH curves of HP 2. FIG. 20C shows pH vs. log [A⁻/HA] relationship of HP 2.

FIG. 21 shows biolfilm eradication against MRSA-2. In FIGS. 21 to 35, “Vanc” denotes vancomycin, “Cipro” denotes ciprofloxacin, “Dapto” denotes daptomycin, and “Linezo” denotes linezolid.

FIG. 22 shows biolfilm eradication against MRSA-2.

FIG. 23 shows biolfilm eradication against MRSA-2.

FIG. 24 shows biolfilm eradication against MRSA-2 (top panel) and biofilm eradication against MRSA BAA-44 (bottom panel).

FIG. 25 shows biolfilm eradication against MRSA BAA-1707.

FIG. 26 shows biolfilm eradication against S. epidermidis (MRSE 35984).

FIG. 27 shows biolfilm eradication against E. faecium (VRE 700221).

FIG. 28 shows a MIC assay against MRSA-2.

FIG. 29 shows a MIC assay against MRSA BAA-44 and BAA-1707.

FIG. 30 shows a MIC assay against S. epidermidis (ATCC 12228).

FIG. 31 shows a MIC assay against S. epidermidis (MRSE 35984).

FIG. 32 shows a MIC assay against E. faecium (VRE 700221).

FIG. 33 shows co-treatment of MRSA-2 with Tiron and CuSO₄.

FIG. 34 shows co-treatment of S. epidermidis (ATCC 12228) with Tiron and CuSO₄.

FIG. 35 shows co-treatment of E. faecium (VRE 700221) with Tiron and CuSO₄.

### DETAILED DESCRIPTION OF CERTAIN EMBODIMENTS OF THE INVENTION

Other antimicrobial phenazine derivatives have been reported in international PCT application publication, WO 2015/100331, published Jul. 2, 2015, which is incorporated herein by reference. The present invention provides, in one aspect, novel phenazine derivatives, such as compounds of Formulae (I′) (e.g., Formula (I)), (II), and (III), and salts (e.g., pharmaceutically acceptable salts), solvates, hydrates, polymorphs, co-crystals, tautomers, stereoisomers, isotopically labeled derivatives, and prodrugs thereof. The compounds of the invention are expected to be antimicrobial agents and, without wishing to be bound by any particular theory, may act by a microbial warfare strategy (e.g., a reactive oxygen species (ROS)-based competition strategy). The present invention also provides compositions including pharmaceutical compositions, kits, uses, and methods that involve the compounds of the invention and may be useful in preventing and/or treating a microbial infection in a subject, inhibiting the growth and/or reproduction of a microorganism (e.g., bacterium, mycobacterium, archaeon, protist, fungus, or parasite), killing a microorganism, inhibiting the formation and/or growth of a biofilm, reducing or removing a biofilm, or disinfecting a surface. In certain embodiments, the microorganism is a bacterium. In certain embodiments, the bacterium is a Gram-positive bacterium (e.g., a species of Staphylococcus or Enterococcus). In certain embodiments, the bacterium is a Gram-negative bacterium (e.g., an Acinetobacter species).

Many past successes in antibiotic discovery have been grounded on microbial warfare agents/strategies from microorganisms. Therefore, future antimicrobial treatments may also depend on the discovery and implementation of innovative microbial-inspired antimicrobial strategies. One such strategy is the use of redox-active phenazine antibiotics by Pseudomonas during competition with other bacteria and fungi through the formation of reactive oxygen species (ROS) (A. Price-Whelan, L. E. P. Dietrich, and D. K. Newman, Nat. Chem. Biol., 2006, 2, 71-78; Z. A. Machan, T. L. Pitt, W. White, D. Watson, G. W. Taylor, P. J. Cole, and R. Wilson, J. Med. Microbiol., 1991, 34, 213-217). One example of this competition is in young cystic fibrosis (CF) patients (Z. A. Machan, T. L. Pitt, W. White, D. Watson, G. W. Taylor, P. J. Cole, and R. Wilson, J. Med. Microbiol., 1991, 34, 213-217). Many times, individuals with CF first develop Staphylococcus aureus lung infections when they are young. As the CF patient ages, Pseudomonas aeruginosa co-infects the lung and successfully competes against S. aureus for this niche using redox-active phenazine antibiotics.

Certain phenazine derivatives, such as compounds 301-305 (shown below) are known antimicrobial agents. Pyocyanin (compound 301) is one of the toxins produced by the Gram negative bacterium Pseudomonas aeruginosa. It is thought that Pseudomonas aeruginosa employs a microbial warfare strategy by producing these toxins in competing with other microorganisms (e.g., other bacteria). Pyocyanin is able to oxidize and reduce other molecules (Hassan et al., J. Bacteriology 1980, 141, 156-163) and can kill microbes competing against Pseudomonas aeruginosa as well as mammalian cells of the lungs that Pseudomonas aeruginosa has infected during cystic fibrosis. Due to its redox-active properties, pyocyanin can generate reactive oxygen species (ROS), which may be toxic to bacteria. It has been reported that the reduction potential and redox-cycling capabilities of phenazine are electronically influenced by functional group substitutions on the phenazinyl ring system (Price-Whelan et al., Nat. Chem. Biol., 2006, 2, 71-78; Wang et al., J Bacteriol., 2010, 192, 365-369). Therefore, the redox-active properties of a phenazine derivative may be altered by structurally modifying the phenazine derivative. However, there is no teaching or suggestion in the art on how a known phenazine may be structurally modified to improve its properties, such as antimicrobial activity.

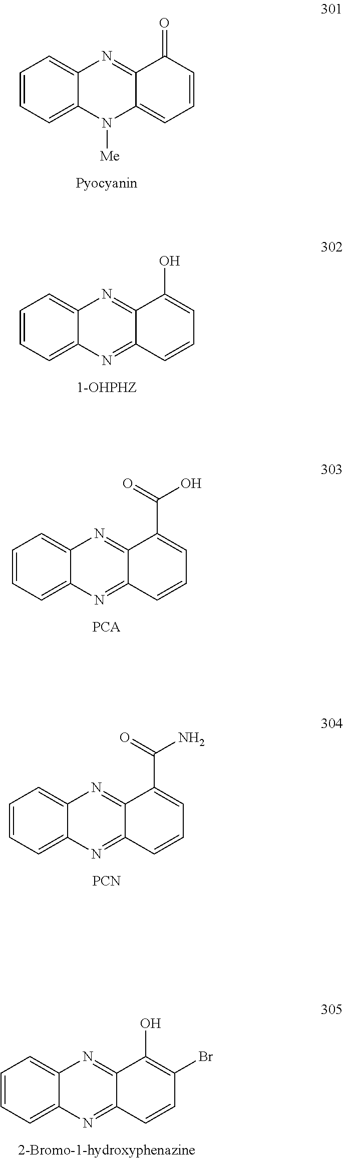

In certain embodiments, the compounds of the invention are improved phenazine derivatives and showed unexpected and superior properties compared to known phenazine derivatives, such as enhanced inhibitory activity against bacteria, e.g., Staphylococcus aureus (S. aureus), Staphylococcus epidermidis (S. epidermidis), and/or Enterococcus faecium. Staphylococcus aureus is a human pathogen that is notorious for life-threatening drug-resistant infections in hospitals and the community (H. F. Chambers and F. R. DeLeo, Nat. Rev. Microbiol., 2009, 7, 629-641). In the United States alone, there are more annual deaths from methicillin-resistant Staphylococcus aureus (MRSA) related microbial infections than AIDS (IDSA Policy Paper d CID 2011:52 (Suppl 5) d S397). Staphylococcus epidermidis is also a pathogen of great importance as it is particularly prevalent in persistent microbial infections associated with catheters (I. Uckay, D. Pittet, P. Vaudaux, H. Sax, D. Lew, and F. Waldvogel, Ann. Med., 2009, 41, 109-119).

Without wishing to be bound by any particular theory, it is thought that the compounds of the invention may act by a microbial warfare strategy (e.g., an ROS-based competition strategy) similar to the one employed by Pseudomonas aeruginosa. The inventive compounds may be capable of undergoing reduction and oxidation (redox) reactions and forming ROS in, near, or around a microorganism (e.g., bacterium, mycobacterium, archaeon, protist, fungus, or parasite). An inventive compound may accept a single electron, yielding a relatively stable anion radical, and may readily undergo a redox cycle. A compound of the invention may be reduced by the nicotinamide adenine dinucleotide (NADH⁺) in a microorganism and may divert electron flow within the microorganism from the normal cytochrome pathway to an ROS-producing pathway. As a result, the production of ROS, such as O₂⁻ and H₂O₂, which are toxic to the microorganism, may be increased.

Furthermore, compounds disclosed herein may be effective agents for the inhibition of biofilm growth and/or clearance of existing biofilms. Bacterial biofilms are surface-attached bacterial communities that are encased within a secreted matrix of biomolecules (e.g., extracellular DNA, proteins, polysaccharides) known as the extracellular polymeric substance (EPS). Bacterial cells within a biofilm take on a completely different physiology than their free-swimming planktonic counterparts and are notorious for being highly resistant to conventional antibiotic treatments and host immune responses (Donlan, R. M. and Costerton, J. W. Clin. Microbiol. Rev. 2002, 15, 167-193). The National Institutes of Health has reported that biofilms are present in up to 80% of all bacterial infections. Unfortunately, biofilms are notorious for their resistance to conventional antibiotic treatments, and therefore our current arsenal of antibiotics does not include agents that effectively target biofilm machinery or clear established biofilms in a clinical setting. Such antibiofilm agents would lead to significant breakthroughs in how bacterial infections are treated and would result in the effective treatment of many life-threatening bacterial infections.

Bacterial biofilm formation is governed by a signaling process known as quorum sensing, which is used by bacteria to monitor population density and control bacterial virulence (Camilli, A. and Bassler, B. L. Science 2006, 311, 1113-1116; Ng, W.-L. and Bassler, B. L. Annu. Rev. Genet. 2009, 43, 197-222). Quorum sensing is used by free-swimming, individual planktonic bacteria to coordinate the simultaneous attachment and colonization of a surface followed by biofilm formation and maturation. The coordinated surface attachment of bacteria overwhelms immune responses mounted by host organisms, enabling the successful colonization of surfaces (e.g., tissue surfaces) by bacteria. Bacterial biofilms are known to be greater than 1000-fold more resistant to conventional antibiotics when compared to their planktonic counterparts. Therapeutic strategies targeting quorum sensing and/or biofilm formation and dispersion phenotypes have become a promising antibacterial strategy as small molecules capable of inhibiting bacterial biofilm formation via non-growth inhibitory mechanisms or clearing pre-formed bacterial biofilms are of clinical importance. Without wishing to be bound by any particular theory, compounds described herein may function by disrupting quorum sensing, leading to inhibitors of biofilm formation and clearing of pre-formed biofilms.

The inventive compounds preferably have minimal to no adverse side effects. In certain embodiments, the compounds exhibit low cytotoxicity against mammalian (e.g., human) cells. In certain embodiments, the compounds show low hemolysis activity.

### Compounds

One aspect of the invention relates to compounds that are believed to be antimicrobial agents. In certain embodiments, the compounds of the invention are compound of Formula (I′):

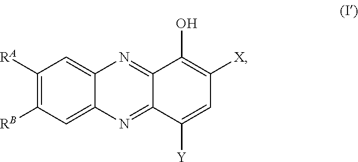

and salts (e.g., pharmaceutically acceptable salts), solvates, hydrates, polymorphs, co-crystals, tautomers, stereoisomers, isotopically labeled derivatives, and prodrugs thereof, wherein:

X is hydrogen, halogen, substituted or unsubstituted alkyl, substituted or unsubstituted alkenyl, or substituted or unsubstituted alkynyl;

Y is halogen, substituted or unsubstituted alkyl, substituted or unsubstituted alkenyl, or substituted or unsubstituted alkynyl;

R^{A} is hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR¹, —N(R¹)₂, —SR¹, —CN, —SCN, —C(═NR¹)R¹, —C(═NR¹)OR¹, —C(═NR¹)N(R¹)₂, —C(═O)R¹, —C(═O)OR¹, —C(═O)N(R¹)₂, —NO₂, —NR C(═O)R, —NR¹C(═O)OR¹, NR¹C(═O)N(R¹)₂, —OC(═O)R¹, —OC(═O)OR¹, or —OC(═O)N(R¹)₂, wherein each instance of R¹ is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R¹ are joined to form a substituted or unsubstituted heterocyclic or substituted or unsubstituted heteroaryl ring; and

R^{B} is hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR², —N(R²)₂, —SR², —CN, —SCN, —C(═NR²)R², —C(═NR²)OR², —C(═NR²)N(R²)₂, —C(═O)R², —C(═O)OR², —C(═O)N(R²)₂, —NO₂, —NR²C(═O)R², —NR²C(═O)OR², —NR²C(═O)N(R²)₂, —OC(═O)R², —OC(═O)OR², or —OC(═O)N(R²)₂, wherein each instance of R² is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R² are joined to form a substituted or unsubstituted heterocyclic or substituted or unsubstituted heteroaryl ring;

or R^{A} and R^{B} are joined to form a substituted or unsubstituted phenyl ring;

provided that:

at least one of X and Y is halogen; and

the compound is not of the formula:

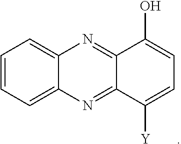

In certain embodiments, the compounds of the invention are compounds of Formula (I):

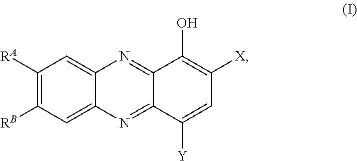

and salts (e.g., pharmaceutically acceptable salts), solvates, hydrates, polymorphs, co-crystals, tautomers, stereoisomers, isotopically labeled derivatives, and prodrugs thereof, wherein:

X is hydrogen or halogen;

Y is halogen;

R^{A} is hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR¹, —N(R¹)₂, —SR¹, —CN, —SCN, —C(═NR¹)R¹, —C(═NR¹)OR¹, —C(═NR¹)N(R¹)₂, —C(═O)R¹, —C(═O)OR¹, —C(═O)N(R¹)₂, —NO₂, —NR C(═O)R, —NR¹C(═O)OR¹, —NR¹C(═O)N(R¹)₂, —OC(═O)R¹, —OC(═O)OR¹, or —OC(═O)N(R¹)₂, wherein each instance of R¹ is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R¹ are joined to form a substituted or unsubstituted heterocyclic or substituted or unsubstituted heteroaryl ring; and

R^{B} is hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR², —N(R²)₂, —SR², —CN, —SCN, —C(═NR²)R², —C(═NR²)OR², —C(═NR²)N(R²)₂, —C(═O)R², —C(═O)OR², —C(═O)N(R²)₂, —NO₂, —NR²C(═O)R², —NR²C(═O)OR², —NR²C(═O)N(R²)₂, —OC(═O)R², —OC(═O)OR², or —OC(═O)N(R²)₂, wherein each instance of R² is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R² are joined to form a substituted or unsubstituted heterocyclic or substituted or unsubstituted heteroaryl ring;

or R^{A} and R^{B} are joined to form a substituted or unsubstituted phenyl ring; and provided that the compound is not of the formula:

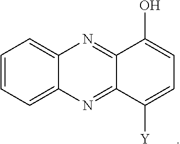

Formula (I′) (e.g., Formula (I)) includes substituent X on the phenazinyl ring. In certain embodiments, X is hydrogen. In certain embodiments, X is halogen. In certain embodiments, X is F. In certain embodiments, X is Cl. In certain embodiments, X is Br. In certain embodiments, X is I. In certain embodiments, X is substituted or unsubstituted alkyl (e.g., substituted or unsubstituted C₁₋₆ alkyl). In certain embodiments, X is Me. In certain embodiments, X is substituted methyl (e.g., —CH₂F, —CHF₂, —CF₃, or Bn). In certain embodiments, X is Et, substituted ethyl (e.g., fluorinated ethyl (e.g., perfluoroethyl)), Pr, substituted propyl (e.g., fluorinated propyl (e.g., perfluoropropyl)), Bu, substituted butyl (e.g., fluorinated butyl (e.g., perfluorobutyl)), unsubstituted pentyl, substituted pentyl (e.g., fluorinated pentyl (e.g., perfluoropentyl)), unsubstituted hexyl, or substituted hexyl (e.g., fluorinated hexyl (e.g., perfluorohexyl)). In certain embodiments, X is substituted or unsubstituted alkenyl (e.g., substituted or unsubstituted C₂₋₆ alkenyl). In certain embodiments, X is substituted or unsubstituted vinyl. In certain embodiments, X is unsubstituted allyl. In certain embodiments, X is substituted allyl. In certain embodiments, X is substituted or unsubstituted alkynyl (e.g., substituted or unsubstituted C₂₋₆ alkynyl).

Formula (I′) (e.g., Formula (I)) also includes substituent Y on the phenazinyl ring. In certain embodiments, Y is halogen. In certain embodiments, Y is F. In certain embodiments, Y is C₁. In certain embodiments, Y is Br. In certain embodiments, Y is I. In certain embodiments, Y is substituted or unsubstituted alkyl (e.g., substituted or unsubstituted C₁₋₆ alkyl). In certain embodiments, Y is Me. In certain embodiments, Y is substituted methyl (e.g., —CH₂F, —CHF₂, —CF₃, or Bn). In certain embodiments, Y is Et, substituted ethyl (e.g., fluorinated ethyl (e.g., perfluoroethyl)), Pr, substituted propyl (e.g., fluorinated propyl (e.g., perfluoropropyl)), Bu, substituted butyl (e.g., fluorinated butyl (e.g., perfluorobutyl)), unsubstituted pentyl, substituted pentyl (e.g., fluorinated pentyl (e.g., perfluoropentyl)), unsubstituted hexyl, or substituted hexyl (e.g., fluorinated hexyl (e.g., perfluorohexyl)). In certain embodiments, Y is n-Bu. In certain embodiments, Y is substituted or unsubstituted alkenyl (e.g., substituted or unsubstituted C₂₋₆ alkenyl). In certain embodiments, Y is substituted or unsubstituted vinyl. In certain embodiments, Y is unsubstituted allyl. In certain embodiments, Y is substituted allyl. In certain embodiments, Y is substituted or unsubstituted alkynyl (e.g., substituted or unsubstituted C₂₋₆ alkynyl).

In certain embodiments, X is hydrogen; and Y is F. In certain embodiments, X is hydrogen; and Y is Cl. In certain embodiments, X is hydrogen; and Y is Br. In certain embodiments, X is hydrogen; and Y is I. In certain embodiments, X is Cl; and Y is F. In certain embodiments, both X and Y are Cl. In certain embodiments, X is Cl; and Y is Br. In certain embodiments, X is Cl; and Y is I. In certain embodiments, X is Br; and Y is F. In certain embodiments, X is Br; and Y is Cl. In certain embodiments, both X and Y are Br. In certain embodiments, X is Br; and Y is I. In certain embodiments, X is I; and Y is F. In certain embodiments, X is I; and Y is Cl. In certain embodiments, X is I; and Y is Br. In certain embodiments, both X and Y are I. In certain embodiments, X is halogen; and Y is substituted or unsubstituted alkyl (e.g., substituted or unsubstituted C₁₋₆ alkyl). In certain embodiments, X is halogen; and Y is substituted or unsubstituted alkenyl (e.g., substituted or unsubstituted C₂₋₆ alkenyl). In certain embodiments, X is substituted or unsubstituted alkyl (e.g., substituted or unsubstituted C₁₋₆ alkyl); and Y is halogen. In certain embodiments, X is substituted or unsubstituted alkenyl (e.g., substituted or unsubstituted C₂₋₆ alkenyl); and Y is halogen. In certain embodiments, X is halogen; and X and Y are the same. In certain embodiments, X is halogen; and X and Y are not the same. In certain embodiments, at least one of X and Y is halogen. In certain embodiments, each X and Y is halogen.

Formula (I′) (e.g., Formula (I)) also includes substituent R^{A} on the phenazinyl ring. In certain embodiments, R^{A} is hydrogen. In certain embodiments, R^{A} is not hydrogen. In certain embodiments, R^{A} is halogen. In certain embodiments, R^{A} is F. In certain embodiments, R^{A} is C₁. In certain embodiments, R^{A} is Br. In certain embodiments, R^{A} is I. In certain embodiments, R^{A} is substituted or unsubstituted alkyl. In certain embodiments, R^{A} is substituted or unsubstituted C₁₋₆ alkyl. In certain embodiments, R^{A} is Me. In certain embodiments, R^{A} is substituted methyl (e.g., —CH₂F, —CHF₂, —CF₃, or Bn). In certain embodiments, R^{A} is Et, substituted ethyl (e.g., fluorinated ethyl (e.g., perfluoroethyl)), Pr, substituted propyl (e.g., fluorinated propyl (e.g., perfluoropropyl)), Bu, or substituted butyl (e.g., fluorinated butyl (e.g., perfluorobutyl)). In certain embodiments, R^{A} is substituted or unsubstituted alkenyl (e.g., substituted or unsubstituted C₂₋₆ alkenyl). In certain embodiments, R^{A} is substituted or unsubstituted alkynyl (e.g., substituted or unsubstituted C₂₋₆ alkynyl). In certain embodiments, R^{A} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, R^{A} is substituted or unsubstituted cyclopropyl, substituted or unsubstituted cyclobutyl, substituted or unsubstituted cyclopentyl, or substituted or unsubstituted cyclohexyl. In certain embodiments, R^{A} is substituted or unsubstituted heterocyclyl (e.g., substituted or unsubstituted, 3- to 7-membered, monocyclic heterocyclyl comprising zero, one, or two double bonds in the heterocyclic ring system, wherein one, two, or three atoms in the heterocyclic ring system are independently nitrogen, oxygen, or sulfur). In certain embodiments, R^{A} is substituted or unsubstituted oxetanyl, substituted or unsubstituted tetrahydrofuranyl, substituted or unsubstituted pyrrolidinyl, substituted or unsubstituted tetrahydropyranyl, substituted or unsubstituted piperidinyl, substituted or unsubstituted morpholinyl, or substituted or unsubstituted piperazinyl. In certain embodiments, R^{A} is substituted or unsubstituted aryl (e.g., substituted or unsubstituted, 6- to 10-membered aryl). In certain embodiments, R^{A} is unsubstituted phenyl. In certain embodiments, R^{A} is substituted phenyl. In certain embodiments, R^{A} is substituted or unsubstituted naphthyl. In certain embodiments, R^{A} is substituted or unsubstituted heteroaryl. In certain embodiments, R^{A} is substituted or unsubstituted, 5- to 6-membered, monocyclic heteroaryl, wherein one, two, three, or four atoms in the heteroaryl ring system are independently nitrogen, oxygen, or sulfur. In certain embodiments, R^{A} is substituted or unsubstituted, 9- to 10-membered, bicyclic heteroaryl, wherein one, two, three, or four atoms in the heteroaryl ring system are independently nitrogen, oxygen, or sulfur. In certain embodiments, R^{A} is —OR¹ (e.g., —OH, —O(substituted or unsubstituted C₁₋₆ alkyl) (e.g., —OMe, —OCF₃, —OEt, —OPr, —OBu, or —OBn), or —O(substituted or unsubstituted phenyl) (e.g., —OPh)). In certain embodiments, R^{A} is —OMe. In certain embodiments, R^{A} is —SR¹ (e.g., —SH, —S(substituted or unsubstituted C₁₋₆ alkyl) (e.g., —SMe, —SEt, —SPr, —SBu, or —SBn), or —S(substituted or unsubstituted phenyl) (e.g., —SPh)). In certain embodiments, R^{A} is —N(R¹)₂ (e.g., —NH₂, —NH(substituted or unsubstituted C₁₋₆ alkyl) (e.g., —NHMe), or —N(substituted or unsubstituted C₁₋₆ alkyl)-(substituted or unsubstituted C₁₋₆ alkyl) (e.g., —NMe₂)). In certain embodiments, R^{A} is —CN or —SCN. In certain embodiments, R^{A} is —NO₂. In certain embodiments, R^{A} is —C(═NR¹)R¹, —C(═NR¹)OR¹, or —C(═NR¹)N(R¹)₂. In certain embodiments, R^{A} is —C(═O)R¹ (e.g., —C(═O)(substituted or unsubstituted alkyl) (e.g., —C(═O)Me) or —C(═O)(substituted or unsubstituted phenyl)). In certain embodiments, R^{A} is —C(═O)OR¹ (e.g., —C(═O)OH, —C(═O)O(substituted or unsubstituted alkyl) (e.g., —C(═O)OMe), or —C(═O)O(substituted or unsubstituted phenyl)). In certain embodiments, R^{A} is —C(═O)N(R¹)₂ (e.g., —C(═O)NH₂, —C(═O)NH(substituted or unsubstituted alkyl) (e.g., —C(═O)NHMe), —C(═O)NH(substituted or unsubstituted phenyl), —C(═O)N(substituted or unsubstituted alkyl)-(substituted or unsubstituted alkyl), or —C(═O)N(substituted or unsubstituted phenyl)-(substituted or unsubstituted alkyl)). In certain embodiments, R^{A} is —NR¹C(═O)R¹ (e.g., —NHC(═O)(substituted or unsubstituted C₁₋₆ alkyl) (e.g., —NHC(═O)Me) or —NHC(═O)(substituted or unsubstituted phenyl)). In certain embodiments, R^{A} is —NR¹C(═O)OR¹. In certain embodiments, R^{A} is —NR¹C(═O)N(R¹)₂ (e.g., —NHC(═O)NH₂, —NHC(═O)NH(substituted or unsubstituted C₁₋₆ alkyl) (e.g., —NHC(═O)NHMe)). In certain embodiments, R^{A} is —OC(═O)R¹ (e.g., —OC(═O)(substituted or unsubstituted alkyl) or —OC(═O)(substituted or unsubstituted phenyl)), —OC(═O)OR¹ (e.g., —OC(═O)O(substituted or unsubstituted alkyl) or —OC(═O)O(substituted or unsubstituted phenyl)), or —OC(═O)N(R¹)₂ (e.g., —OC(═O)NH₂, —OC(═O)NH(substituted or unsubstituted alkyl), —OC(═O)NH(substituted or unsubstituted phenyl), —OC(═O)N(substituted or unsubstituted alkyl)-(substituted or unsubstituted alkyl), or —OC(═O)N(substituted or unsubstituted phenyl)-(substituted or unsubstituted alkyl)).

Formula (I′) (e.g., Formula (I)) may include one or more instances of substituent R¹. When Formula (I′) (e.g., Formula (I)) includes two or more instances of R¹, any two instances of R¹ may be the same or different from each other. In certain embodiments, at least one instance of R¹ is H. In certain embodiments, each instance of R¹ is H. In certain embodiments, at least one instance of R¹ is substituted or unsubstituted alkyl (e.g., substituted or unsubstituted C₁₋₆ alkyl (e.g., Me)). In certain embodiments, at least one instance of R¹ is substituted or unsubstituted acyl, substituted or unsubstituted alkenyl (e.g., substituted or unsubstituted C₂₋₆ alkenyl), substituted or unsubstituted alkynyl (e.g., substituted or unsubstituted C₂₋₆ alkynyl), substituted or unsubstituted carbocyclyl (e.g., substituted or unsubstituted, 3- to 7-membered, monocyclic carbocyclyl comprising zero, one, or two double bonds in the carbocyclic ring system), substituted or unsubstituted heterocyclyl (e.g., substituted or unsubstituted, 3- to 7-membered, monocyclic heterocyclyl comprising zero, one, or two double bonds in the heterocyclic ring system, wherein one, two, or three atoms in the heterocyclic ring system are independently nitrogen, oxygen, or sulfur), substituted or unsubstituted aryl (e.g., substituted or unsubstituted phenyl), substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur), a nitrogen protecting group (e.g., Bn, Boc, Cbz, Fmoc, trifluoroacetyl, triphenylmethyl, acetyl, or Ts) when attached to a nitrogen atom, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom. In certain embodiments, two instances of R¹ are joined to form a substituted or unsubstituted heterocyclic or substituted or unsubstituted heteroaryl ring.

Formula (I′) (e.g., Formula (I)) also includes substituent R^{B} on the phenazinyl ring. In certain embodiments, R^{B} is hydrogen. In certain embodiments, R^{B} is not hydrogen. In certain embodiments, R^{B} is halogen. In certain embodiments, R^{B} is F. In certain embodiments, R^{B} is Cl. In certain embodiments, R^{B} is Br. In certain embodiments, R^{B} is I. In certain embodiments, R^{B} is substituted or unsubstituted alkyl. In certain embodiments, R^{B} is substituted or unsubstituted C₁₋₆ alkyl. In certain embodiments, R^{B} is Me. In certain embodiments, R^{B} is substituted methyl (e.g., —CH₂F, —CHF₂, —CF₃, or Bn). In certain embodiments, R^{B} is Et, substituted ethyl (e.g., fluorinated ethyl (e.g., perfluoroethyl)), Pr, substituted propyl (e.g., fluorinated propyl (e.g., perfluoropropyl)), Bu, or substituted butyl (e.g., fluorinated butyl (e.g., perfluorobutyl)). In certain embodiments, R^{B} is substituted or unsubstituted alkenyl (e.g., substituted or unsubstituted C₂₋₆ alkenyl). In certain embodiments, R^{B} is substituted or unsubstituted alkynyl (e.g., substituted or unsubstituted C₂₋₆ alkynyl). In certain embodiments, R^{B} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, R^{B} is substituted or unsubstituted cyclopropyl, substituted or unsubstituted cyclobutyl, substituted or unsubstituted cyclopentyl, or substituted or unsubstituted cyclohexyl. In certain embodiments, R^{B} is substituted or unsubstituted heterocyclyl (e.g., substituted or unsubstituted, 3- to 7-membered, monocyclic heterocyclyl comprising zero, one, or two double bonds in the heterocyclic ring system, wherein one, two, or three atoms in the heterocyclic ring system are independently nitrogen, oxygen, or sulfur). In certain embodiments, R^{B} is substituted or unsubstituted oxetanyl, substituted or unsubstituted tetrahydrofuranyl, substituted or unsubstituted pyrrolidinyl, substituted or unsubstituted tetrahydropyranyl, substituted or unsubstituted piperidinyl, substituted or unsubstituted morpholinyl, or substituted or unsubstituted piperazinyl. In certain embodiments, R^{B} is substituted or unsubstituted aryl (e.g., substituted or unsubstituted, 6- to 10-membered aryl). In certain embodiments, R^{B} is unsubstituted phenyl. In certain embodiments, R^{B} is substituted phenyl. In certain embodiments, R^{B} is substituted or unsubstituted naphthyl. In certain embodiments, R^{B} is substituted or unsubstituted heteroaryl. In certain embodiments, R^{B} is substituted or unsubstituted, 5- to 6-membered, monocyclic heteroaryl, wherein one, two, three, or four atoms in the heteroaryl ring system are independently nitrogen, oxygen, or sulfur. In certain embodiments, R^{B} is substituted or unsubstituted, 9- to 10-membered, bicyclic heteroaryl, wherein one, two, three, or four atoms in the heteroaryl ring system are independently nitrogen, oxygen, or sulfur. In certain embodiments, R^{B} is —OR² (e.g., —OH, —O(substituted or unsubstituted C₁₋₆ alkyl) (e.g., —OMe, —OCF₃, —OEt, —OPr, —OBu, or —OBn), or —O(substituted or unsubstituted phenyl) (e.g., —OPh)). In certain embodiments, R^{B} is —OMe. In certain embodiments, R^{B} is —SR² (e.g., —SH, —S(substituted or unsubstituted C₁₋₆ alkyl) (e.g., —SMe, —SEt, —SPr, —SBu, or —SBn), or —S(substituted or unsubstituted phenyl) (e.g., —SPh)). In certain embodiments, R^{B} is —N(R²)₂ (e.g., —NH₂, —NH(substituted or unsubstituted C₁₋₆ alkyl) (e.g., —NHMe), or —N(substituted or unsubstituted C₁₋₆ alkyl)-(substituted or unsubstituted C₁₋₆ alkyl) (e.g., —NMe₂)). In certain embodiments, R^{B} is —CN or —SCN. In certain embodiments, R^{B} is —NO₂. In certain embodiments, R^{B} is —C(═NR²)R², —C(═NR²)OR², or —C(═NR²)N(R²)₂. In certain embodiments, R^{B} is —C(═O)R² (e.g., —C(═O)(substituted or unsubstituted alkyl) (e.g., —C(═O)Me) or —C(═O)(substituted or unsubstituted phenyl)). In certain embodiments, R^{B} is —C(═O)OR² (e.g., —C(═O)OH, —C(═O)O(substituted or unsubstituted alkyl) (e.g., —C(═O)OMe), or —C(═O)O(substituted or unsubstituted phenyl)). In certain embodiments, R^{B} is —C(═O)N(R²)₂ (e.g., —C(═O)NH₂, —C(═O)NH(substituted or unsubstituted alkyl) (e.g., —C(═O)NHMe), —C(═O)NH(substituted or unsubstituted phenyl), —C(═O)N(substituted or unsubstituted alkyl)-(substituted or unsubstituted alkyl), or —C(═O)N(substituted or unsubstituted phenyl)-(substituted or unsubstituted alkyl)). In certain embodiments, R^{B} is —NR²C(═O)R² (e.g., —NHC(═O)(substituted or unsubstituted C₁₋₆ alkyl) (e.g., —NHC(═O)Me) or —NHC(═O)(substituted or unsubstituted phenyl)). In certain embodiments, R^{B} is —NR²C(═O)OR². In certain embodiments, R^{B} is —NR²C(═O)N(R²)₂ (e.g., —NHC(═O)NH₂, —NHC(═O)NH(substituted or unsubstituted C₁₋₆ alkyl) (e.g., —NHC(═O)NHMe)). In certain embodiments, R^{B} is —OC(═O)R² (e.g., —OC(═O)(substituted or unsubstituted alkyl) or —OC(═O)(substituted or unsubstituted phenyl)), —OC(═O)OR² (e.g., —OC(═O)O(substituted or unsubstituted alkyl) or —OC(═O)O(substituted or unsubstituted phenyl)), or —OC(═O)N(R²)₂ (e.g., —OC(═O)NH₂, —OC(═O)NH(substituted or unsubstituted alkyl), —OC(═O)NH(substituted or unsubstituted phenyl), —OC(═O)N(substituted or unsubstituted alkyl)-(substituted or unsubstituted alkyl), or —OC(═O)N(substituted or unsubstituted phenyl)-(substituted or unsubstituted alkyl)).

Formula (I′) (e.g., Formula (I)) may include one or more instances of substituent R². When Formula (I′) (e.g., Formula (I)) includes two or more instances of R², any two instances of R² may be the same or different from each other. In certain embodiments, at least one instance of R² is H. In certain embodiments, each instance of R² is H. In certain embodiments, at least one instance of R² is substituted or unsubstituted alkyl (e.g., substituted or unsubstituted C₁₋₆ alkyl (e.g., Me)). In certain embodiments, at least one instance of R² is substituted or unsubstituted acyl, substituted or unsubstituted alkenyl (e.g., substituted or unsubstituted C₂₋₆ alkenyl), substituted or unsubstituted alkynyl (e.g., substituted or unsubstituted C₂₋₆ alkynyl), substituted or unsubstituted carbocyclyl (e.g., substituted or unsubstituted, 3- to 7-membered, monocyclic carbocyclyl comprising zero, one, or two double bonds in the carbocyclic ring system), substituted or unsubstituted heterocyclyl (e.g., substituted or unsubstituted, 3- to 7-membered, monocyclic heterocyclyl comprising zero, one, or two double bonds in the heterocyclic ring system, wherein one, two, or three atoms in the heterocyclic ring system are independently nitrogen, oxygen, or sulfur), substituted or unsubstituted aryl (e.g., substituted or unsubstituted phenyl), substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur), a nitrogen protecting group (e.g., Bn, Boc, Cbz, Fmoc, trifluoroacetyl, triphenylmethyl, acetyl, or Ts) when attached to a nitrogen atom, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom. In certain embodiments, two instances of R² are joined to form a substituted or unsubstituted heterocyclic or substituted or unsubstituted heteroaryl ring.

In certain embodiments, both R^{A} and R^{B} are H. In certain embodiments, each of R^{A} and R^{B} is independently hydrogen or halogen. In certain embodiments, each of R^{A} and R^{B} is independently hydrogen, halogen, or substituted or unsubstituted C₁₋₆ alkyl (e.g., Me). In certain embodiments, each of R^{A} and R^{B} is independently hydrogen, halogen, substituted or unsubstituted C₁₋₆ alkyl (e.g., Me), or —O(substituted or unsubstituted C₁₋₆ alkyl) (e.g., —OMe). In certain embodiments, R^{A} is H; and R^{B} is halogen (e.g., F, Cl, Br, or I). In certain embodiments, R^{A} is H; and R^{B} is substituted or unsubstituted C₁₋₆ alkyl (e.g., Me). In certain embodiments, R^{A} is halogen (e.g., F, Cl, Br, and I); and R^{B} is H. In certain embodiments, R^{A} is substituted or unsubstituted C₁₋₆ alkyl; and R^{B} is H. In certain embodiments, both R^{A} and R^{B} are halogen. In certain embodiments, both R^{A} and R^{B} are halogen; and R^{A} and R^{B} are the same. In certain embodiments, both R^{A} and R^{B} are halogen; and R^{A} and R^{B} are not the same. In certain embodiments, each of R^{A} and R^{B} is independently Cl, Br, or I. In certain embodiments, both R^{A} and R^{B} are C₁. In certain embodiments, R^{A} is Cl; and R^{B} is Br. In certain embodiments, R^{A} is Cl; and R^{B} is I. In certain embodiments, R^{A} is Br; and R^{B} is C₁. In certain embodiments, both R^{A} and R^{B} are Br. In certain embodiments, R^{A} is Br; and R^{B} is I. In certain embodiments, R^{A} is I; and R^{B} is C₁. In certain embodiments, R^{A} is I; and R^{B} is Br. In certain embodiments, both R^{A} and R^{B} are I. In certain embodiments, both R^{A} and R^{B} are substituted or unsubstituted C₁₋₆ alkyl (e.g., Me). In certain embodiments, R^{A} is substituted or unsubstituted C₁₋₆ alkyl (e.g., Me); and R^{B} is halogen (e.g., Cl, Br, or I). In certain embodiments, R^{A} is halogen (e.g., Cl, Br, or I); and R^{B} is substituted or unsubstituted C₁₋₆ alkyl (e.g., Me). In certain embodiments, R^{A} is —O(substituted or unsubstituted C₁₋₆ alkyl) (e.g., —OMe); and R^{B} is hydrogen, halogen, substituted or unsubstituted C₁₋₆ alkyl (e.g., Me), or —O(substituted or unsubstituted C₁₋₆ alkyl) (e.g., —OMe). In certain embodiments, R^{A} is hydrogen, halogen, substituted or unsubstituted C₁₋₆ alkyl (e.g., Me), or —O(substituted or unsubstituted C₁₋₆ alkyl) (e.g., —OMe); and R^{B} is —O(substituted or unsubstituted C₁₋₆ alkyl) (e.g., —OMe).

In certain embodiments, a compound of the invention is not of the formula:

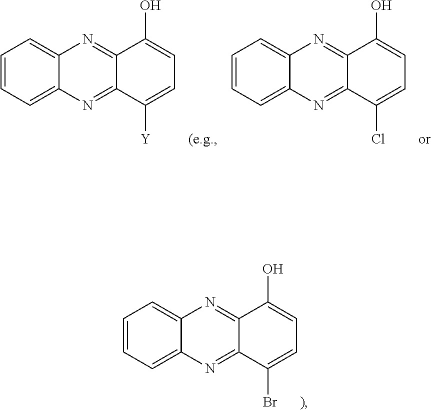

or a pharmaceutically acceptable salt thereof.

In certain embodiments, a compound of the invention is not of the formula:

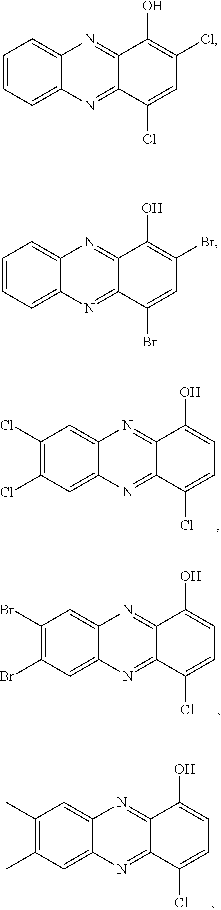 [SMILES: Oc1c(Cl)cc(Cl)c2nc3ccccc3nc12 ; Oc1c(Br)cc(Br)c2nc3ccccc3nc12 ; Oc1ccc(Cl)c2nc3cc(Cl)c(Cl)cc3nc12 ; Oc1ccc(Cl)c2nc3cc(Br)c(Br)cc3nc12 ; Cc1cc2nc3c(O)ccc(Cl)c3nc2cc1C]

or a pharmaceutically acceptable salt thereof.

In certain embodiments, at least one of R^{A} and R^{B} is not hydrogen. In certain embodiments, both R^{A} and R^{B} are not hydrogen.

In certain embodiments, a compound of the invention is not of the formula:

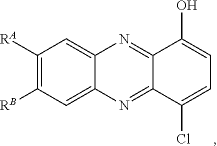

or a pharmaceutically acceptable salt thereof, wherein each of R^{A} and R^{B} is independently halogen or unsubstituted C₁₋₆ alkyl.

In certain embodiments, a compound of the invention is not of the formula:

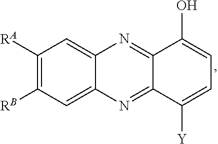

or a pharmaceutically acceptable salt thereof, wherein each of R^{A} and R^{B} is independently Cl, Br, or Me.

In certain embodiments, the compound of Formula (I′) (e.g., Formula (I)) is of the formula:

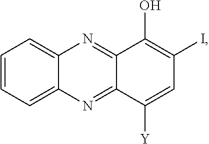

or a pharmaceutically acceptable salt, solvate, hydrate, polymorph, co-crystal, tautomer, stereoisomer, isotopically labeled derivative, or prodrug thereof.

In certain embodiments, the compound of Formula (I′) (e.g., Formula (I)) is of the formula:

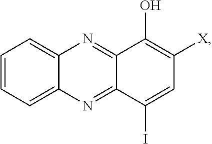

or a pharmaceutically acceptable salt, solvate, hydrate, polymorph, co-crystal, tautomer, stereoisomer, isotopically labeled derivative, or prodrug thereof.

In certain embodiments, the compound of Formula (I′) (e.g., Formula (I)) is of the formula:

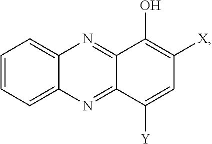

or a pharmaceutically acceptable salt, solvate, hydrate, polymorph, co-crystal, tautomer, stereoisomer, isotopically labeled derivative, or prodrug thereof, wherein:

X is halogen; and

X and Y are not the same.

In certain embodiments, the compound of Formula (I′) (e.g., Formula (I)) is of the formula:

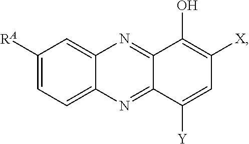

or a pharmaceutically acceptable salt, solvate, hydrate, polymorph, co-crystal, tautomer, stereoisomer, isotopically labeled derivative, or prodrug thereof, wherein R^{A} is not hydrogen (e.g., wherein R^{A} is halogen or substituted or unsubstituted C₁₋₆ alkyl).

In certain embodiments, the compound of Formula (I′) (e.g., Formula (I)) is of the formula:

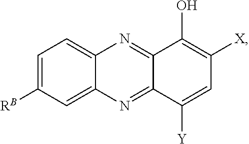

or a pharmaceutically acceptable salt, solvate, hydrate, polymorph, co-crystal, tautomer, stereoisomer, isotopically labeled derivative, or prodrug thereof, wherein R^{B} is not hydrogen (e.g., wherein R^{B} is halogen or substituted or unsubstituted C₁₋₆ alkyl).

In certain embodiments, the compound of Formula (I′) (e.g., Formula (I)) is of the formula:

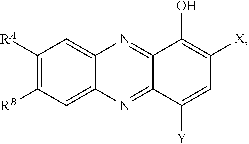

or a pharmaceutically acceptable salt, solvate, hydrate, polymorph, co-crystal, tautomer, stereoisomer, isotopically labeled derivative, or prodrug thereof, wherein both R^{A} and R^{B} are not hydrogen (e.g., wherein R^{A} and R^{B} is independently halogen or substituted or unsubstituted C₁₋₆ alkyl).

In certain embodiments, the compound of Formula (I′) (e.g., Formula (I)) is of the formula:

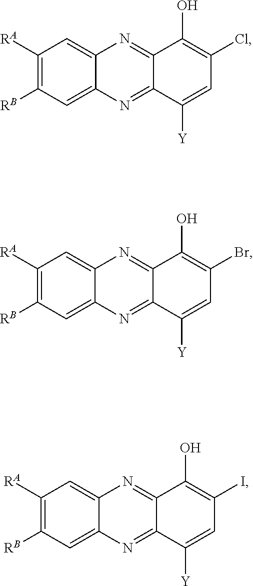

or a pharmaceutically acceptable salt, solvate, hydrate, polymorph, co-crystal, tautomer, stereoisomer, isotopically labeled derivative, or prodrug thereof, wherein both R^{A} and R^{B} are not hydrogen (e.g., wherein R^{A} and R^{B} is independently halogen or substituted or unsubstituted C₁₋₆ alkyl).

In certain embodiments, the compound of Formula (I′) (e.g., Formula (I)) is of the formula:

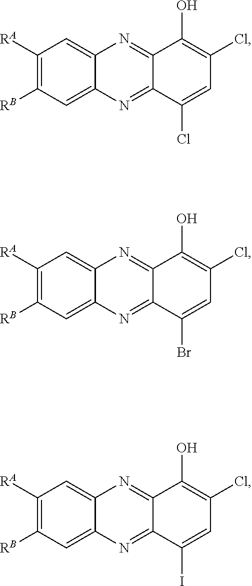

or a pharmaceutically acceptable salt, solvate, hydrate, polymorph, co-crystal, tautomer, stereoisomer, isotopically labeled derivative, or prodrug thereof, wherein both R^{A} and R^{B} are not hydrogen (e.g., wherein R^{A} and R^{B} is independently halogen or substituted or unsubstituted C₁₋₆ alkyl).

In certain embodiments, the compound of Formula (I′) (e.g., Formula (I)) is of the formula:

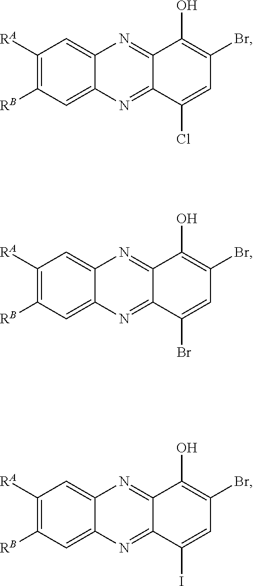

or a pharmaceutically acceptable salt, solvate, hydrate, polymorph, co-crystal, tautomer, stereoisomer, isotopically labeled derivative, or prodrug thereof, wherein both R^{A} and R^{B} are not hydrogen (e.g., wherein R^{A} and R^{B} is independently halogen or substituted or unsubstituted C₁₋₆ alkyl).

In certain embodiments, the compound of Formula (I′) (e.g., Formula (I)) is of the formula:

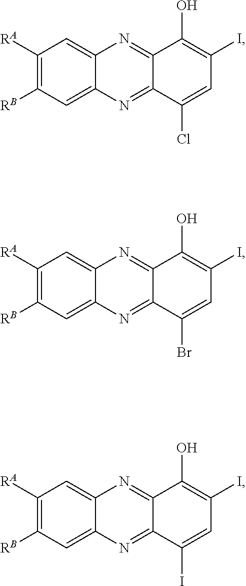

or a pharmaceutically acceptable salt, solvate, hydrate, polymorph, co-crystal, tautomer, stereoisomer, isotopically labeled derivative, or prodrug thereof, wherein both R^{A} and R^{B} are not hydrogen (e.g., wherein R^{A} and R^{B} is independently halogen or substituted or unsubstituted C₁₋₆ alkyl).

In certain embodiments, the compound of Formula (I′) (e.g., Formula (I)) is of the formula:

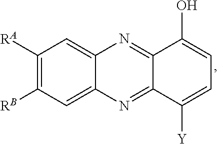

or a pharmaceutically acceptable salt, solvate, hydrate, polymorph, co-crystal, tautomer, stereoisomer, isotopically labeled derivative, or prodrug thereof, wherein both R^{A} and R^{B} are not hydrogen (e.g., wherein R^{A} and R^{B} is independently halogen or substituted or unsubstituted C₁₋₆ alkyl).

In certain embodiments, the compound of Formula (I′) (e.g., Formula (I)) is of the formula:

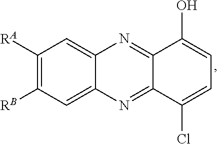

or a pharmaceutically acceptable salt, solvate, hydrate, polymorph, co-crystal, tautomer, stereoisomer, isotopically labeled derivative, or prodrug thereof, wherein both R^{A} and R^{B} are not hydrogen (e.g., wherein R^{A} and R^{B} is independently halogen or substituted or unsubstituted C₁₋₆ alkyl).

In certain embodiments, the compound of Formula (I′) (e.g., Formula (I)) is of the formula:

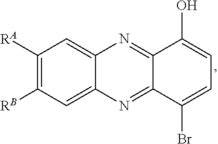

or a pharmaceutically acceptable salt, solvate, hydrate, polymorph, co-crystal, tautomer, stereoisomer, isotopically labeled derivative, or prodrug thereof, wherein both R^{A} and R^{B} are not hydrogen (e.g., wherein R^{A} and R^{B} is independently halogen or substituted or unsubstituted C₁₋₆ alkyl).

In certain embodiments, the compound of Formula (I′) (e.g., Formula (I)) is of the formula:

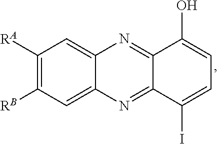

or a pharmaceutically acceptable salt, solvate, hydrate, polymorph, co-crystal, tautomer, stereoisomer, isotopically labeled derivative, or prodrug thereof, wherein both R^{A} and R^{B} are not hydrogen (e.g., wherein R^{A} and R^{B} is independently halogen or substituted or unsubstituted C₁₋₆ alkyl).

In certain embodiments, the compound of Formula (I′) (e.g., Formula (I)) is of the formula:

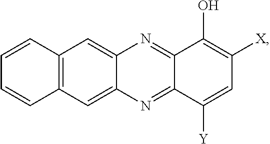

or a pharmaceutically acceptable salt, solvate, hydrate, polymorph, co-crystal, tautomer, stereoisomer, isotopically labeled derivative, or prodrug thereof.

Exemplary compounds of Formula (I′) (e.g., Formula (I)) include:

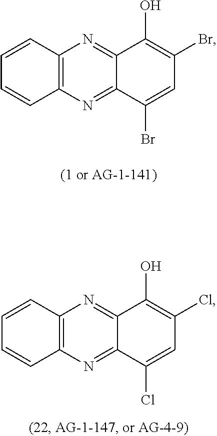 [SMILES: Oc1c(Br)cc(Br)c2nc3ccccc3nc12 ; Oc1c(Cl)cc(Cl)c2nc3ccccc3nc12]

and pharmaceutically acceptable salts, solvates, hydrates, polymorphs, co-crystals, tautomers, stereoisomers, isotopically labeled derivatives, and prodrugs thereof.

Exemplary compounds of Formula (I′) (e.g., Formula (I)) also include:

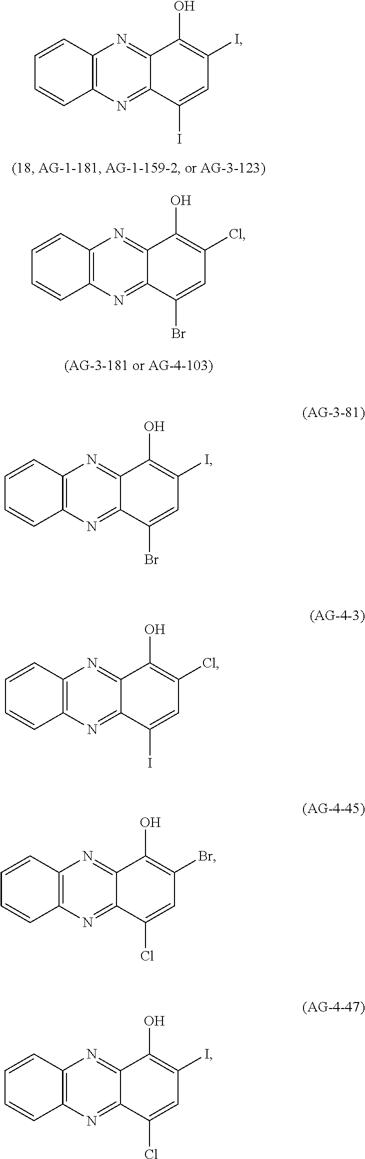 [SMILES: Oc1c(I)cc(I)c2nc3ccccc3nc12 ; Oc1c(Cl)cc(Br)c2nc3ccccc3nc12 ; Oc1c(I)cc(Br)c2nc3ccccc3nc12 ; Oc1c(Cl)cc(I)c2nc3ccccc3nc12 ; Oc1c(Br)cc(Cl)c2nc3ccccc3nc12 ; Oc1c(I)cc(Cl)c2nc3ccccc3nc12]

and pharmaceutically acceptable salts, solvates, hydrates, polymorphs, co-crystals, tautomers, stereoisomers, isotopically labeled derivatives, and prodrugs thereof.

Exemplary compounds of Formula (I′) (e.g., Formula (I)) also include:

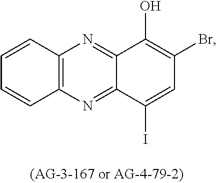 [SMILES: Oc1c(Br)cc(I)c2nc3ccccc3nc12]

and pharmaceutically acceptable salts, solvates, hydrates, polymorphs, co-crystals, tautomers, stereoisomers, isotopically labeled derivatives, and prodrugs thereof.

Exemplary compounds of Formula (I′) (e.g., Formula (I)) also include:

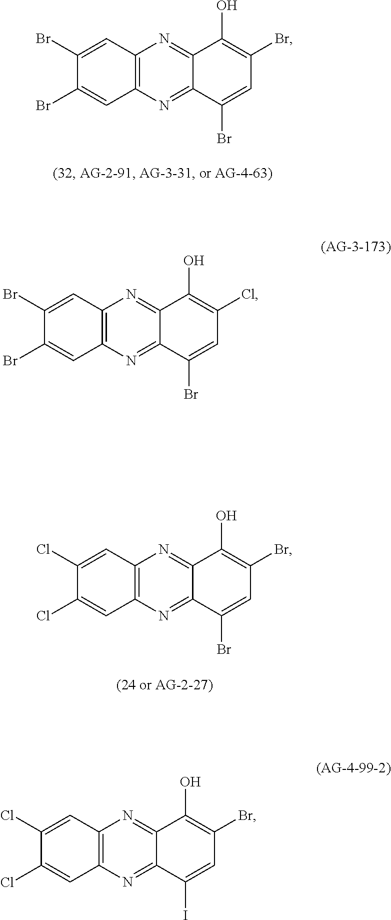 [SMILES: Oc1c(Br)cc(Br)c2nc3cc(Br)c(Br)cc3nc12 ; Oc1c(Cl)cc(Br)c2nc3cc(Br)c(Br)cc3nc12 ; Oc1c(Br)cc(Br)c2nc3cc(Cl)c(Cl)cc3nc12 ; Oc1c(Br)cc(I)c2nc3cc(Cl)c(Cl)cc3nc12]

and pharmaceutically acceptable salts, solvates, hydrates, polymorphs, co-crystals, tautomers, stereoisomers, isotopically labeled derivatives, and prodrugs thereof.

Exemplary compounds of Formula (I′) (e.g., Formula (I)) also include:

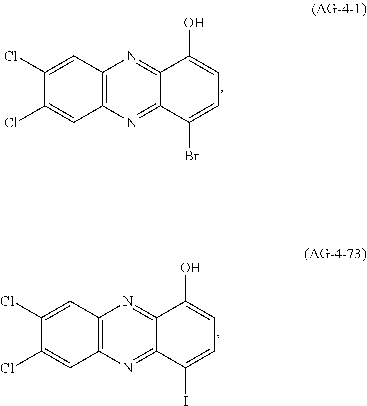 [SMILES: Oc1ccc(Br)c2nc3cc(Cl)c(Cl)cc3nc12 ; Oc1ccc(I)c2nc3cc(Cl)c(Cl)cc3nc12]

and pharmaceutically acceptable salts, solvates, hydrates, polymorphs, co-crystals, tautomers, stereoisomers, isotopically labeled derivatives, and prodrugs thereof.

Exemplary compounds of Formula (I′) (e.g., Formula (I)) also include:

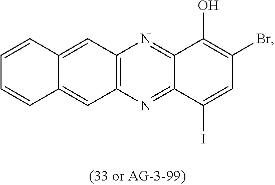 [SMILES: Oc1c(Br)cc(I)c2nc3cc4ccccc4cc3nc12]

and pharmaceutically acceptable salts, solvates, hydrates, polymorphs, co-crystals, tautomers, stereoisomers, isotopically labeled derivatives, and prodrugs thereof.

Exemplary compounds of Formula (I′) also include:

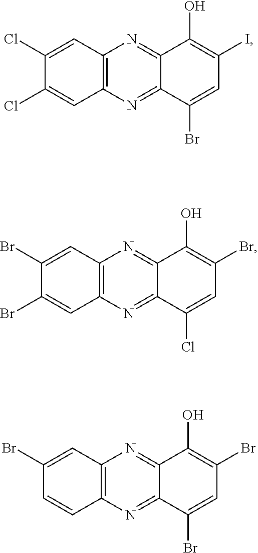 [SMILES: Oc1c(I)cc(Br)c2nc3cc(Cl)c(Cl)cc3nc12 ; Oc1c(Br)cc(Cl)c2nc3cc(Br)c(Br)cc3nc12 ; Oc1c(Br)cc(Br)c2nc3ccc(Br)cc3nc12]

and pharmaceutically acceptable salts, solvates, hydrates, polymorphs, co-crystals, tautomers, stereoisomers, isotopically labeled derivatives, and prodrugs thereof.

Exemplary compounds of Formula (I′) also include:

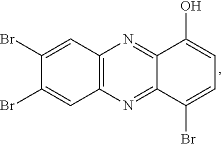 [SMILES: Oc1ccc(Br)c2nc3cc(Br)c(Br)cc3nc12]

and pharmaceutically acceptable salts, solvates, hydrates, polymorphs, co-crystals, tautomers, stereoisomers, isotopically labeled derivatives, and prodrugs thereof.

Exemplary compounds of Formula (I′) also include:

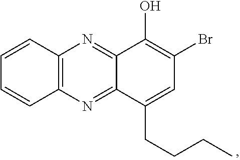 [SMILES: CCCCc1cc(Br)c(O)c2nc3ccccc3nc12]

and pharmaceutically acceptable salts, solvates, hydrates, polymorphs, co-crystals, tautomers, stereoisomers, isotopically labeled derivatives, and prodrugs thereof.

Exemplary compounds of Formula (I′) also include:

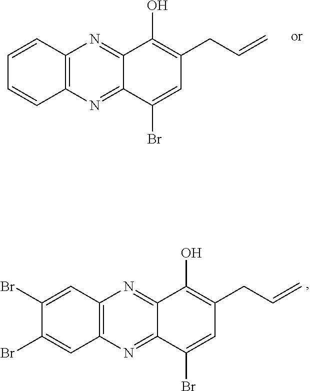 [SMILES: C=CCc1cc(Br)c2nc3ccccc3nc2c1O ; C=CCc1cc(Br)c2nc3cc(Br)c(Br)cc3nc2c1O]

and pharmaceutically acceptable salts, solvates, hydrates, polymorphs, co-crystals, tautomers, stereoisomers, isotopically labeled derivatives, and prodrugs thereof.

In certain embodiments, the compounds of the invention are compounds of Formula (II):

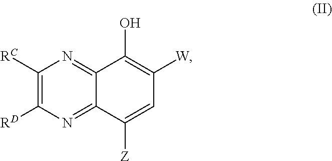

and pharmaceutically acceptable salts, solvates, hydrates, polymorphs, co-crystals, tautomers, stereoisomers, isotopically labeled derivatives, and prodrugs thereof, wherein:

W is hydrogen or halogen;

Z is halogen;

R^{C} is hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR³, —N(R³)₂, —SR³, —CN, —SCN, —C(═NR³)R³, —C(═NR³)OR³, —C(═NR³)N(R³)₂, —C(═O)R³, —C(═O)OR³, —C(═O)N(R³)₂, —NO₂, —NR³C(═O)R³, —NR³C(═O)OR³, —NR³C(═O)N(R³)₂, —OC(═O)R³, —OC(═O)OR³, or —OC(═O)N(R³)₂, wherein each instance of R³ is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R³ are joined to form a substituted or unsubstituted heterocyclic or substituted or unsubstituted heteroaryl ring; and

R^{D} is hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR⁴, —N(R⁴)₂, —SR⁴, —CN, —SCN, —C(═NR⁴)R⁴, —C(═NR⁴)OR⁴, —C(═NR⁴)N(R⁴)₂, —C(═O)R⁴, —C(═O)OR⁴, —C(═O)N(R⁴)₂, —NO₂, —NR⁴C(═O)R⁴, —NR⁴C(═O)OR⁴, —NR⁴C(═O)N(R⁴)₂, —OC(═O)R⁴, —OC(═O)OR⁴, or —OC(═O)N(R⁴)₂, wherein each instance of R⁴ is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R⁴ are joined to form a substituted or unsubstituted heterocyclic or substituted or unsubstituted heteroaryl ring;

provided that the compound is not of the formula

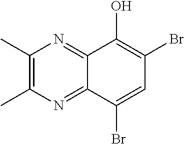

### (AG-4-41).

In certain embodiments, the compounds of the invention are compounds of Formula of Formula (III):

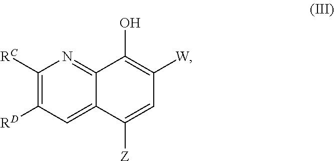

and pharmaceutically acceptable salts, solvates, hydrates, polymorphs, co-crystals, tautomers, stereoisomers, isotopically labeled derivatives, and prodrugs thereof, wherein:

W is hydrogen or halogen;

Z is halogen;

R^{C} is hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR³, —N(R³)₂, —SR³, —CN, —SCN, —C(═NR³)R³, —C(═NR³)OR³, —C(═NR³)N(R³)₂, —C(═O)R³, —C(═O)OR³, —C(═O)N(R³)₂, —NO₂, —NR³C(═O)R³, —NR³C(═O)OR³, —NR³C(═O)N(R³)₂, —OC(═O)R³, —OC(═O)OR³, or —OC(═O)N(R³)₂, wherein each instance of R³ is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R³ are joined to form a substituted or unsubstituted heterocyclic or substituted or unsubstituted heteroaryl ring; and

R^{D} is hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR⁴, —N(R⁴)₂, —SR⁴, —CN, —SCN, —C(═NR⁴)R⁴, —C(═NR⁴)OR⁴, —C(═NR⁴)N(R⁴)₂, —C(═O)R⁴, —C(═O)OR⁴, —C(═O)N(R⁴)₂, —NO₂, —NR⁴C(═O)R⁴, —NR⁴C(═O)OR⁴, —NR⁴C(═O)N(R⁴)₂, —OC(═O)R⁴, —OC(═O)OR⁴, or —OC(═O)N(R⁴)₂, wherein each instance of R⁴ is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R⁴ are joined to form a substituted or unsubstituted heterocyclic or substituted or unsubstituted heteroaryl ring.

Formula (II) or (III) includes substituent W on the quinoxalinyl ring. In certain embodiments, W is hydrogen. In certain embodiments, W is halogen. In certain embodiments, W is F. In certain embodiments, W is C₁. In certain embodiments, W is Br. In certain embodiments, W is I.

Formula (II) or (III) also includes substituent Z on the quinoxalinyl ring. In certain embodiments, Z is F. In certain embodiments, Z is C₁. In certain embodiments, Z is Br. In certain embodiments, Z is I.

In certain embodiments, W is Cl; and Z is F. In certain embodiments, both W and Z and C₁. In certain embodiments, W is Cl; and Z is Br. In certain embodiments, W is Cl; and Z is I. In certain embodiments, W is Br; and Z is F. In certain embodiments, W is Br; and Z is C₁. In certain embodiments, both W and Z are Br. In certain embodiments, W is Br; and Z is I. In certain embodiments, W is I; and Z is F. In certain embodiments, W is I; and Z is C₁. In certain embodiments, W is I; and Z is Br. In certain embodiments, both W and Z are I. In certain embodiments, W and Z are the same. In certain embodiments, W and Z are not the same.

Formula (II) or (III) also includes substituent R^{C} on the quinoxalinyl ring. In certain embodiments, R^{C} is hydrogen. In certain embodiments, R^{C} is not hydrogen. In certain embodiments, R^{C} is halogen. In certain embodiments, R^{C} is F. In certain embodiments, R^{C} is C₁. In certain embodiments, R^{C} is Br. In certain embodiments, R^{C} is I. In certain embodiments, R^{C} is substituted or unsubstituted alkyl. In certain embodiments, R^{C} is substituted or unsubstituted C₁₋₆ alkyl. In certain embodiments, R^{cc} is Me. In certain embodiments, R^{C} is substituted methyl (e.g., —CH₂F, —CHF₂, —CF₃, or Bn). In certain embodiments, R^{C} is Et, substituted ethyl (e.g., fluorinated ethyl (e.g., perfluoroethyl)), Pr, substituted propyl (e.g., fluorinated propyl (e.g., perfluoropropyl)), Bu, or substituted butyl (e.g., fluorinated butyl (e.g., perfluorobutyl)). In certain embodiments, R^{C} is substituted or unsubstituted alkenyl (e.g., substituted or unsubstituted C₂₋₆ alkenyl). In certain embodiments, R^{C} is substituted or unsubstituted alkynyl (e.g., substituted or unsubstituted C₂₋₆ alkynyl). In certain embodiments, R^{C} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, R^{C} is substituted or unsubstituted cyclopropyl, substituted or unsubstituted cyclobutyl, substituted or unsubstituted cyclopentyl, or substituted or unsubstituted cyclohexyl. In certain embodiments, R^{C} is substituted or unsubstituted heterocyclyl (e.g., substituted or unsubstituted, 3- to 7-membered, monocyclic heterocyclyl comprising zero, one, or two double bonds in the heterocyclic ring system, wherein one, two, or three atoms in the heterocyclic ring system are independently nitrogen, oxygen, or sulfur). In certain embodiments, R^{C} is substituted or unsubstituted oxetanyl, substituted or unsubstituted tetrahydrofuranyl, substituted or unsubstituted pyrrolidinyl, substituted or unsubstituted tetrahydropyranyl, substituted or unsubstituted piperidinyl, substituted or unsubstituted morpholinyl, or substituted or unsubstituted piperazinyl. In certain embodiments, R^{C} is substituted or unsubstituted aryl (e.g., substituted or unsubstituted, 6- to 10-membered aryl). In certain embodiments, R^{C} is unsubstituted phenyl. In certain embodiments, R^{C} is substituted phenyl. In certain embodiments, R^{C} is substituted or unsubstituted naphthyl. In certain embodiments, R^{C} is substituted or unsubstituted heteroaryl. In certain embodiments, R^{C} is substituted or unsubstituted, 5- to 6-membered, monocyclic heteroaryl, wherein one, two, three, or four atoms in the heteroaryl ring system are independently nitrogen, oxygen, or sulfur. In certain embodiments, R^{C} is substituted or unsubstituted, 9- to 10-membered, bicyclic heteroaryl, wherein one, two, three, or four atoms in the heteroaryl ring system are independently nitrogen, oxygen, or sulfur. In certain embodiments, R^{C} is —OR³ (e.g., —OH, —O(substituted or unsubstituted C₁₋₆ alkyl) (e.g., —OMe, —OCF₃, —OEt, —OPr, —OBu, or —OBn), or —O(substituted or unsubstituted phenyl) (e.g., —OPh)). In certain embodiments, R^{C} is —SR³ (e.g., —SH, —S(substituted or unsubstituted C₁₋₆ alkyl) (e.g., —SMe, —SEt, —SPr, —SBu, or —SBn), or —S(substituted or unsubstituted phenyl) (e.g., —SPh)). In certain embodiments, R^{C} is —N(R³)₂ (e.g., —NH₂, —NH(substituted or unsubstituted C₁₋₆ alkyl) (e.g., —NHMe), or —N(substituted or unsubstituted C₁₋₆ alkyl)-(substituted or unsubstituted C₁₋₆ alkyl) (e.g., —NMe₂)). In certain embodiments, R^{C} is —CN or —SCN. In certain embodiments, R^{C} is —NO₂. In certain embodiments, R^{C} is —C(═NR³)R³, —C(═NR³)OR³, or —C(═NR³)N(R³)₂. In certain embodiments, R^{C} is —C(═O)R³ (e.g., —C(═O)(substituted or unsubstituted alkyl) (e.g., —C(═O)Me) or —C(═O)(substituted or unsubstituted phenyl)). In certain embodiments, R^{C} is —C(═O)OR³ (e.g., —C(═O)OH, —C(═O)O(substituted or unsubstituted alkyl) (e.g., —C(═O)OMe), or —C(═O)O(substituted or unsubstituted phenyl)). In certain embodiments, R^{C} is —C(═O)N(R³)₂ (e.g., —C(═O)NH₂, —C(═O)NH(substituted or unsubstituted alkyl) (e.g., —C(═O)NHMe), —C(═O)NH(substituted or unsubstituted phenyl), —C(═O)N(substituted or unsubstituted alkyl)-(substituted or unsubstituted alkyl), or —C(═O)N(substituted or unsubstituted phenyl)-(substituted or unsubstituted alkyl)). In certain embodiments, R^{C} is —NR³C(═O)R³ (e.g., —NHC(═O)(substituted or unsubstituted C₁₋₆ alkyl) (e.g., —NHC(═O)Me) or —NHC(═O)(substituted or unsubstituted phenyl)). In certain embodiments, R^{C} is —NR³C(═O)OR³. In certain embodiments, R^{C} is —NR³C(═O)N(R³)₂ (e.g., —NHC(═O)NH₂, —NHC(═O)NH(substituted or unsubstituted C₁₋₆ alkyl) (e.g., —NHC(═O)NHMe)). In certain embodiments, R^{C} is —OC(═O)R³ (e.g., —OC(═O)(substituted or unsubstituted alkyl) or —OC(═O)(substituted or unsubstituted phenyl)), —OC(═O)OR³ (e.g., —OC(═O)O(substituted or unsubstituted alkyl) or —OC(═O)O(substituted or unsubstituted phenyl)), or —OC(═O)N(R³)₂ (e.g., —OC(═O)NH₂, —OC(═O)NH(substituted or unsubstituted alkyl), —OC(═O)NH(substituted or unsubstituted phenyl), —OC(═O)N(substituted or unsubstituted alkyl)-(substituted or unsubstituted alkyl), or —OC(═O)N(substituted or unsubstituted phenyl)-(substituted or unsubstituted alkyl)).

Formula (II) or (III) may include one or more instances of substituent R³. When Formula (II) or (III) includes two or more instances of R³, any two instances of R³ may be the same or different from each other. In certain embodiments, at least one instance of R³ is H. In certain embodiments, each instance of R³ is H. In certain embodiments, at least one instance of R³ is substituted or unsubstituted alkyl (e.g., substituted or unsubstituted C₁₋₆ alkyl (e.g., Me)). In certain embodiments, at least one instance of R³ is substituted or unsubstituted acyl, substituted or unsubstituted alkenyl (e.g., substituted or unsubstituted C₂₋₆ alkenyl), substituted or unsubstituted alkynyl (e.g., substituted or unsubstituted C₂₋₆ alkynyl), substituted or unsubstituted carbocyclyl (e.g., substituted or unsubstituted, 3- to 7-membered, monocyclic carbocyclyl comprising zero, one, or two double bonds in the carbocyclic ring system), substituted or unsubstituted heterocyclyl (e.g., substituted or unsubstituted, 3- to 7-membered, monocyclic heterocyclyl comprising zero, one, or two double bonds in the heterocyclic ring system, wherein one, two, or three atoms in the heterocyclic ring system are independently nitrogen, oxygen, or sulfur), substituted or unsubstituted aryl (e.g., substituted or unsubstituted phenyl), substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur), a nitrogen protecting group (e.g., Bn, Boc, Cbz, Fmoc, trifluoroacetyl, triphenylmethyl, acetyl, or Ts) when attached to a nitrogen atom, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom. In certain embodiments, two instances of R³ are joined to form a substituted or unsubstituted heterocyclic or substituted or unsubstituted heteroaryl ring.

Formula (II) or (III) also includes substituent R^{D} on the quinoxalinyl ring. In certain embodiments, R^{D} is hydrogen. In certain embodiments, R^{D} is not hydrogen. In certain embodiments, R^{D} is halogen. In certain embodiments, R^{D} is F. In certain embodiments, R^{D} is C₁. In certain embodiments, R^{D} is Br. In certain embodiments, R^{D} is I. In certain embodiments, R^{D} is substituted or unsubstituted alkyl. In certain embodiments, R^{D} is substituted or unsubstituted C₁₋₆ alkyl. In certain embodiments, R^{D} is Me. In certain embodiments, R^{D} is substituted methyl (e.g., —CH₂F, —CHF₂, —CF₃, or Bn). In certain embodiments, R^{D} is Et, substituted ethyl (e.g., fluorinated ethyl (e.g., perfluoroethyl)), Pr, substituted propyl (e.g., fluorinated propyl (e.g., perfluoropropyl)), Bu, or substituted butyl (e.g., fluorinated butyl (e.g., perfluorobutyl)). In certain embodiments, R^{D} is substituted or unsubstituted alkenyl (e.g., substituted or unsubstituted C₂₋₆ alkenyl). In certain embodiments, R^{D} is substituted or unsubstituted alkynyl (e.g., substituted or unsubstituted C₂₋₆ alkynyl). In certain embodiments, R^{D} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, R^{D} is substituted or unsubstituted cyclopropyl, substituted or unsubstituted cyclobutyl, substituted or unsubstituted cyclopentyl, or substituted or unsubstituted cyclohexyl. In certain embodiments, R^{D} is substituted or unsubstituted heterocyclyl (e.g., substituted or unsubstituted, 3- to 7-membered, monocyclic heterocyclyl comprising zero, one, or two double bonds in the heterocyclic ring system, wherein one, two, or three atoms in the heterocyclic ring system are independently nitrogen, oxygen, or sulfur). In certain embodiments, R^{D} is substituted or unsubstituted oxetanyl, substituted or unsubstituted tetrahydrofuranyl, substituted or unsubstituted pyrrolidinyl, substituted or unsubstituted tetrahydropyranyl, substituted or unsubstituted piperidinyl, substituted or unsubstituted morpholinyl, or substituted or unsubstituted piperazinyl. In certain embodiments, R^{D} is substituted or unsubstituted aryl (e.g., substituted or unsubstituted, 6- to 10-membered aryl). In certain embodiments, R^{D} is unsubstituted phenyl. In certain embodiments, R^{D} is substituted phenyl. In certain embodiments, R^{D} is substituted or unsubstituted naphthyl. In certain embodiments, R^{D} is substituted or unsubstituted heteroaryl. In certain embodiments, R^{D} is substituted or unsubstituted, 5- to 6-membered, monocyclic heteroaryl, wherein one, two, three, or four atoms in the heteroaryl ring system are independently nitrogen, oxygen, or sulfur. In certain embodiments, R^{D} is substituted or unsubstituted, 9- to 10-membered, bicyclic heteroaryl, wherein one, two, three, or four atoms in the heteroaryl ring system are independently nitrogen, oxygen, or sulfur. In certain embodiments, R^{D} is —OR⁴ (e.g., —OH, —O(substituted or unsubstituted C₁₋₆ alkyl) (e.g., —OMe, —OCF₃, —OEt, —OPr, —OBu, or —OBn), or —O(substituted or unsubstituted phenyl) (e.g., —OPh)). In certain embodiments, R^{D} is —SR⁴ (e.g., —SH, —S(substituted or unsubstituted C₁₋₆ alkyl) (e.g., —SMe, —SEt, —SPr, —SBu, or —SBn), or —S(substituted or unsubstituted phenyl) (e.g., —SPh)). In certain embodiments, R^{D} is —N(R⁴)₂ (e.g., —NH₂, —NH(substituted or unsubstituted C₁₋₆ alkyl) (e.g., —NHMe), or —N(substituted or unsubstituted C₁₋₆ alkyl)-(substituted or unsubstituted C₁₋₆ alkyl) (e.g., —NMe₂)). In certain embodiments, R^{D} is —CN or —SCN. In certain embodiments, R^{D} is —NO₂. In certain embodiments, R^{D} is —C(═NR⁴)R⁴, —C(═NR⁴)OR⁴, or —C(═NR⁴)N(R⁴)₂. In certain embodiments, R^{D} is —C(═O)R⁴ (e.g., —C(═O)(substituted or unsubstituted alkyl) (e.g., —C(═O)Me) or —C(═O)(substituted or unsubstituted phenyl)). In certain embodiments, R^{D} is —C(═O)OR⁴ (e.g., —C(═O)OH, —C(═O)O(substituted or unsubstituted alkyl) (e.g., —C(═O)OMe), or —C(═O)O(substituted or unsubstituted phenyl)). In certain embodiments, R^{D} is —C(═O)N(R⁴)₂ (e.g., —C(═O)NH₂, —C(═O)NH(substituted or unsubstituted alkyl) (e.g., —C(═O)NHMe), —C(═O)NH(substituted or unsubstituted phenyl), —C(═O)N(substituted or unsubstituted alkyl)-(substituted or unsubstituted alkyl), or —C(═O)N(substituted or unsubstituted phenyl)-(substituted or unsubstituted alkyl)). In certain embodiments, R^{D} is —NR⁴C(═O)R⁴ (e.g., —NHC(═O)(substituted or unsubstituted C₁₋₆ alkyl) (e.g., —NHC(═O)Me) or —NHC(═O)(substituted or unsubstituted phenyl)). In certain embodiments, R^{D} is —NR⁴C(═O)OR⁴. In certain embodiments, R^{D} is —NR⁴C(═O)N(R⁴)₂ (e.g., —NHC(═O)NH₂, —NHC(═O)NH(substituted or unsubstituted C₁₋₆ alkyl) (e.g., —NHC(═O)NHMe)). In certain embodiments, R^{D} is —OC(═O)R⁴ (e.g., —OC(═O)(substituted or unsubstituted alkyl) or —OC(═O)(substituted or unsubstituted phenyl)), —OC(═O)OR⁴ (e.g., —OC(═O)O(substituted or unsubstituted alkyl) or —OC(═O)O(substituted or unsubstituted phenyl)), or —OC(═O)N(R⁴)₂ (e.g., —OC(═O)NH₂, —OC(═O)NH(substituted or unsubstituted alkyl), —OC(═O)NH(substituted or unsubstituted phenyl), —OC(═O)N(substituted or unsubstituted alkyl)-(substituted or unsubstituted alkyl), or —OC(═O)N(substituted or unsubstituted phenyl)-(substituted or unsubstituted alkyl)).

Formula (II) or (III) may include one or more instances of substituent R⁴. When Formula (II) or (III) includes two or more instances of R⁴, any two instances of R⁴ may be the same or different from each other. In certain embodiments, at least one instance of R⁴ is H. In certain embodiments, each instance of R⁴ is H. In certain embodiments, at least one instance of R⁴ is substituted or unsubstituted alkyl (e.g., substituted or unsubstituted C₁₋₆ alkyl (e.g., Me)). In certain embodiments, at least one instance of R⁴ is substituted or unsubstituted acyl, substituted or unsubstituted alkenyl (e.g., substituted or unsubstituted C₂₋₆ alkenyl), substituted or unsubstituted alkynyl (e.g., substituted or unsubstituted C₂₋₆ alkynyl), substituted or unsubstituted carbocyclyl (e.g., substituted or unsubstituted, 3- to 7-membered, monocyclic carbocyclyl comprising zero, one, or two double bonds in the carbocyclic ring system), substituted or unsubstituted heterocyclyl (e.g., substituted or unsubstituted, 3- to 7-membered, monocyclic heterocyclyl comprising zero, one, or two double bonds in the heterocyclic ring system, wherein one, two, or three atoms in the heterocyclic ring system are independently nitrogen, oxygen, or sulfur), substituted or unsubstituted aryl (e.g., substituted or unsubstituted phenyl), substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur), a nitrogen protecting group (e.g., Bn, Boc, Cbz, Fmoc, trifluoroacetyl, triphenylmethyl, acetyl, or Ts) when attached to a nitrogen atom, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom. In certain embodiments, two instances of R⁴ are joined to form a substituted or unsubstituted heterocyclic or substituted or unsubstituted heteroaryl ring.

In certain embodiments, R^{C} is hydrogen, and R^{D} is not hydrogen. In certain embodiments, R^{D} is hydrogen, and R^{C} is not hydrogen. In certain embodiments, each one of R^{C} and R^{D} is not hydrogen. In certain embodiments, both R^{C} and R^{D} are H. In certain embodiments, both R^{C} and R^{D} are halogen (e.g., Cl, Br, or I). In certain embodiments, both R^{C} and R^{D} are substituted or unsubstituted alkyl (e.g., substituted or unsubstituted C₁₋₆ alkyl (e.g., Me)). In certain embodiments, each of R^{C} and R^{D} is independently hydrogen or substituted or unsubstituted alkyl (e.g., substituted or unsubstituted C₁₋₆ alkyl (e.g., Me)). In certain embodiments, each of R^{C} and R^{D} is independently hydrogen, halogen (e.g., Cl, Br, or I), or substituted or unsubstituted alkyl (e.g., substituted or unsubstituted C₁₋₆ alkyl (e.g., Me)). In certain embodiments, each of R^{C} and R^{D} is independently halogen (e.g., Cl, Br, or I) or substituted or unsubstituted alkyl (e.g., substituted or unsubstituted C₁₋₆ alkyl (e.g., Me)).

In certain embodiments, the compound of the invention is not of the formula:

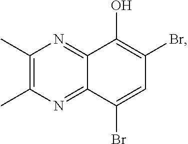

or a pharmaceutically acceptable salt thereof. In certain embodiments, at least one of R^{C} and R^{D} is not unsubstituted C₁₋₆ alkyl (e.g., Me). In certain embodiments, only one of R^{C} and R^{D} is not unsubstituted C₁₋₆ alkyl (e.g., Me). In certain embodiments, at least one of W and Z is not Br.

Exemplary compounds of Formula (II) include:

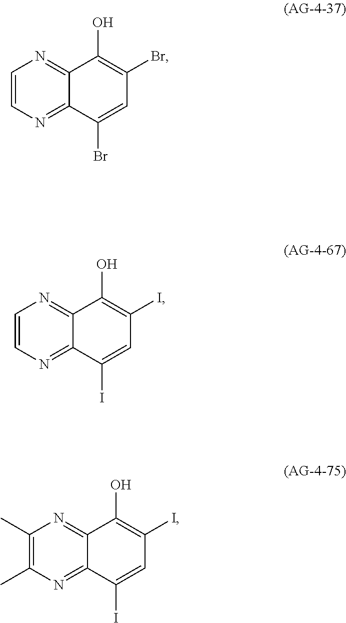

and pharmaceutically acceptable salts, solvates, hydrates, polymorphs, co-crystals, tautomers, stereoisomers, isotopically labeled derivatives, and prodrugs thereof.

Exemplary compounds of Formula (II) further include:

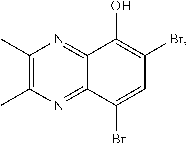

and pharmaceutically acceptable salts, solvates, hydrates, polymorphs, co-crystals, tautomers, stereoisomers, isotopically labeled derivatives, and prodrugs thereof.

Exemplary compounds of Formula (III) include:

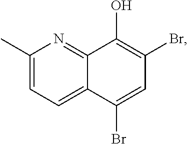

and pharmaceutically acceptable salts, solvates, hydrates, polymorphs, co-crystals, tautomers, stereoisomers, isotopically labeled derivatives, and prodrugs thereof.

In certain embodiments, the compounds of the invention are the compounds described herein, and salts (e.g., pharmaceutically acceptable salts), solvates, hydrates, polymorphs, co-crystals, tautomers, stereoisomers, isotopically labeled derivatives, and prodrugs thereof. In certain embodiments, the compounds of the invention are the compounds described herein, and pharmaceutically acceptable salts thereof. In certain embodiments, the compounds of the invention are the compounds described herein, and pharmaceutically acceptable salts thereof. In certain embodiments, the compounds of the invention are the compounds of Formula (I′), and pharmaceutically acceptable salts, solvates, hydrates, polymorphs, co-crystals, tautomers, stereoisomers, isotopically labeled derivatives, and prodrugs thereof. In certain embodiments, the compounds of the invention are the compounds of Formula (I′), and salts (e.g., pharmaceutically acceptable salts) thereof. In certain embodiments, the compounds of the invention are the compounds of Formula (I), and pharmaceutically acceptable salts, solvates, hydrates, polymorphs, co-crystals, tautomers, stereoisomers, isotopically labeled derivatives, and prodrugs thereof. In certain embodiments, the compounds of the invention are the compounds of Formula (I), and salts (e.g., pharmaceutically acceptable salts) thereof. In certain embodiments, the compounds of the invention are the compounds of Formula (II), and pharmaceutically acceptable salts, solvates, hydrates, polymorphs, co-crystals, tautomers, stereoisomers, isotopically labeled derivatives, and prodrugs thereof. In certain embodiments, the compounds of the invention are the compounds of Formula (II), and salts (e.g., pharmaceutically acceptable salts) thereof. In certain embodiments, the compounds of the invention are the compounds of Formula (III), and pharmaceutically acceptable salts, solvates, hydrates, polymorphs, co-crystals, tautomers, stereoisomers, isotopically labeled derivatives, and prodrugs thereof. In certain embodiments, the compounds of the invention are the compounds of Formula (III), and salts (e.g., pharmaceutically acceptable salts) thereof.

In certain embodiments, the compounds of the invention are substantially pure. In certain embodiments, a compound of the invention is at least about 90%, at least about 95%, at least about 97%, at least about 98%, at least about 99%, or at least about 99.5% free of impurities.

The compounds of the invention have been found to be antimicrobial agents (e.g., antibacterial agents). Without wishing to be bound by a particular theory, the compounds of the invention may be redox-active and may generate reactive oxygen species (ROS). The inventive compounds may thus act as microbial warfare agents and inhibit the growth and/or reproduction of or kill a microorganism (e.g., bacterium, mycobacterium, archaeon, protist, fungus, or parasite) by oxidizing and/or reducing molecules (e.g., catalase, cytokine, nicotinamide adenine dinucleotide phosphate (NADPH), and nicotinamide adenine dinucleotide phosphate (NADP⁺)) in, near, or around the microorganism. The activity of a compound of the invention against a microorganism may be measured by the minimum inhibitory concentration (MIC) of the compound in inhibiting the viability, growth, or replication of the microorganism. In certain embodiments, the MIC of a compound of the invention is an MIC in inhibiting the viability the microorganism. In certain embodiments, the MIC value of an inventive compound in inhibiting a microorganism is at most about 1 nM, at most about 3 nM, at most about 10 nM, at most about 30 nM, at most about 100 nM, at most about 300 nM, at most about 1 μM, at most about 3 μM, at most about 10 μM, at most about 30 μM, or at most about 100 μM. In certain embodiments, the MIC value of an inventive compound in inhibiting a microorganism is at least about 1 nM, at least about 3 nM, at least about 10 nM, at least about 30 nM, at least about 100 nM, at least about 300 nM, at least about 1 μM, at least about 3 μM, at least about 10 μM, or at least about 30 μM. In certain embodiments, MIC values are measured according to the guidelines of the Clinical and Laboratory Standards Institute (CLSI) (which is incorporated herein by reference) (e.g., a broth microdilution method). In certain embodiments, MIC values are measured by a method described herein.

The activity of a compound of the invention against a microorganism may also be measured by the half maximal inhibitory concentration (IC₅₀) of the compound in inhibiting the viability, growth, or replication of the microorganism. In certain embodiments, the IC₅₀ of a compound of the invention is an MIC in inhibiting the viability the microorganism. In certain embodiments, the IC₅₀ value of an inventive compound in inhibiting a microorganism is at most about 1 nM, at most about 3 nM, at most about 10 nM, at most about 30 nM, at most about 100 nM, at most about 300 nM, at most about 1 μM, at most about 3 μM, at most about 10 μM, at most about 30 μM, or at most about 100 μM. In certain embodiments, the IC₅₀ value of an inventive compound in inhibiting a microorganism is at least about 1 nM, at least about 3 nM, at least about 10 nM, at least about 30 nM, at least about 100 nM, at least about 300 nM, at least about 1 μM, at least about 3 μM, at least about 10 μM, or at least about 30 μM. In certain embodiments, IC₅₀ values are measured according to the guidelines of the CLSI (e.g., a microdilution method). In certain embodiments, IC₅₀ values are measured by a method described herein.

The compounds of the invention may selectively inhibit the growth and/or reproduction of or kill a microorganism. In certain embodiments, a compound of the invention is more active in inhibiting the growth and/or reproduction of or killing a first microorganism (e.g., a microorganism described herein) than in inhibiting the growth and/or reproduction of or killing a host cell. In certain embodiments, a compound of the invention is more active in inhibiting the growth and/or reproduction of or killing a first microorganism than in inhibiting the growth and/or reproduction of or killing a second microorganism. The selectivity of an inventive compound in inhibiting the growth and/or reproduction of or killing a first microorganism over a host cell or a second microorganism may be determined by the quotient of the MIC value of the inventive compound in inhibiting the growth and/or reproduction of or killing the host cell or second microorganism over the MIC value of the inventive compound in inhibiting the growth and/or reproduction of or killing the first microorganism. The selectivity of an inventive compound in inhibiting the growth and/or reproduction of or killing a first microorganism over a host cell or a second microorganism may also be determined by the quotient of the IC₅₀ value of the inventive compound in inhibiting the growth and/or reproduction of or killing the host cell or second microorganism over the IC₅₀ value of the inventive compound in inhibiting the growth and/or reproduction of or killing the first microorganism. In certain embodiments, the selectivity of an inventive compound in inhibiting the growth and/or reproduction of or killing a first microorganism over a host cell or a second microorganism is at least about 3-fold, at least about 10-fold, at least about 30-fold, at least about 100-fold, at least about 1,000-fold, at least about 10,000-fold, or at least about 100,000-fold.

The compounds of the invention may show low cytotoxicity toward mammalian cells (e.g., cytotoxicity IC₅₀ against HeLa cells being greater than 100 μM). The compounds of the invention may show low hemolysis activity (e.g., not more than 1%, not more than 2%, not more than 4%, or not more than 6% hemolysis of red blood cells (RBCs) when treated with the compound at 200 μM).

### Compositions, Kits, and Administration

The present invention also provides compositions (e.g., pharmaceutical compositions) comprising a compound of the invention (e.g., a compound of Formula (I′) (e.g., Formula (I)), (II), or (III), or pharmaceutically acceptable salts thereof), and optionally an excipient (e.g., pharmaceutically acceptable excipient).

In certain embodiments, a composition of the invention is useful for disinfecting a surface. In certain embodiments, the compound of the invention is provided in an effective amount in the composition. In certain embodiments, the amount of the compound included in the composition is effective for killing at least 80%, at least 90%, at least 95%, at least 99%, at least 99.9%, or at least 99.99% of the microorganisms on the surface. In certain embodiments, the amount of the compound included in the composition is effective for killing at most 90%, at most 95%, at most 99%, at most 99.9%, at most 99.99%, or at most 99.999% of the microorganisms on the surface. A composition of the invention may include one or more excipients (e.g., water, detergent, bleach, surfactant) (e.g., pharmaceutically acceptable excipients).

In certain embodiments, a composition of the invention is a pharmaceutical composition comprising a compound of the invention and optionally a pharmaceutically acceptable excipient. In certain embodiments, the compound of the invention is provided in an effective amount in the pharmaceutical composition. In certain embodiments, the effective amount of the compound is a therapeutically effective amount. In certain embodiments, the effective amount of the compound is a prophylactically effective amount. The pharmaceutical compositions of the invention may be useful in the inventive methods. In certain embodiments, the pharmaceutical compositions are useful in treating a microbial infection (e.g., a bacterial infection or mycobacterial infection). In certain embodiments, the pharmaceutical compositions are useful in preventing a microbial infection (e.g., a bacterial infection or mycobacterial infection). In certain embodiments, the pharmaceutical compositions are useful in inhibiting the growth of a microorganism (e.g., a microorganism described herein). In certain embodiments, the pharmaceutical compositions are useful in inhibiting the reproduction of a microorganism. In certain embodiments, the pharmaceutical compositions are useful in killing a microorganism. In certain embodiments, the pharmaceutical compositions are useful in inhibiting the formation and/or growth of a biofilm. In certain embodiments, the pharmaceutical compositions are useful in reducing or removing a biofilm. In certain embodiments, the pharmaceutical compositions are useful in disinfecting a surface. In certain embodiments, the pharmaceutical compositions are useful in cleaning a surface.

Pharmaceutical compositions described herein can be prepared by any method known in the art of pharmacology. In general, such preparatory methods include the steps of bringing the compound of the invention (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carb oxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60), sorbitan tristearate (Span 65), glyceryl monooleate, sorbitan monooleate (Span 80)), polyoxyethylene esters (e.g., polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g., Cremophor™), polyoxyethylene ethers, (e.g., polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F-68, Poloxamer-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethyl cellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents such as Cremophor™, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a microbial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are typically suppositories which can be prepared by mixing the conjugates of this invention with suitable non-irritating excipients or carriers such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active ingredient can be admixed with at least one inert diluent such as sucrose, lactose or starch. Such dosage forms may comprise, as is normal practice, additional substances other than inert diluents, e.g., tableting lubricants and other tableting aids such a magnesium stearate and microcrystalline cellulose. In the case of capsules, tablets, and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes.

Dosage forms for topical and/or transdermal administration of a compound of this invention may include ointments, pastes, creams, lotions, gels, powders, solutions, sprays, inhalants and/or patches. Generally, the active ingredient is admixed under sterile conditions with a pharmaceutically acceptable carrier and/or any needed preservatives and/or buffers as can be required. Additionally, the present invention contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Jet injection devices which deliver liquid vaccines to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable. Alternatively or additionally, conventional syringes can be used in the classical mantoux method of intradermal administration.

Formulations suitable for topical administration include, but are not limited to, liquid and/or semi liquid preparations such as liniments, lotions, oil-in-water and/or water-in-oil emulsions such as creams, ointments, and/or pastes, and/or solutions and/or suspensions. Topically-administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition of the invention can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions of the invention formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition of the invention. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition of the invention can be prepared, packaged, and/or sold in a formulation for buccal administration. Such formulations may, for example, be in the form of tablets, and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition of the invention can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are contemplated as being within the scope of this invention.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions of the present invention will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a compound required to achieve an effective amount will vary from subject to subject, depending, for example, on species, age, and general condition of a subject, severity of the side effects or disorder, identity of the particular compound(s), mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

It will be also appreciated that a compound or composition, as described herein, can be administered in combination with one or more additional pharmaceutical agents. In certain embodiments, the additional pharmaceutical agent is different from a compound of the invention, or a pharmaceutically acceptable salt, solvate, hydrate, polymorph, co-crystal, tautomer, stereoisomer, isotopically labeled derivative, or prodrug thereof. The compounds or compositions can be administered in combination with additional pharmaceutical agents to improve their potency, efficacy, and/or bioavailability, reduce and/or modify their metabolism, inhibit their excretion, and/or modify their distribution within the body. It will also be appreciated that the therapy employed may achieve a desired effect for the same disorder, and/or it may achieve different effects. In certain embodiments, the combination of a compound of the invention and an additional pharmaceutical agent shows a synergistic effect.

The compound or composition can be administered concurrently with, prior to, or subsequent to, one or more additional pharmaceutical agents, which are different from the compound or composition and may be useful as, e.g., combination therapies. Pharmaceutical agents include therapeutically active agents. Pharmaceutical agents also include prophylactically active agents. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the inventive compound with the additional pharmaceutical agents and/or the desired therapeutic and/or prophylactic effect to be achieved. In general, it is expected that the additional pharmaceutical agents in combination be utilized at levels that do not exceed the levels at which they are utilized individually. In some embodiments, the levels utilized in combination will be lower than those utilized individually.

Exemplary additional pharmaceutical agents include, but are not limited to, antibiotics (e.g., antibacterial agents, antiviral agents, anti-fungal agents), anti-inflammatory agents, anti-pyretic agents, and pain-relieving agents. Pharmaceutical agents include small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a chelator of a metal ion or metal atom. In certain embodiments, the additional pharmaceutical agent is a chelator of a divalent metal ion (e.g., Mg(II), Ca(II), Sr(II), Mn(II), Fe(II), Co(II), Ni(II), Cu(II), or Zn(II)). In certain embodiments, the additional pharmaceutical agent is a chelator of Cu(II), Mg(II), or Fe(II). In certain embodiments, the additional pharmaceutical agent is di sodium 4, 5-dihydroxy-1,3-benzenedisulfonate (TIRON). In certain embodiments, the additional pharmaceutical agent is 2,2′-dipyridyl, desferrioxamine (DFO, DESFERAL), deferasirox (EXJADE), deferiprone (L1, FERRIPROX), FERALEX-G, CaNa₃DTPA, dexrazoxane, a phosphorothioate-oligonucleotide, desferrithiocin, or desazadesferrithiocin, or a derivative thereof. In certain embodiments, the additional pharmaceutical agent is an antibiotic. In certain embodiments, the additional pharmaceutical agent is an antibiotic effective against a microorganism described herein. In certain embodiments, the additional pharmaceutical agent is an antibiotic effective against a bacterium. In certain embodiments, the additional pharmaceutical agent is an antibiotic effective against a Gram-positive bacterium (e.g., a Staphylococcus species or Enterococcus species). In certain embodiments, the additional pharmaceutical agent is an antibiotic effective against a Gram-negative bacterium (e.g., an Acinetobacter species). In certain embodiments, the additional pharmaceutical agent is an antibiotic effective against a multidrug-resistant bacterium. In certain embodiments, the additional pharmaceutical agent is a β-lactam antibiotic. In certain embodiments, the additional pharmaceutical agent is a penicillin (e.g., a penam, such as an aminopenicillin (e.g., amoxicillin, an ampicillin (e.g., pivampicillin, hetacillin, bacampicillin, metampicillin, talampicillin), epicillin), a carboxypenicillin (e.g., a carbenicillin (e.g., carindacillin), ticarcillin, temocillin), a ureidopenicillin (e.g., azlocillin, piperacillin, mezlocillin), a mecillinam (e.g., pivmecillinam), sulbenicillin, benzylpenicillin, clometocillin, benzathine benzylpenicillin, procaine benzylpenicillin, azidocillin, penamecillin, phenoxymethylpenicillin, propicillin, benzathine phenoxymethylpenicillin, pheneticillin, a cloxacillin (e.g., dicloxacillin, flucloxacillin), oxacillin, methicillin, nafcillin), a penem (e.g., faropenem), a carbapenem (e.g., biapenem, ertapenem, an antipseudomonal (e.g., doripenem, imipenem, meropenem), panipenem), a cephalosporin (e.g., a cephem, such as cefazolin, cefacetrile, cefadroxil, cefalexin, cefaloglycin, cefalonium, cefaloridine, cefalotin, cefapirin, cefatrizine, cefazedone, cefazaflur, cefradine, cefroxadine, ceftezole, cefaclor, cefamandole, cefminox, cefonicid, ceforanide, cefotiam, cefprozil, cefbuperazone, cefuroxime, cefuzonam, a cephamycin (e.g, cefoxitin, cefotetan, cefmetazole), a carbacephem (e.g., loracarbef), cefixime, ceftriaxone, an antipseudomonal (e.g., ceftazidime, cefoperazone), cefcapene, cefdaloxime, cefdinir, cefditoren, cefetamet, cefmenoxime, cefodizime, cefotaxime, cefpimizole, cefpiramide, cefpodoxime, cefsulodin, cefteram, ceftibuten, ceftiolene, ceftizoxime, an oxacephem (e.g., flomoxef, latamoxef), cefepime, cefozopran, cefpirome, cefquinome, ceftobiprole, ceftaroline fosamil, ceftiofur, cefquinome, cefovecin), a monobactam (e.g., aztreonam, tigemonam, carumonam, nocardicin A), an aminoglycoside (e.g., amikacin, gentamicin, kanamycin, neomycin, netilmicin, tobramycin, paromomycin, spectinomycin), an ansamycin (e.g., geldanamycin, herbimycin, rifaximin), a glycopeptide (e.g., teicoplanin, vancomycin, telavancin), a lincosamide (e.g., clindamycin, lincomycin), a lipopeptide (e.g., daptomycin), a macrolide (e.g., azithromycin, clarithromycin, dirithromycin, erythromycin, roxithromycin, troleandomycin, telithromycin, spiramycin), a nitrofuran (e.g., furazolidone, nitrofurantoin), an oxazolidonone (e.g., linezolid, posizolid, radezolid, torezolid), a polypeptide (e.g., bacitracin, colistin, polymyxin B), a quinolone (e.g., ciprofloxacin, enoxacin, gatifloxacin, levofloxacin, lomefloxacin, moxifloxacin, nalidixic acid, norfloxacin, ofloxacin, trovafloxacin, grepafloxacin, sparfloxacin, temafloxacin), a sulfonamide (e.g., mafenide, sulfacetamide, sulfadiazine, silver sulfadiazine, sulfadimethoxine, sulfamethizole, sulfamethoxazole, sulfanilimide, sulfasalazine, sulfisoxazole, trimethoprim, sulfonamidochrysoidine), a tetracycline (e.g., demeclocycline, doxycycline, minocycline, oxytetracycline, tetracycline), clofazimine, dapsone, capreomycin, cycloserine, ethambutol, ethionamide, isoniazid, pyrazinamide, rifampin, rifabutin, rifapentine, streptomycin, arsphenamine, chloramphenicol, fosfomycin, fusidic acid, metronidazole, mupirocin, platensimycin, quinupristin/dalfopristin, thiamphenicol, tigecycline, tinidazole, or trimethoprim. In certain embodiments, the additional pharmaceutical agent is an antiviral agent. In certain embodiments, the additional pharmaceutical agent is (−)-Oseltamivir, β-D-ribofuranose, 1-acetate 2,3,5-tribenzoate, 1-Docosanol, 2-Amino-6-chloropurine, 5-Iodo-2′-deoxyuridine, 6-Chloropurine, Abacavir sulfate, Abacavir-epivir mixt., Acyclovir, Acyclovir sodium, Adefovir dipivoxil, Amantadine (e.g., Amantadine hydrochloride), Amantadine hydrochloride, anti-HIV agent (e.g., Abacavir, Amprenavir, Atazanavir, Azidothymidine, Bryostatin (e.g., Bryostatin 1, Bryostatin 10, Bryostatin 11, Bryostatin 12, Bryostatin 13, Bryostatin 14, Bryostatin 15, Bryostatin 16, Bryostatin 17, Bryostatin 18, Bryostatin 19, Bryostatin 2, Bryostatin 20, Bryostatin 3, Bryostatin 4, Bryostatin 5, Bryostatin 6, Bryostatin 7, Bryostatin 8, Bryostatin 9), Dideoxycytidine, Dideoxyinosine, Efavirenz, Indinavir, Lamivudine, Lopinavir, Nevirapine, Ritonavir, Saquinavir, Stavudine, Tenofovir), Azauridine, ombivir, Deoxynojirimycin, Docosanol, Fomivirsen sodium, Foscarnet, Ganciclovir, Integrase inhibitors (e.g., 5CITEP, Chloropeptin I, Complestatin, Dolutegravir, Elvitegravir, L 708906, L 731988, MK 2048, Raltegravir, Raltegravir potassium), MK 5172, MK 8742, Palivizumab, Pegylated interferon alfa-2b, Phosphonoacetic acid, Ribavirin, Simeprevir, Sofosbuvir, Tubercidin, Vidarabine, or virus entry inhibitor (e.g., Enfuvirtide, Maraviroc). In certain embodiments, the additional pharmaceutical agent is a fungicide. In certain embodiments, the additional pharmaceutical agent is (−)-Fumagillin, (−)-Metalaxyl, 1,2, 5-Fluorocytosine, Acrisorcin, Anilazine, Antifouling agent, Azoxystrobin, Benomyl, Bordeaux mixture, Captan, Carbendazim, Caspofungin acetate, Chlorothalonil, Clotrimazole, Dichlofluanid, Dinocap, Dodine, Fenhexamid, Fenpropimorph, Ferbam, Fluconazole, Fosetyl Al, Griseofulvin, Guanidine (e.g., Agmatine, Amiloride hydrochloride, Biguanide (e.g., Imidodicarbonimidic diamide, N,N-dimethyl-,hydrochloride (1:1) (e.g., Metformin hydrochloride), Metformin), Cimetidine, Guanethidine, Guanfacine, Guanidine, Guanidinium, Methylguanidine, Sulfaguanidine), Iprobenfos, Iprodione, Isoprothiolane, Itraconazole, Ketoconazole, Mancozeb, Metalaxyl, Metiram, Miconazole, Natamycin, Nystatin, Oxycarboxine, Pentachloronitrobenzene, Prochloraz, Procymidone, Propiconazole, Pyrazophos, Reduced viscotoxin A3, Salicylanilide, Tebuconazole, Terbinafine, Thiabendazole, Thiophanate, Thiophanate methyl, Triadimefon, Vinclozolin, or Voriconazole. In certain embodiments, the additional pharmaceutical agent is a protozoacide. In certain embodiments, the additional pharmaceutical agent is Amebicide, Antimalarial (e.g., Artemisinin, Chloroquine (e.g., Chloroquine phosphate), Mefloquine, Sulfadoxine), Coccidiostat, Leishmanicide, Trichomonacide, or Trypanosomicide (e.g., Eflornithine). In certain embodiments, the additional pharmaceutical agent is a parasiticide. In certain embodiments, the additional pharmaceutical agent is antihelmintic (e.g., Abamectin, Dimethylformocarb othialdine, Niclosamide, Schistosomicide), protozoacide (e.g., Amebicide, antimalarial (e.g., Artemisinin, chloroquine (e.g., chloroquine phosphate), Mefloquine, Sulfadoxine), coccidiostat, leishmanicide, trichomonacide, or trypanosomicide (e.g., Eflornithine)).

In certain embodiments, the pharmaceutical composition is substantially free (e.g., at least 70% free, at least 80% free, at least 90% free, at least 95% free, at least 99% free, or at least 99.9% free) of a metal ion or metal atom. In certain embodiments, the pharmaceutical composition is substantially free of a divalent metal ion (e.g., Mg(II), Ca(II), Sr(II), Mn(II), Fe(II), Co(II), Ni(II), Cu(II), or Zn(II)). In certain embodiments, the pharmaceutical composition is substantially free of Cu(II), Mg(II), or Fe(II).

Also encompassed by the invention are kits (e.g., pharmaceutical packs). The kits provided may comprise a compound or composition (e.g., pharmaceutical composition) of the invention and a container (e.g., a vial, ampule, bottle, syringe, dispenser package, tube, inhaler, and/or other suitable container). In some embodiments, a kit of the invention further includes a second container comprising an excipient (e.g., pharmaceutically acceptable excipient) for dilution or suspension of an inventive compound or composition. In some embodiments, the compound or composition of the invention provided in a first container and a second container are combined to form one unit dosage form.

In one aspect, the present invention provides kits including a first container comprising a compound or composition of the invention. In certain embodiments, a kit of the invention includes a first container comprising a compound of Formula (I′) (e.g., Formula (I)), (II), or (III), or a pharmaceutically acceptable salt thereof, or a composition thereof.

In certain embodiments, the kits are useful in treating a microbial infection in a subject in need thereof. In certain embodiments, the kits are useful in preventing a microbial infection in a subject in need thereof. In certain embodiments, the microbial infection is a bacterial infection. In certain embodiments, the bacterial infection is an infection caused by a Gram-positive bacterium. In certain embodiments, the bacterial infection is an infection caused by a Gram-negative bacterium. In certain embodiments, the microbial infection is a mycobacterial infection. In certain embodiments, the kits are useful in inhibiting the growth of a microorganism. In certain embodiments, the kits are useful in inhibiting the reproduction of a microorganism. In certain embodiments, the kits are useful in killing a microorganism. In certain embodiments, the kits are useful in inhibiting the formation and/or growth of a biofilm. In certain embodiments, the kits are useful in reducing or removing a biofilm. In certain embodiments, the kits are useful in disinfecting a surface. In certain embodiments, the kits are useful for screening a library of compounds to identify a compound that is useful in the methods of the invention. In certain embodiments, the kit further includes instructions for using the compound or pharmaceutical composition included in the kit (e.g., for administering to a subject in need of treatment of a microbial infection a compound or pharmaceutical composition of the invention, for contacting a microorganism with a compound or pharmaceutical composition of the invention, or for contacting a biofilm with a compound or pharmaceutical composition of the invention). The kits may also include information as required by a regulatory agency such as the U.S. Food and Drug Administration (FDA). In certain embodiments, the information included in the kits is prescribing information. In certain embodiments, the kits and instructions provide for treating a microbial infection in a subject in need thereof. In certain embodiments, the kits and instructions provide for preventing a microbial infection in a subject in need thereof. In certain embodiments, the kits and instructions provide for inhibiting the growth of a microorganism. In certain embodiments, the kits and instructions provide for inhibiting the reproduction of a microorganism. In certain embodiments, the kits and instructions provide for killing a microorganism. In certain embodiments, the kits and instructions provide for inhibiting the formation and/or growth of a biofilm. In certain embodiments, the kits and instructions provide for reducing or removing a biofilm. In certain embodiments, the kits and instructions provide for disinfecting a surface. In certain embodiments, the kits and instructions provide for screening a library of compounds to identify a compound that is useful in the methods of the invention. The kit of the invention may include one or more additional agents described herein (e.g., additional pharmaceutical agents) as a separate composition.

### Methods of Treatment and Uses

Current antibiotics operate primarily through growth-dependent mechanisms and effectively target rapidly-dividing bacteria; however, non-replicative bacteria (e.g., dormant persister cells, bacterial biofilms) display high levels of antibiotic tolerance contributing to persistent and recurring bacterial infection.¹⁻³ In recent years, the knowledge of bacterial biofilms (surface-attached bacterial communities with altered physiologies, gene expression profiles and growth-rates)^{4,5} and persister cells⁶ has grown considerably, yet the ability to target persistent bacterial phenotypes remains an unmet challenge. In order to target and eradicate non-replicating bacteria, innovative strategies to identify antibacterial agents that operate through growth-independent mechanisms may be employed.

Although there has been much interest in identifying non-growth altering biofilm inhibitors and dispersal agents over the past two decades,⁷ few classes of biofilm-eradicating agents are known. Biofilm-eradicating agents typically operate through the disruption of bacterial membranes (e.g., antimicrobial peptides,^{8,9} quaternary ammonium cations/QACs¹⁰). Although these compounds are indeed valuable, new biofilm-eradicating agents with complementary modes of action are of great importance and have multiple therapeutic applications to address persistent bacterial infections.

Considering the marine environment as an extensive source of microbial diversity and new antibacterial agents,¹¹ it stands to reason that such sources are fertile grounds for the discovery of biofilm-eradicating agents. Despite marine sources being largely unexplored, several classes of chemically diverse quorum sensing (the bacterial signaling process that controls biofilm formation and maintenance) modulators¹²⁻¹⁵ and biofilm inhibitors/dispersal agents have been identified from marine organisms.^{7b,16}

It has recently been discovered that HP 202, a synthetic analogue of marine phenazine antibiotic 201, displayed biofilm eradication activities against MRSA with a minimum biofilm eradication concentration (MBEC) of 150±50 μM,¹⁷ which is on pace with the most potent eradicating agents reported.¹⁰

The mechanism of 202 was investigated, since 2-bromo-1-hydroxyphenazine 201 belongs to the family of redox-active phenazine antibiotics produced by Pseudomonas and Streptomyces bacteria.¹⁸ In addition, one of the goals was to synthesize more efficient biofilm-eradicating agents, target persister cells in non-biofilm cultures and evaluate HP analogues against the slow-growing pathogen Mycobacterium tuberculosis (MtB). Compounds that can effectively eradicate biofilms, persister cells and MtB are promising agents to address problems associated with bacterial persistence.

The present invention also provides methods for treating a microbial infection (e.g., bacterial infection or mycobacterial infection) in a subject in need thereof. In certain embodiments, the microbial infection is treated by the inventive methods. In certain embodiments, the present invention further provides methods for preventing a microbial infection (e.g., bacterial infection or mycobacterial infection) in a subject in need thereof. In certain embodiments, the microbial infection is prevented by the inventive methods.

In certain embodiments, the subject described herein is an animal. In certain embodiments, the subject is a non-human animal. In certain embodiments, the subject is a mammal. In certain embodiments, the subject is a human. In certain embodiments, the subject is a human with cystic fibrosis. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent, dog, or non-human primate. In certain embodiments, the subject is a non-human transgenic animal, such as a transgenic mouse or transgenic pig.

In certain embodiments, the methods of the invention include administering to a subject in need thereof an effective amount of a compound or pharmaceutical composition of the invention. In certain embodiments, the methods of the invention include administering to a subject in need thereof an effective amount of a compound of Formula (I′) (e.g., Formula (I)), (II), or (III), or a pharmaceutically acceptable salt thereof, or a pharmaceutical composition thereof. In certain embodiments, the methods of the invention include administering to a subject in need thereof a therapeutically effective amount of a compound of the invention, or a pharmaceutical composition thereof. In certain embodiments, the methods of the invention include administering to a subject in need thereof a prophylactically effective amount of a compound of the invention, or a pharmaceutical composition thereof.

In certain embodiments, the microbial infection that is treated and/or prevented by the inventive methods or using the inventive compounds or pharmaceutical compositions thereof is caused by a multidrug-resistant microorganism and/or a microorganism resistant to methicillin, penicillin, ciprofloxacin, rifampin, vancomycin, daptomycin, linezolid, an antibiotic described herein, or a combination thereof. In certain embodiments, the microbial infection is a microbial respiratory tract infection. In certain embodiments, the microbial infection is microbial pneumonia. In certain embodiments, the microbial infection is microbial sinusitis. In certain embodiments, the microbial infection is tuberculosis (TB). In certain embodiments, the microbial infection is microbial Crohn's disease, paratuberculosis, Buruli ulcer, leprosy, or aquarium granuloma. In certain embodiments, the microbial infection is a microbial gastrointestinal tract infection. In certain embodiments, the microbial infection is microbial diarrhea. In certain embodiments, the microbial infection is a microbial urogenital tract infection. In certain embodiments, the microbial infection is a microbial bloodstream infection. In certain embodiments, the microbial infection is microbial hemolytic uremic syndrome. In certain embodiments, the microbial infection is microbial endocarditis. In certain embodiments, the microbial infection is a microbial ear infection. In certain embodiments, the microbial infection is a microbial skin infection (e.g., microbial acne vulgaris). In certain embodiments, the microbial infection is a microbial oral infection. In certain embodiments, the microbial infection is a microbial dental infection. In certain embodiments, the microbial infection is gingivitis. In certain embodiments, the microbial infection is dental plaque caused by a microorganism. In certain embodiments, the microbial infection is microbial meningitis. In certain embodiments, the microbial infection is a microbial wound or surgical site infection. In certain embodiments, the microbial infection is a microbial burn wound infection. In certain embodiments, the microbial infection is a microbial infection associated with cystic fibrosis. In certain embodiments, the microbial infection is a microbial infection associated with an implanted device. In certain embodiments, the microbial infection is a microbial infection associated with a dental implant. In certain embodiments, the microbial infection is a microbial infection associated with a catheter. In certain embodiments, the microbial infection is a microbial infection associated with a heart valve. In certain embodiments, the microbial infection is a microbial infection associated with an intrauterine device. In certain embodiments, the microbial infection is a microbial infection associated with a joint prosthesis. In certain embodiments, the microbial infection is a bacterial infection. In certain embodiments, the bacterial infection is caused by a Gram-positive bacterium (e.g., a Gram-positive bacterium described herein). In certain embodiments, the bacterial infection is caused by a Gram-negative bacterium (e.g., a Gram-negative bacterium described herein). In certain embodiments, the bacterial infection is caused by a multidrug-resistant bacterium. In certain embodiments, the bacterial infection is caused by a strain of Staphylococcus aureus. In certain embodiments, the bacterial infection is a methicillin-resistant Staphylococcus aureus (MRSA)-related infection. In certain embodiments, the bacterial infection is caused by a strain of Staphylococcus epidermidis (e.g., MRSE). In certain embodiments, the bacterial infection is an MRSE-related infection. In certain embodiments, the bacterial infection is caused by a strain ofEnterococcusfaecium. In certain embodiments, the bacterial infection is caused by Acinetobacter baumannii (A. baumannii). In certain embodiments, the microbial infection is a mycobacterial infection. In certain embodiments, the microbial infection is caused by a mycobacterium (e.g., a strain of Mycobacterium tuberculosis). In certain embodiments, the microbial infection is caused by an archaeon. In certain embodiments, the microbial infection is caused by a protist. In certain embodiments, the microbial infection is caused by a protozoon. In certain embodiments, the microbial infection is caused by an alga. In certain embodiments, the microbial infection is caused by a fungus. In certain embodiments, the microbial infection is caused by yeast. In certain embodiments, the microbial infection is caused by a mold. In certain embodiments, the microbial infection is caused by a parasite. In certain embodiments, the microbial infection is a microbial infection associated with a biofilm.

Another aspect of the present invention relates to methods of inhibiting the growth of a microorganism using a compound of the invention, or a pharmaceutical composition thereof. In certain embodiments, an inventive method selectively inhibits the growth of a first microorganism (e.g., a microorganism described herein), compared to the inhibition of the growth of a host cell or a second microorganism. In certain embodiments, the growth of a microorganism is inhibited by the inventive methods. In certain embodiments, the growth of a first microorganism is selectively inhibited by the inventive methods, compared to the inhibition of the growth of a host cell or a second microorganism.

Another aspect of the present invention relates to methods of inhibiting the reproduction of a microorganism using a compound of the invention, or a pharmaceutical composition thereof. In certain embodiments, an inventive method selectively inhibits the reproduction of a first microorganism (e.g., a microorganism described herein), compared to the inhibition of the reproduction of a host cell or a second microorganism. In certain embodiments, the reproduction of a microorganism is inhibited by the inventive methods. In certain embodiments, the reproduction of a first microorganism is selectively inhibited by the inventive methods, compared to the inhibition of the reproduction of a host cell or a second microorganism.

Another aspect of the present invention relates to methods of inhibiting the viability of a microorganism using a compound of the invention, or a pharmaceutical composition thereof. In certain embodiments, an inventive method selectively inhibits the viability of a first microorganism (e.g., a microorganism described herein), compared to the inhibition of the viability of a host cell or a second microorganism. In certain embodiments, the viability of a microorganism is inhibited by the inventive methods. In certain embodiments, the viability of a first microorganism is selectively inhibited by the inventive methods, compared to the inhibition of the viability of a host cell or a second microorganism.

Another aspect of the present invention relates to methods of killing a microorganism using a compound of the invention, or a pharmaceutical composition thereof. In certain embodiments, an inventive method selectively kills a first microorganism (e.g., a microorganism described herein), compared to the killing of a host cell or a second microorganism. In certain embodiments, a microorganism is killed by the inventive methods. In certain embodiments, a first microorganism is selectively killed by the inventive methods, compared to the killing of a host cell or a second microorganism.

In certain embodiments, the methods of the invention include contacting a microorganism with an effective amount of a compound or pharmaceutical composition of the invention. In certain embodiments, the methods of the invention include contacting a microorganism with an effective amount of a compound of Formula (I′) (e.g., Formula (I)), (II), or (III), or a pharmaceutically acceptable salt thereof, or a pharmaceutical composition thereof. In certain embodiments, the methods of the invention include contacting a microorganism with a therapeutically effective amount of a compound of the invention, or a pharmaceutical composition thereof. In certain embodiments, the methods of the invention include contacting a microorganism with a prophylactically effective amount of a compound of the invention, or a pharmaceutical composition thereof.

In a growth process of a microorganism (e.g., a bacterium), the microorganism may secrete viscous substances to form a biofilm. A biofilm is typically formed on a living or non-living, solid or liquid surface. In certain embodiments, a biofilm is formed on the surface of a biological sample (e.g., a tooth, oral soft tissue, middle ear, gastrointestinal tract, urogenital tract, respiratory tract, or eye). In certain embodiments, a biofilm is formed on the surface of an implanted device (e.g., a dental implant, catheter, heart valve, intrauterine device, or joint prosthesis). In certain embodiments, the biofilm is in vitro. In certain embodiments, the biofilm is in vivo. In certain embodiments, the biofilm described herein comprises a microorganism. In certain embodiments, the biofilm comprises a microorganism (e.g., bacterium). In certain embodiments, the biofilm comprises a strain of Staphylococcus aureus (e.g., a methicillin-resistant strain of Staphylococcus aureus). In certain embodiments, the biofilm comprises a strain of Staphylococcus epidermidis (e.g., a strain of MRSE). Free-floating microorganisms may accumulate on a surface, and the resulting biofilm may grow. In a biofilm, the concentration of microorganisms may be high and/or the resistance of the microorganisms in the biofilm to antimicrobial agents may be high. Antimicrobials may be inactivated or fail to penetrate into the biofilm. Therefore, microbial infections associated with a biofilm (e.g., microbial infections caused by a biofilm) are typically more difficult to treat than microbial infections not associated with a biofilm.

Another aspect of the present invention relates to methods of inhibiting the formation of a biofilm using a compound of the invention, or a pharmaceutical composition thereof. In certain embodiments, the formation of a biofilm is inhibited by the inventive methods.

Another aspect of the present invention relates to methods of inhibiting the growth of a biofilm using a compound of the invention, or a pharmaceutical composition thereof. In certain embodiments, the growth of a biofilm is inhibited by the inventive methods.

Another aspect of the present invention relates to methods of reducing a biofilm using a compound of the invention, or a pharmaceutical composition thereof. In certain embodiments, a biofilm is reduced by the inventive methods, e.g., reduced by at least 10%, at least 20%, at least 30%, at least 50%, at least 70%, at least 90%, at least 99%, at least 99.9%, or at least 99.99%, in terms of the volume of the biofilm. In certain embodiments, a biofilm is reduced by the inventive methods by not more than 10%, not more than 20%, not more than 30%, not more than 50%, not more than 70%, not more than 90%, not more than 99%, not more than 99.9%, or not more than 99.99%, in terms of the volume of the biofilm. In certain embodiments, a biofilm is reduced by the inventive methods by at least 10%, at least 20%, at least 30%, at least 50%, at least 70%, at least 90%, at least 99%, at least 99.9%, or at least 99.99%, in terms of the number of microorganisms (e.g., bacteria) in the biofilm. In certain embodiments, a biofilm is reduced by the inventive methods by not more than 10%, not more than 20%, not more than 30%, not more than 50%, not more than 70%, not more than 90%, not more than 99%, not more than 99.9%, or not more than 99.99%, in terms of the number of microorganisms (e.g., bacteria) in the biofilm.

Another aspect of the present invention relates to methods of removing a biofilm (e.g., eradicating a biofilm (e.g., reducing the volume of the biofilm by at least 99% and/or killing essentially all (e.g., at least 99%) of the microorganisms (e.g., bacteria) in the biofilm)) using a compound of the invention, or a pharmaceutical composition thereof. In certain embodiments, a biofilm is removed by the inventive methods. In certain embodiments, a biofilm reduced or removed by a method of the invention does not regrow one day, two days, four days, one week, two weeks, three weeks, or one month subsequent to the biofilm being subject to the method.

In certain embodiments, the methods of the invention include contacting a biofilm with an effective amount of a compound or pharmaceutical composition of the invention. In certain embodiments, the methods of the invention include contacting a biofilm with an effective amount of a compound of Formula (I′) (e.g., Formula (I)), (II), or (III), or a pharmaceutically acceptable salt thereof, or a pharmaceutical composition thereof. In certain embodiments, the methods of the invention include contacting a biofilm with a therapeutically effective amount of a compound of the invention, or a pharmaceutical composition thereof. In certain embodiments, the methods of the invention include contacting a biofilm with a prophylactically effective amount of a compound of the invention, or a pharmaceutical composition thereof.

Another aspect of the present invention relates to methods of disinfecting a surface, the methods including contacting the surface with an effective amount of a compound or composition (e.g., pharmaceutical composition) of the invention. In certain embodiments, the number of viable microorganisms on the surface is reduced after the surface is contacted with the compound or composition. In certain embodiments, the surface is a biological surface, such as skin (e.g., skin of: the hands, feet, arms, legs, face, neck, torso, or cavity (e.g., oral cavity)) of a subject. In certain embodiments, the surface is a non-biological surface (e.g., a surface in a household, industrial, or medical setting, such as a surface of: a kitchen, bathroom, table top, floor, wall, window, utensil, cutlery, crockery, or medical device). A non-biological surface may be a surface of a solid material, such as plastic, wood, bamboo, metal, ceramic, glass, concrete, stone, paper, fabric, or a combination thereof. A non-biological surface may be painted or non-painted, or coated or non-coated. In certain embodiments, the amount of the compound or composition is effective for killing at least 80%, at least 90%, at least 95%, at least 99%, at least 99.9%, or at least 99.99% of the microorganisms on the surface.

In certain embodiments, the microorganism described herein is a bacterium. In certain embodiments, the microorganism is multidrug-resistant. In certain embodiments, the microorganism is resistant to methicillin, penicillin, ciprofloxacin, rifampin, vancomycin, daptomycin, linezolid, or a combination thereof. In certain embodiments, the microorganism is associated with a biofilm (e.g., present in and/or on a biofilm, able to form a biofilm, and/or able to increase the size of a biofilm). In certain embodiments, the bacterium is a Gram-positive bacterium. In certain embodiments, the bacterium is a multidrug-resistant bacterium. In certain embodiments, the bacterium is a Staphylococcus species. In certain embodiments, the bacterium is a Staphylococcus aureus (S. aureus) strain (e.g., ATCC 25923). In certain embodiments, the bacterium is methicillin-resistant Staphylococcus aureus (MRSA). In certain embodiments, the bacterium is the methicillin-resistant Staphylococcus aureus clinical isolate (MRSA-2, a clinical isolate from a patient treated at Shands Hospital; obtained from the Emerging Pathogens Institute at the University of Florida), such as the methicillin-resistant Staphylococcus aureus clinical isolate reported in Abouelhassan et al., Bioorg. Med. Chem. Lett., 2014, 24, 5076. In certain embodiments, the bacterium is a Staphylococcus epidermidis (S. epidermidis) strain (e.g., ATCC 12228 or ATCC 35984). In certain embodiments, the bacterium is an MRSE strain. In certain embodiments, the bacterium is a Staphylococcus auricularis, Staphylococcus carnosus, Staphylococcus condimenti, Staphylococcus massiliensis, Staphylococcus piscifermentans, Staphylococcus simulans, Staphylococcus capitis, Staphylococcus caprae, Staphylococcus saccharolyticus, Staphylococcus devriesei, Staphylococcus haemolyticus, Staphylococcus hominis, Staphylococcus chromogenes, Staphylococcus felis, Staphylococcus delphini, Staphylococcus hyicus, Staphylococcus intermedius, Staphylococcus lutrae, Staphylococcus microti, Staphylococcus muscae, Staphylococcus pseudintermedius, Staphylococcus rostri, Staphylococcus schleiferi, Staphylococcus lugdunensis, Staphylococcus arlettae, Staphylococcus cohnii, Staphylococcus equorum, Staphylococcus gallinarum, Staphylococcus kloosii, Staphylococcus leei, Staphylococcus nepalensis, Staphylococcus saprophyticus, Staphylococcus succinus, Staphylococcus xylosus, Staphylococcus fleurettii, Staphylococcus lentus, Staphylococcus sciuri, Staphylococcus stepanovicii, Staphylococcus vitulinus, Staphylococcus simulans, Staphylococcus pasteuri, or Staphylococcus warneri strain. In certain embodiments, the bacterium is a Streptococcus species. In certain embodiments, the bacterium is a Streptococcus agalactiae, Streptococcus anginosus, Streptococcus bovis, Streptococcus canis, Streptococcus constellatus, Streptococcus dysgalactiae, Streptococcus equinus, Streptococcus iniae, Streptococcus intermedius, Streptococcus mitis, Streptococcus mutans, Streptococcus oralis, Streptococcus parasanguinis, Streptococcus peroris, Streptococcus pneumoniae, Streptococcus pseudopneumoniae, Streptococcus pyogenes, Streptococcus ratti, Streptococcus salivarius, Streptococcus tigurinus, Streptococcus thermophilus, Streptococcus sanguinis, Streptococcus sobrinus, Streptococcus suis, Streptococcus uberis, Streptococcus vestibularis, Streptococcus viridans, or Streptococcus zooepidemicus strain. In certain embodiments, the bacterium is an Enterococcus species. In certain embodiments, the bacterium is an Enterococcus avium, Enterococcus durans, Enterococcusfaecalis, Enterococcus faecium, Enterococcus gallinarum, Enterococcus hirae, or Enterococcus solitarius strain. In certain embodiments, the bacterium is an Enterococcus faecium strain (e.g., a vancomycin-resistant strain of Enterococcus faecium (VRE); ATCC 700221). In certain embodiments, the bacterium is a Listeria species. In certain embodiments, the bacterium is a Listeria fleischmannii, Listeria grayi, Listeria innocua, Listeria ivanovii, Listeria marthii, Listeria monocytogenes, Listeria rocourtiae, Listeria seeligeri, Listeria weihenstephanensis, or Listeria welshimeri strain. In certain embodiments, the bacterium is a Clostridium species. In certain embodiments, the bacterium is a Clostridium acetobutylicum, Clostridium argentinense, Clostridium aerotolerans, Clostridium baratii, Clostridium beijerinckii, Clostridium bifermentans, Clostridium botulinum, Clostridium butyricum, Clostridium cadaveris, Clostridium cellulolyticum, Clostridium chauvoei, Clostridium clostridioforme, Clostridium colicanis, Clostridium difficile, Clostridium estertheticum, Clostridiumfallax, Clostridium feseri, Clostridium formicaceticum, Clostridium histolyticum, Clostridium innocuum, Clostridium kluyveri, Clostridium ljungdahlii, Clostridium lavalense, Clostridium leptum, Clostridium novyi, Clostridium oedematiens, Clostridium paraputrificum, Clostridium perfringens (Alias: Clostridium welchii), Clostridium phytofermentans, Clostridium piliforme, Clostridium ragsdalei, Clostridium ramosum, Clostridium scatologenes, Clostridium septicum, Clostridium sordellii, Clostridium sporogenes, Clostridium sticklandii, Clostridium tertium, Clostridium tetani, Clostridium thermocellum, Clostridium thermosaccharolyticum, or Clostridium tyrobutyricum strain. In certain embodiments, the bacterium is a Gram-negative bacterium. In certain embodiments, the bacterium is a bacterium described herein, provided that the bacterium is not a Gram-negative bacterium. In certain embodiments, the Gram-negative bacterium is an Escherichia species. In certain embodiments, the Gram-negative bacterium is an Escherichia coli (E. coli) strain (e.g., ATCC 33475, K-12, CFT073, ATCC 43895). In certain embodiments, the Gram-negative bacterium is an Escherichia albertii strain, Escherichia blattae strain, Escherichia fergusonii strain, Escherichia hermannii strain, or Escherichia vulneris strain. In certain embodiments, the Gram-negative bacterium is a Pseudomonas species. In certain embodiments, the Gram-negative bacterium is a Pseudomonas aeruginosa strain. In certain embodiments, the Gram-negative bacterium is a Pseudomonas alcaligenes strain, Pseudomonas anguilliseptica strain, Pseudomonas argentinensis strain, Pseudomonas borbori strain, Pseudomonas citronellolis strain, Pseudomonas flavescens strain, Pseudomonas mendocina strain, Pseudomonas nitroreducens strain, Pseudomonas oleovorans strain, Pseudomonas pseudoalcaligenes strain, Pseudomonas resinovorans strain, Pseudomonas straminea strain, Pseudomonas chlororaphis strain, Pseudomonas fluorescens strain, Pseudomonas pertucinogena strain, Pseudomonas putida strain, Pseudomonas stutzeri strain, or Pseudomonas syringae strain. In certain embodiments, the Gram-negative bacterium is a Klebsiella species. In certain embodiments, the Gram-negative bacterium is a Klebsiella granulomatis strain, Klebsiella oxytoca strain, Klebsiella pneumoniae strain, Klebsiella terrigena strain, or Klebsiella planticola strain. In certain embodiments, the Gram-negative bacterium is a strain ofKlebsiella pneumoniae (K. pneumoniae). In certain embodiments, the Gram-negative bacterium is a Salmonella species. In certain embodiments, the Gram-negative bacterium is a Salmonella bongori strain or Salmonella enterica strain, e.g., Salmonella typhi. In certain embodiments, the Gram-negative bacterium is an Acinetobacter species. In certain embodiments, the Gram-negative bacterium is an Acinetobacter baumannii strain. In certain embodiments, the Gram-negative bacterium is an Acinetobacter baylyi strain, Acinetobacter bouvetii strain, Acinetobacter calcoaceticus strain, Acinetobacter gerneri strain, Acinetobacter grimontii strain, Acinetobacter haemolyticus strain, Acinetobacter johnsonii strain, Acinetobacter junii strain, Acinetobacter lwoffii strain, Acinetobacter parvus strain, Acinetobacter pittii strain, Acinetobacter radioresistens strain, Acinetobacter schindleri strain, Acinetobacter tandoii strain, Acinetobacter tjernbergiae strain, Acinetobacter towneri strain, Acinetobacter ursingii strain, or Acinetobacter gyllenbergii strain. In certain embodiments, the microorganism is a mycobacterium. In certain embodiments, the microorganism is a strain of Mycobacterium tuberculosis. In certain embodiments, the microorganism is a strain of: Mycobacterium bovis, Mycobacterium bovis BCG, Mycobacterium africanum, Mycobacterium canetti, Mycobacterium caprae, Mycobacterium microti, Mycobacterium Pinnipedii, Mycobacterium avium, Mycobacterium avium paratuberculosis, Mycobacterium avium silvaticum, Mycobacterium avium hominissuis, Mycobacterium colombiense, Mycobacterium indicus pranii, Mycobacterium gastri, Mycobacterium kansasii, Mycobacterium hiberniae, Mycobacterium nonchromogenicum, Mycobacterium terrae, Mycobacterium triviale, Mycobacterium ulcerans, Mycobacterium pseudoshottsii, Mycobacterium shottsii, Mycobacterium triplex, Mycobacterium genavense, Mycobacterium florentinum, Mycobacterium lentiflavum, Mycobacterium palustre, Mycobacterium kubicae, Mycobacterium parascrofulaceum, Mycobacterium heidelbergense, Mycobacterium interjectum, Mycobacterium simiae, Mycobacterium bohemicum, Mycobacterium botniense, Mycobacterium branderi, Mycobacterium celatum, Mycobacterium chimaera, Mycobacterium conspicuum, Mycobacterium cookii, Mycobacterium doricum, Mycobacterium farcinogenes, Mycobacterium haemophilum, Mycobacterium heckeshornense, Mycobacterium intracellulare, Mycobacterium lacus, Mycobacterium leprae, Mycobacterium lepraemurium, Mycobacterium lepromatosis, Mycobacterium malmoense, Mycobacterium marinum, Mycobacterium monacense, Mycobacterium montefiorense, Mycobacterium murale, Mycobacterium nebraskense, Mycobacterium saskatchewanense, Mycobacterium scrofulaceum, Mycobacterium shimoidei, Mycobacterium szulgai, Mycobacterium tusciae, Mycobacterium xenopi, Mycobacterium yongonense, Mycobacterium intermedium, Mycobacterium abscessus, Mycobacterium chelonae, Mycobacterium bolletii, Mycobacterium fortuitum, Mycobacterium fortuitum subsp. acetamidolyticum, Mycobacterium boenickei, Mycobacterium peregrinum, Mycobacterium porcinum, Mycobacterium senegalense, Mycobacterium septicum, Mycobacterium neworleansense, Mycobacterium houstonense, Mycobacterium mucogenicum, Mycobacterium mageritense, Mycobacterium brisbanense, Mycobacterium cosmeticum, Mycobacterium parafortuitum, Mycobacterium austroafricanum, Mycobacterium diernhoferi, Mycobacterium hodleri, Mycobacterium neoaurum, Mycobacterium frederiksbergense, Mycobacterium aurum, Mycobacterium vaccae, Mycobacterium chitae, Mycobacterium fallax, Mycobacterium confluentis, Mycobacterium flavescens, Mycobacterium madagascariense, Mycobacterium phlei, Mycobacterium smegmatis Mycobacterium goodii, Mycobacterium wolinskyi, Mycobacterium thermoresistibile, Mycobacterium gadium, Mycobacterium komossense, Mycobacterium obuense, Mycobacterium sphagni, Mycobacterium agri, Mycobacterium aichiense, Mycobacterium alvei, Mycobacterium arupense, Mycobacterium brumae, Mycobacterium canariasense, Mycobacterium chubuense, Mycobacterium conceptionense, Mycobacterium duvalii, Mycobacterium elephantis, Mycobacterium gilvum, Mycobacterium hassiacum, Mycobacterium holsaticum, Mycobacterium immunogenum, Mycobacterium massiliense, Mycobacterium moriokaense, Mycobacterium psychrotolerans, Mycobacterium pyrenivorans, Mycobacterium vanbaalenii, Mycobacterium pulveris, Mycobacterium arosiense, Mycobacterium aubagnense, Mycobacterium caprae, Mycobacterium chlorophenolicum, Mycobacterium fluoroanthenivorans, Mycobacterium kumamotonense, Mycobacterium novocastrense, Mycobacterium parmense, Mycobacterium phocaicum, Mycobacterium poriferae, Mycobacterium rhodesiae, Mycobacterium seoulense, or Mycobacterium tokaiense.

In certain embodiments, the microorganism described herein is an archaeon. In certain embodiments, the microorganism is a protist. In certain embodiments, the microorganism is a protozoon. In certain embodiments, the microorganism is an alga. In certain embodiments, the microorganism is a fungus. In certain embodiments, the microorganism is yeast. In certain embodiments, the microorganism is a mold. In certain embodiments, the microorganism is a parasite.

In certain embodiments, the microorganism described herein is in vitro. In certain embodiments, the microorganism is in vivo.

In certain embodiments, a method of the invention is an in vitro method. In certain embodiments, a method of the invention is an in vivo method.

In another aspect, the present invention provides uses of the compounds, compositions, and pharmaceutical compositions of the invention for manufacturing a medicament for treating a microbial infection (e.g., bacterial infection or mycobacterial infection).

In another aspect, the present invention provides uses of the compounds, compositions, and pharmaceutical compositions of the invention for manufacturing a medicament for preventing a microbial infection (e.g., bacterial infection or mycobacterial infection).

In another aspect, the present invention provides the compounds, compositions, and pharmaceutical compositions of the invention for use in treating a microbial infection (e.g., bacterial infection or mycobacterial infection).

In another aspect, the present invention provides the compounds, compositions, and pharmaceutical compositions of the invention for use in preventing a microbial infection (e.g., bacterial infection or mycobacterial infection).

### EXAMPLES

In order that the invention described herein may be more fully understood, the following examples are set forth. The synthetic and biological examples described in this application are offered to illustrate the compounds, pharmaceutical compositions, and methods provided herein and are not to be construed in any way as limiting their scope. A compound described herein may be referred to by using two or more different compound numbers. A compound described herein may be tested two or more times under the same or different conditions for determining a property and, therefore, may show different values of the property.

### Example 1. Synthesis of the Compounds

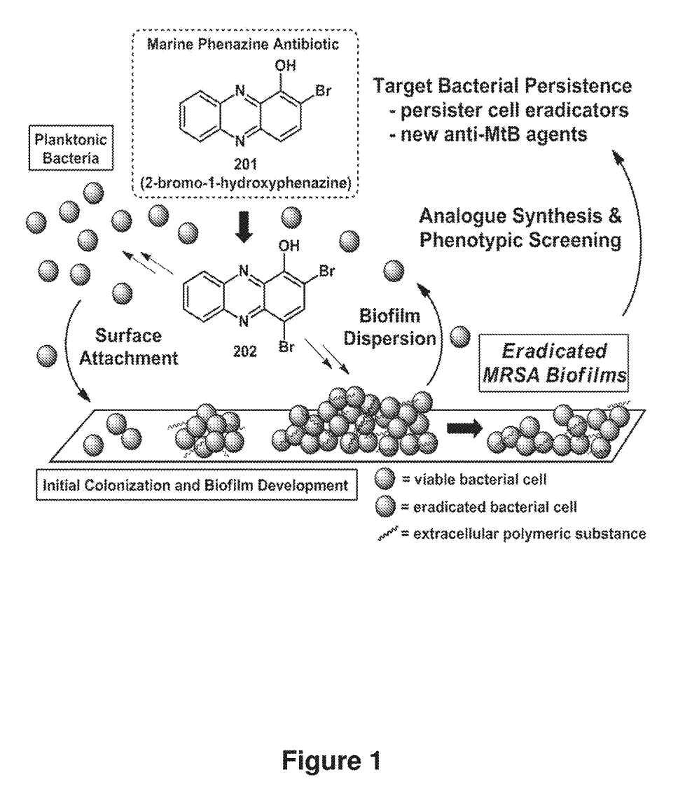

The relationships between the compound numbers referenced in any one of Examples 1 and 2 (including FIGS. 1 to 6E) and the compound structures referenced in any one of Examples 1 and 2 (including FIGS. 1 to 6E) are applicable to any one of Example 1 and 2 (including FIGS. 1 to 6E).

The compounds provided herein can be prepared from readily available starting materials using the following general methods and procedures. It will be appreciated that where typical or preferred process conditions (e.g., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

All reactions were carried out under an inert atmosphere (e.g., atmosphere of argon) unless otherwise specified. Anhydrous solvents were transferred via syringe to flame-dried glassware, which was cooled under a stream of dry argon. Anhydrous tetrahydrofuran, acetonitrile, diethyl ether, dichloromethane, toluene, and all chemical reagents for synthesis were used without further purification. Analytical thin layer chromatography (TLC) was performed using 250 Lm silica gel 60 F254 pre-coated plates (EMD Chemicals Inc.). Flash column chromatography was performed using 230-400 Mesh 60 Å silica gel (Sorbent Technologies). All melting points (MPs) were obtained, uncorrected, using a Mel-Temp capillary melting point apparatus from Laboratory Services, Inc.

NMR experiments were recorded using broadband probes on a Varian Mercury-Plus-400 spectrometer via VNMR-J software (400 MHz for ¹H and 100 MHz for ¹³C) and a Bruker Avance-III-500 spectrometer via TOPSPIN software (500 MHz for ¹H and 126 MHz for ¹³C). All spectra have been formatted and presented using MESTRENOVA (Mnova) software. Spectra were obtained in the following solvents (reference peaks also included for ¹H and ¹³C NMRs): CDCl₃ (¹H NMR: 7.26 ppm; ¹³C NMR: 77.23 ppm), d₆-DMSO (¹H NMR: 2.50 ppm; ¹³C NMR: 39.52 ppm), CD₃OD (¹H NMR: 3.31 ppm; ¹³C NMR: 49.00 ppm), d₆-benzene (¹H NMR: 7.16 ppm; ¹³C NMR: 128.06 ppm). NMR samples where the respective solvent peaks were buried in the sample signals were referenced with TMS at 0.00 ppm for ¹H NMR experiments. NMR experiments were performed at room temperature unless otherwise indicated. Chemical shift values (δ) are reported in parts per million (ppm) for all ¹H NMR and ¹³C NMR spectra. ¹H NMR multiplicities are reported as: s=singlet, d=doublet, t=triplet, q=quartet, hept=heptet, m=multiplet, and br=broad. High-resolution mass spectra were obtained for new compounds from the Mass Spectrometry Facility in the Chemistry Department at the University of Florida.

All compounds were stored as DMSO stocks at room temperature in the absence of light for several months at a time without observing any loss in biological activity. To ensure compound integrity of the DMSO stock solutions, the DMSO stocks of the compounds were not subjected to freeze-thaw cycles.

The compounds of the invention can be prepared using previously reported synthetic protocols (e.g., E. Breitmaier, J Org. Chem., 1976, 41, 2104-2108; D. L. Vivan, Nature, 1956, 178, 753; M. Conda-Sheridan et al., J. Med. Chem., 2010, 53, 8688-8699; G. W. Rewcastle et al., J. Med. Chem., 1987, 30, 843-851; international PCT application publication, WO 2015/100331, published Jul. 2, 2015; each of which is incorporated herein by reference). In one set of experiments, select compounds of the invention were prepared according to the methods shown in Schemes 1 and 2.

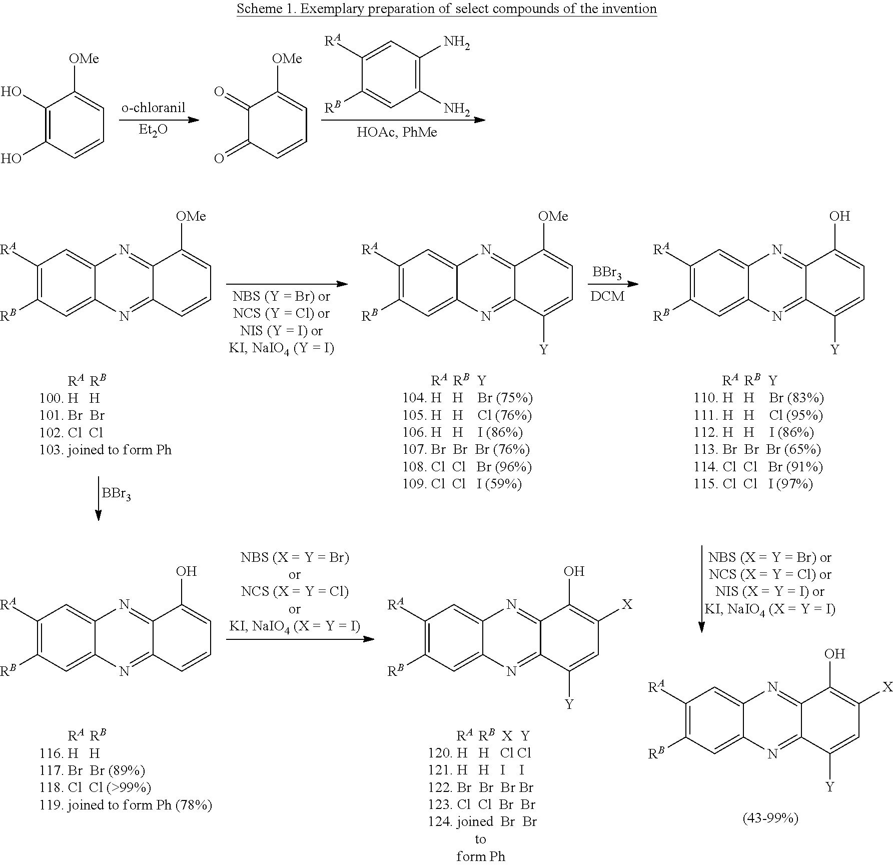

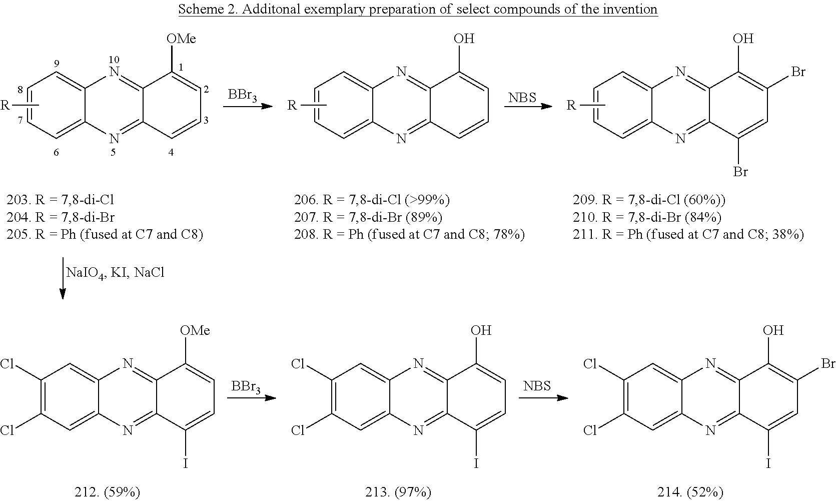

For example, a library of five HP analogues was prepared that contained various substitutions in the 7- and 8-position of the phenazine related to previous work by Cushman¹⁹ (Scheme 2). Demethylation of 1-methoxyphenazines 203-205 proceeded smoothly using boron tribromide (BBr₃) to afford 1-hydroxyphenazines 206-208 (78-99%), which were then brominated using N-bromosuccinimide (NBS) to yield 209-211 (38-84% yield). 1-Methoxyphenazine 203 was selectively iodinated at the 4-position using sodium periodate (NaIO₄)/potassium iodide (KI)/sodium chloride (NaCl) to afford 212 (59% yield), followed by demethylation using BBr₃ to give 213 (97% yield). A final bromination reaction at the 2-position of phenazine 213 afforded mixed HP 214 (52% yield).

Details of the exemplary preparations of select compounds of the invention are shown below.

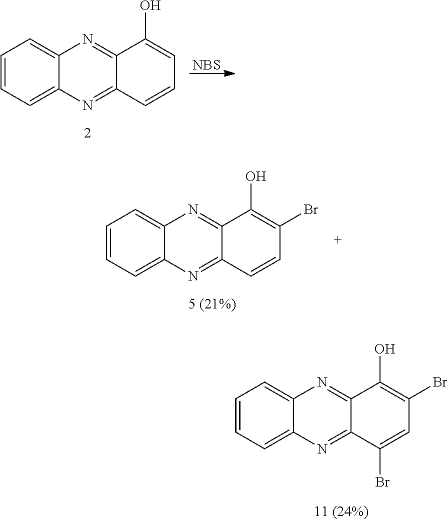

### 2-Bromophenazin-1-ol (5) and 2,4-dibromophenazin-1-ol (11)

1-Hydroxyphenazine (120 mg, 0.612 mmol) and N-bromosuccinimide (NBS, 120 mg, 0.730 mmol) were dissolved in 12 mL toluene and heated at 50° C. for 5 hours. The reaction contents were then concentrated, taken up in dichloromethane, and adsorbed onto silica for purification. Column chromatography eluting with dichloromethane furnished 35 mg (21% yield) of 2-bromophenazin-1-ol 5 as a yellow solid and 51 mg (24% yield) of 2,4-dibromophenazin-1-ol 11 as a yellow solid. These two products were optimally separated on TLC and column chromatography using 85:15 hexanes:ethyl acetate.

### 2-Bromophenazin-1-ol (5)

¹H NMR (400 MHz, CDCl₃): δ 8.52 (br s, 1H), 8.30-8.18 (m, 2H), 7.94-7.82 (m, 3H), 7.71 (d, J=9.5 Hz, 1H). ¹³C NMR (100 MHz, CDCl₃): δ 149.3, 144.3, 143.0, 141.5, 135.5, 134.5, 131.5, 131.3, 130.0, 129.3, 121.0, 103.7. HRMS (DART): m/z calc. for C₁₂H₇N₂OBr [M+H]⁺: 274.9815, found: 274.9824.

### 2,4-Dibromophenazin-1-ol (11)

¹H NMR (400 MHz, CDCl₃): δ 8.54 (br s, 1H), 8.43-8.38 (m, 1H), 8.30-8.24 (m, 2H), 7.98-7.90 (m, 2H); (400 MHz, d₆-DMSO) δ 11.58 (s, 1H), 8.44 (s, 1H), 8.40-8.31 (m, 2H), 8.11-8.03 (m, 2H). ¹³C NMR (100 MHz, CDCl₃): δ 149.3, 144.3, 141.6, 140.1, 137.4, 134.4, 132.3, 131.9, 130.4, 129.0, 113.1, 103.2. HRMS (DART): m/z calc. for C₁₂H₆N₂OBr₂ [M+H]⁺: 354.8900, found: 354.8909.

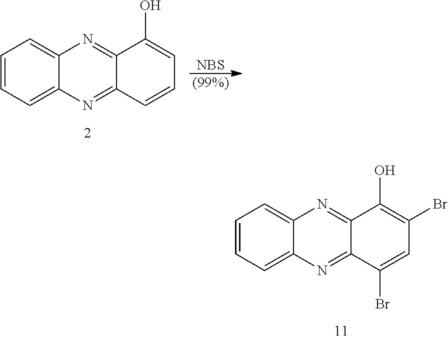

### 2,4-Dibromophenazin-1-ol (11)

1-Hydroxyphenazine (81 mg, 0.412 mmol) was dissolved in 8 mL toluene and treated with N-bromosuccinimide (162 mg, 0.906 mmol). The reaction was heated to 50° C. for 5.5 hours. The reaction was then allowed to cool to room temperature before being concentrated under reduced pressure. The residue was then adsorbed onto silica using dichloromethane and concentrated under reduced pressure before being applied to a column. Column chromatography using dichloromethane to elute delivered 145 mg (99% yield) 2,4-dibromophenazin-1-ol 11 as a yellow solid. In one experiment, 1.106 grams of 2,4-dibromophenazin-1-ol was synthesized starting from 795 milligrams of 1-hydroxyphenazine (77% yield).

¹H NMR (400 MHz, CDCl₃): δ 8.54 (br s, 1H), 8.43-8.38 (m, 1H), 8.30-8.24 (m, 2H), 7.98-7.90 (m, 2H).

¹³C NMR (100 MHz, CDCl₃): δ 149.3, 144.3, 141.6, 140.1, 137.4, 134.4, 132.3, 131.9, 130.4, 129.0, 113.1, 103.2.

HRMS (DART): m/z calc. for C₁₂H₆N₂OBr₂ [M+H]⁺: 354.8900, found: 354.8909.

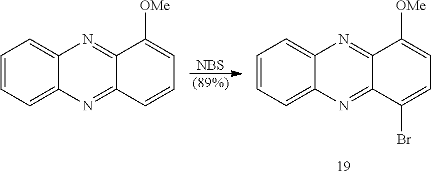

### 1-Bromo-4-methoxyphenazine (19)

1-Methoxyphenazine (60 mg, 0.29 mmol) was dissolved in a 1:1 toluene:acetonitrile solution (10 mL) and treated with N-bromosuccinimide (53 mg, 0.30 mmol). The resulting reaction mixture was heated to 50° C. and allowed to stir for 14 hours. The reaction was then cooled, adsorbed onto silica gel, and purified via column chromatography eluting with 3:1 hexanes:ethyl acetate to give 73 mg (89% yield) of 1-bromo-4-methoxyphenazine 19 as a yellow solid.

¹H NMR (400 MHz, CDCl₃): δ 8.36-8.25 (m, 2H), 7.96 (d, J=8.2 Hz, 1H), 7.86-7.75 (m, 2H), 6.84 (d, J=8.2 Hz, 1H), 4.07 (s, 3H).

¹³C NMR (100 MHz, CDCl₃): δ 155.1, 143.6, 142.3, 141.0, 137.1, 133.2, 131.4, 131.1, 129.9, 129.8, 114.2, 106.9, 56.7.

HRMS (DART): m/z calc. for C₁₃H₁₀N₂OBr [M+H]⁺: 288.9971, found: 288.9979.

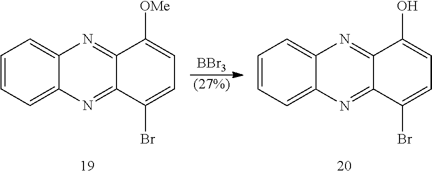

### 4-Bromophenazin-1-ol (20)

1-Bromo-4-methoxyphenazine (25 mg, 0.086 mmol) was dissolved in 2 mL dichloromethane, and cooled to −78° C. Boron tribromide (0.26 mL, 1.0 M in dichloromethane) was then added to the reaction, and the resulting mixture was allowed to warm to room temperature overnight. The reaction was then refluxed for 1 hour, allowed to cool, and quenched with 3 mL of a saturated aqueous solution of sodium bicarbonate. The resulting mixture was extracted with dichloromethane (3×20 mL). The combined organic layers were dried with sodium sulfate, filtered, and concentrated under reduced pressure. The crude material was subjected to column chromatography eluting with 3:1 hexanes:ethyl acetate to deliver 6.3 mg (27% yield) of 4-bromophenazin-1-ol 20 as a yellow solid.

¹H NMR (400 MHz, CDCl₃): δ 8.42 (m, 1H), 8.26 (m, 1H), 8.22 (s, 1H), 8.09 (d, J=8.1 Hz, 1H), 7.96-7.86 (m, 2H), 7.15 (d, J=8.1 Hz, 1H).

¹³C NMR (100 MHz CDCl₃): δ 151.8, 144.6, 141.5, 141.0, 135.2, 134.7, 131.6 (2), 130.4, 129.0, 112.3, 109.6.

HRMS (DART): m/z calc. for C₁₂H₈N₂OBr [M+H]⁺: 274.9815, found: 274.9819.

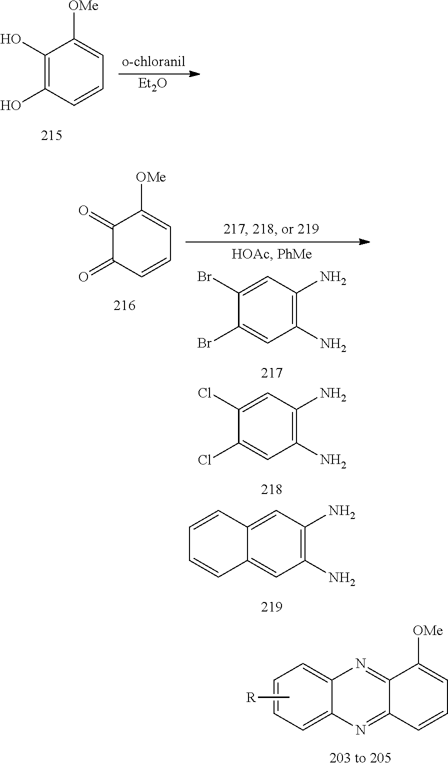

### General Procedure for the Synthesis of 1-Methoxyphenazines (203 to 205)

In a round-bottom flask, 3-methoxycatechol 215 (1.40 g, 10 mmol) was dissolved in diethyl ether (30 mL), then cooled to −78° C. Tetrachloro-o-benzoquinone (3.07 g, 12.5 mmol) was then added, and the solution and allowed to stir at −78° C. for 4 hours. The reaction mixture was then filtered twice under reduced pressure to afford benzoquinone 216 as a dark brown solid, which was used without further purification. Compound 216 was added to a 250 mL round-bottom flask containing 1:1 glacial acetic acid:toluene solution (70 mL) with phenylenediamine (217, 218, or 219) (5 mmol). The resulting reaction mixture was allowed to stir for 24 hours at room temperature. Upon completion of the reaction, the mixture was neutralized with an aqueous solution of saturated sodium bicarbonate, washed with brine, and then extracted with dichloromethane. The organic layers were dried with sodium sulfate, filtered and concentrated under reduced pressure to afford the crude product, which was then purified via column chromatography using 100% dichloromethane as the eluent to afford 1-methoxyphenazines 203-205 in 76 to 86% yield. NOTE: This procedure was a modification of a previously published protocol.²⁸

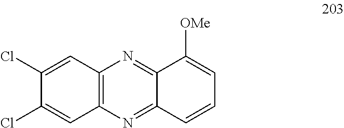 [SMILES: COc1cccc2nc3cc(Cl)c(Cl)cc3nc12]

Yield:

86% yield; 1.49 g of 203 was isolated as a yellow solid.

¹H NMR (400 MHz, CDCl₃): δ 8.53 (s, 1H), 8.35 (s, 1H), 7.80-7.76 (m, 2H), 7.09 (dd, J=4.3, 4.3 Hz, 1H), 4.17 (s, 3H).

¹³C NMR (100 MHz, CDCl₃): δ 155.3, 144.8, 142.2, 140.8, 137.4, 136.0, 135.4, 131.7, 130.5, 129.8, 121.6, 107.4, 56.8.

MP: 253-255° C.; literature value: 245-247° C.²⁸

Note: NMR spectra matched those previously reported.²⁸

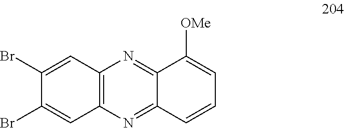 [SMILES: COc1cccc2nc3cc(Br)c(Br)cc3nc12]

Yield:

78% yield; 1.09 g of 204 was isolated as a yellow solid.

¹H NMR (400 MHz, CDCl₃): δ 8.72 (s, 1H), 8.54 (s, 1H), 7.81-7.73 (m, 2H), 7.08 (dd, J=6.0, 2.7 Hz, 1H), 4.16 (s, 3H).

¹³C NMR (100 MHz, CDCl₃): δ 155.3, 144.8, 142.5, 141.1, 137.4, 134.0, 133.3, 131.8, 128.1, 127.4, 121.6, 107.5, 56.8.

HRMS (DART): calc. for C₁₃H₉Br₂N₂O [M+H]⁺: 366.9076, found: 366.9081.

MP: 231-233° C.

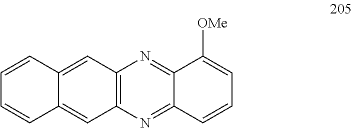 [SMILES: COc1cccc2nc3cc4ccccc4cc3nc12]

Yield:

76% yield; 906.6 mg of 205 was isolated as a red solid.

¹H NMR (400 MHz, CDCl₃): δ 9.08 (s, 1H), 8.89 (s, 1H), 8.17-8.01 (m, 2H), 7.82 (dd, J=9.1, 1.1 Hz, 1H), 7.73 (dd, J=9.1, 7.4 Hz, 1H), 7.58-7.50 (m, 2H), 7.01 (dd, J=7.4, 1.1 Hz, 1H), 4.21 (s, 3H).

MP: 223-225° C.; literature value: 245-247° C.²⁸

Note: ¹H NMR was identical to previously reported spectra.²⁸

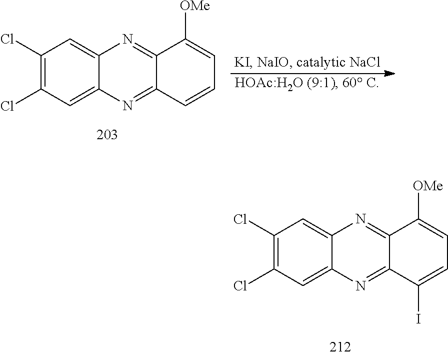

### Synthesis of 7,8-dichloro-4-iodo-1-methoxyphenazine (212)

To a mixture of 7,8-dichloro-1-methoxyphenazine 203 (472 mg, 1.69 mmol) in a 9:1 solution of acetic acid:water (30 mL) was added sodium chloride (690 mg, 11.8 mmol), sodium periodate (1.26 g, 5.91 mmol), and potassium iodide (981 mg, 5.91 mmol). The reaction was heated to 60° C. and allowed to stir for 24 hours. After completion of the reaction (monitored by TLC using 100% dichloromethane), the reaction contents were then transferred to a separatory funnel containing a solution of saturated sodium bicarbonate, and then the aqueous phase was extracted with dichloromethane. The organic layers were dried with anhydrous sodium sulfate, filtered, and then the filtrate was concentrated under reduced pressure. The remaining crude solid was adsorbed onto silica gel and purified via column chromatography using 100% hexanes to first elute purple side products, then 100% dichloromethane to elute the product, which was obtained as a yellow solid (59%, 407 mg).

¹H NMR (400 MHz, d₆-DMSO): δ 8.67 (s, 1H), 8.59 (s, 1H), 8.52 (d, J=8.2 Hz, 1H), 7.16 (d, J=8.2 Hz, 1H), 4.07 (s, 3H).

¹³C NMR (100 MHz, d₆-DMSO): δ 155.8, 142.3, 141.8, 141.6, 140.5, 137.0, 134.9, 134.2, 129.9, 129.5, 110.0, 90.0, 56.3.

HRMS (DART): calc. for C₁₃H₈C₁₂IN₂O [M+H]⁺: 404.9053, found: 404.9047.

MP: 238-240° C.

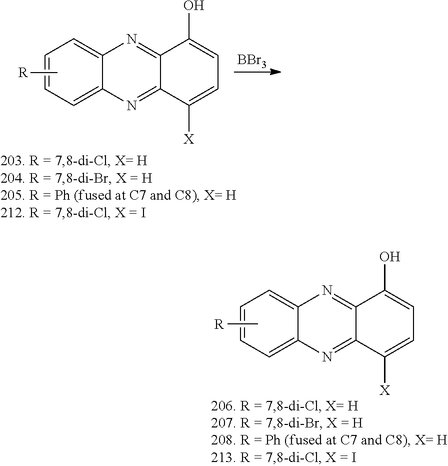

### General Demethylation Procedure

To a round bottom flask was added the desired 1-methoxyphenazine (1.25 mmol) dissolved in 15 mL anhydrous dichloromethane. The resulting mixture was cooled to −78° C. before dropwise addition of 1 M (7.5 ml, 7.5 mmol) boron tribromide solution in dichloromethane. The reaction was left to stir at −78° C. for 1 hour and then allowed to reach ambient temperature for reaction overnight. The reaction was then heated to reflux for 2 to 8 hours until complete (monitored by TLC). The reaction contents were then transferred to a separatory funnel containing an aqueous solution of saturated sodium bicarbonate, and then the aqueous layer was extracted with dichloromethane. The organic layers were dried with sodium sulfate, filtered through cotton, and the filtrate was concentrated under reduced pressure. The remaining crude solid was purified via column chromatography using 100% dichloromethane to elute pure products.

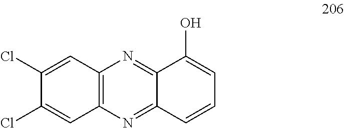 [SMILES: Oc1cccc2nc3cc(Cl)c(Cl)cc3nc12]

Yield:

>99% yield; 224.1 mg of 206 was isolated as an orange solid.

¹H NMR (400 MHz, d₆-DMSO): δ 8.57 (s, 1H), 8.55 (s, 1H), 7.84 (dd, J=8.8, 7.5 Hz, 1H), 7.68 (d, J=8.8 Hz, 1H), 7.24 (d, J=7.5 Hz, 1H).

MP: >260° C.; literature value: 245-247° C.²⁸

Note: ¹H NMR and melting point matched those previously reported.²⁸

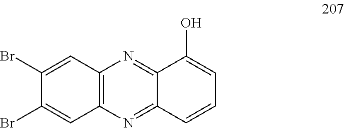 [SMILES: Oc1cccc2nc3cc(Br)c(Br)cc3nc12]

Yield:

81% yield; 223.3 mg of 207 was isolated as an orange solid.

¹H NMR (400 MHz, d₆-DMSO): δ 8.67 (s, 1H), 8.67 (2) (s, 1H), 7.84 (dd, J=8.8, 7.5 Hz, 1H), 7.67 (d, J=8.8 Hz, 1H), 7.24 (d, J=7.5 Hz, 1H).

¹³C NMR (100 MHz, d₆-DMSO): δ 153.5, 144.0, 141.7, 140.1, 136.2, 133.1, 133.0, 132.9, 126.6, 125.8, 118.9, 111.2.

HRMS (DART): calc. for C₁₂H₇Br₂N₂O [M+H]⁺: 352.8920, found: 352.8933.

MP: 252-254° C.

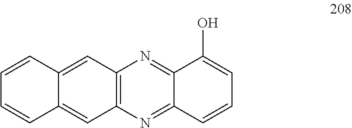 [SMILES: Oc1cccc2nc3cc4ccccc4cc3nc12]

Yield:

78% yield; 234.8 mg of 208 was isolated as a red solid.

¹H NMR (400 MHz, d₆-DMSO): δ 10.67 (s, 1H), 9.03 (d, J=1.2 Hz, 1H), 8.98 (d, J=1.2 Hz, 1H), 8.36-8.20 (m, 2H), 7.78 (dd, J=9.0, 7.4 Hz, 1H), 7.68 (dd, J=9.0, 1.2 Hz, 1H), 7.65-7.54 (m, 2H), 7.14 (dd, J=7.4, 1.2 Hz, 1H).

¹³C NMR (100 MHz, d₆-DMSO): δ 153.4, 144.8, 139.5, 138.0, 137.6, 134.2, 133.8, 132.5, 128.5, 128.4, 127.5, 127.2, 127.1, 126.9, 119.4, 109.7.

HRMS (DART): calc. for C₁₆H₁₁N₂O [M+H]⁺: 247.0866, found: 247.0870.

MP: 177-179° C.

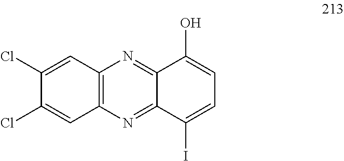 [SMILES: Oc1ccc(I)c2nc3cc(Cl)c(Cl)cc3nc12]

Yield:

97% yield; 336.8 mg of 213 was isolated as an orange solid.

¹H NMR (400 MHz, CDCl₃): δ 8.58 (s, 1H), 8.40 (s, 1H), 8.38 (d, J=8.0 Hz, 1H), 8.07 (s, 1H), 7.09 (d, J=8.0 Hz, 1H).

¹³C NMR (100 MHz, CDCl₃): δ 152.8, 143.2, 143.0, 142.5, 140.0, 136.8, 136.8, 135.3, 130.4, 129.0, 111.8, 88.2.

HRMS (DART): calc. for C₁₂H₆Cl₂IN₂O [M+H]⁺: 390.8896, found: 390.8886.

MP: 208-210° C.

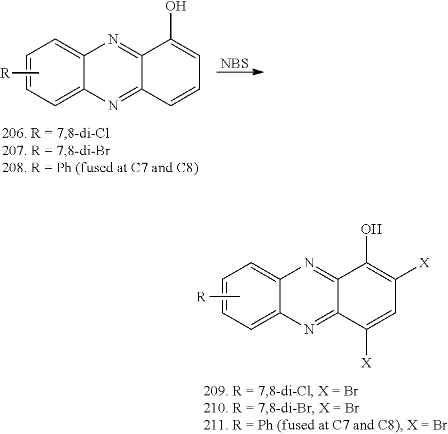

### General Procedure for Synthesis of 2,4-dihalo-1-hydroxyphenazines (209-211)

1-Hydroxyphenazines (0.20 mmol) and N-bromosuccinimide (0.42 mmol) were dissolved in 5 mL of dichloromethane, and the resulting mixture was allowed to stir at room temperature for 1 hour. The reaction contents were then concentrated, adsorbed onto silica gel, and purified via column chromatography using dichloromethane to elute 2,4-dihalo-1-hydroxyphenazines 209-211 (38 to 84% yield). Note: These reactions were typically run on a 30-100 mg scale.

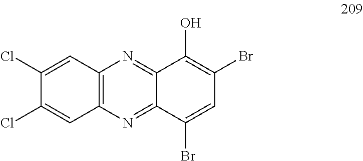 [SMILES: Oc1c(Br)cc(Br)c2nc3cc(Cl)c(Cl)cc3nc12]

Yield:

60% yield; 68.9 mg of 209 was isolated as a dark yellow solid.

¹H NMR (400 MHz, d₆-DMSO): δ 11.71 (s, 1H), 8.66 (s, 1H), 8.52 (s, 1H), 8.48 (s, 1H).

¹³C NMR (100 MHz, d₆-DMSO): δ 151.0, 141.4, 140.0, 140.0, 137.9, 136.0, 135.2, 135.0, 130.1, 129.3, 111.4, 105.7.

HRMS (DART): calc. for C₁₂H₅Br₂C₂N₂O [M+H]⁺: 420.8140, found: 420.8138.

MP: 249-251° C.

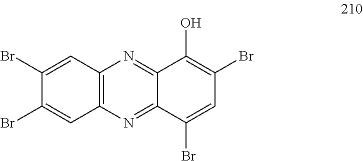 [SMILES: Oc1c(Br)cc(Br)c2nc3cc(Br)c(Br)cc3nc12]

Yield:

84% yield; 103.9 mg of 210 was isolated as a dark orange solid.

¹H NMR (400 MHz, d₆-DMSO): δ 11.57 (br. s, 1H), 8.60 (s, 1H), 8.54 (s, 1H), 8.38 (s, 1H).

¹³C NMR (100 MHz, d₆-DMSO): δ 150.9, 141.5, 140.1, 139.7, 137.9, 135.8, 133.0, 132.4, 128.1, 128.1, 111.4, 105.6.

HRMS (DART): calc. for C₁₂H₅Br₄N₂O [M+H]⁺: 508.7130, found: 508.7112.

MP: 247-249° C.

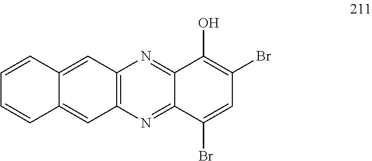 [SMILES: Oc1c(Br)cc(Br)c2nc3cc4ccccc4cc3nc12]

Yield:

38% yield; 36.0 mg of 211 was isolated as a red solid.

¹H NMR (400 MHz, d₆-DMSO): δ 11.61 (br. s, 1H), 9.12 (s, 1H), 9.08 (s, 1H), 8.39 (s, 1H), 8.37-8.28 (m, 2H), 7.72-7.64 (m, 2H).

¹³C NMR (100 MHz, d₆-DMSO): δ 150.8, 140.3, 139.0, 137.7, 137.2, 136.8, 134.8, 134.7, 128.6, 128.6, 127.8, 127.7, 127.7, 127.2, 111.8, 103.7.

HRMS (DART): calc. for C₁₆H₉Br₂N₂O [M+H]⁺: 402.9076, found: 402.9086.

MP: 223-225° C.

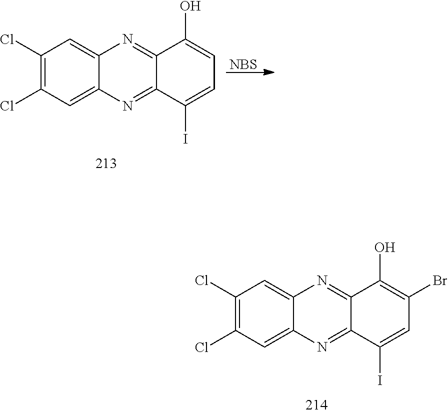

### General Procedure for Synthesis of 2-dibromo-7,8-dichloro-4-iodo-1-hydroxyphenazine (214)

7,8-Dichloro-4-iodo-1-hydroxyphenazine 213 (115 mg, 0.296 mmol) and N-bromosuccinimide (52.6 mg, 0.296 mmol) were dissolved in 20 mL of dichloromethane, and the resulting mixture was allowed to stir at room temperature for 1 hour. The reaction contents were then concentrated, adsorbed onto silica gel, and purified via column chromatography using dichloromethane to elute the product, which was obtained as an orange solid (32%, 43.6 mg).

¹H NMR (400 MHz, d₆-DMSO): δ 11.66 (br. s, 1H), 8.62 (s, 1H), 8.60 (s, 1H), 8.50 (s, 1H).

¹³C NMR (100 MHz, d₆-DMSO): δ 151.6, 144.1, 141.8, 141.7, 140.0, 135.7, 135.0, 134.8, 129.9, 129.1, 106.7, 89.0.

HRMS (DART): calc. for C₁₂H₅BrCl₂IN₂O [M+H]⁺: 468.8002, found: 468.8002.

MP: 229-231° C.

Exemplary calculated properties of select compounds of the invention are shown in Table 1.

TABLE 1
Exemplary calculated properties of select compounds of the
invention

| | Topological polar | | |
|---|---|---|---|
| Compound # | surface area (tPSA) | cLogP | pKₐ |
| 1 or | 44.95 | 4.68162 | 5.613 |
| AG-1-141 | | | |
| 22, | 44.95 | 4.33162 | 5.632 |
| AG-1-147, or | | | |
| AG-4-9 | | | |
| 18, | 44.95 | 5.13162 | 4.692 |
| AG-1-181, | | | |
| AG-1-159-2, or | | | |
| AG-3-123 | | | |
| AG-3-181 or | 44.95 | 4.48162 | 5.645 |
| AG-4-103 | | | |
| AG-3-81 | 44.95 | 4.87162 | 4.896 |
| AG-3-167 or | 44.95 | 4.94162 | 5.409 |
| AG-4-79-2 | | | |
| AG-4-3 | 44.95 | 4.74162 | 5.441 |
| AG-4-45 | 44.95 | 4.53162 | 5.600 |
| AG-4-47 | 44.95 | 4.72162 | 4.882 |
| 32, | 44.95 | 6.21054 | 5.391 |
| AG-2-91, | | | |
| AG-3-31, or | | | |
| AG-4-63 | | | |
| AG-3-173 | 44.95 | 6.01054 | 5.423 |
| AG-4-53 | 33.95 | 4.59798 | |
| AG-4-85 | 44.95 | 4.50481 | 7.393 |
| AG-4-1 | 44.95 | 5.39724 | 6.891 |
| AG-4-73 | 44.95 | 5.65724 | 6.687 |
| 24 or | 44.95 | 5.99054 | 5.385 |
| AG-2-27 | | | |
| AG-4-99-2 | 44.95 | 6.25054 | 5.181 |
| 33 or | 44.95 | 5.85562 | 5.408 |
| AG-3-99 | | | |
| AG-4-37 | 44.95 | 3.08762 | 6.192 |
| AG-4-67 | 44.95 | 3.53762 | 5.277 |
| AG-4-41 | 44.95 | 4.03562 | 6.075 |
| AG-4-75 | 44.95 | 4.48562 | 5.160 |

The references cited in Example 1 are included in Example 2. In Table 1, the different compound numbers in the same cell of Table 1 refer to the same compound.

### Example 2. Biological Assays of the Compounds

### Antibiotic Susceptibility Tests (MIC Assay Protocol)

The minimum inhibitory concentration (MIC) for each compound described herein was determined by the broth microdilution method as recommended by the Clinical and Laboratory Standards Institute (CLSI) (Clinical and Laboratory Standards Institute. 2009. Methods for dilution antimicrobial susceptibility tests for bacteria that grow aerobically; approved standard, eighth edition (M7-A8)). In a 96-well plate, eleven two-fold serial dilutions of each compound were made in a final volume of 100 μL Luria Broth (one column served as a blank; see the MIC assay described herein). Each well was inoculated with 10⁵ bacterial cells at the initial time of incubation, prepared from a fresh log phase culture (OD₆₀₀ of 0.5 to 1.0 depending on bacterial strain). The MIC was defined as the lowest concentration of a compound that prevented bacterial growth after incubating of 16 to 18 hours at 37° C. (MIC values were supported by spectrophotometric readings at OD₆₀₀). The concentration range tested for each compound during this study was 0.10 to 100 μM. DMSO served as the vehicle and negative control in each microdilution MIC assay. DMSO was serially diluted at the same concentration as the compounds with a top concentration of 1% v/v. Bacterial strains used included methicillin-resistant Staphylococcus aureus (MRSA) (Clinical Isolates from Shands Hospital in Gainesville, Fla.: MRSA-2, MRSA-1), methicillin-resistant Staphylococcus epidermidis (MRSE strain ATCC 35984; methicillin-sensitive strain ATCC 12228), vancomycin-resistant Enterococcus faecium (VRE; ATCC 700221). Exemplary results are shown in Tables 2A and 2B.

TABLE 2A
Minimum inhibitory concentrations (MICs, in μM) of select
compounds of the invention against select microorganisms

| | | | | | Enterococcus |
|---|---|---|---|---|---|
| | | | Staphylococcus | Staphylococcus | faecium |
| | | | epidermidis | epidermidis | (ATCC |
| Compound | MRSA-2 | MRSA-1 | (ATCC 12228) | (ATCC 35984) | 700221) |
| AG-1-141 | 1.56 | 1.56 | 1.17^{a} | 1.56 | 6.25 |
| AG-1-147 or | 3.13** | 3.13 | 3.13 | 3.13 | 12.5 |
| AG-4-9 | | | | | |
| AG-1-181 or | 3.13 | 0.78-1.56 | 1.56 | 0.78-1.56 | 6.25 |
| AG-3-123 | | | | | |
| AG-3-181 or | 1.56-3.13 | | 3.13 | 1.56 | 6.25 |
| AG-4-103 | | | | | |
| AG-3-81 or | 2.25^{a} | | 1.56 | 3.13-6.25 | 6.25 |
| AG-4-29 | | | | | |
| AG-3-167 or | 0.78 | 0.78 | 0.78 | 1.56 | 6.25 |
| AG-4-79-2 | | | | | |
| AG-4-3 | 1.56 | 1.56 | 1.56 | 1.56 | 6.25 |
| AG-4-45 | 1.56 | | 1.56-3.13 | 1.56 | 6.25 |
| AG-4-47 | 25 | | 25 | 25 | 6.25 |
| AG-2-91, | 6.25 | 12.5 | 0.05-0.1 | 1.56-3.13 | 0.39 |
| AG-3-31, or | | | | | |
| AG-4-63 | | | | | |
| AG-3-173 | 12.5 | 6.25 | 0.05-0.1 | 0.2-0.39 | 0.39 |
| AG-2-27 | 4.69^{a} | 12.5 | 0.05-0.1 | 0.2-0.39 | 0.39 |
| AG-4-99-2 | | | | | <0.1 |
| AG-4-53 | >100 | | >100 | >100 | >100 |
| AG-4-85 | >100 | | >100 | >100 | >100 |
| AG-4-1 | 0.78 | | 3.13 | 1.56 | 6.25 |
| AG-4-73 | 0.39 | | 0.39 | 0.78 | |
| AG-3-99 | >100* | 12.5 | 12.5 | 12.5-25 | 6.25-12.5 |
| AG-4-37 | 25 | | 12.5-25 | 12.5-25 | 100 |
| AG-4-67 | | | 6.25 | 3.13 | 12.5 |
| AG-4-41 | 6.25-12.5 | | 12.5 | 6.25-12.5 | 25 |
| AG-4-75 | 1.56 | | 1.56-3.13 | 0.78 | 6.25 |
| Ciprofloxacin | >100 | | 0.78 | | |
| Vancomycin | 0.78 | | 1.17^{a} | | >100 |

In Table 2A, the different compound numbers in the same cell of Table 2A refer to the same compound.

TABLE 2B
Additional minimum inhibitory concentrations (MICs, in μM) of
select compounds of the invention against select microorganisms

| | | | | Enterococcus |
|---|---|---|---|---|
| | | | Staphylococcus | faecium |
| | | MRSA- | epidermidis | (ATCC |
| Compound | MRSA-2 | 1 | (ATCC 35984) | 700221) |
| 202 | 1.56 | 1.17^{a} | 1.56 | 6.25 |
| 209 | 4.69^{a} | 12.5 | 0.30^{a} | 0.39 |
| 210 | 6.25 | 12.5 | 2.35^{a} | 0.39 |
| 211 | >100* | 12.5 | 18.8^{a} | 9.38^{a} |
| 213 | 0.39 | 0.2 | 0.78 | 3.13 |
| 214 | 3.13 | 0.10^{b} | 3.13 | 0.10^{b} |
| Vancomycin | 0.59^{a} | 0.39 | 0.78 | >100 |
| Linezolid | 4.69^{a} | 3.13 | 3.13 | 3.13 |
| Daptomycin | 4.69^{a} | 6.25 | 12.5 | |
| Ciprofloxacin | >100 | 1.56 | 0.2 | |

^{a}Midpoint value (2-fold range in MIC).
^{b}Lowest concentration tested.
*Partial growth inhibition at the highest concentration tested.

### Cytotoxicity

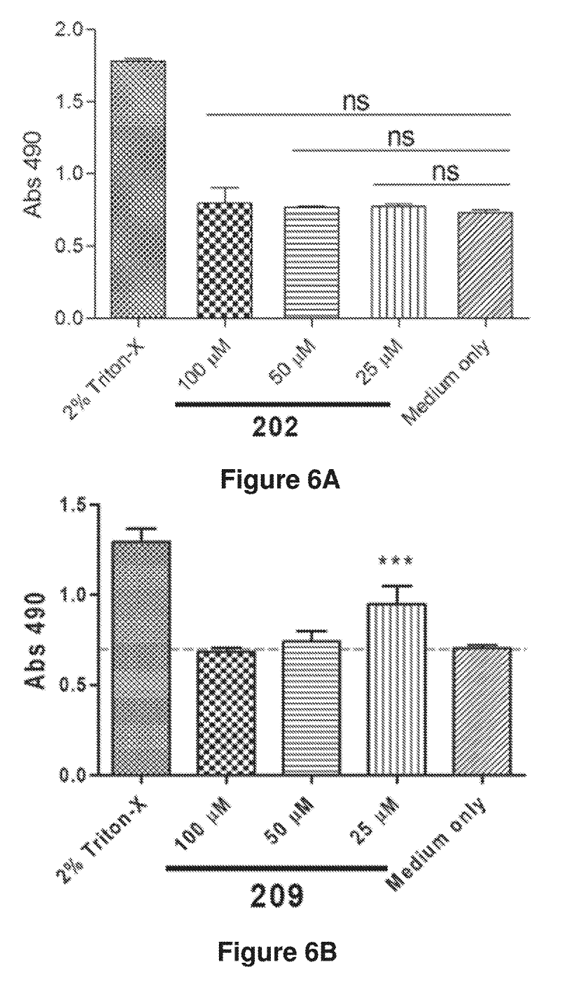

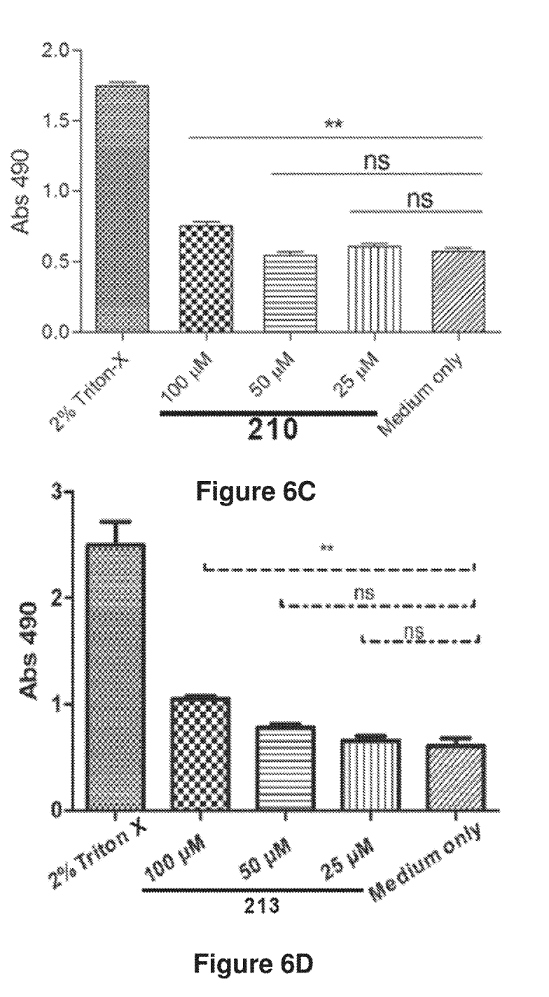

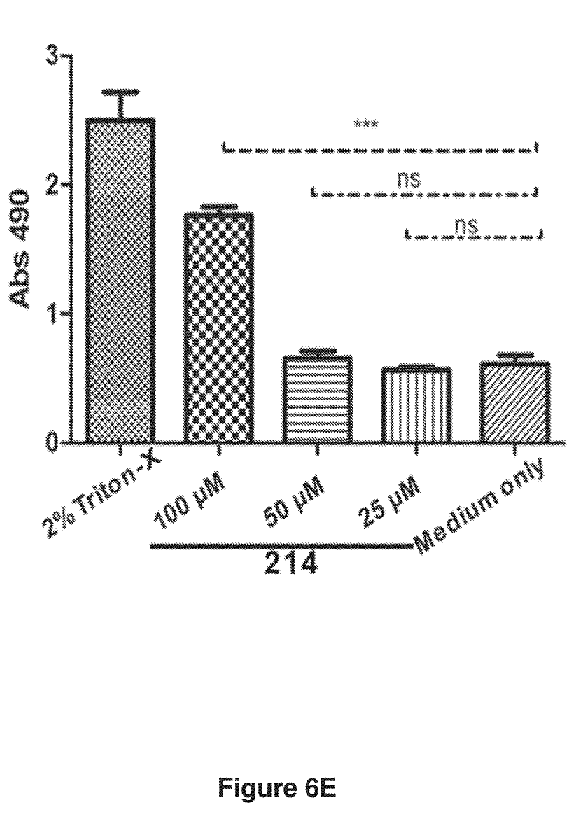

The cytotoxicities of select compounds of the invention against HeLa cells were determined. HeLa cytotoxicity was assessed using the LDH release assay described by CYTOTOX96 (Promega G1780). HeLa cells were grown in Dulbecco's Modified Eagle Medium (DMEM; Gibco) supplemented with 10% Fetal Bovine Serum (FBS) at 37° C. with 5% CO₂. When the HeLa cultures exhibited 70-80% confluence, halogenated phenazines were then diluted by DMEM (10% FBS) at concentrations of 25, 50, and 100 μM and added to HeLa cells. Triton X-100 (at 2% v/v) was used as the positive control for maximum lactate dehydrogenate (LDH) activity in this assay (e.g., complete cell death), while “medium only” lanes served as negative control lanes (e.g., no cell death). DMSO was used as the vehicle control. HeLa cells were treated with compounds for 24 hours, and then 50 μL of the supernatant was transferred into a fresh 96-well plate where 50 μL of the reaction mixture was added to the 96-well plate and incubated at room temperature for 30 minutes. Finally, Stop Solution (50 μL) was added to the incubating plates, and the absorbance was measured at 490 nm. Exemplary results are shown in Table 3 and FIGS. 6A to 6E.

### Hemolysis Activity

The hemolysis activities of select compounds of the invention were determined. As previously described,³² freshly drawn human red blood cells (hRBC with ethylenediaminetetraacetic acid (EDTA) as an anticoagulant) were washed with Tris-buffered saline (0.01M Tris-base, 0.155 M sodium chloride (NaCl), pH 7.2) and centrifuged for 5 minutes at 3,500 rpm. The washing was repeated three times with the buffer. In 96-well plate, test compounds were added to the buffer from DMSO stocks. Then 2% hRBCs (50 μL) in buffer were added to test compounds to give a final concentration of 200 μM. The plate was then incubated for 1 hour at 37° C. After incubation, the plate was centrifuged for 5 minutes at 3,500 rpm. Then 80 μL of the supernatant was transferred to another 96-well plate, and the optical density (OD) was read at 405 nm. DMSO served as the negative control (0% hemolysis), while Triton X served as the positive control (100% hemolysis). The percent of hemolysis was calculated as (OD₄₀₅ of the compound−OD₄₀₅ of DMSO)/(OD₄₀₅ of Triton X−OD₄₀₅ of the buffer). Exemplary results are shown in Table 3.

TABLE 3
Cytotoxicities against HeLa cells and hemolysis activities of
select compounds of the invention

| | Cytotoxicity against | |
|---|---|---|
| | HeLa cells | % Hemolysis |
| Compound # | IC₅₀ (μM) | (200 μM) |
| AG-1-141 | >100 | ≤1 |
| AG-1-147 or AG-4-9 | | ≤1 |
| AG-1-181 or | >100 | ≤1 |
| AG-3-123 | | |
| AG-3-181 or AG-4-103 | >100 | ≤1 |
| AG-3-81 or AG-4-29 | | 1.3 |
| AG-3-167 or AG-4-79-2 | >100 | 2.9 |
| AG-4-3 | >100 | 5.1 |
| AG-4-45 | >100 | ≤1 |
| AG-4-47 | | ≤1 |
| AG-2-91, | >100 | ≤1 |
| AG-3-31, or | | |
| AG-4-63 | | |
| AG-3-173 | >100 | 2.1 |
| AG-2-27 | >100 | ≤1 |
| AG-4-99-2 | | 2.7 |
| AG-4-53 | | 1.5 |
| AG-4-85 | | ≤1 |
| AG-4-1 | | 2.7 |
| AG-4-73 | | 1.4 |
| AG-3-99 | | ≤1 |
| AG-4-37 | | 1.8 |
| AG-4-67 | >100 | ≤1 |
| AG-4-41 | | ≤1 |
| AG-4-75 | | 1.2 |
| Ciprofloxacin | | 1.8 |
| Vancomycin | | ≤1 |
| Linezolid | | ≤1 |
| Daptomycin | | 1.7 |
| Rifampin | | 8.0 |

In Table 3, the different compound numbers in the same cell of Table 3 refer to the same compound.

### Biofilm Inhibition Protocol

A serial two-fold dilution of 2× compound concentration was made in 100 L tryptic soy broth (TSB) medium with 0.5% glucose were delivered into 0.1% gelatin (Millipore) coated 96-well tissue culture plates. The same volume of DMSO (vehicle control), was used as a negative control and did not go over 1% v/v in biofilm inhibition assays. To each well, 100 μL of TSB with 0.5% glucose containing 2×10⁶ CFU/mL methicillin resistant Staphylococcus aureus (MRSA), Staphylococcus epidermidis (ATCC 35984), or Enterococcus faecium (ATCC 700221) cells, prepared from fresh culture (OD₆₀₀ of 0.8), was added. The plates were incubated at 37° C. for 24 hours. The wells were gently rinsed by submerging the entire plates in a tub of cold, running tap water. The wells were then fixed with 200 μL methanol for 15 minutes. After the plates were air dried, the biofilms were stained with 100 μL of 1% crystal violet for 10 minutes. The plates were again rinsed with running water. After drying in air, quantitative assessment of biofilm formation was obtained by extracting the crystal violet associated with the remaining biofilm with 100 μL per well of the following bleaching solution (methanol:glacial acetic acid:water (v/v/v)=4:1:5). This bleaching solution dissolved the bound crystal violet and produced a violet-colored solution in each well. The intensity of coloration was determined by measuring the absorbance at 540 nm. Exemplary results are shown in Table 4.

TABLE 4
Minimum bactericidal concentrations (MBCs, in μM), minimum
biofilm eradication concentrations (MBECs, in μM), and MBEC:MBC
ratios of select compounds of the invention against select
microorganisms

| | | | Staphylococcus epidermidis | |
|---|---|---|---|---|
| | MRSA-2 | | (ATCC 35984) | |
| | | MBEC:MBC | | MBEC:MBC |
| Compound | MBC/MBEC | ratio | MBC/MBEC | ratio |
| AG-1-141 | 15.6/93.8^{a} | 6.0 | | |
| AG-1-147 or | 62.5/>1000 | >16.0 | | |
| AG-4-9 | | | | |
| AG-3-123 | 31.3/93.8^{a} | 3.0 | | |
| AG-3-181 or | 62.5/>1000 | >16.0 | | |
| AG-4-103 | | | | |
| AG-3-81 or | 46.9^{a}/250 | 5.3 | | |
| AG-4-29 | | | | |
| AG-3-167 or | 31.3/62.5 | 2.0 | | |
| AG-4-79-2 | | | | |
| AG-4-3 | 15.6/125 | 8.0 | | |
| AG-4-45 | 46.9^{a}/375 | 8.0 | | |
| AG-4-47 | 93.8^{a}/250 | 2.7 | | |
| AG-2-91, | 37.5^{a}/50 | 1.3 | 1.56/6.25 | 4.0 |
| AG-3-31, or | | | | |
| AG-4-63 | | | | |
| AG-3-173 | 25^{b}/25 | 1.0 | 1.56/3.13 | 2.0 |
| AG-2-27 | 12.5/50 | 4.0 | 1.56/3.13 | 2.0 |
| AG-4-1 | 37.5^{a}/37.5^{a} | 1.0 | | |
| AG-4-73 | 25/37.5 | 1.5 | | |
| AG-3-99 | 200/>200 | >1.0 | | |
| AG-4-37 | 1500^{a}/>2000 | >1.3 | | |
| AG-4-67 | >2000*/>2000 | | | |
| AG-4-41 | 125^{b}/1500 | 12.0 | | |
| AG-4-75 | 93.8^{a}/93.8^{a} | 1.0 | | |
| N-acetyl- | >2000/>2000 | | | |
| cysteine | | | | |
| (NAC) | | | | |
| Vancomycin | 5.9^{a}/>2000 | >339 | | |
| Daptomycin | 62.5^{b}/>2000 | >32 | | |
| Linezolid | 15.5/>2000 | >129 | | |

In Table 4, the different compound numbers in the same cell of Table 4 refer to the same compound.

Mycobacterium tuberculosis (M. tuberculosis) MIC Assay

M. tuberculosis H37Ra (ATCC 25177) was inoculated in 10 ml Middlebrook 7H9 medium and allowed to grow for two weeks. The culture was then diluted with fresh medium until an OD₆₀₀ of 0.01 was reached. Aliquots of 200 μl were then added to each well of a 96-well plate starting from the second column. Test compounds were dissolved in DMSO at final concentration of 10 mM. 7.5 μl of each compound solution along with DMSO (negative control) and streptomycin (positive control-40 mg/ml stock solution) were added to 1.5 ml of the Mycobacterium diluted cultures, resulting in 50 μM final concentration of each halogenated phenazine analogues and 340 μM for streptomycin. The final DMSO concentration was maintained at 0.5%. Aliquots of 400 μl were added to wells of the first column of the 96-well plate and serially diluted two-fold (200 μl) per well across the plate to obtain final concentrations that ranges from 0.024 to 50 μM for the test compounds and 0.16 to 340 μM for streptomycin. Three rows were reserved for each compound. The plates were then incubated at 37° C. for seven days. Minimum inhibitory concentrations (Mtb MICs) were reported as the lowest concentration at which no bacterial growth was observed. OD₆₀₀ absorbance was recorded using SPECTRAMAX M5 (Molecular Devices). Data obtained from three independent experiments were analyzed using Excel. Exemplary results are shown in Table 5.

### Calgary Biofilm Device (CBD) Assays

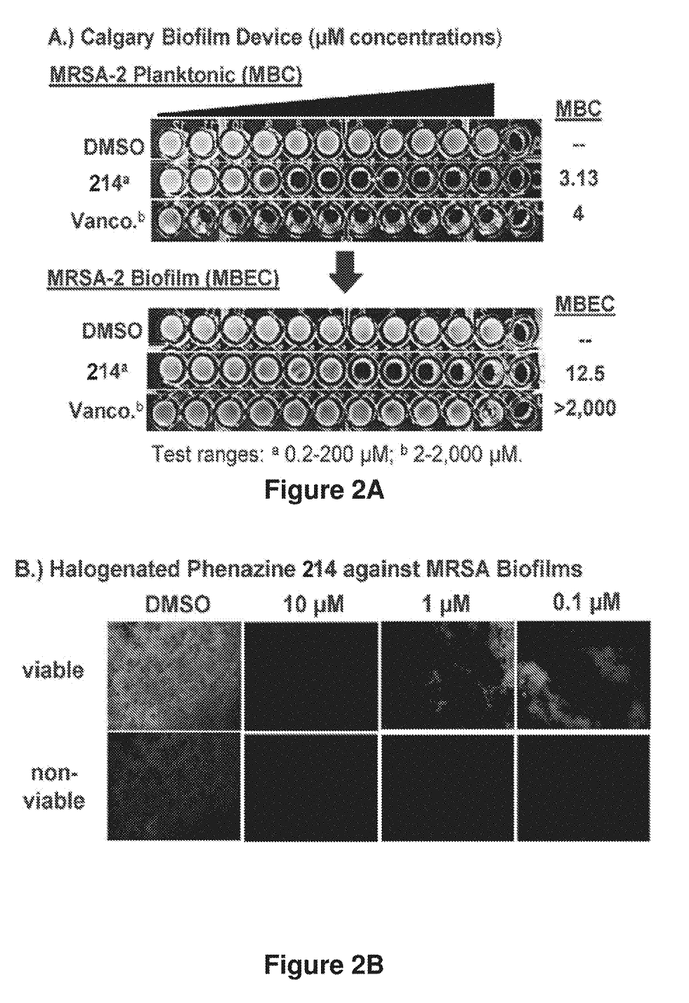

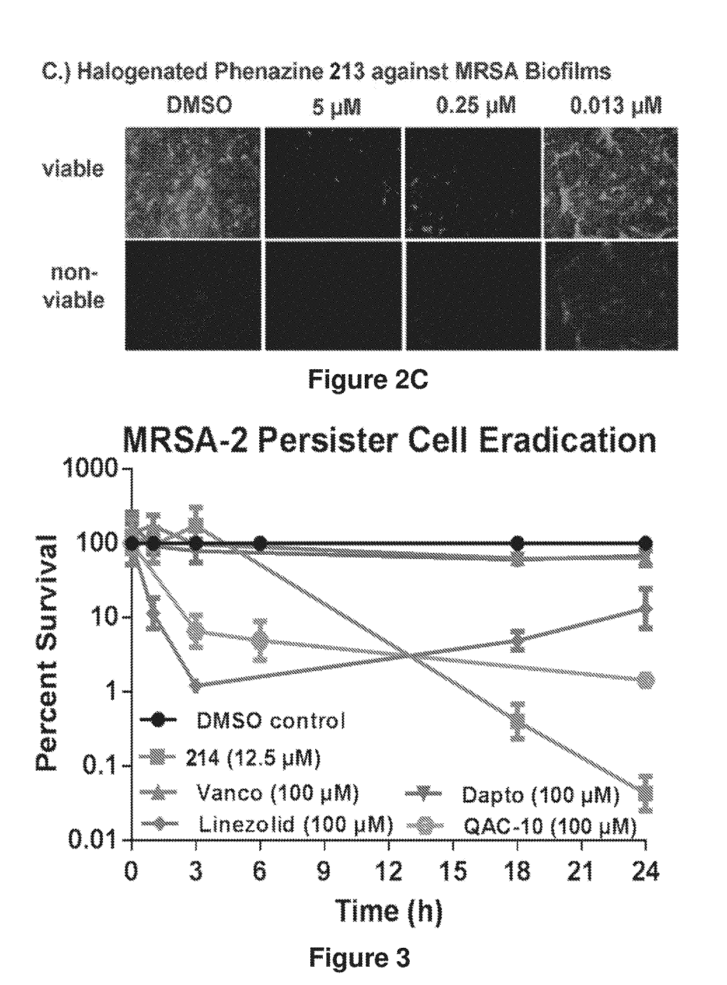

HP analogues, front-running MRSA treatments (vancomycin, daptomycin, linezolid), and control compounds were evaluated for bacterial biofilm eradication activity against MRSA-2 using the Calgary Biofilm Device (CBD),20 which allows biofilms to be established on pegs that are submerged in inoculated media in 96-well plates. Pegs with established biofilms are then transferred to a second 96-well plate containing serial dilutions of test compounds (e.g., the HPs) for biofilm eradication. Following compound treatment, pegs are transferred to fresh media to allow viable biofilms to recover (grow and disperse) resulting in turbid wells (FIG. 2A). During these investigations, it was found that CBD assays were superior to biofilm eradication assays that regrow biofilms on the inside of microtiter wells.¹⁷ The CBD allows for the determination of biofilm (MBEC) and planktonic (minimum bactericidal concentration or MBC) killing dynamics from a single experiment. Typically, MBEC and MIC values were compared using different assays^{10,20} (e.g., bacterial density, media, incubation times), which had an impact on these values.

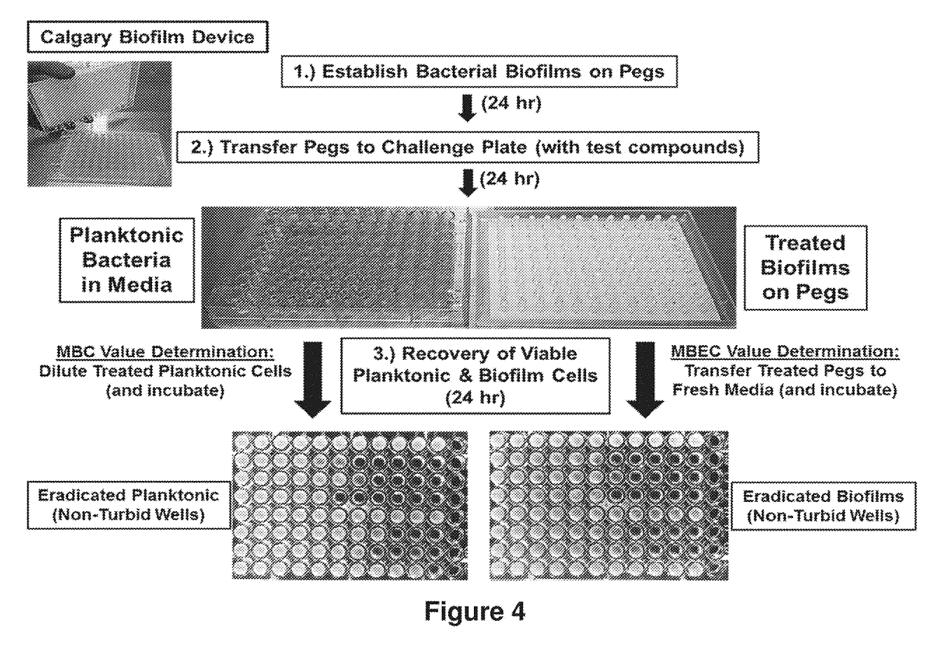

In one set of experiments, biofilm eradication experiments were performed using the Calgary Biofilm Device to determine MBC/MBEC values for various compounds of interest (Innovotech, product code: 19111) (FIG. 4). The Calgary device (96-well plate with lid containing pegs to establish biofilms on) was inoculated with 125 μL of a mid-log phase culture diluted 1,000-fold in tryptic soy broth with 0.5% glucose (TSBG) to establish bacterial biofilms after incubation at 37° C. for 24 hours. The lid of the Calgary device was then removed, washed and transferred to another 96-well plate containing 2-fold serial dilutions of the test compounds (the “challenge plate”). The total volume of media with compound in each well in the challenge plate is 150 μL. The Calgary device was then incubated at 37° C. for 24 hours. The lid was then removed from the challenge plate and MBC/MBEC values were determined using different final assays.

To determine MBC values, 20 μL of the challenge plate was transferred into a fresh 96-well plate containing 180 μL TSBG and incubated overnight at 37° C. The MBC values were determined as the concentration giving a lack of visible bacterial growth (e.g., turbidity).

To determine MBEC values, the Calgary device lid (with attached pegs/treated biofilms) was transferred to a new 96-well plate containing 150 μL of fresh TSBG media in each well and incubated for 24 hours at 37° C. to allow viable biofilms to grow and disperse resulting in turbidity after the incubation period. MBEC values were determined as the lowest test concentration that resulted in eradicated biofilm (e.g., wells that had no turbidity after final incubation period).

MRSA-2, S. epidermidis (ATCC 35984), and VRE (ATCC 700221) were tested using these assay parameters.

In CBD assays against MRSA-2 (clinical isolate, Shands Hospital; Gainesville, Fla.), HP 202 exhibited MBC values of 31.3 μM and MBEC values of 93.8 μM (Table 5). Several new HP analogues showed improved biofilm eradication activities against MRSA-2 biofilms with HP 214 being the most potent analogue reporting an MBEC of 12.5 μM (MBC=3.13 μM; FIG. 2A). Despite the improved biofilm eradication potencies for HP 209, 210, 213 and 214, phenazine 211 was inactive as a biofilm eradicator at the highest concentration tested (MBEC>200 M). Active HP biofilm eradicators demonstrated near equipotent killing of MRSA-2 biofilm and planktonic cells, which is a desirable characteristic of a biofilm-eradicating agent, by reporting MBEC:MBC ratios between 1.0 and 4.0.

TABLE 5
Exemplary results of biological assays of HP analogues, conventional
antibiotics, and other control agents.^{d}

| | | MRSA-2 | | | % | | HeLa |
|---|---|---|---|---|---|---|---|
| | MRSA-2 | stationary | MRSE | VRE | Hemolysis | Mtb | Cytotoxicity |
| Compound | MBC/MBEC | killing | MBC/MBEC | MBC/MBEC | at 200 μM | MIC | IC₅₀ |
| 202 | 31.3^{b}/93.8^{a} | | 23.5^{a}/250^{b} | 23.5^{a}/9.38^{a} | <1 | 25 | >100 |
| 209 | 18.8^{a}/50 | | 1.56/4.69^{a} | 0.39/0.39 | <1 | 6.25 | >100 |
| 210 | 25/25 | | 1.56/6.25 | 0.39/0.39 | <1 | >50 | >100 |
| 211 | 200/>200 | | | | <1 | | |
| 213 | 25/37.5^{a} | | 9.38^{a}/75 | | 1.4 | 3.13 | >100 |
| 214 | 3.13/12.5 | >3 log | 1.56/1.56 | 0.20^{c}/0.20^{c} | 2.7 | 12.5 | >50 |
| | | (12.5 μM) | | | | | |
| Vancomycin | 3.0^{a}/>2,000 | none (100 μM) | 3.0^{a}/>2,000 | >200/150^{b} | <1 | | |
| Daptomycin | 62.5^{b}/>2,000 | none (100 μM) | | | 1.7 | | |
| Linezolid | 15.6/>2,000 | ~1 log | | | <1 | | |
| | | (100 μM) | | | | | |
| QAC-10 | 31.3^{b}/125 | ~2 log | | 3.0^{a}/3.0^{a} | >99 | | |
| | | (100 μM) | | | | | |
| CCCP | 31.3/1,000 | | | | 3.5 | | |

^{a}Midpoint value for a 2-fold range in independent experiments.
^{b}Midpoint value for a 4-fold range in independent experiments.
^{c}Lowest concentration tested; All MBC/MBEC values were obtained from 2 to 6 independent experiments.
^{d}All concentrations are reported as μM. Percent hemolysis (red blood cell lysis) and HeLa cytotoxicity (24 hour; LDH assay) were determined using a spectrophotometer (96-well plates).

The HPs demonstrated enhanced biofilm eradication activities against methicillin-resistant Staphylococcus epidermidis (MRSE; ATCC 35984) and vancomycin-resistant Enterococcus faecium (ATCC 700221) compared to MRSA-2 (Table 5). HPs 209, 210, 213, and 214 showed improved eradication activities against MRSE/VRE biofilms compared to 202. HP 214 demonstrated the most potent eradication activities against MRSE (MBEC=1.56 μM; 160-fold more potent than HP 202) and VRE (MBEC=0.20 μM; 47-fold more potent than HP 202) and displayed the most potent biofilm-eradicating activities reported to date.

Front-running MRSA treatments vancomycin, daptomycin and linezolid, were evaluated alongside the HP analogues in CBD assays and, despite these antibiotics demonstrating moderate to excellent potency against MRSA-2 planktonic cells, all conventional antibiotics were unable to eradicate biofilms at the highest concentration tested (MBEC>2,000 μM). As with MRSA-2, vancomycin reported potent bactericidal concentrations (MBC=3.0 μM; Table 5) against MRSE planktonic cells in CBD assays, yet was unable to eradicate biofilms against both pathogens (MBEC>2,000 μM; Table 5). The corresponding MBEC:MBC ratios of this small panel of anti-MRSA antibiotics against MRSA-2 were between >32 and >667 demonstrating the high level of antibiotic tolerance by these biofilms.

Two known biofilm eradicators were evaluated as positive controls, including QAC-10 (membrane disruptor)¹⁰ and carbonyl cyanide m-chlorophenyl hydrazone (CCCP; proton ionophore)²² (Table 5). Both controls demonstrated biofilm eradication against MRSA-2 with QAC-10 being significantly more potent than CCCP. HP analogues demonstrated enhanced biofilm eradication potencies up to 10-fold against MRSA-2 and up to 15-fold against VRE biofilms compared to QAC-10 (Table 5).

All compounds were screened for hemolytic activity against red blood cells at 200 μM (single concentration). QAC-10 reported >99% hemolysis at 200 μM, a feature associated with membrane-lysing agents. HP analogues did not demonstrate hemolytic activity at 200 μM (<3% hemolysis, Table 5). Due to the drastic differences in hemolytic activities between HP analogues and QAC-10, it was hypothesized that HP analogues may not eradicate biofilms through disruption of bacterial membranes.

HP analogues and antibiotics were also evaluated against persistent bacteria (e.g., MRSA-2 persister cells and MtB) in non-biofilm cultures. Stationary cultures of S. aureus are known to consist of high populations of metabolically dormant persister cells.^{23,24} When stationary cultures of MRSA-2 were treated with HP 214 and front-running MRSA antibiotics (vancomycin, daptomycin, and linezolid), only 214 demonstrated a dramatic killing effect (>99.9%/>3-log reduction of viable cells) that continued to increase over the 24 hour experiment against MRSA-2 persisters at 12.5 μM (FIG. 3; Table 5). Vancomycin and daptomycin were unable to kill MRSA-2 persister cells at 100 μM (>100-fold the MIC value for vancomycin), while linezolid showed initial killing of MRSA-2 persisters at 100 μM (2-log reduction after 3 hours) despite MRSA-2 recovering to an overall 1 log reduction of viable stationary cells after 24 hours. In addition, QAC-10 was evaluated as a positive control at 100 μM, and rapid and sustained killing of 2-3 logs against stationary MRSA-2 cells was observed (FIG. 3; Table 5).

Since HP analogues proved to be effective at eradicating non- or slow-growing bacterial biofilms and stationary cultures, HP analogues were evaluated against the slow-growing human pathogen M. tuberculosis (MtB). Tuberculosis continues to be the leading cause of death by bacterial infection worldwide,²⁵ largely due to its persistent nature. In addition, phenazine small molecules have been reported with potent antibacterial activities against MtB.²⁶ A small panel of HP analogues were tested against M. tuberculosis H37Ra. HP 202 showed a moderate MIC value of 25 M against M. tuberculosis, while 213 demonstrated the most potent anti-tuberculosis activity in the HP panel, with an MIC of 3.13 μM. Streptomycin was used as a positive control in these assays and reported an MIC of 1.32 μM against M. tuberculosis H37Ra.

Biofilm-eradicating HP analogues were evaluated for mammalian cytotoxicity in 24 hour lactate dehydrogenase (LDH) release assays against HeLa cells at 25, 50, and 100 μM. Four of the five biofilm-eradicating agents (HPs 202, 209, 210, 213) reported IC₅₀ values >100 μM, while HP 214 reported an IC₅₀ value >50 μM (Table 5). The HeLa cytotoxicity for HPs, taken together with the lack of toxicity against human red blood cells at 200 μM, indicates that the mechanism for HP analogues may be selective for bacterial cells over mammalian cells. This general lack of toxicity corroborates the in vivo studies described herein, which supports previous work that demonstrates HP 202 is safely administered to mice at 200 mg/kg per day for four days.²⁷

### Live/Dead Staining (Fluorescence Microscopy) of HP-Treated MRSA-2 Biofilms

To support the CBD assay findings, LIVE/DEAD experiments were performed with potent biofilm eradicators 213 and 214 (FIGS. 2B and 2C). A mid-log culture of MRSA-2 was diluted 1:1,000-fold and 500 μL was transferred to each compartment of a 4 compartment CELLVIEW dish (Greiner Bio-One 627871). The dish was then incubated for 24 hours at 37° C. After this time, the cultures were removed and the plate was washed with 0.9% saline. The dish was then treated with the compounds in fresh media at various concentrations. DMSO was used as the negative control in this assay. The dish was incubated with the compound for 24 hours at 37° C. After this time, the cultures were removed and the dish was washed with 0.9% saline for 2 minutes. Saline was then removed and 500 μL of the stain (Live/Dead BacLight Viability Kit, Invitrogen) were added for 15 minutes and left in the dark. After this time, the stain was removed, and the dish was washed twice with 0.9% saline. Then the dish was fixed with 500 μL 4% paraformaldehyde in PBS for 30 minutes. Images of remaining MRSA-2 biofilms were then taken with a fluorescence microscope. All data were analyzed using Image J software.

Interestingly, Live/Dead staining of MRSA-2 biofilms treated with 214 showed a significant amount of biofilm clearance at 0.1 μM against MRSA-2 (FIG. 2B). Similar MRSA-2 biofilm clearance was observed at 0.25 μM, and biofilm killing was observed at 0.013 μM with 213 (red or bright signal; FIG. 2C). The tested HPs were able to effectively clear and kill biofilms.

Co-Treatment with Tiron

The HP analogues are derived from a larger class of redox-active phenazine antibiotics, which are believed to demonstrate antimicrobial activities through the generation of superoxide radicals.¹⁸ When HP analogues were co-treated with tiron, a superoxide radical quenching agent,²¹ the antibacterial activities of HP analogues were not reduced against MRSA-2, MRSE and/or VRE (Table 6). 8-Hydroxyquinoline was used as a positive control in tiron-quenching experiments and showed a complete loss of antibacterial activity against MRSA-2. Halogenated phenazines were co-treated with tiron in MIC assays to determine if select halogenated phenazine compounds demonstrated antibacterial activities as a result of redox-activity. Tiron suppression of antibacterial activities of these HP analogues was not observed, but a loss in antibacterial activity of 8-hydroxyquinoline (a redox-active control) was observed.

TABLE 6
HP compounds and 8-hydroxyquinoline (positive-control) tested
with and without Tiron against MRSA, S. epidermidis, and VRE.

| | | | |
|---|---|---|---|
| | MIC | MIC with Tiron | |
| Compound | (μM) | (μM) | Fold Δ |

MRSA-2

| | | | |
|---|---|---|---|
| 202 | 1.17^{a} | 1.17^{a} | n.a. |
| 209 | 12.5 | 0.10^{b} | −125 |
| 213 | 0.59^{a} | 0.20 | −3 |
| 8-HQ | 6.25 | >100 | +16 |

S. epidermidis 12228

| | | | |
|---|---|---|---|
| 202 | 1.56 | 1.17^{a} | n.a. |
| 209 | 0.1^{b} | 0.1^{b} | n.a. |
| 213 | 0.39 | 0.30^{a} | n.a. |
| 8-HQ | 3.13 | >100 | >+32 |

VRE 700221

| | | | |
|---|---|---|---|
| 202 | 6.25 | 6.25 | n.a. |
| 209 | 0.39 | 0.39 | n.a. |
| 213 | 3.13 | 3.13 | n.a. |
| 8-HQ | 18.8^{a} | >100 | >+12 |

^{a}Corresponds to the midpoint value (2-fold range in MIC) of a two independent experiments.
^{b}Lowest concentration tested.
“Fold Δ” denotes the change in antibacterial activity according to MIC values.
“+” denotes increase in MIC value/loss of antibacterial activity.
“−” denotes decrease in MIC value/increase of antibacterial activity.
“n.a.” denotes insignificant changes in MIC values (≤2-fold changes).

### MRSA-2 Persister Cell Kill Kinetics (Killing of Stationary Cultures)

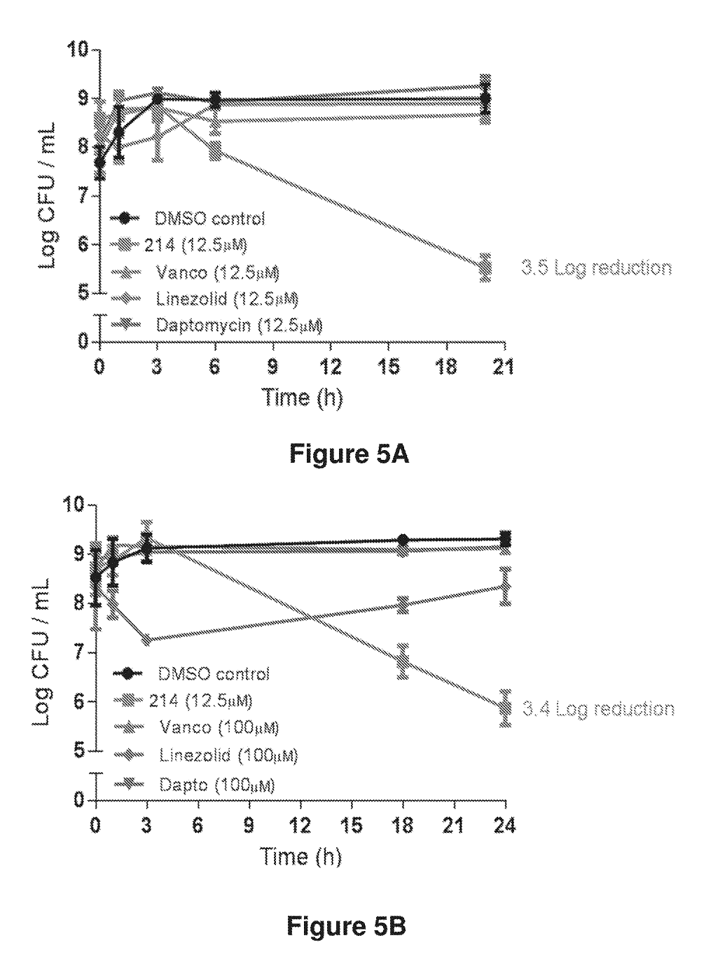

An overnight culture of MRSA-2 was diluted in fresh TSBG (1:13 to 1:20 fold) and allowed to grow with shaking. Once the culture reached stationary phase (4-6 hours), compound 214, MRSA antibiotic (vancomycin, daptomycin, linezolid), or QAC-10 were added at a final test concentration of 12.5 μM or 100 μM. The cultures were incubated with shaking at 250 rpm, and aliquots were removed and plated out at different time points. Colony forming units (CFU) per milliliter data was recorded and plotted using GRAPHPAD PRISM 6.0. Exemplary results are shown in FIGS. 5A and 5B.

### Mouse Toxicity Evaluation

HPs 202 and 209 were dissolved in corn oil at 6.25 mM and 1.0 mM, respectively. Groups of five C57BL/6 mice (Charles River Laboratories International, Inc.; mice were eight weeks old) were treated once daily with HPs 202 (22.1 mg/kg) and 209 (4.2 mg/kg) for 7 days via oral gavage (0.25 mL of formulated corn oil for 25 gram mouse on average). Treated mice experienced no adverse side effects (e.g., changes in weight, seizure, death).

### REFERENCES

[1] K. Lewis, Annu. Rev. Microbiol. B 2010, 64, 357-372.
[2] N. Q. Balaban, J. Merrin, R. Chait, L. Kowalik, S. Leibler, Science 2004, 305, 1622-1625.
[3] B. P. Conlon, Bioessays 2014, 36, 991-996.
[4] M. Kostakioti, M. Hadjifrangiskou, S. J. Hultgren, Cold Spring Harb. Perspect. Med. 2013, 3, a010306.
[5] T. Bjarnsholt, APMIS 2013, 121 (Suppl. 136), 1-54.
[6] K. Lewis, Nat. Rev. Microbiol. 2007, 5, 48-56.
[7] (a) M. H. Fletcher, M. C. Jennings, W. M. Wuest, Tetrahedron 2014, 70, 6373-6383; (b) R. J. Worthington, J. R. Richards, C. Melander, Org. Biomol. Chem. 2012, 10, 7457-7474.
[8] G. H. De Zoysa, A. J. Cameron, V. V. Hegde, S. Raghothama, V. Sarojini, J Med. Chem. 2015, 58, 625-639.
[9] J. Hoque, M. M. Konai, S. Gonuguntla, G. B. Manjunath, S. Samaddar, V. Yarlagadda, J. Haldar, J. Med. Chem. 2015, 58, 5486-5500.
[10] M. C. Jennings, L. E. Ator, T. J. Paniak, K. P. C. Minbiole, W. M. Wuest, ChemBioChem 2014, 15, 2211-2215.
[11] C. C. Hughes, W. Fenical, Chem. Eur. J. 2010, 16, 12512-12525.
[12] W.-L. Ng, B. L. Bassler, Annu. Rev. Genet. 2009, 43, 197-222.
[13] M. Hentzer, H. Wu, J. B. Anderson, K. Riedel, T. B. Rasmussen, N. Bagge, N. Kumar, M. A. Schembri, Z. Song, P. Kristoffersen, M. Manefield, J. W. Costerton, S. Molin, L. Eberl, P. Stienberg, S. Kjelleberg, N. H\acute{ø}iby, M. Givskov, EMBO J. 2003, 22, 3803-3815.
[14] H. Wu, Z. Song, M. Hentzer, J. B. Anderson, S. Molin, M. Givskov, N. H\acute{ø}iby, J. Antimicrob. Chemother. 2004, 53, 1054-1061.
[15] J. C. Kwan, T. Meickle, D. Ladwa, M. Teplitski, V. J. Paul, H. Luesch Mol. BioSyst. 2011, 7, 1205-1216.
[16] G. Navarro, A. T. Cheng, K. C. Peach, W. M. Bray, V. S. Bernan, F. H. Yildiz, R. G. Linington, Antimicrob. Agents Chemother. 2014, 58, 1092-1099.
[17] A. T. Garrison, F. Bai, Y. Abouelhassan, N. G. Paciaroni, S. Jin, R. W. Huigens III, RSC Adv. 2015, 5, 1120-1124.
[18] A. Price-Whelan, L. E. P. Dietrich, D. K. Newman, Nat. Chem. Biol. 2006, 2, 71-78.
[19] M. Conda-Sheridan, L. Marler, E. J. Park, T. P. Kondratyuk, K. Jermihov, A. D. Mesecar, J. M. Pezzuto, R. N. Asolkar, W. Fenical, M. Cushman, J. Med. Chem. 2010, 53, 8688-8699.
[20] H. Ceri, M. E. Olson, C. Stremick, R. R. Read, D. Morck, A. Buret, J. Clin. Microbiol. 1999, 37, 1771-1776.
[21] Taiwo, F. A. Spectroscopy, 2008, 22, 491-498.
[22] Y. J. Eun, M. H. Foss, D. Kiekebusch, D. A. Pauw, W. M. Westler, M. Thanbichler, D. B. Weibel, J. Am. Chem. Soc. 2012, 134, 11322-11325.
[23] I. Keren, N. Kaldalu, A. Spoering, Y. Wang, K. Lewis, FEMS Microbiol. Lett. 2004, 230, 13-18.
[24] S. Lechner, K. Lewis, R. Bertram, J. Mol. Microbiol. Biotechnol. 2012, 22, 235-244.
[25] D. B. Young, M. D. Perkins, K. Duncan, C. E. Barry, J. Clin. Invest. 2008, 118, 1255-1265.
[26] D. Zhang, Y. Liu, C. Zhang, H. Zhang, B. Wang, J. Xu, L. Fu, D. Yin, C. B. Cooper, Z. Ma, Y. Lu, H. Huang, Molecules 2014, 19, 4380-4394.
[27] L. Marler, M. Conda-Sheridan, M. A. Cinelli, A. E. Morrell, M. Cushman, L. Chen, K. Huang, R. Van Breemen, J. M. Pezzuto, Anticancer Res. 2010, 30, 4873-4882.
[28] Conda-Sheridan, M.; Marler, L.; Park, E. J.; Kondratyuk, T. P.; Jermihov, K.; Mesecar, A. D.; Pezzuto, J. M.; Asolkar, R. N.; Fenical, W.; Cushman, M. J Med. Chem. 2010, 53, 8688-8699.
[29] Emmanuvel, L.; Shukla, R. K.; Sudalai, A.; Gurunath, S.; Sivaram, S. Tetrahedron Lett. 2006, 47, 4793-4796.
[30] Clinical and Laboratory Standards Institute. 2009. Methods for dilution antimicrobial susceptibility tests for bacteria that grow aerobically; approved standard, 8th edition (M7-M8), Clinical and Laboratory Standard, Wayne, Pa., 2009.
[31] Abouelhassan, Y.; Garrison, A. T.; Bai, F.; Norwood IV, V. M.; Nguyen, M. T.; Jin, S.; Huigens III, R. W. ChemMedChem, 2015, 10, 1157-1162.
[32] Stringer, J. R.; Bowman, M. D.; Weisblum, B.; Blackwell, H. ACS Comb. Sci. 2011, 13, 175-180.

### Example 3

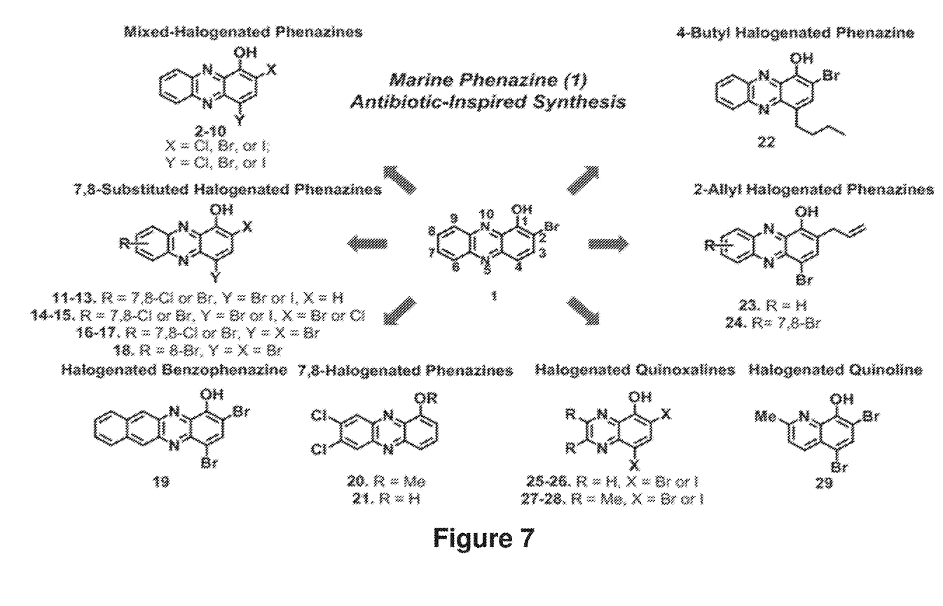

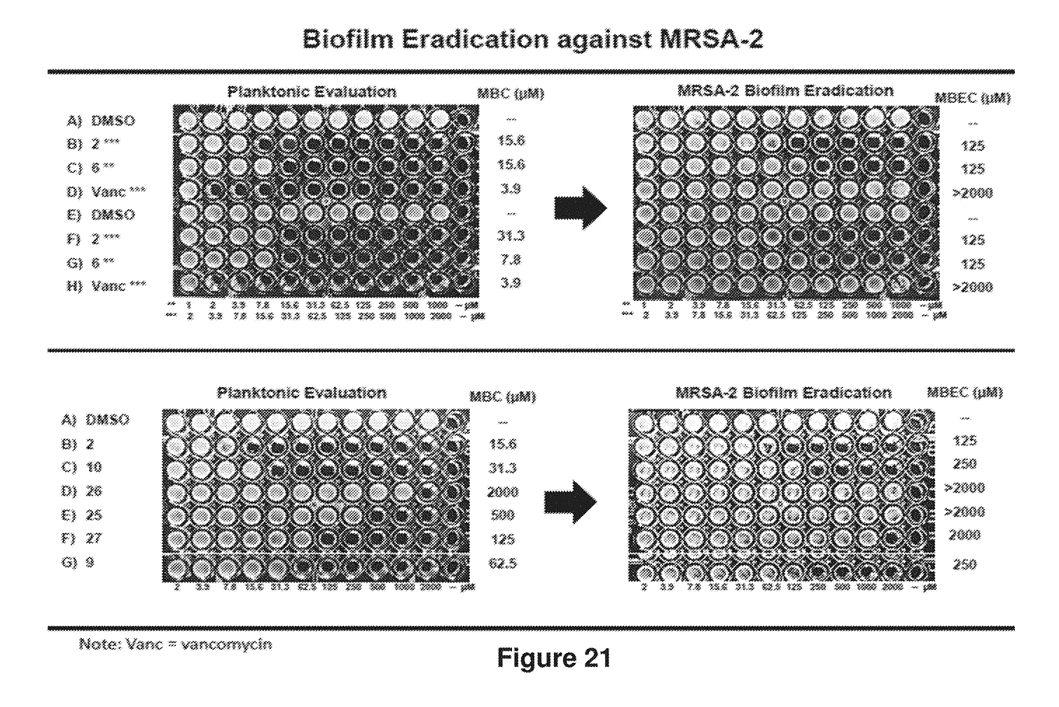

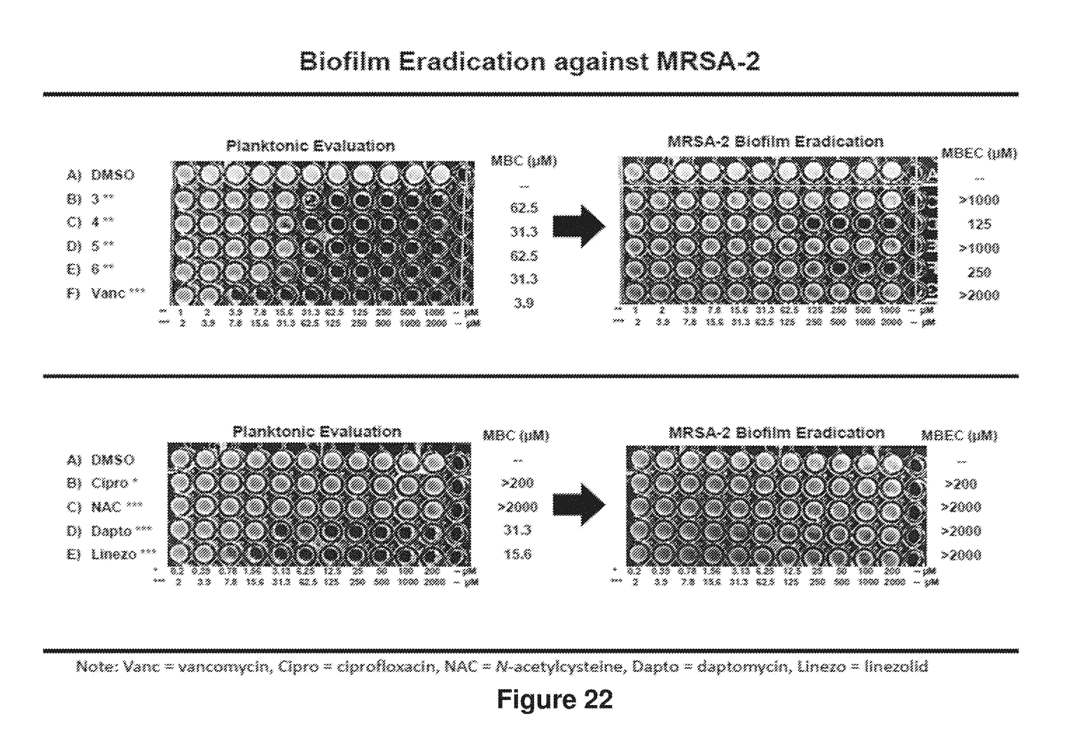

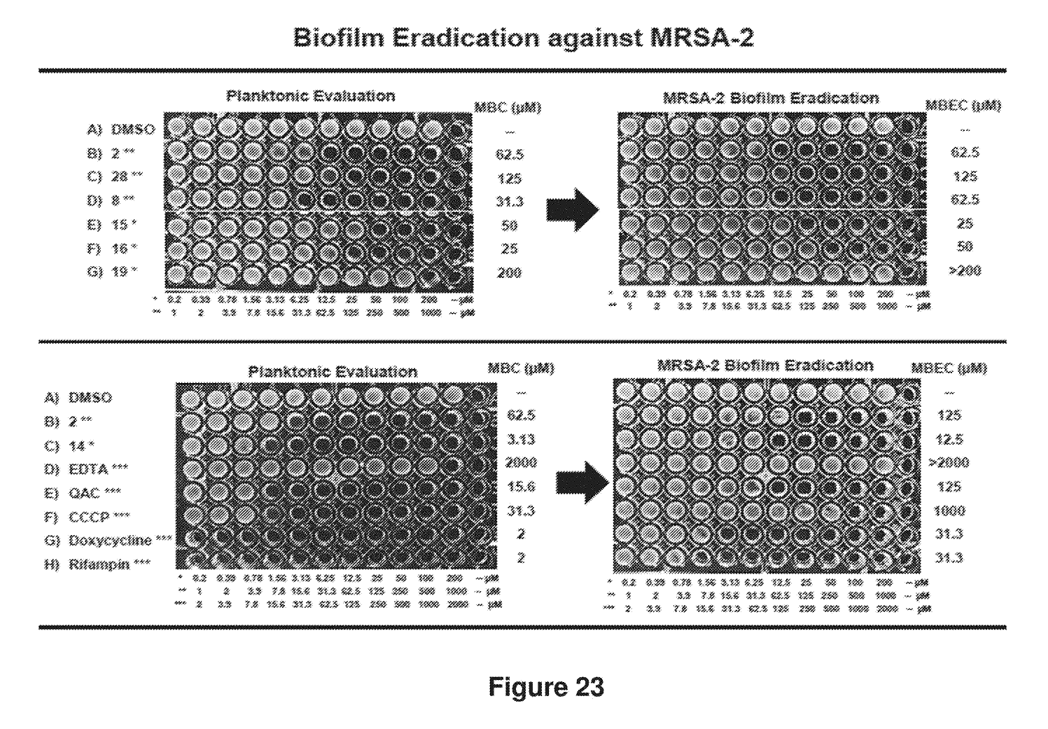

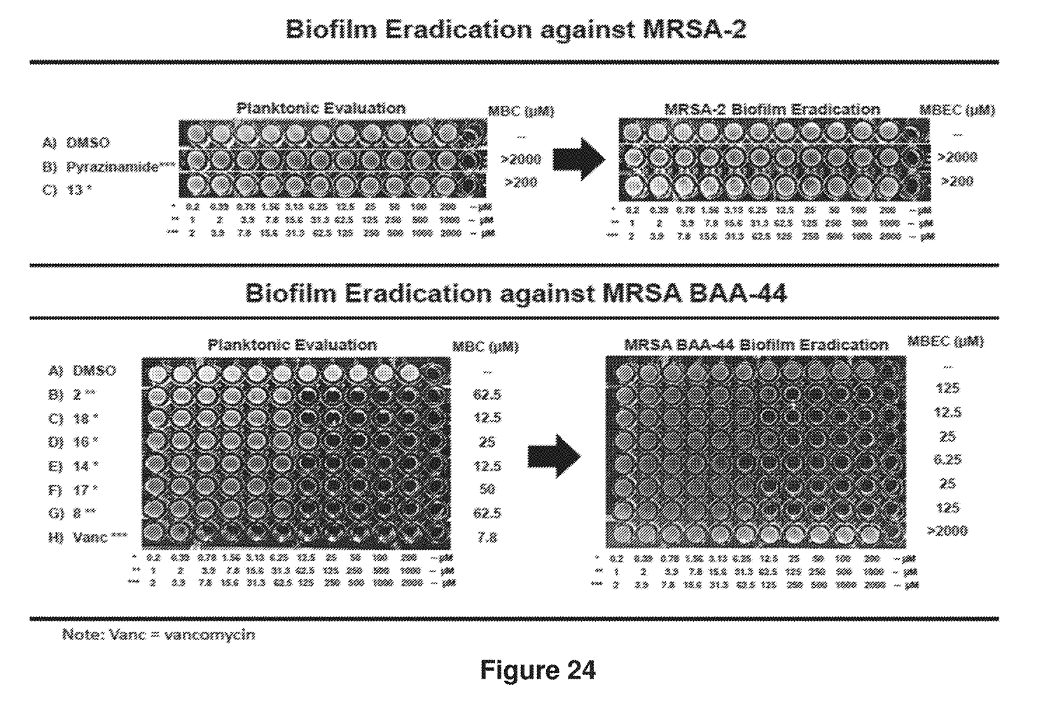

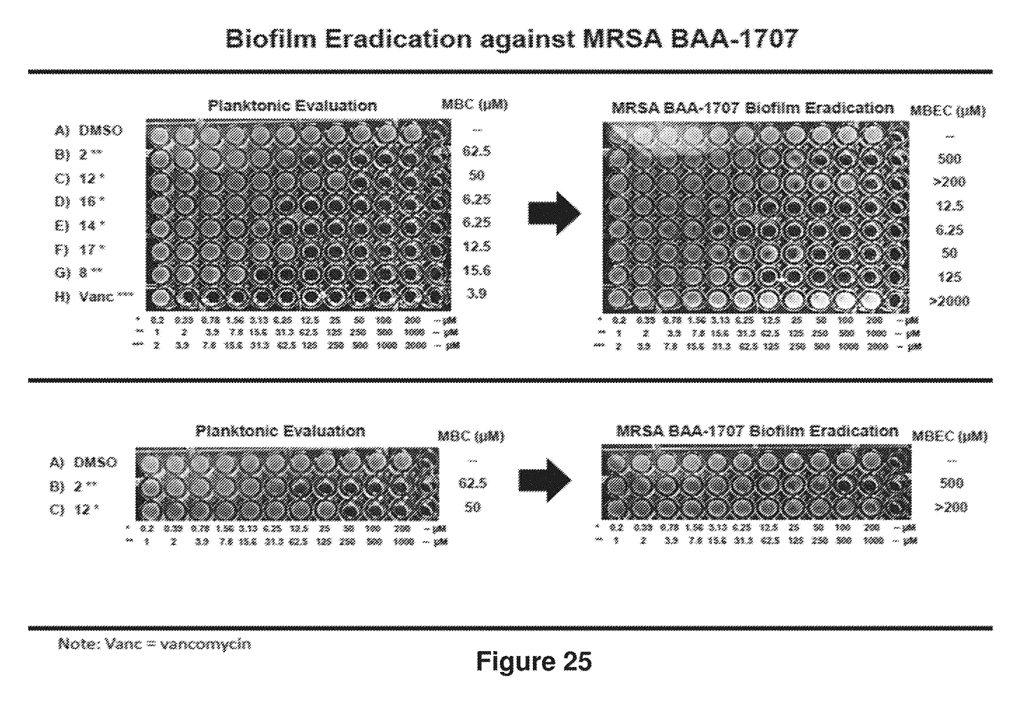

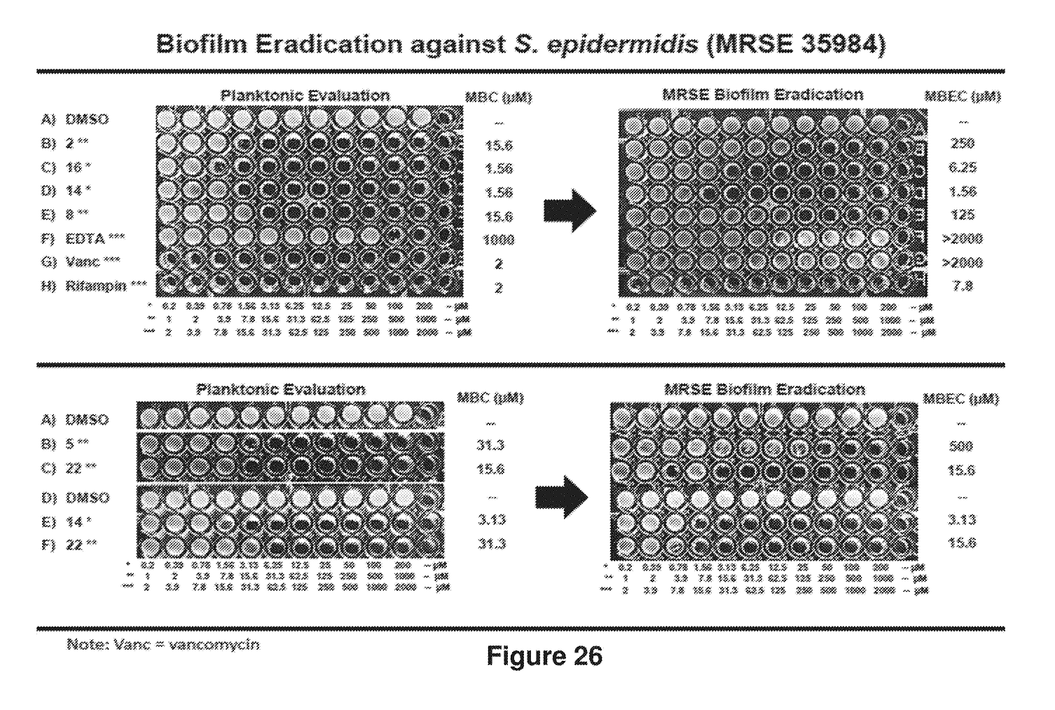

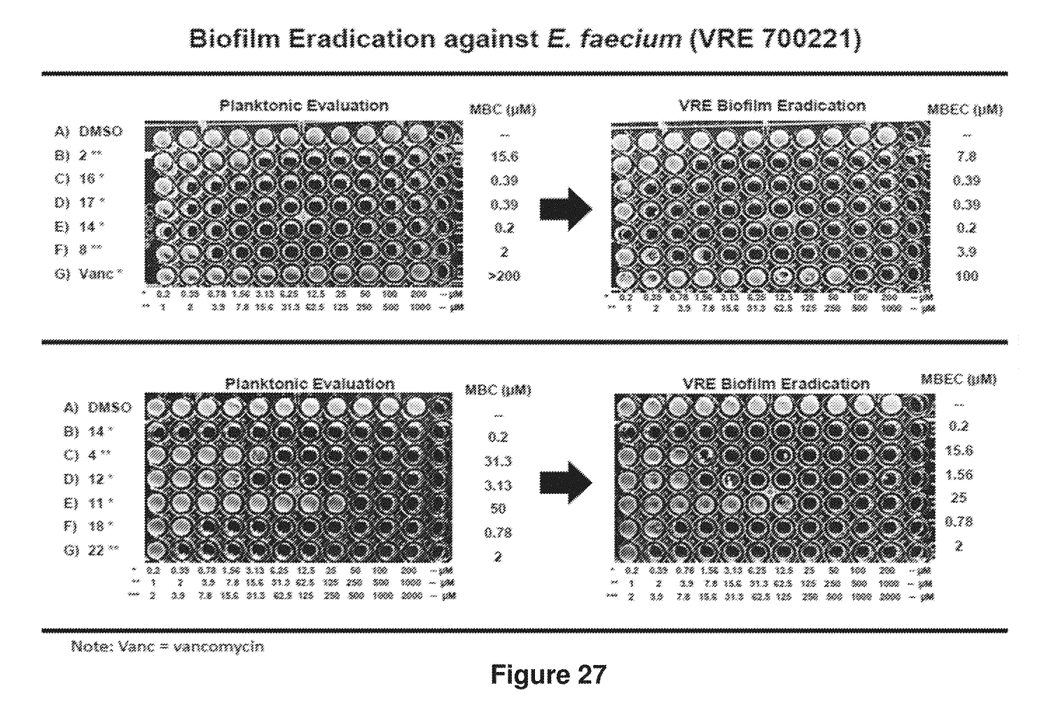

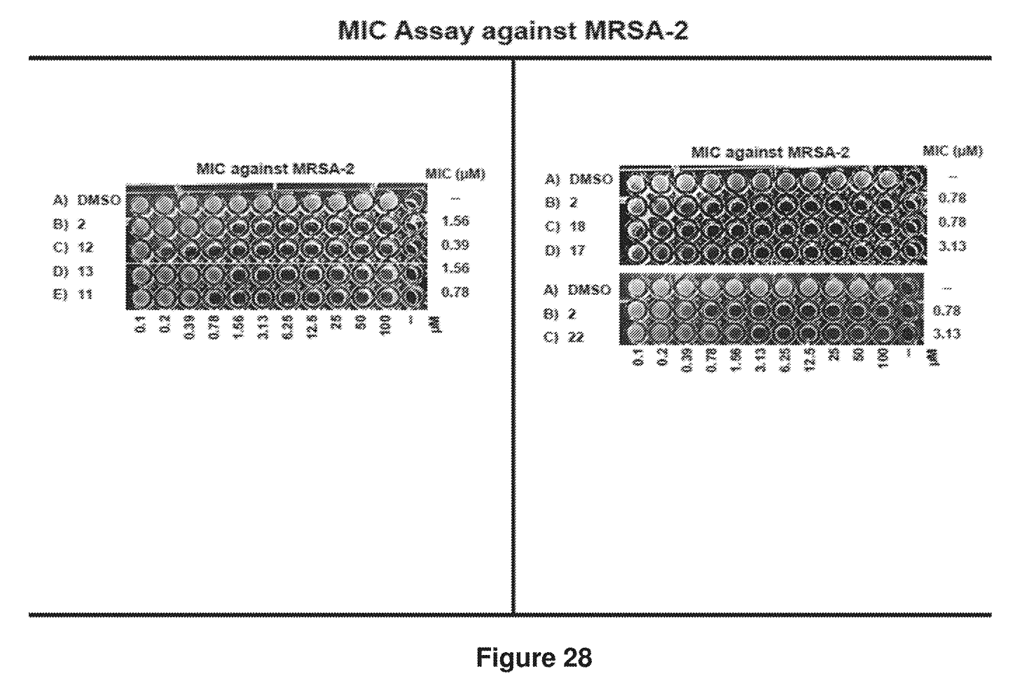

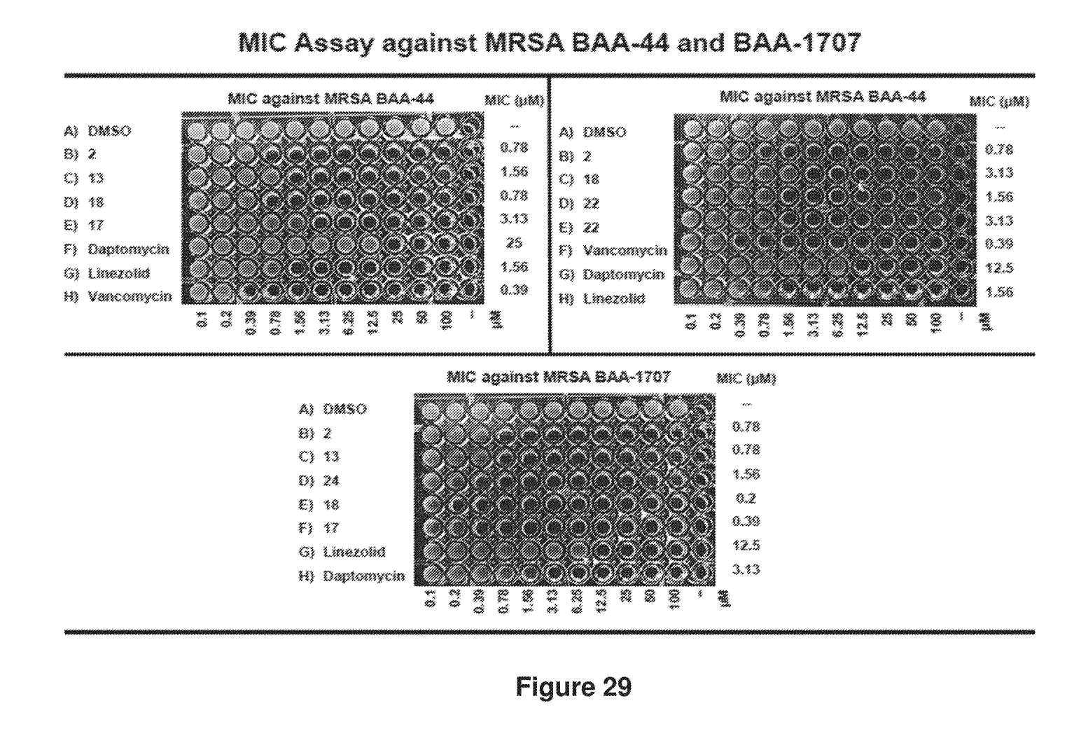

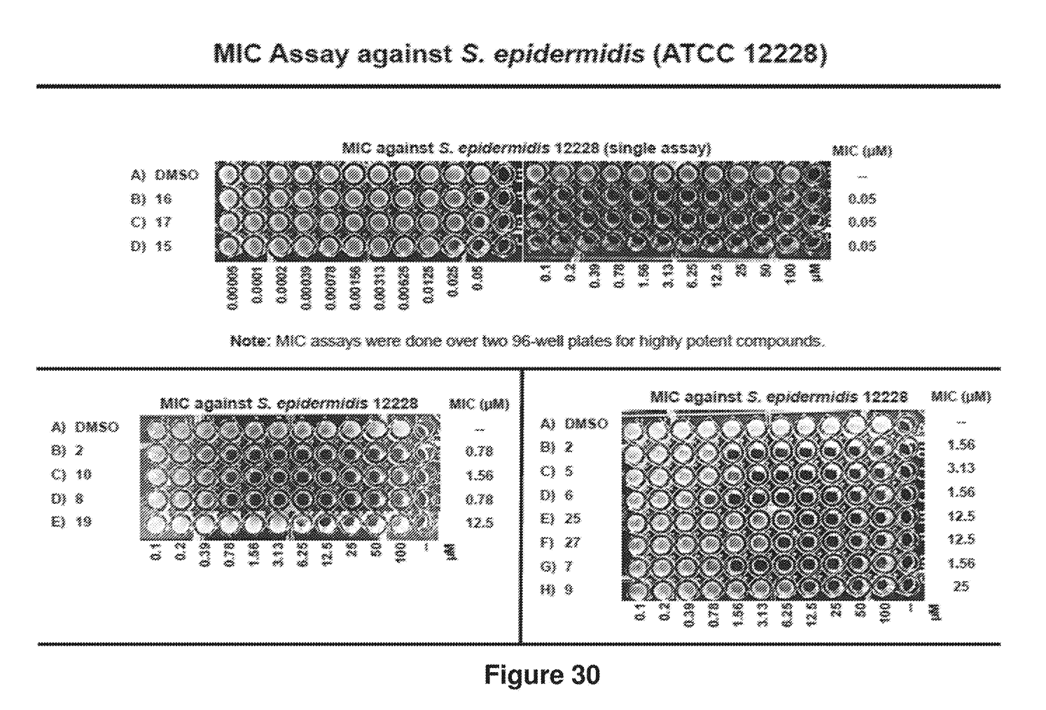

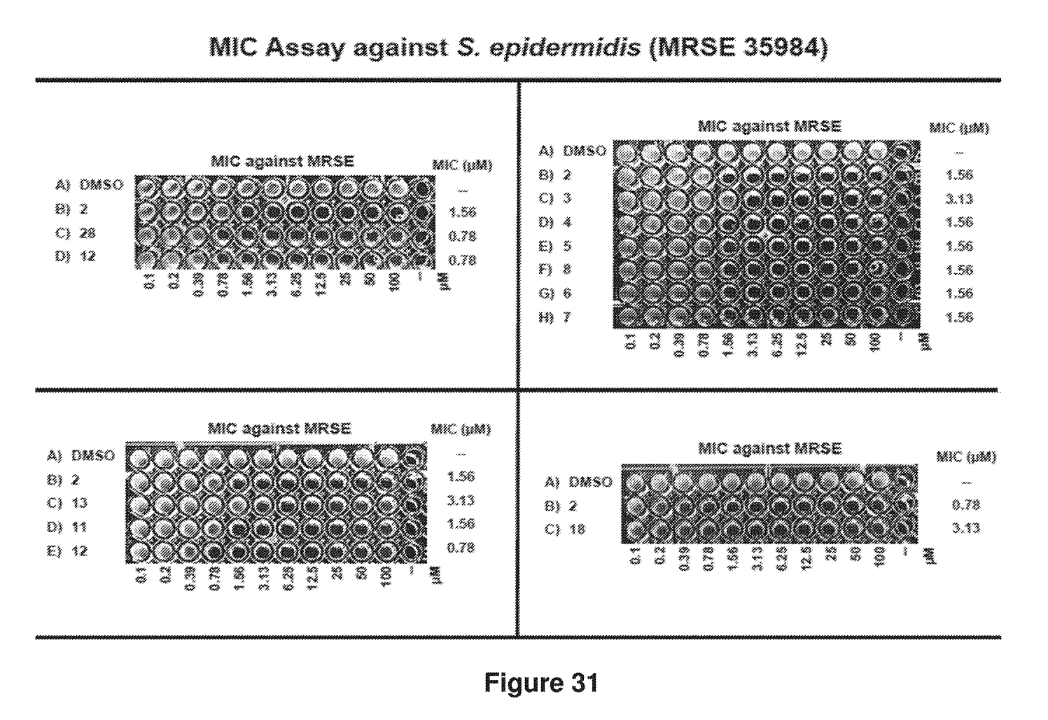

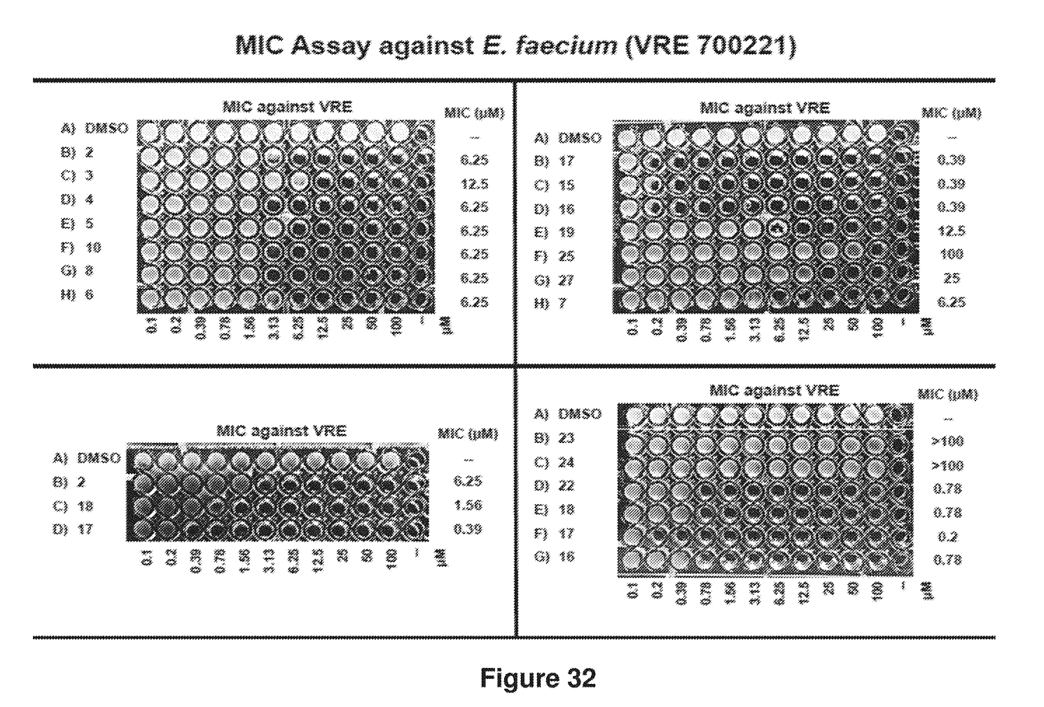

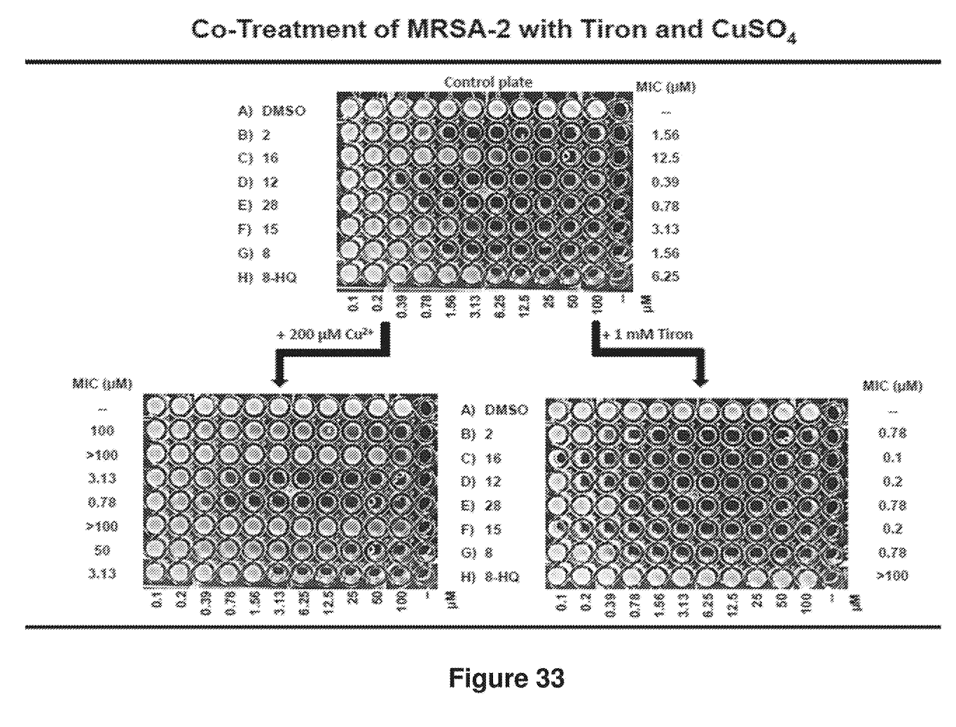

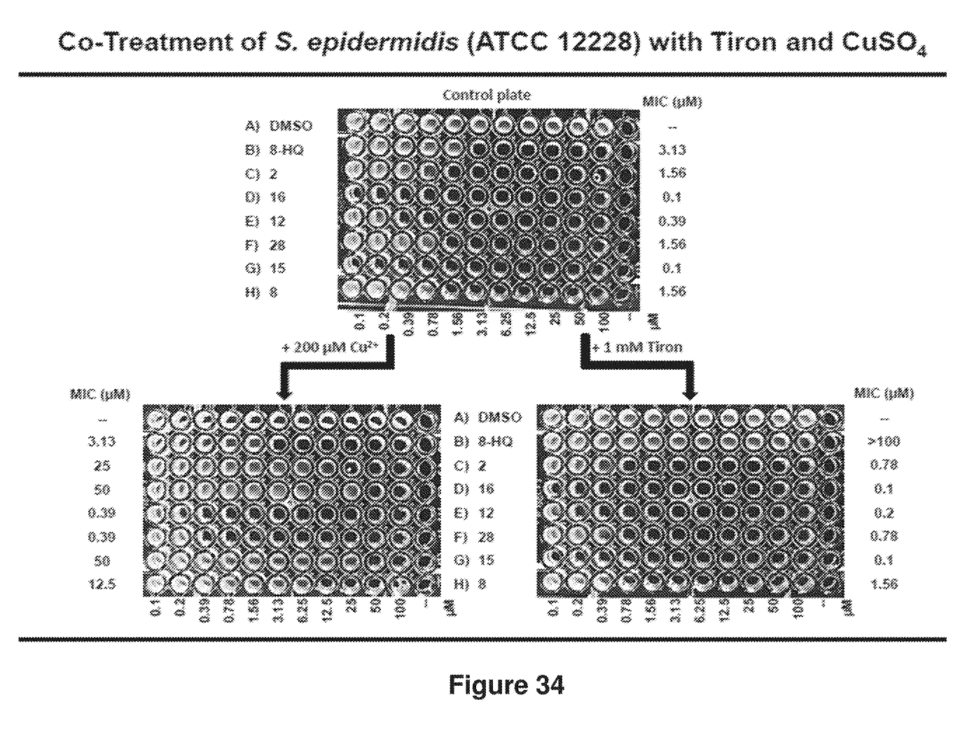

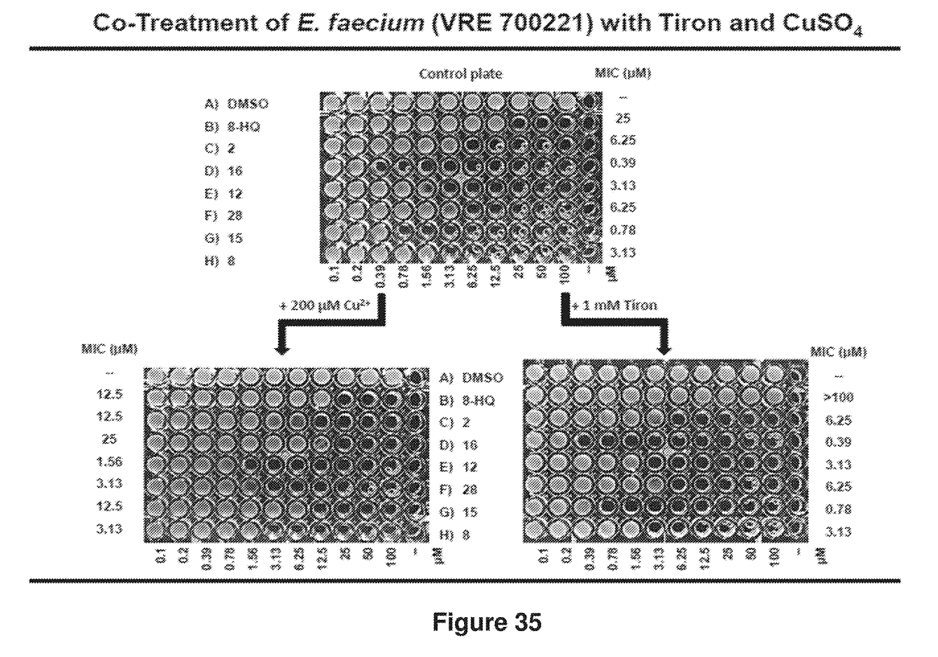

The relationships between the compound numbers referenced in Example 3 (including FIGS. 7 to 35) and the compound structures referenced in Example 3 (including FIGS. 7 to 35) are applicable to Example 3 (including FIGS. 7 to 35).

Persistent bacteria, including persister cells within surface-attached biofilms and slow-growing pathogens lead to chronic infections that are tolerant to antibiotics. Here, we describe the structure-activity relationships of a series of halogenated phenazines (HP) inspired by 2-bromo-1-hydroxyphenazine 1. Using multiple synthetic pathways, we probed diverse substitutions of the HP scaffold in the 2-, 4-, 7- and 8-positions providing critical information regarding their antibacterial and bacterial eradication profiles. Halogenated phenazine 14 proved to be the most potent biofilm-eradicating agent (≥99.9% persister cell killing) against MRSA (MBEC<10 μM), MRSE (MBEC=2.35 μM) and VRE (MBEC=0.20 μM) biofilms while 11 and 12 demonstrated excellent antibacterial activity against M. tuberculosis (MIC=3.13 μM). Unlike antimicrobial peptide mimics that eradicate biofilms through the general lysing of membranes, HPs do not lyse red blood cells. HPs are promising agents that effectively target persistent bacteria while demonstrating negligible toxicity against mammalian cells.

Current antibiotics operate primarily through growth-dependent mechanisms and suffer from an inability to effectively treat persistent and chronic infections involving bacterial biofilms (surface-attached communities)¹⁻⁴ and Mycobacterium tuberculosis (MtB).⁵ Bacteria prefer to exist in biofilms that are composed of specialized, non-replicating persister cells encased within an extracellular matrix of biomolecules and demonstrate tolerance toward every class of antibiotic therapy.^{4,6,7} Antibiotic tolerance is an innate bacterial phenotype attributed to metabolically dormant persister cells and distinct from acquired antibiotic resistance gained by rapidly-dividing, free-floating (planktonic) bacteria induced by antibiotic treatment.^{6,8,9} Chronic and recurring bacterial infections are attributed to antibiotic tolerance which results in an estimated 17 million new biofilm infections and >500,000 annual deaths in the United States.^{10,11} An unproductive antibiotic pipeline over the last 45 years has led to many pharmaceutical companies eliminating or significantly downsizing antibacterial discovery programs.¹² In addition, we have only begun to understand the importance of biofilms in human health over the last 20 years, and currently there are no biofilm- or persister-eradicating therapeutics available to effectively treat chronic infections. In order to address persistent and biofilm-associated bacterial infections, innovative strategies are required to identify novel small molecules capable of targeting and killing non-replicating persister cells through unique, growth-independent mechanisms.

Many efforts have been made to modulate bacterial biofilms through the perturbation of bacterial signaling (quorum sensing) processes¹³ or the identification of non-growth altering biofilm inhibitors and dispersal agents¹¹; however, very few classes of biofilm-eradicating agents have been reported.¹⁴⁻¹⁸ Unlike biofilm inhibitors and dispersal agents, biofilm-eradicating agents kill persister cells within surface-attached biofilms and have the potential to be stand-alone therapeutics for the treatment of biofilm-associated infections. The most prevalent class of biofilm-eradicating agents are the antimicrobial peptides (AMPs) and mimics thereof, which operate through bacterial membrane disruption and lysis.¹⁴⁻¹⁸ The development of AMP-based antibacterial therapeutics is indeed promising; however, effective AMPs must target bacterial membranes over mammalian cell membranes to reduce or eliminate potential human toxicity concerns. New biofilm-eradicating agents that operate through complimentary mechanisms are critical to address biofilm-associated bacterial infections and have the potential to address other problems associated with chronic bacterial infections, such as targeting slow-growing Mycobacterium infections.

The marine environment is an extensive source of microbial diversity and new antibacterial agents,¹⁹ thus it is fertile ground for the discovery of antibacterial agents that operate through new modes of action and could lead to effective biofilm-eradicating agents. Marine sources have provided diverse classes of natural products able to modulate quorum sensing,²⁰⁻²³ inhibit biofilm formation and/or disperse established biofilms.^{11,24} Recently, our group found that 2-bromo-1-hydroxyphenazine 1²⁵ (FIG. 7), a phenazine originally isolated by Cushman and co-workers from a marine Streptomyces species, demonstrates potent antibacterial activity against Staphylococcus aureus and S. epidermidis (minimum inhibitory concentration or MIC of 6.25 μM; 1.7 μg/mL).²⁶ The antibacterial potency of this marine phenazine antibiotic is enhanced with a second bromine atom installed at the 4-position of the phenazine heterocycle (2^{25,26}; MIC 0.78-1.56 μM, or 0.27-0.55 μg/mL against S. aureus and S. epidermidis).²⁶ Halogenated phenazine (HP) 2 displayed effective eradication activity against methicillin-resistant Staphylococcus aureus (MRSA) biofilms (minimum biofilm eradication concentration or MBEC ˜150 μM).²⁷ In addition, we recently reported the identification of a small series of HP analogues that demonstrated potent biofilm eradication activities (MBEC=0.2-12.5 μM) against several maj or human pathogens, including: MRSA, MRSE and VRE.²⁸ Here, we report the full account of our investigations detailing the chemical synthesis, biological evaluation and structure-activity relationships of a series of 29 HP analogues.

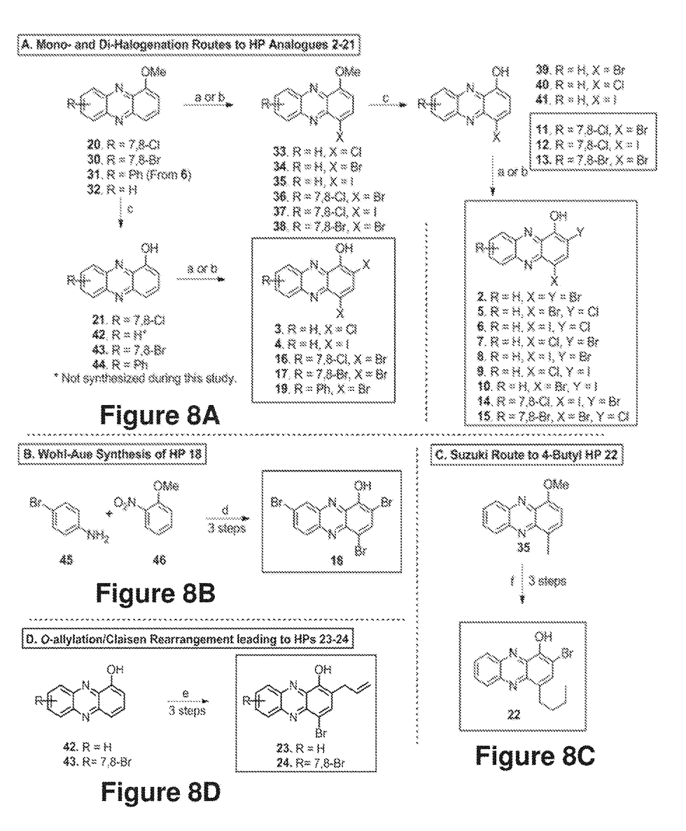

Our chemical synthesis efforts were motivated by our preliminary success in identifying potent biofilm-eradicating agents, and thus, newly synthesized HP analogues would be evaluated in an array of biological assays. Our initial goals were to synthesize a focused, yet diverse set of marine phenazine analogues with a primary emphasis on functionalizing the 7- and 8-positions (FIG. 7) of the phenazine heterocycle. These synthetic goals can be achieved through the following transformations: (1) condensation between a quinone and phenylenediamine^{25,28} and (2) Wohl-Aue²⁹ condensation between anilines and nitroarenes. In addition, we were interested in probing alternative substitution patterns with various combinations of halogens and alkyl substituents of the phenazine scaffold which required multiple synthetic pathways (FIGS. 8A to 8D (Schemes 3A to 3D)).

We began our chemistry efforts by synthesizing dichlorinated and diiodinated versions of HP 2 (FIG. 8A). Although the dibromination reaction of 1-hydroxyphenazine was high-yielding when using N-bromosuccinimide (NBS; 99% yield),²⁶ the synthesis of 2,4-dichloro-1-hydroxyphenazine 3 and 2,4-diiodo-1-hydroxyphenazine 4 from 1-hydroxyphenazine 42 gave considerably lower yields. Chlorination at the 2- and 4-positions of 1-hydroxyphenazine using N-chlorosuccinimide (NCS) proceeded in a 47% yield to give 2 while iodination using N-iodosuccinimide (NIS) yielded 3 in only 19%. We also synthesized a series of mixed halogenated analogues 5 to 10 via a regioselective monohalogenation reaction at the 4-position of 1-methoxyphenazine 32 to give phenazines 33 to 35 (68-76% yield), followed by demethylation using boron tribromide (BBr₃) to give phenazine intermediates 39 to 41 (86-97% yield). A final halogenation reaction at the 2-position of 1-hydroxyphenazines 39 to 41 yielded mixed halogenated analogues 5 to 10 (34-91% yield).

1-Methoxyphenazines 20, 30, and 31 (synthesized via quinone-phenylenediamine condensation) were demethylated using boron tribromide to afford 1-hydroxyphenazines 21, 43, and 44 in 78-99% yield (FIG. 8A). A dibromination reaction at the 2- and 4-positions of the phenazine heterocycle was carried out using two equivalents ofN-bromosuccinimide (NBS) to afford HP analogues 16, 17, and 19 in 38-84% yield.²⁸ This synthetic approach enabled us to access key HP analogues with diversity at the 7- and 8-positions of the phenazine ring; however, the dibromination reaction with NBS proved to be troublesome and gave inconsistent results (e.g., low yields, impurities difficult to remove) during these studies.

To circumvent the problems encountered during the dihalogenation reaction, we used 1-methoxyphenazines 20, 30, and 31 to sequentially install a single halogen atom at the 4-position of the phenazine ring upon treatment with one equivalent of halogenating agent (NCS, NBS, NIS, or KI/NaIO₄) to give phenazines 36 to 38 in 59-96% yield (FIG. 8A). Following this monohalogenation reaction at the 4-position, 1-methoxyphenazines 36 to 38 were demethylated using boron tribromide to yield 1-hydroxyphenazines 11 to 13 in 91-97% yield. Final halogenation at the 2-position of the phenazine heterocycle cleanly afforded HP analogues 14 and 15 in 52% and 65% yields, respectively.²⁸

### General Procedure for Mono-Halogenation of the 4-Position of 1-Methoxyphenazines (33, 34, 36, and 38)

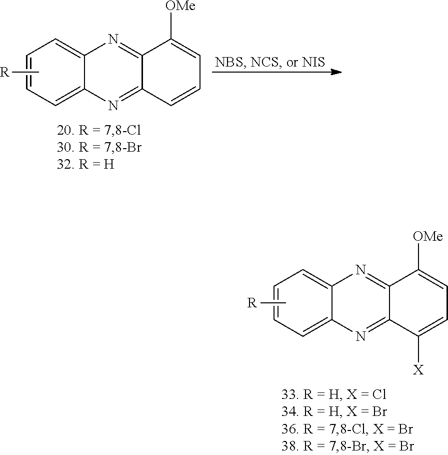

7,8-Dichloro-1-methoxyphenazine 20 (151 mg, 0.54 mmol) was dissolved in dichloromethane (15 mL) before N-bromosuccinimide (106 mg, 0.60 mmol) was added, and the reaction was brought to reflux. The mixture was left to stir overnight until complete (monitored by TLC with dichloromethane). At this time, the reaction was concentrated and adsorbed onto silica gel (via dissolving the crude reaction contents and silica gel in dichloromethane, then concentrating via ROTAVAP) and purified via column chromatography using dichloromethane to elute pure 4-bromo-7,8-dichloro-1-methoxyphenazine 36, which was isolated as a dark yellow solid (96%, 185 mg). Notes: Analogous procedures were used for the chlorination and iodination of 1-methoxyphenazines using N-chlorosuccinimide or N-iodosuccinimide respectively. We observed the formation of side products 33a and 33b during the synthesis of 33. The synthesis of 38 yielded demethylated and ortho-brominated side product 17 in trace amounts. Compound 34 has been previously reported.¹

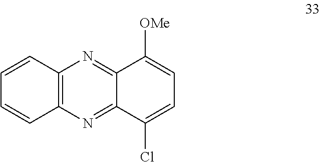 [SMILES: COc1ccc(Cl)c2nc3ccccc3nc12]

Yield:

76% yield; 280 mg of 33 was isolated OMe as a yellow solid. ¹H NMR (400 MHz, CDCl₃): δ 8.19-8.08 (m, 2H), 7.71-7.61 (m, 2H), 7.53 (d, J=8.2 Hz, 1H), 6.66 (d, J=8.2 Hz, 1H), 3.90 (s, 3H). ¹³C NMR (100 MHz, CDCl₃): δ 154.0, 142.9, 141.8, 139.9, 136.6, 131.1, 130.8, 129.6, 129.5, 129.2, 123.5, 105.7, 56.3. HRMS (DART): calc. for C₁₃H₁₀C₁N₂O [M+H]⁺: 245.0476, found: 245.0484. MP: 149-151° C.

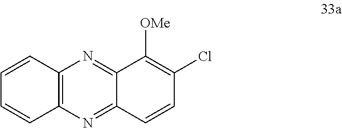 [SMILES: COc1c(Cl)ccc2nc3ccccc3nc12]

Yield:

12% yield; 70.9 mg of 33a was isolated as a yellow solid. ¹H NMR (400 MHz, CDCl₃): δ 8.31 (m, 1H), 8.17 (m, 1H), 7.94 (d, J=9.4 Hz, 1H), 7.87-7.79 (m, 2H), 7.75 (d, J=9.4 Hz, 1H), 4.30 (s, 3H). ¹³C NMR (100 MHz, CDCl₃): δ 151.6, 143.3, 143.2, 142.9, 139.2, 134.5, 132.3, 131.1, 130.2, 129.6, 127.0, 126.0, 62.7. HRMS (DART): calc. for C₁₃H₁₀ClN₂O [M+H]⁺: 245.0476, found: 245.0479. MP: 139-141° C.

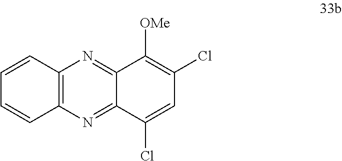 [SMILES: COc1c(Cl)cc(Cl)c2nc3ccccc3nc12]

Yield:

6% yield; 42.6 mg of 33b was isolated as a yellow solid. ¹H NMR (400 MHz, CDCl₃): δ 8.37-8.30 (m, 2H), 7.93 (s, 1H), 7.91-7.87 (m, 2H), 4.30 (s, 3H). ¹³C NMR (100 MHz, CDCl₃): δ 151.0, 143.0, 142.9, 139.6, 139.3, 132.0, 131.7, 131.1, 130.1, 130.0, 128.6, 126.2, 62.9. HRMS (DART): calc. for C₁₃H₉Cl₂N₂O [M+H]⁺: 279.0086, found: 279.0097. MP: 182-184° C.

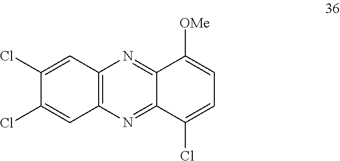 [SMILES: COc1ccc(Cl)c2nc3cc(Cl)c(Cl)cc3nc12]

Yield:

96% yield; 185 mg of 36 was isolated as a dark yellow solid. ¹H NMR (400 MHz, CDCl₃): δ 8.56 (s, 1H), 8.54 (s, 1H), 8.11 (d, J=8.3 Hz, 1H), 6.99 (d, J=8.3 Hz, 1H), 4.17 (s, 3H). ¹³C NMR (100 MHz, CDCl₃): δ 155.3, 142.2, 141.6, 140.9, 137.8, 136.8, 136.4, 134.4, 130.2, 130.2, 114.3, 107.9, 57.0. HRMS (DART): calc. for C₁₃H₈BrCl₂N₂O [M+H]⁺: 356.9192, found: 356.9194. MP: 199-201° C.

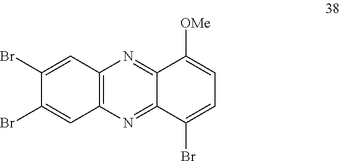 [SMILES: COc1ccc(Br)c2nc3cc(Br)c(Br)cc3nc12]

Yield:

86% yield; 275 mg of 38 was isolated as a dark yellow solid. ¹H NMR (400 MHz, CDCl₃): δ 8.75 (s, 1H), 8.72 (s, 1H), 8.10 (d, J=8.3 Hz, 1H), 6.98 (d, J=8.3 Hz, 1H), 4.16 (s, 3H). ¹³C NMR (100 MHz, CDCl₃): δ 155.3, 142.5, 141.6, 141.2, 137.8, 134.5, 133.7, 133.6, 129.1, 128.6, 114.4, 107.9, 57.0. HRMS (DART): calc. for C₁₃H₈Br₃N₂O [M+H]⁺: 444.8181, found: 444.8181. MP: 241-243° C.

General Condensation Procedure for the Synthesis of 1-methoxyphenazines (20, 30, and 31)

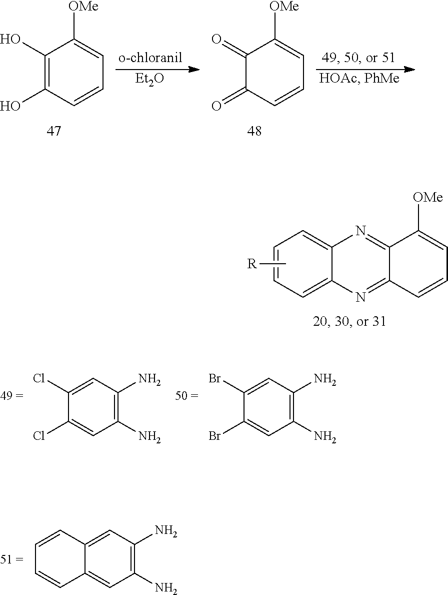

In a round-bottom flask, 3-methoxycatechol 47 (1.73 g, 12.3 mmol) was dissolved in diethyl ether (35 mL), then cooled to −78° C. in a dry ice bath. Tetrachloro-o-benzoquinone (3.19 g, 12.5 mmol) was added, and the reaction was stirred for 4 hours at a constant −78° C. The reaction mixture was filtered twice under vacuum to afford benzoquinone 48 as a dark brown solid, which was used without further purification. Compound 3 was added to a 250 mL round-bottom flask containing 49 (1.29 g, 7.3 mmol) in glacial acetic acid (35 mL) and toluene (35 mL). The reaction was allowed to stir for 24 hours at room temperature. The mixture was neutralized with a solution of saturated sodium bicarbonate, washed with brine, and then extracted with dichloromethane. The combined organic layers were dried with sodium sulfate, filtered, and then removed in vacuo. The resulting crude solid was purified via column chromatography using dichloromethane as the eluent to afford pure compound 20 as a yellow solid (86%, 1.49 g). Note: Analogous procedures were used to synthesize 30 and 31.

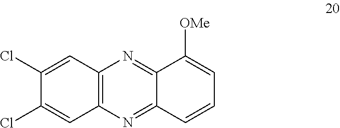 [SMILES: COc1cccc2nc3cc(Cl)c(Cl)cc3nc12]

Yield: 86% yield; 1.49 g of 20 was isolated as a yellow solid. ¹H NMR (400 MHz, CDCl₃): δ 8.53 (s, 1H), 8.35 (s, 1H), 7.80-7.76 (m, 2H), 7.09 (dd, J=4.3, 4.3 Hz, 1H), 4.17 (s, 3H). ¹³C NMR (100 MHz, CDCl₃): δ 155.3, 144.8, 142.2, 140.8, 137.4, 136.0, 135.4, 131.7, 130.5, 129.8, 121.6, 107.4, 56.8. MP: 253-255° C., lit. 245-247° C.²⁵ Note: NMR spectra match those previously reported.²⁵

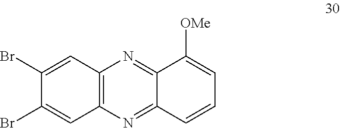 [SMILES: COc1cccc2nc3cc(Br)c(Br)cc3nc12]

Yield:

78% yield; 1.09 g of 30 was isolated as a yellow solid. ¹H NMR (400 MHz, CDCl₃): δ 8.53 (s, 1H), 8.35 (s, 1H), 7.80-7.76 (m, 2H), 7.09 (dd, J=4.3, 4.3 Hz, 1H), 4.17 (s, 3H). ¹³C NMR (100 MHz, CDCl₃): δ 155.3, 144.8, 142.2, 140.8, 137.4, 136.0, 135.4, 131.7, 130.5, 129.8, 121.6, 107.4, 56.8. MP: 253-255° C., lit. 245-247° C.¹ Note: NMR spectra match those previously reported.²⁵

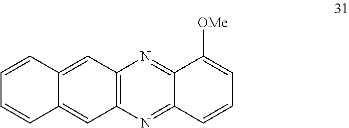 [SMILES: COc1cccc2nc3cc4ccccc4cc3nc12]

Yield:

76% yield; 907 mg of 31 was isolated as a red solid. ¹H NMR (400 MHz, CDCl₃): δ 9.08 (s, 1H), 8.89 (s, 1H), 8.17-8.01 (m, 2H), 7.82 (dd, J=9.1, 1.1 Hz, 1H), 7.73 (dd, J=9.1, 7.4 Hz, 1H), 7.58-7.50 (m, 2H), 7.01 (dd, J=7.4, 1.1 Hz, 1H), 4.21 (s, 3H). MP: 223-225° C., lit. 245-247° C. Note: ¹H NMR was identical to previously reported spectra.²⁵

To gain further insight into the structure-activity relationships of HP antibacterial agents, we employed multiple routes to generate new analogues for our biological studies. In the first route, we utilized a Wohl-Aue reaction between 4-bromoaniline 45 and 2-nitroanisole 46 which afforded 8-bromo-1-hydroxyphenazine 57 in 2% yield after refluxing with potassium hydroxide (KOH) in toluene. Despite this low yield, we were able to advance 57 through the BBr₃ demethylation/dibromination route to afford HP 18 in 45% yield over the final 2 steps (FIG. 8B). For the second synthetic pathway, we carried out a Suzuki coupling between 35 and n-butylboronic acid pinacol ester using 20 mol % tetrakis(triphenylphosphine)palladium(0), followed by subsequent BBr₃ demethylation and NBS bromination reactions to afford lone HP analogue 22 in 17% yield over 3 steps (FIG. 8C). For the third synthetic route, we employed an O-allylation/Claisen rearrangement sequence leading to 2-allylated HP analogues 23 (85% over 3 steps) and 24 (34% over 3 steps) (FIG. 8D). Finally, we synthesized a small series of halogenated quinoxalines (25 to 28). Although these diverse synthetic routes have not been exhausted, we were able to make several HP analogues that were critical in probing various positions of the HP scaffold.

### Antibacterial Evaluation and HeLa Cytotoxicity Studies

Our biological investigations were initiated with the screening of our HP-inspired library (including halogenated quinoline 29) in MIC assays against a panel of pathogenic bacteria, including: S. aureus, S. epidermidis, Enterococcus faecium, Acinetobacter baumannii, Pseudomonas aeruginosa, Klebsiella pneumoniae and Escherichia coli. Lead antibacterial agents from these initial studies were then evaluated against M. tuberculosis (H37Ra). Several bacterial strains used in this panel were drug-resistant (e.g., MRSA-2, MRSA BAA-44, MRSA BAA-1707, S. epidermidis 35984 are methicillin-resistant; E. faecium 700221 is vancomycin-resistant; Table 7).

TABLE 7
Summary of gram-positive antibacterial activities (MIC values
reported) and HeLa cell cytotoxicity of HP analogues. All biological results in this
table are reported in micromolar (μM) concentrations.

| | | | | | | | | HeLa |
|---|---|---|---|---|---|---|---|---|
| | | MRSA | MRSA | | | | | Cytotox. |
| | | BAA- | BAA- | S. epidermidis | MRSE | VRE | M. tuberculosis | IC₅₀ |
| Compound | MRSA-2 | 1707 | 44 | 12228 | 35984 | 700221 | H37Ra | (μM) |
| 2 | 1.56 | 1.17^{a} | 1.17^{a} | 1.17^{a} | 1.56 | 6.25 | 25 | >100 |
| 3 | 3.13 | 3.13 | 3.13 | 3.13 | 3.13 | 12.5 | — | — |
| 4 | 3.13 | 3.13 | 3.13 | 1.56 | 1.17^{a} | 6.25 | — | >100 |
| 5 | 2.35^{a} | 1.56 | 3.13 | 3.13 | 1.56 | 6.25 | — | >100 |
| 6 | 1.56 | 1.56 | 1.56 | 1.56 | 1.56 | 6.25 | — | >100 |
| 7 | 1.56 | 0.78 | 1.56 | 2.35^{a} | 1.56 | 6.25 | — | >100 |
| 8 | 0.78 | 1.17^{a} | 1.17^{a} | 0.78 | 1.56 | 6.25 | — | >100 |
| 9 | 25 | 3.13 | 12.5 | 25 | 25 | 6.25 | — | — |
| 10 | 2.35^{a} | 0.78 | 0.78 | 1.56 | 4.69^{a} | 6.25 | — | — |
| 11 | 0.78 | 0.30^{a} | 1.17^{a} | 3.13 | 1.56 | 6.25 | 3.13 | >100 |
| 12 | 0.39 | 0.15^{a} | 0.39 | 0.39 | 0.78 | 3.13 | 3.13 | >100 |
| 13 | 1.56 | 0.56^{a} | 1.56 | 2.35^{a} | 3.13 | 6.25 | — | — |
| 14 | 3.13 | 0.30^{a} | 3.13 | 0.10^{b} | 3.13 | 0.10^{b} | 12.5 | >50^{c} |
| 15 | 12.5 | 18.8^{a} | 25 | 0.08^{a} | 0.30^{a} | 0.39 | — | >100 |
| 16 | 4.69^{a} | 25 | 50 | 0.08^{a} | 0.30^{a} | 0.39 | 6.25 | >100 |
| 17 | 6.25 | 12.5 | 25 | 0.08^{a} | 2.35^{a} | 0.39 | >50 | >100 |
| 18 | 0.78 | 0.2 | 0.78 | 3.13 | 3.13 | 0.78 | — | — |
| 19 | >100 | 50 | 75^{a} | 12.5 | 18.8^{a} | 9.38^{a} | — | — |
| 20 | >100 | — | — | >100 | >100 | >100 | — | — |
| 21 | >100 | — | — | >100 | >100 | >100 | — | — |
| 22 | 3.13 | 1.56 | 2.35^{a} | 1.56 | 0.59^{a} | 0.78 | — | — |
| 23 | >100 | — | — | >100 | — | >100 | — | — |
| 24 | >100 | — | — | >100 | — | >100 | — | — |
| 25 | 25 | 6.25 | 12.5 | 18.8^{a} | 18.8^{a} | 100 | — | >100 |
| 26 | 3.13 | 1.56 | 1.56 | 6.25 | 3.13 | 12.5 | — | — |
| 27 | 9.38^{a} | 4.69^{a} | 6.25 | 12.5 | 9.38^{a} | 25 | — | — |
| 28 | 1.56 | 0.78 | 1.56 | 2.35^{a} | 0.78 | 6.25 | >50 | >100 |
| 29 | 0.78 | 0.78 | 0.78 | 0.78 | 0.30^{a} | 2.35^{a} | 25 | >100 |
| Vancomycin | 0.59^{a} | 0.39 | 0.39 | 1.17^{a} | 0.78 | >100 | — | — |
| Linezolid | 3.13 | 12.5 | 1.56 | 1.56 | 3.13 | 3.13 | — | — |
| Daptomycin | 4.69^{a} | 3.13 | 18.8^{a} | 6.25 | 12.5 | — | — | — |
| Rifampin | 0.10^{b} | — | — | 0.10^{b} | 0.10^{b} | — | — | — |
| Ciprofloxacin | >100 | — | — | 0.78 | 0.2 | — | — | >100 |
| Streptomycin | — | — | — | — | — | — | 1.32 | — |

Notes:
^{a}Midpoint value (2-fold range in MIC).
^{b}Lowest concentration tested.
^{c}No HeLa cell death at 50 μM with ~70% cell death at 100 μM.
MIC values were obtained from 2-6 independent experiments.

### Minimum Inhibitory Concentration (MIC) Susceptibility Assay (in 96-Well Plate)

The minimum inhibitory concentration (MIC) for each phenazine analogue was determined by the broth microdilution method as recommended by the Clinical and Laboratory Standards Institute (CLSI).⁵² In a 96-well plate, eleven two-fold serial dilutions of each compound were made in a final volume of 100 μL Luria Broth. Each well was inoculated with ˜10⁵ bacterial cells at the initial time of incubation, prepared from a fresh log phase culture (OD₆₀₀ of 0.5 to 1.0 depending on bacterial strain). The MIC was defined as the lowest concentration of compound that prevented bacterial growth after incubating 16 to 18 hours at 37° C. (MIC values were supported by spectrophotometric readings at OD₆₀₀). The concentration range tested for each phenazine analogue/antibacterial during this study was 0.10 to 100 μM. DMSO served as our vehicle and negative control in each microdilution MIC assay. DMSO was serially diluted with a top concentration of 1% v/v. All compounds were tested in two independent experiments, active compounds were tested in a third independent experiment (lead compounds were tested in more assays as positive controls during these studies).

MIC Assay with Tiron or Metal (II) Cation Co-Treatment

Halogenated phenazines were co-treated with tiron in MIC assays to determine if select halogenated phenazine compounds demonstrated antibacterial activities as a result of redox-activity. We did not observe tiron suppression of antibacterial activities of these HP analogues, but we did observe a loss in antibacterial activity of 8-hydroxyquinoline (a redox-active control). Results from these experiments are summarized in Tables 10 and 11. Metal (II) cation studies were performed in a similar setup to the standard MIC assay, with the addition of 200 μM of the metal salt (Copper (II) sulfate, Magnesium (II) sulfate and Ammonium iron (II) sulfate hexahydrate) to the LB medium in the MIC assays.⁴² These data were obtained from three independent experiments. Table 12 shows MIC values after addition of 1 mM Tiron and 200 μM CuSO₄ along with the fold change.

TABLE 10
MIC values for select compounds tested against a panel of gram-
negative bacteria.

| | A. baumannii | | K. pneumoniae | E. coli |
|---|---|---|---|---|
| | 19606 | PAO1 | 13883 | UAEC-1 |
| Compound | MIC (μM) | MIC (μM) | MIC (μM) | MIC (μM) |
| 2 | 100 | >100 | >100 | >100 |
| 8 | 50 | >100 | >100 | >100 |
| 11 | >100 | >100 | >100 | >100 |
| 12 | >100 | >100 | >100 | >100 |
| 13 | >100 | >100 | >100 | >100 |
| 14 | >100 | — | — | — |
| 19 | >100 | >100 | >100 | >100 |
| 20 | >100 | >100 | >100 | >100 |
| 21 | >100 | >100 | >100 | >100 |
| 25 | 6.25 | >100 | 37.5^{a} | 12.5 |
| 27 | >100 | >100 | >100 | >100 |
| 28 | >100 | >100 | >100 | >100 |
| Ciprofloxacin | 6.25 | 0.78 | 0.78 | 0.15^{a} |

Note:
^{a}Represents the mid-point of a two-fold range of values.

During initial MIC assays, 4,7,8-trihalogenated phenazine 12 proved to be the most potent analogue against methicillin-resistant S. aureus (MRSA) planktonic cells by reporting MIC values of 0.15-0.59 μM (Table 7 and Table 11 for a panel of five additional MRSA isolates). HP 12 also demonstrated potent antibacterial activity against methicillin-sensitive S. epidermidis (MIC=0.39 μM; ATCC 12228) and methicillin-resistant S. epidermidis (MIC=0.78 μM; MRSE ATCC 35984). This was our first encounter of an active HP analogue that did not possess a bromine atom in the 2-position of the phenazine. Based on this finding, the two chlorine atoms in the 7- and 8-positions of 12 override the previously established structure-activity relationship (SAR) requirements of having a bromine in the 2-position to be an active antibacterial agent. In addition, HP analogues 7, 8, 11, 14, and 18 demonstrated sub-micromolar growth inhibition activities against at least one MRSA strain, while 12 and 18 demonstrated this level of antibacterial potency against all three MRSA strains (MIC 0.15-0.78 μM; Table 7). Quinoxaline analogues 25-28 demonstrated a range of antibacterial activities against MRSA strains (MIC 0.78-25 μM, Table 7), with 28 proving to be the most potent quinoxaline analogue. Select HP analogues (11, 12, 14, 22, 26, and 28; Table 11 were evaluated against a panel of five additional MRSA isolates and reported potent anti-MRSA activities.

TABLE 11
MICs (reported in μM) for select compounds against S. aureus
clinical isolates.

| Compound | MRSA-1 | SA 129 | SA 138 | SA 147 | SA 156 |
|---|---|---|---|---|---|
| 11 | 0.39 | 0.78 | 0.78 | 1.17^{a} | 1.17^{a} |
| 12 | 0.30^{a} | 0.39 | 0.59^{a} | 0.59^{a} | 0.39 |
| 14 | 0.10 | 1.56 | 0.59^{a} | 0.30^{a} | 0.30^{a} |
| 22 | 0.78 | 1.17^{a} | 3.13 | 0.59^{a} | 0.59^{a} |
| 26 | 1.56 | 6.25 | 6.25 | 6.25 | 3.13 |
| 28 | 1.56 | 2.35^{a} | 2.35^{a} | 1.56 | 1.56 |
| Vancomycin | 0.39 | 0.78 | 1.17^{a} | 1.17^{a} | 0.78 |
| Methicillin | 37.5^{a} | 25 | 25 | 6.25 | 50 |

Note:
^{a}Represents the mid-point of a two-fold range of values. We have determined methicillin to have an MIC of 0.8 to 1.56 μM against methicillin-sensitive S. aureus strains. All MIC values are from three independent experiments.

TABLE 12
MIC values (reported in μM) after addition of 1 mM Tiron and
200 μM CuSO₄ along with the fold change.

| | | | | | |
|---|---|---|---|---|---|
| | | MIC with | | MIC with | |
| Compound | MIC | Tiron | Fold Δ | Cu²⁺ | Fold Δ |

S. epidermidis 12228

| | | | | | |
|---|---|---|---|---|---|
| 2 | 1.56 | 1.17^{a} | n.a. | 50 | +32 |
| 8 | 1.56 | 1.17^{a} | n.a. | 12.5 | +8 |
| 12 | 0.39 | 0.30^{a} | n.a. | 0.39 | n.a. |
| 13 | 0.10^{b} | 0.10^{b} | n.a. | 50 | +500 |
| 15 | 0.10^{b} | 0.10^{b} | n.a. | 50 | +500 |
| 28 | 1.56 | 1.17^{a} | n.a. | 0.39 | −4 |
| 8-HQ | 3.13 | >100 | >+32 | 3.13 | n.a. |

E. faecium 700221

| | | | | | |
|---|---|---|---|---|---|
| 2 | 6.25 | 6.25 | n.a. | 12.5 | n.a. |
| 8 | 3.13 | 3.13 | n.a. | 3.13 | n.a. |
| 12 | 3.13 | 3.13 | n.a. | 1.56 | n.a. |
| 13 | 0.39 | 0.39 | n.a. | 25 | +64 |
| 15 | 0.78 | 0.78 | n.a. | 12.5 | +16 |
| 28 | 6.25 | 6.25 | n.a. | 2.35^{a} | n.a. |
| 8-HQ | 18.8^{a} | >100 | >+12 | 18.8^{a} | n.a. |

Note:
“Fold Δ” denotes the change in antibacterial activity according to MIC values;
(+) for increase in MIC value/loss of antibacterial activity;
(−) for decrease in MIC value/increase of antibacterial activity;
(n.a.) means insignificant changes in MIC values (≤2-fold changes).
^{a}represents the mid-point of a two-fold range of values.

HP analogues that had potent antibacterial agents against MRSA strains demonstrated similar antibacterial activities against S. epidermidis. Against methicillin-sensitive S. epidermidis 12228, we observed a 12- to 15-fold increase in antibacterial potency for halogenated phenazines 14-17 (MIC 0.08-0.10 μM; Table 7) compared to parent HP 2 (MIC 1.17 μM). HP analogues 15, 16, and 22 (MIC 0.30-0.59 μM) proved to be the most potent antibacterial agents against methicillin-resistant S. epidermidis (MRSE 35984).

When evaluated for antibacterial activity against VRE, HPs 14 to 18 and 22 demonstrated an 8- to 64-fold increase in antibacterial activities (MIC 0.10-0.39 M) compared to parent HP 2 (MIC=6.25 μM; 2.2 μg/mL) with 14 proving to be the most potent analogue (MIC=0.10 μM; 0.05 μg/mL). HP analogues 11 and 12 (MIC 3.13-6.25 μM) demonstrated near equipotent antibacterial activities compared to 2 against VRE, despite demonstrating improvements in antibacterial activities against staphylococcal pathogens. The remaining HP analogues demonstrated antibacterial activities against VRE comparable to parent HP 2 (Table 7).

There is a critical need to identify new antibacterial agents that target the slow-growing pathogen Mycobacterium tuberculosis (MtB), which results in 1.5 million deaths each year worldwide.^{5,30} Mycobacterium pathogens are difficult to treat as these persistent bacteria are involved in many chronic and recurring infections,^{5,31} similar to bacterial biofilms. We tested a small panel of our most potent HP analogues against M. tuberculosis (MtB) H37Ra (ATCC 25177) in MIC assays (Table 7). HP 2 demonstrated moderate antibacterial activity against M. tuberculosis with an MIC value of 25 μM. Four halogenated phenazine analogues were identified to be more potent than 2, including 14 (MIC 12.5 μM), 16 (MIC 6.25 μM) with the most potent analogues 11 and 12 reporting MIC values of 3.13 μM against M. tuberculosis. HP analogues 17 and 28 were found to be inactive (MIC>50 μM) against M. tuberculosis in these assays. HPs 11 and 12 demonstrated antibacterial potency against M. tuberculosis near that of streptomycin (MIC 1.32 μM; positive control). Select HPs possess impressive antibacterial activities that could be useful in treating life-threatening MtB infections.

Following our investigations with gram-positive pathogens and M. tuberculosis, we evaluated 12 HP analogues against gram-negative pathogens, including: A. baumannii (ATCC 19606), P. aeruginosa (PAO1), K. pneumoniae (ATCC 13883) and E. coli (UAEC-1, clinical isolate from a bloodstream infection at the University of Arkansas Med. School). Quinoxaline analogue 25 was the only HP analogue to demonstrate good antibacterial activities against the gram-negative pathogens A. baumannii 19606 (MIC 6.25 μM) and E. coli UAEC-1 (MIC 12.5 μM) with moderate antibacterial activity against K. pneumoniae 13883 (MIC 37.5 μM). All 12 HP analogues evaluated in this panel, including quinoxaline 25, proved to be inactive against P. aeruginosa (PAO1; MIC>100 μM).

Select HP analogues were then evaluated for mammalian cytotoxicity in lactate dehydrogenate (LDH) release assays³² against HeLa cells to determine if HP analogues target bacteria and not mammalian cells, a critical aspect of developing new antibacterial agents (Table 7). In LDH release assays, all 15 HP analogues that were evaluated against HeLa cells demonstrated excellent cytotoxicity profiles with several HP analogues not showing any observable cytotoxicity at 100 μM. Only HP 14 demonstrated cytotoxicity at 100 μM; however, no cytotoxicity was observed for this analogue at 50 μM. Considering the sub-micromolar antibacterial activity of many HP analogues against MRSA, MRSE and VRE, our HeLa cell data demonstrates that HP analogues demonstrate a very high degree of specificity targeting bacterial cells over mammalian cells, which is promising for this new class of antibacterial agents.

### Bacterial Biofilm Eradication and Hemolysis Studies

Following the initial antibacterial assessment, 24 HP analogues were advanced to biofilm eradication studies against clinical isolate MRSA-2 alongside conventional antibiotics and other controls agents (e.g., QAC-10). For biofilm eradication assays, we used the Calgary Biofilm Device (CBD)³³⁻³⁵ which allows bacterial biofilms to be established on pegs that are anchored to the lid of a 96-well plate and submerged in inoculated media. Pegs (e.g., CBD lid) with established bacterial biofilms are then transferred to a second 96-well plate containing serial dilutions of test compound (e.g., halogenated phenazines and conventional antibiotics) for biofilm eradication. After compound treatment, the CBD pegs are then transferred to a third 96-well plate containing only fresh media to allow any viable biofilms to recover and disperse planktonic cells from the viable biofilm, resulting in turbid wells. Microtiter wells that are not turbid result from eradicated biofilms (the lowest test concentration that results in a lack of turbidity is considered to be a compound's minimal biofilm eradication concentration or MBEC).

Using the CBD assay, we were able to determine the relative killing dynamics for a compound against both planktonic and biofilm cells in a single assay as the CBD assay can also be used to determine minimum bactericidal concentrations from the media in the test plate (MBC; planktonic killing; Table 8). In addition to MRSA-2, we evaluated panels of 8, 7, 18, and 14 HP analogues and various control compounds (e.g., conventional antibiotics) against MRSA BAA-1707, MRSA BAA-44, MRSE 35984 and VRE 700221 biofilms using the CBD assay, respectively (Table 8). We found the CBD assay to be highly robust for these phenotypic screening purposes^{28,36} in addition to being useful in quantifying biofilm (persister) cell killing with select compounds from treated and untreated CBD pegs.

TABLE 8
Summary of biofilm eradication studies against MRSA, MRSE,
and VRE biofilms. All biological results in this table are reported in micromolar (μM)
concentrations.

| | | MRSA | MRSA | | | |
|---|---|---|---|---|---|---|
| | | BAA-1707 | BAA-44 | MRSE 35984 | VRE 700221 | % |
| | MRSA-2 | MBC/ | MBC/ | MBC/ | MBC/ | Hemolysis |
| Compound | MBC/MBEC | MBEC | MBEC | MBEC | MBEC | at 200 μM |
| 2 | 15.6/93.8^{a} | 62.5/375^{a} | 62.5/188^{a} | 23.5^{a}/250^{b} | 23.5^{a}/9.38^{a} | ≤1 |
| 3 | 62.5/>1000 | — | — | — | — | ≤1 |
| 4 | 31.3/93.8^{a} | — | — | 31.3^{b}/23.5^{a} | 23.5^{a}/15.6 | ≤1 |
| 5 | 62.5/>1000 | — | — | 31.3/375^{a} | 11.7^{a}/11.7^{a} | ≤1 |
| 6 | 15.6/125 | — | — | — | — | 5.1 |
| 7 | 46.9^{a}/375 | — | — | 11.7^{a}/46.9^{a} | 7.8^{b}/3.9 | ≤1 |
| 8 | 31.3/62.5 | 23.5^{a}/93.8^{a} | 46.9^{a}/93.8^{a} | 11.7^{a}/188^{a} | 3.0^{a}/5.9^{a} | 2.9 |
| 9 | 93.8^{a}/250 | — | — | 125/250 | — | ≤1 |
| 10 | 46.9^{a}/250 | — | — | — | — | 1.3 |
| 11 | 37.5^{a}/37.5^{a} | — | — | 37.5^{a}/100 | 100^{b}/25 | 2.7 |
| 12 | 25/37.5 | 75^{a}/>200 | 75^{a}/>200 | 9.38^{a}/75^{a} | 3.13/1.56 | 1.4 |
| 13 | >200/>200 | — | — | >200/>200 | >200/>200 | ≤1 |
| 14 | 3.13/9.38^{a} | 4.69^{a}/6.25 | 12.5/9.38^{a} | 1.56/2.35^{a} | 0.20^{c}/0.20^{c} | 2.7 |
| 15 | 25^{b}/25 | — | — | 1.56/3.13 | — | 2.1 |
| 16 | 18.8^{a}/50 | 6.25/12.5 | 18.8^{a}/12.5 | 1.56/4.69^{a} | 0.39/0.39 | ≤1 |
| 17 | 37.5^{a}/50 | 6.25/50 | 50/37.5^{a} | 1.56/6.25 | 0.39/0.39 | ≤1 |
| 18 | 50/25 | 12.5/12.5 | 9.38^{a}/9.38^{a} | 37.5^{a}/25 | 1.56^{b}/0.59^{a} | 2.1 |
| 19 | 200/>200 | — | — | — | — | ≤1 |
| 22 | 46.9^{a}/188^{a} | 46.9^{a}/62.5 | — | 11.7^{a}/23.5^{a} | 2.0/2.0 | 11.5 |
| 25 | 1500^{a}/>2000 | — | — | — | — | 1.8 |
| 26 | >2000/>2000 | — | — | 750^{a}/>1000 | — | ≤1 |
| 27 | 125^{b}/1500 | — | — | — | — | ≤1 |
| 28 | 93.8^{a}/93.8^{a} | — | — | 23.5^{a}/125 | — | 1.2 |
| 29 | 23.5^{a}/188^{a} | — | — | 9.38^{a}/93.8^{a} | 2.0/1.5^{a} | ≤1 |
| QAC-10 | 31.3^{b}/125 | — | — | 31.3/31.3 | 3.0^{a}/3.0^{a} | >99 |
| CCCP | 31.3/1000 | — | — | 31.3/93.8^{a} | — | 3.5 |
| NAC | >2000/>2000 | — | — | >2000/>2000 | >2000/>2000 | — |
| Pyrazinamide | >2000/>2000 | — | — | — | — | — |
| Vancomycin | 3.0^{b}/>2000 | 3.9/>2000 | 7.8/>2000 | 3.0^{a}/>2000 | >200/150 | ≤1 |
| Daptomycin | 62.5^{b}/>2000 | 125/>2000 | — | — | — | 1.7 |
| Linezolid | 15.6/>2000 | 31.3/>2000 | — | — | 469^{b}/1.56 | ≤1 |
| Doxycycline | 2.0/46.9^{a} | — | — | — | — | — |
| Rifampin | 2.0/46.9^{a} | — | — | 3.0^{a}/15.6^{b} | — | — |
| EDTA | 2000/>2000 | — | — | 1000/>2000 | — | 3.0 |

Notes:
^{a}midpoint value for independent experiments that gave a 2-fold range;
^{b}corresponds to a 4-fold range in independent experiments;
^{c}lowest concentration tested;
MBC/MBEC values were obtained from 2-6 independent experiments.

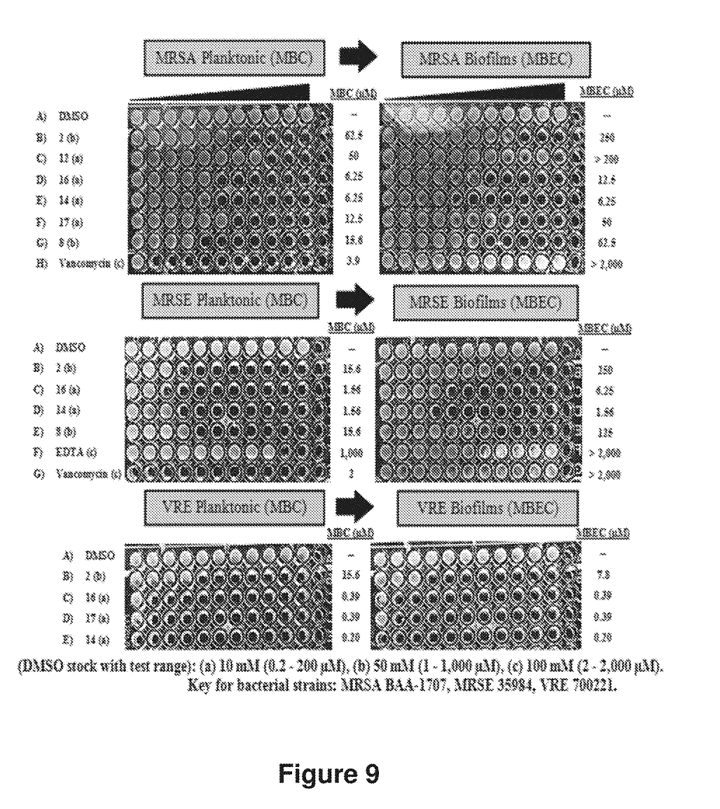

Of the 24 HP analogues that were evaluated against MRSA-2, eight analogues demonstrated more potent biofilm-eradicating activity than HP 2 (MBEC=93.8 μM; Table 8), including: 8, 11, 12, 14, 15, 16, 17, and 18. Seven of the eight more potent HP analogues contain additional halogen atom substitutions in the 7- and/or 8-positions of the phenazine heterocycle. Several of these HP analogues were then evaluated against BAA-1707 (FIG. 9) and BAA-44, all proving to be potent eradicating agents against these MRSA biofilms. HP analogue 14 (2-bromo-7,8-dichloro-4-iodo-1-hydroxyphenazine) reported the most potent activity against MRSA biofilms with MBEC values of 6.25-9.38 μM (e.g., 10- to 60-fold more potent than parent HP 2) while HP analogues 16 to 18 also demonstrated potent biofilm-eradicating activities against all MRSA strains (MBEC=9.38-50 μM). At the MBEC value against MRSA-2, HP 14 eradicates >3-log (>99.9%) of persister cells within the corresponding biofilms (viable biofilm cell count determined from CBD peg; FIG. 11).

Front-running antibiotics used to treat MRSA infections (e.g., vancomycin, daptomycin, and linezolid) were tested alongside our HP analogues. Vancomycin, daptomycin, and linezolid were unable to eradicate MRSA biofilms at the highest test concentrations (MBEC>2,000 μM; FIG. 9; Table 8) despite demonstrating moderate to potent planktonic killing in the same experiment (MBC 3.0-125 μM). These results are illustrative of the innate ability of biofilms to tolerate high levels of antibiotics despite having susceptible planktonic counterparts. During these investigations, doxycycline and rifampin were both found to eradicate MRSA-2 biofilms in CBD assays (MBEC=46.9 μM); however, the MBEC:MBC ratio for these conventional antibiotics is >23 compared to MBEC:MBC ratios of 1-6 for most active HP analogues in the same assays (Table 8).

In addition to conventional antibiotics, select biofilm-eradicating agents and persister cell killing agents were also evaluated in our CBD assays as positive controls to determine the effectiveness of our HP analogues on a head-to-head basis. This panel of known biofilm- or persister-eradicating agents includes: QAC-10 (AMP mimic; membrane-lysing agent),¹⁷ carbonyl cyanide m-chlorophenyl hydrazine (CCCP; membrane-active ionophore)³⁷, N-acetyl cysteine (NAC; antioxidant)³⁸ and pyrazinamide (persister killer).^{4,39,40} From this panel, we found QAC-10 to have the most potent biofilm eradication activities (MBEC=125 μM) against MRSA-2. CCCP reported weak biofilm eradication activities against MRSA-2 (MBEC=1,000 μM), while NAC and pyrazinamide did not show any biofilm eradication activities (MBEC>2,000 μM) using the CBD assay. Compared to our lead HP 14, this small panel of known biofilm-eradicating agents and persister cell killers were found to be 12- to >200-fold less potent at eradicating MRSA-2 biofilms (Table 8).

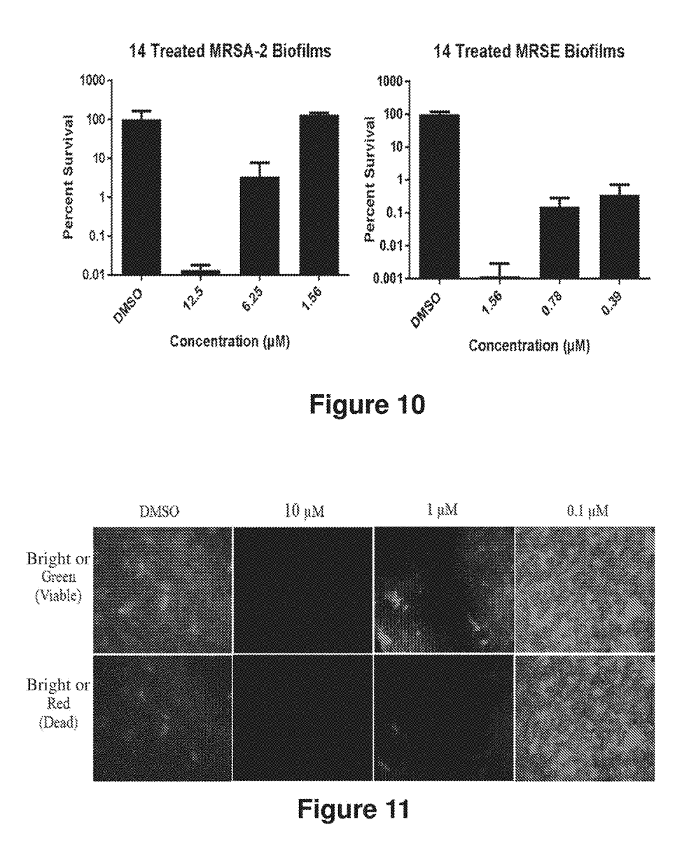

From the panel of 18 HP analogues that were evaluated against MRSE biofilms, 13 demonstrated more potent biofilm eradication activities than HP 2 (MBEC=250 μM; Table 8). As with our MRSA biofilm eradication studies, we found HP 14 to demonstrate unparalleled biofilm-eradicating activities against MRSE (MBEC=2.35 μM; >100-fold more potent than 2; FIG. 9). At the MBEC values against MRSE, HPs 14 and 16 eradicated >4-log (>99.99%) of persister cells within the corresponding biofilms while demonstrating 1-2 log reduction in viable biofilm cells at sub-MBEC values (viable biofilm cells determined from CBD peg; FIG. 10; see 16). Several HP analogues (15, 16, 17, and 18) that possess halogen atoms in the 7- and/or 8-position of the phenazine heterocycle proved to eradicate MRSE biofilms with a high level of potency (MBEC=3.13-25 μM). In addition, HPs 4 and 22 (non-substituted analogues in the 7- and 8-position) reported MBEC values of 23.5 μM against MRSE biofilms. In these investigations, MRSE biofilms did show some susceptibility to various control compounds (QAC-10, MBEC=31.3 μM; CCCP, MBEC=93.8 μM; rifampin, MBEC=15.6 μM); however, vancomycin was unable to eradicate MRSE biofilms at the highest concentration tested (MBEC>2,000 μM; Table 8).

VRE biofilms were found to be highly sensitive to several of our HP analogues, including parent HP 2 (MBEC=9.38 μM, FIG. 9). Nine of the 14 HP analogues we tested demonstrated more potency compared to 2, with 4 HP analogues (14 and 16 to 18) reporting sub-micromolar MBEC values against VRE biofilms (Table 8). HP 14 demonstrated the most potent biofilm eradication activity against VRE biofilms and gave an MBEC value of 0.20 μM (47-fold more potent than 2). QAC-10 potently eradicates VRE biofilms (MBEC=3.0 μM) similar to previous reports;¹⁷ however, NAC demonstrates no biofilm eradication activity in our CBD assays, despite a previous report of E. faecium biofilm eradication.38

In addition to Calgary Biofilm Device experiments, we evaluated select HPs against staphylococcal biofilms in live/dead staining experiments. HP 14 was added to 24 hour old MRSE biofilms at 0.1, 1, and 10 μM and allowed to incubate at 37° C. for an additional 24 hours. Following this, images of the treated and untreated S. epidermidis biofilm were taken using fluorescence microscopy (FIG. 11). Halogenated phenazine 14 removed all detectable MRSE biofilm cells from the glass surface at 10 μM and a significant amount of the biofilm cells at 1 μM. Interestingly, at 0.1 μM of 14 a significant amount of MRSE biofilm cells were eradicated, however, not cleared from within the biofilm. We previously observed HPs 12 and 14 to clear MRSA-2 biofilms at sub-micromolar concentrations, well below their corresponding MBEC values in CBD experiments.²⁸ It appears that the tested halogenated phenazine analogues are able to eradicate bacterial biofilms, in part, due to an effective clearance mechanism.

HP analogues were also evaluated for hemolysis activity against human red blood cells (RBCs; Table 8) at 200 μM to probe if HP analogues operated through a membrane-lysing mechanism, typical of antimicrobial peptides.¹⁷ We found HP analogues do not generally lyse RBCs as only analogues 6 (5%) and 22 (12%) demonstrated low levels of hemolytic activity at 200 μM while the remainder of the HP analogues demonstrated insignificant hemolysis (e.g., <3%). We found AMP-mimic and known membrane-lysing agent QAC-10¹⁷ to cause >99% hemolysis of RBCs in these assays alongside our HP analogues. This hemolysis data provides further support that HP analogues preferentially target bacterial cells over mammalian cell types, similar to our observations with HeLa cells.

### Structure-Activity Relationships (SAR) and Profiles of Halogenated Phenazine Analogues

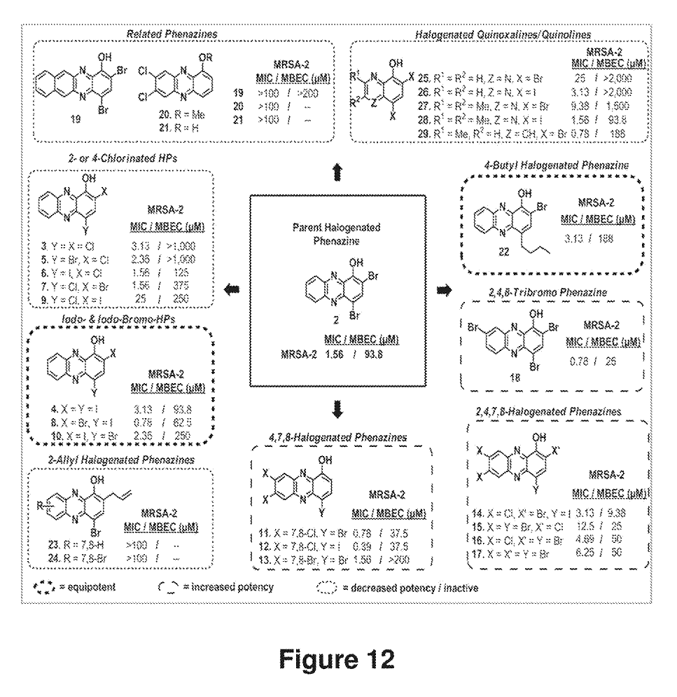

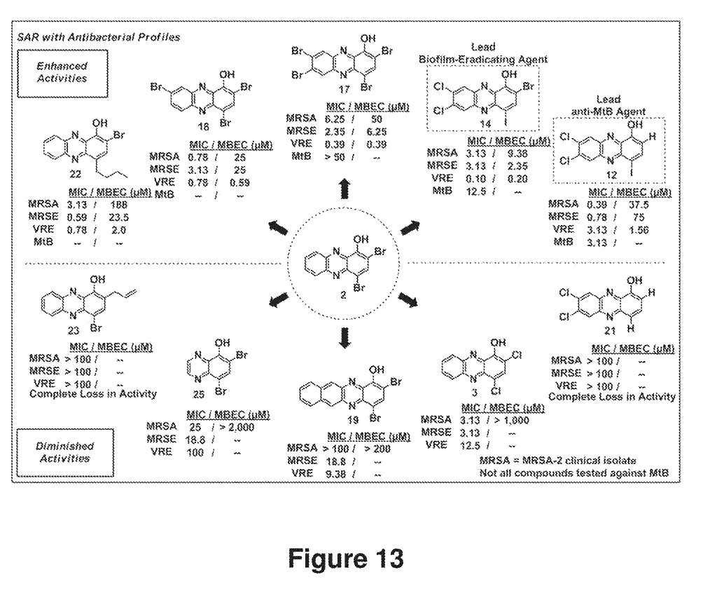

Our focused, yet structurally diverse sub-classes of halogenated phenazines (2 to 24), quinoxalines (25 to 28) and quinoline (29) small molecules (FIG. 12) provided significant structure-activity relationship details for this subset of potent biofilm-eradicating agents while illuminating interesting biological profiles (FIG. 13). Active HP analogues demonstrated good to outstanding gram-positive antibacterial and biofilm eradication activities. In addition, HP analogues demonstrated potent antibacterial activities for select HPs against M. tuberculosis (Table 7, FIG. 13). Interestingly, all HP analogues tested in Example 3 were found to be completely inactive against our panel of gram-negative bacteria.

Chlorine atom substitution at the 2- and/or 4-position of 2 resulted in a loss of MRSA-2 biofilm eradication activities (see HP 3, 5, 7, and 9; FIG. 12) with the only exception to this trend being HP 6, which demonstrated essentially equipotent biofilm eradication activities compared to 2. 2,4-Diiodo-1-hydroxyphenazine 4 maintained biofilm eradication activities compared to 2, while 2-bromo-4-iodo-1-hydroxyphenazine 8 gave a slight (<2-fold) increase in biofilm eradication activities against MRSA-2. Reversing the mixed halogenated pattern of 8 to afford HP 10 (4-bromo-2-iodo-1-hydroxyphenazine) resulted in a 4-fold reduction in biofilm eradication activity between these two mixed HP analogues.

The 7- and 8-positions of the HP scaffold (e.g., structure 2) proved to be critical for potent biofilm eradication activities as the introduction of a single bromine atom at the 8-position of HP 18 resulted in a 4-fold increase in biofilm eradication potency against MRSA-2 compared to 2 (FIGS. 12 and 13). HP 18 also proved to be 10- to 30-fold more potent at eradicating biofilms against our panel of MRSA (BAA-1707 and BAA-44), MRSE and VRE strains compared to 2 (Table 8). Introducing a fourth bromine atom at the 7-position (from HP 18) corresponds to HP 17, which resulted in a 2-fold loss of biofilm eradication activity against MRSA-2 compared to 18. This loss in activity was also observed in the other two MRSA strains (Table 8); however, HP 17 proved to be more potent than 18 against MRSE and VRE biofilms (FIG. 13). 7,8-Dichlorohalogenated phenazine analogues 15 and 16 demonstrated equipotent biofilm eradication potencies to HP 17 and 18 against MRSA-2. HP 16 also demonstrated potent biofilm eradication activities against MRSE and VRE that were on pace with 17. HP analogue 14 is very similar to 16 except the 2-bromine atom is exchanged with a 2-iodide atom, which resulted in a significant increase in biofilm-eradicating potencies across all MRSA, MRSE, and VRE strains tested. This single halogen atom difference resulted in a 5-fold increase in potency for HP 14 against MRSA-2 compared to HP 16, and a 10-fold increase in biofilm eradication activity compared to parent HP 2. HP 14 demonstrated the most potent biofilm eradication across the entire panel of gram-positive pathogens, corresponding to a 60-fold increase against MRSA BAA-1707, 100-fold increase against MRSE, and 47-fold increase in VRE biofilm eradication activities compared to parent HP 2 (FIG. 12). Interestingly, dibrominated benzophenazine analogue 19 (containing a fused aromatic ring at the 7- and 8-position of the HP scaffold) did not show any biofilm eradication activities at the highest concentration tested, resulting in a >2- to >20-fold loss in activity compared to active HPs 2 and 14, respectively (Table 8).

From our previous studies,²⁶ we determined that the bromine atom in the 2-position of the HP scaffold is crucial for antibacterial activities; however, when the 7- and 8-positions of the HP scaffold are chlorinated, HP analogues bearing a bromine (11) or iodide (12) atom in the 4-position without a halogen atom in the 2-position demonstrate potent biofilm eradication activities overriding our previous SAR knowledge of this class of antibacterial agents (FIG. 13). Despite HP 11 and 12 being 3-fold more potent than parent HP 2, these analogues were less active than lead HP 14 against MRSA-2 biofilms (FIG. 12). HP 11 and 12 demonstrated moderate eradication activities compared to other HP analogues against MRSE and VRE biofilms. Although HPs 11 and 12 demonstrated a moderate level ofbiofilm eradication, these analogues were found to possess the most potent antibacterial activities for any HPs evaluated against M. tuberculosis. Alternatively, when the two chlorine atoms in the 7- and 8-position of 11 and 12 were replaced with bromine atoms (giving HP 13), there was a significant loss in activity as no eradication was observed against MRSA, MRSE, and VRE biofilms. Removal of the 4-halogen atom of 7,8-dichlorinated analogues 11 and 12 resulted in inactive HP 21, demonstrating the necessity for the 4-position to be halogenated in this HP sub-series (FIG. 13).

We expanded our library to include alkylated HP analogues, and we were delighted to discover that 4-butyl HP analogue 22 (mono-brominated only at 2-position) demonstrated potent biofilm eradication activities during these investigations (FIGS. 12 and 13). Against MRSA-2, HP 22 demonstrated a 2-fold reduction in biofilm eradication activities compared to 2; however, this trend was reversed as 22 was found to be more potent than 2 against MRSA-1707 (6-fold increase in potency), VRE (5-fold increase in potency), and MRSE (10-fold increase in potency) biofilms. This was the first time that a monohalogenated HP analogue demonstrated biofilm eradication activities and sets the stage for future analogue synthesis. Interestingly, switching the positions of the alkyl and bromine groups results in a complete loss of antibacterial activity (e.g., HPs 23 and 24).

Simplified HP scaffolds, including quinoxalines 25 to 28 and quinoline 29, led to analogues with reduced or no biofilm eradication activities against MRSA-2 (FIGS. 12 and 13). From the quinoxaline series, unsubstituted halogenated quinoxalines 25 and 26 were inactive as biofilm eradicating agents at the highest concentrations tested, which are >20-fold higher than active concentrations for HP 2. 2,3-Dimethylquinoxaline analogues gave interesting results with dibrominated analogue 27 demonstrating weak biofilm eradication activities while the diiodinated analogue 28 was equipotent with HP 2 as a biofilm eradicator against MRSA-2. Halogenated quinoline 29 demonstrated a slight loss in biofilm eradication activities against MRSA-2 compared to 2; however, 29 is more potent than 2 against MRSE and VRE biofilms. Our group is also developing halogenated quinoline antibacterial and antibiofilm agents as an elaboration of our halogenated phenazine program.^{36,41,42}

### Preliminary Mechanistic Insights

Phenazine antibiotics, which include 1, are class of redox-active metabolites produced by Pseudomonas and Streptomyces bacteria.^{43,44} It has been proposed that the central ring of the phenazine heterocycle undergoes a redox reaction that generates superoxide anions which, in turn, kills various bacteria and fungi.⁴⁴ Several active HP analogues were assayed in MIC experiments against MRSA-2 in combination with tiron (1 mM, which is non-toxic to MRSA-2; Table 9), a superoxide quenching agent⁴⁵ (S. epidermidis 12228 and VRE were also evaluated). In these experiments, tiron did not suppress the antibacterial activity of HP analogues despite eliminating the antibacterial activity of 8-hydroxyquinoline (8-HQ; positive control).²⁸ In analogous experiments with other free radical scavengers, we found that thiourea (500 μM), manganese (III) tetrakis (4-benzoic acid)porphyrin (MnTBAP; 1 mM), or ascorbic acid (1 mM) did not suppress the antibacterial activities of HP 2. Based on these findings, we conclude that HP analogues may not operate primarily through a redox-based mechanism.

TABLE 9
Preliminary mechanistic studies, determination of solubility and
pKₐ for select HP analogues.

| | MRSA-2 (concentrations in μM) | | | | | | | | |
|---|---|---|---|---|---|---|---|---|---|
| | | MIC | | | | | | | |
| | | with | | MIC with | | MIC | | [aq. | |
| Compound | MIC | Tiron | Fold Δ | Cu²⁺ | Fold Δ | with Fe²⁺ | Fold Δ | soluble]^{c} | pKₐ^{d} |
| 2 | 1.17^{a} | 1.17^{a} | n.a. | 75^{a} | +48 | 3.13 | +2 | 250 μM | 6.74 |
| 8 | 1.17^{a} | 0.78 | n.a. | 50 | +32 | 6.25 | +5 | — | — |
| 11 | 0.78 | — | — | — | — | — | — | 200 μM^{b} | |
| 12 | 0.59^{a} | 0.20 | −3 | 2.35^{a} | +6 | 3.13 | +5 | 200 μM^{b} | 7.70 |
| 13 | 12.5 | 0.10^{b} | −125 | >100 | +8 | 25 | +2 | — | — |
| 14 | 6.25 | — | — | 100 | +16 | 18.8^{a} | +3 | 200 μM^{b} | 6.95 |
| 15 | 3.13 | 0.20 | −16 | >100 | +32 | — | — | — | — |
| 28 | 1.17^{a} | 0.78 | n.a. | 0.78 | n.a. | 4.69^{a} | +4 | — | — |
| 8-HQ | 6.25 | >100 | +16 | 3.13 | n.a. | 6.25 | n.a. | — | — |
| Tetracyc. | 1.17^{a} | — | — | 1.56 | n.a. | 18.8^{a} | +16 | — | — |
| Daptomy. | 6.25 | — | — | 12.5 | n.a. | 6.25 | n.a. | — | — |
| Linezolid | 6.25 | — | — | — | — | 6.25 | n.a. | — | — |
| 4- | — | — | — | — | — | — | — | — | 7.62 |
| nitrophenol | | | | | | | | | |

Note:
“Fold Δ” denotes the change in antibacterial activity according to MIC values;
(+) for increase in MIC value/loss of antibacterial activity;
(−) for decrease in MIC value/increase of antibacterial activity;
(n.a.) corresponds to insignificant changes in MIC values (<2-fold changes);
(—) not tested.
8-HQ is 8-hydroxyquinoline (positive control).
^{a}midpoint value for three independent experiments that gave a 2-fold range,
^{b}highest concentration tested,
^{c}determined under MIC assay conditions (16 hours, 37° C. in media),
^{d}calculated pKₐ values, 4-nitrophenol was used for method validation.
“Tetracyc.” denotes tetracycline.
“Daptomy.” denotes daptomycin.

Halogenated quinolines (e.g., 29) and halogenated phenazines (2 to 22) share several structural features, including a dihalogenated phenolic ring adjacent to the nitrogen atom of a heterocycle. Halogenated quinolines (e.g., 5,7-dihalo-8-hydroxyquinolines) and 8-hydroxyquinolines complex metal(II)-cations allowing an array of diverse bioactivity profiles which have been studied by our group^{36,42} and others.⁴⁶⁻⁴⁹ Biofilm-eradicating HP analogues have a metal-binding moiety that is analogous to 8-hydroxyquinolines where the 1-hydroxyl group and the adjacent phenazine nitrogen atom would form a 5-membered ring upon metal complexation. Recently, Beauvais and co-workers reported that 1-hydroxyphenazine chelates metal ions resulting antifungal activities against Aspergillusfumigatus through iron-starvation.⁵⁰ Although 1-hydroxyphenazine is inactive against staphylococcal pathogens,²⁶ this metal-binding moiety is critical to the potent activities of halogenated phenazines as methylation of the phenolic hydroxyl group in 2 abolishes the antibacterial activity of the HP scaffold.²⁶

We co-treated select HP analogues with 200 μM of copper(II), iron(II) and magnesium(II) in MIC assays against MRSA-2 (Table 9) to determine if certain metal(II) cations would complex to HP analogues and eliminate their antibacterial activities. Upon co-treatment with copper(II), HP analogues demonstrated a significant decrease in antibacterial activities (up to 48-fold loss determined by elevated MIC values by copper(II) co-treatment), while iron(II) slightly decreases antibacterial activity (up to 5-fold elevated MIC values) and magnesium(II) had no effect against MRSA-2 (data not shown). We have observed similar activities with halogenated quinoline small molecules.^{36,42}

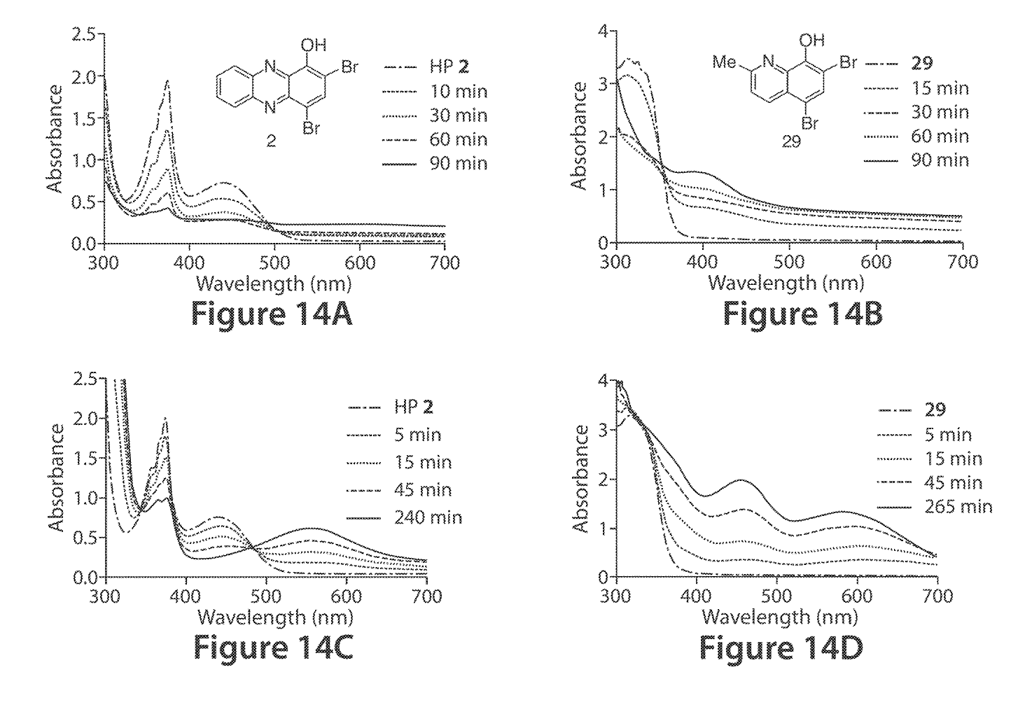

Using UV-Vis spectroscopy, we demonstrated that HP 2 and halogenated quinoline 29 directly bind copper(II) and iron(II) (FIGS. 14A to 14D). When treating HP 2 (λₘₐₓ=440 nm; FIG. 14A) with copper(II), an insoluble complex forms which precipitates out of solution resulting in the disappearance of UV-Vis absorbance. For halogenated quinoline analogue 29 (λₘₐₓ=314 nm; FIG. 14B) we observed a soluble copper(II)-complex resulting in a shift in absorbance to λₘₐₓ=394 nm. We also demonstrate that HP 2 (ligand) binds copper(II) in a 2:1 ligand:copper(II) ratio, analogous to previous findings with 1-hydroxyphenazine⁵⁰ and halogenated quinolines.⁴⁶⁻⁴⁹ In addition, we demonstrated that HP 2 binds iron(II) resulting in a soluble complex and a concomitant shift in absorbance (λₘₐₓ=550 nm; FIG. 14C). We felt that metal binding could be influenced by the acidity of the phenolic proton, which we measured experimentally for HPs 2 (pKₐ=6.74; most acidic, largest shift in MIC with Cu²⁺), 11, 12 (pKa=7.70; least acidic, smallest shift in MIC with Cu²⁺), and 14 (Table 9). Although pKₐ values correlated to changes in MIC with copper(II) co-treatment, changes in MIC values with iron(II) co-treatment had less of a correlation to phenolic acidity of these HP analogues. We then evaluated the general metal-chelating agent ethylenediaminetetraacetic acid (EDTA) in biofilm eradication assays against MRSA-2 to determine if metal sequestration was critical to the observed biofilm eradication activities observed by our HP analogues; however, EDTA demonstrated no biofilm eradication at the highest concentrations tested (MBEC>2,000 μM).

Interestingly, in kill curve experiments against bacteria in exponential-growth phase, our HP analogues demonstrated bacteriostatic activity as the minimum bactericidal concentration (MBC) is >4-fold higher than corresponding MIC values. At higher concentrations, we previously demonstrated that 14 slowly kills stationary MRSA-2 cells unlike QAC-10, which is a rapid killer of stationary MRSA cells.²⁸ From these results, we conclude that HP analogues may operate primarily through a unique metal(II)-dependent mechanism that is critical to persister cells in biofilms; however, detailed mechanistic investigations are required to fully understand our preliminary findings.

### CONCLUSION

Here, we described the chemical synthesis, biological investigations and detailed structure-activity relationships of a new series of HP analogues, inspired by marine phenazine antibiotic 1. Halogenated phenazines demonstrate potent biofilm eradication activities against multiple MRSA, MRSE, and VRE biofilms and may operate through a non-membrane lysing mechanism, similar to antimicrobial peptide mimics. HP 14 proved to be the most potent biofilm-eradicating agent against every drug-resistant isolate tested for biofilm eradication and killed >99.9% of MRSA and MRSE persister cells at the corresponding MBEC value. In addition, HPs 11 and 12 demonstrated potent antibacterial activity against the slow-growing human pathogen M. tuberculosis (MIC=3.13 μM; ˜1 μg/mL). We demonstrated that HP 2 directly binds copper(II) and iron(II), which likely plays a role in the mode of action; however, detailed mechanistic studies are required. Halogenated phenazines are a promising class of biofilm-eradicating agents that effectively target multiple persistent bacterial phenotypes (e.g., biofilms and slow-growing MtB) and could lead to novel therapeutics to treat a spectrum of chronic and recurring bacterial infections.

### Experimental Section

All synthetic reactions were carried out under an inert atmosphere of argon unless otherwise specified. Reagents for chemical synthesis were purchased from commercial sources and used without further purification. Reagents were purchased at ≥95% purity and commercially available controls were used in our biological investigations without further purification. All microwave reactions were carried out in sealed tubes in an Anton Paar Monowave 300 Microwave Synthesis Reactor. A constant power was applied to ensure reproducibility. Temperature control was automated via IR sensor and all indicated temperatures correspond to the maximal temperature reached during each experiment. Analytical thin layer chromatography (TLC) was performed using 250 μm Silica Gel 60 F254 pre-coated plates (EMD Chemicals Inc.). Flash column chromatography was performed using 230-400 Mesh 60 Å Silica Gel from Sorbent Technologies. All melting points were obtained, uncorrected, using a Mel-Temp capillary melting point apparatus from Laboratory Services, Inc.

NMR experiments were recorded using broadband probes on a Varian Mercury-Plus-400 spectrometer via VNMR-J software (400 MHz for ¹H and 100 MHz for ¹³C). Spectra for all new compounds were obtained in the following solvents (reference peaks also included for ¹H and ¹³C NMRs): CDCl₃ (¹H NMR: 7.26 ppm; ¹³C NMR: 77.23 ppm), d₆-DMSO (¹H NMR: 2.50 ppm; ¹³C NMR: 39.52 ppm) and d₄-MeOD (¹H NMR: 3.31 ppm; ¹³C NMR: 49.00 ppm). All NMR experiments were performed at room temperature. Chemical shift values (δ) are reported in parts per million (ppm) for all ¹H NMR and ¹³C NMR spectra. ¹H NMR multiplicities are reported as: s=singlet, br. s=broad singlet, d=doublet, t=triplet, q=quartet, m=multiplet. High-Resolution Mass Spectrometry (HRMS) were obtained for all new compounds from the Chemistry Department at the University of Florida. The purities of all compounds evaluated in biological assays were confirmed to be ≥95% by LC-MS using a Shimadzu Prominence HPLC system, AB Sciex 3200 QTRAP spectrometer, and a Kinetex C18 column (50 mm×2.1 mm×2.6 m) with a 13 minute linear gradient from 10-80% acetonitrile in 0.1% formic acid at a flow rate of 0.25 mL/min.

Bacterial strains used during these investigations include: methicillin-resistant Staphylococcus aureus (Clinical Isolate from Shands Hospital in Gainesville, Fla.: MRSA-2; ATCC strains: BAA-1707, BAA-44) methicillin-resistant Staphylococcus epidermidis (MRSE strain ATCC 35984; methicillin-sensitive strain ATCC 12228), vancomycin-resistant Enterococcus faecium (VRE strain ATCC 700221), Acinetobacter baumannii (ATCC 19606), Pseudomonas aeruginosa (PAO1), Klebsiellapneumoniae (ATCC 13883), and Escherichia coli clinical isolate (UAEC-1). All compounds were stored as DMSO stocks at room temperature in the absence of light for several months at a time without observing any loss in biological activity. To ensure compound integrity of our DMSO stock solutions, we did not subject DMSO stocks of our test compounds to freeze-thaw cycles.

### Chemistry

### General Dihalogenation Procedure at the 2- and 4-Position of the Phenazine Ring (3 and 18)

8-Bromo-1-hydroxyphenazine 57 (43.2 mg, 0.16 mmol) and N-bromosuccinimide, (57.5 mg, 0.32 mmol) were dissolved in dichloromethane (10 mL) and allowed to stir at room temperature for 2 hours. The reaction contents were then concentrated, adsorbed onto silica gel, and purified via column chromatography using dichloromethane to elute 18 as a dark orange solid (46%, 31.3 mg). Note: Analogous reaction conditions were used to synthesize analogue 3.

### 2,4-Dichloro-1-hydroxyphenazine (3)

47% yield; yellow solid. ¹H NMR (400 MHz, CDCl₃): δ 8.40 (m, 1H), 8.36 (br. s, 1H), 8.26 (m, 1H), 7.96-7.89 (m, 2H), 7.91 (s, 1H). ¹³C NMR (100 MHz, CDCl₃): δ 147.0, 143.9, 141.7, 139.0, 134.6, 132.3, 132.0, 131.9, 130.4, 129.1, 123.2, 114.6. HRMS (DART): calc. for C₁₂H₇N₂Cl₂O [M+H]⁺: 264.9930, found: 264.9932. MP: 205-207° C.

### 2,4,8-Tribromo-1-hydroxyphenazine (18)

46% yield; orange solid. ¹H NMR (400 MHz, d₆-DMSO): δ 8.50 (d, J=2.2 Hz, 1H), 8.45 (s, 1H), 8.26 (d, J=9.3 Hz, 1H), 8.15 (dd, J=9.3, 2.2 Hz, 1H). ¹³C NMR (100 MHz, d₆-DMSO): δ 150.9, 141.5, 141.5, 139.6, 137.3, 135.8, 135.3, 131.3, 130.6, 125.7, 111.6, 105.3. HRMS (ESI): calc. for C₁₂H₆Br₃N₂O [M+H]⁺: 430.8025, found: 430.8010. MP: 239-241° C.

### General Dihalogenation Procedure for Synthesis of 3, 18 and 25-28

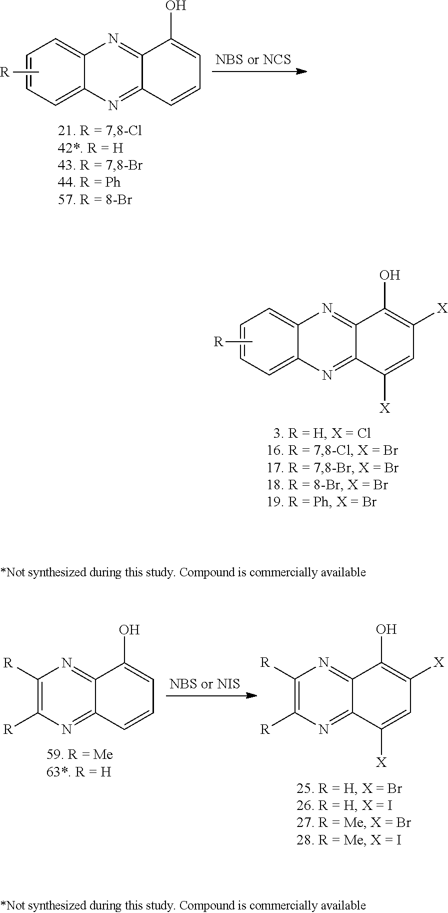

7,8-Dibromo-1-hydroxyphenazine 43 (87.5 mg, 0.24 mmol) and N-bromosuccinimide, (86.2 mg, 0.48 mmol) were dissolved in dichloromethane (15 mL) and allowed to stir at room temperature for 2 hours. The reaction contents were then concentrated, adsorbed onto silica gel, and purified via column chromatography using dichloromethane to elute 2,4,7,8-tetrabromo-1-hydroxyphenazine 17, which was isolated as a dark orange solid (84%, 104 mg).

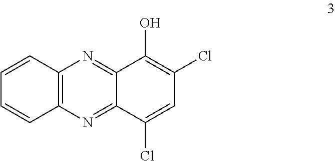 [SMILES: Oc1c(Cl)cc(Cl)c2nc3ccccc3nc12]

Yield:

47% yield; 124 mg of 3 was isolated as a yellow solid. ¹H NMR (400 MHz, CDCl₃): δ 8.40 (m, 1H), 8.36 (br. s, 1H), 8.26 (m, 1H), 7.96-7.89 (m, 2H), 7.91 (s, 1H). ¹³C NMR (100 MHz, CDCl₃): δ 147.0, 143.9, 141.7, 139.0, 134.6, 132.3, 132.0, 131.9, 130.4, 129.1, 123.2, 114.6. HRMS (DART): calc. for C₁₂H₇N₂Cl₂O [M+H]⁺: 264.9930, found: 264.9932. MP: 205-207° C.

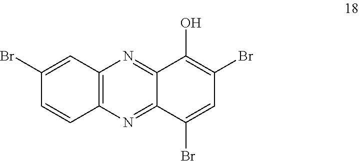 [SMILES: Oc1c(Br)cc(Br)c2nc3ccc(Br)cc3nc12]

Yield:

46% yield; 31.3 mg of 18 was isolated as an orange solid. ¹H NMR (400 MHz, d₆-DMSO): δ 8.50 (d, J=2.2 Hz, 1H), 8.45 (s, 1H), 8.26 (d, J=9.3 Hz, 1H), 8.15 (dd, J=9.3, 2.2 Hz, 1H). ¹³C NMR (100 MHz, d₆-DMSO): δ 150.9, 141.5, 141.5, 139.6, 137.3, 135.8, 135.3, 131.3, 130.6, 125.7, 111.6, 105.3. HRMS (ESI): calc. for C₁₂H₆Br₃N₂O [M+H]⁺: 430.8025, found: 430.8010. MP: 239-241° C.

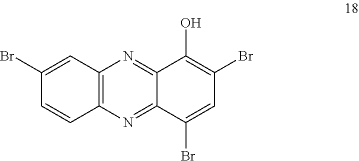 [SMILES: Oc1c(Br)cc(Br)c2nc3ccc(Br)cc3nc12]

Yield:

46% yield; 31.3 mg of 18 was isolated as an orange solid. ¹H NMR (400 MHz, d₆-DMSO): δ 8.50 (d, J=2.2 Hz, 1H), 8.45 (s, 1H), 8.26 (d, J=9.3 Hz, 1H), 8.15 (dd, J=9.3, 2.2 Hz, 1H). ¹³C NMR (100 MHz, d₆-DMSO): δ 150.9, 141.5, 141.5, 139.6, 137.3, 135.8, 135.3, 131.3, 130.6, 125.7, 111.6, 105.3. HRMS (ESI): calc. for C₁₂H₆Br₃N₂O [M+H]⁺: 430.8025, found: 430.8010. MP: 239-241° C.

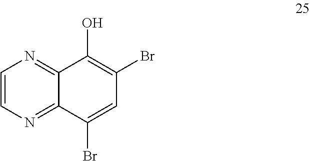 [SMILES: Oc1c(Br)cc(Br)c2nccnc12]

Yield:

60% yield; 54.8 mg of 25 was isolated as a white solid. ¹H NMR (400 MHz, d₆-DMSO): δ 11.59 (s, 1H), 9.09 (d, J=1.8 Hz, 1H), 8.98 (d, J=1.8 Hz, 1H), 8.32 (s, 1H). ¹³C NMR (100 MHz, d₆-DMSO): δ 151.3, 146.7, 144.4, 139.4, 136.1, 134.2, 111.2, 106.3. HRMS (DART): calc. for C₈H₅Br₂N₂O [M+H]⁺: 302.8763, found: 302.8774. MP: 227-229° C.

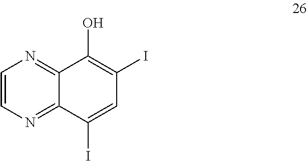 [SMILES: Oc1c(I)cc(I)c2nccnc12]

Yield:

67% yield; 133 mg of 26 was isolated as a white solid. ¹H NMR (400 MHz, d₆-DMSO): δ 11.60 (s, 1H), 9.03 (d, J=1.8 Hz, 1H), 8.90 (d, J=1.8 Hz, 1H), 8.60 (s, 1H). ¹³C NMR (100 MHz, d₆-DMSO): δ 155.1, 147.2, 147.0, 144.3, 142.1, 133.0, 89.1, 82.7. HRMS (DART): calc. for C₈H₅I₂N₂O [M+H]⁺: 398.8486, found: 398.8500. MP: 203-205° C.

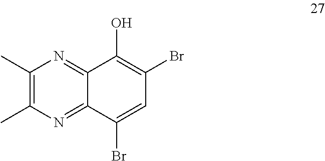

Yield:

95% yield; 142 mg of 27 was isolated as an off-white solid. 1H NMR (400 MHz, CDCl₃): δ 8.07 (br. s, 1H), 7.97 (s, 1H), 2.75 (s, 3H), 2.72 (s, 3H). ¹³C NMR (100 MHz, CDCl₃): δ 155.8, 153.1, 148.8, 138.0, 135.4, 131.3, 112.0, 103.7, 23.5, 22.9. Note: NMR spectra match those previously reported.¹

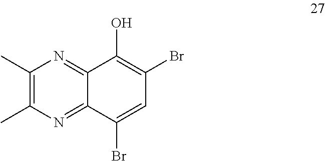

Yield:

95% yield; 142 mg of 27 was isolated as an off-white solid. ¹H NMR (400 MHz, CDCl₃): δ 8.07 (br. s, 1H), 7.97 (s, 1H), 2.75 (s, 3H), 2.72 (s, 3H). ¹³C NMR (100 MHz, CDCl₃): δ 155.8, 153.1, 148.8, 138.0, 135.4, 131.3, 112.0, 103.7, 23.5, 22.9. Note: NMR spectra match those previously reported.¹

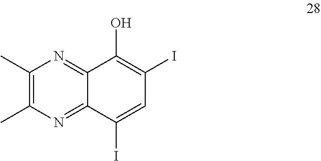

Yield:

72% yield; 84.9 mg of 28 was isolated as an off-white solid. ¹H NMR (400 MHz, CDCl₃): δ 8.39 (s, 1H), 8.31 (br. s, 1H), 2.76 (s, 3H), 2.72 (s, 3H). ¹³C NMR (100 MHz, CDCl₃): δ 156.3, 153.1, 153.0, 146.2, 140.7, 130.1, 88.5, 77.0, 23.4, 22.6. HRMS (DART): calc. for C₁₀H₉I₂N₂O [M+H]⁺: 426.8799, found: 426.8813. MP: 145-147° C.

### Synthesis of 2,4,-Diiodo-1-hydroxyphenazine (4)

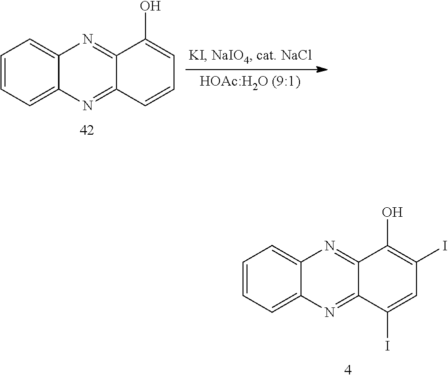 [SMILES: Oc1cccc2nc3ccccc3nc12 ; Oc1c(I)cc(I)c2nc3ccccc3nc12]

To a mixture of 1-hydroxyphenazine 42 (68.1 mg, 0.35 mmol) in a 9:1 solution of glacial acetic acid:water (15 mL) was added sodium chloride (244 mg, 4.17 mmol), sodium periodate (446 mg, 2.08 mmol), and then potassium iodide (241 mg, 2.08 mmol). The reaction was then allowed to stir at room temperature for 8 hours. Upon completion of this reaction (as determined by TLC analysis using dichloromethane), the reaction contents were transferred to a separatory funnel containing a solution of saturated sodium bicarbonate, and the aqueous layer was extracted with dichloromethane. The organic layer was collected, dried with anhydrous sodium sulfate and concentrated in vacuo. The resulting solid was adsorbed onto silica gel (dry loading with dichloromethane) and purified via column chromatography using first hexanes to elute an undesired purple product, followed by dichloromethane to elute pure 4 (19%, 79.2 mg), which was isolated as a yellow solid. ¹H NMR (400 MHz, CDCl₃): δ 8.78 (br. s, 1H), 8.65 (s, 1H), 8.40 (m, 1H), 8.27 (m, 1H), 7.97-7.88 (m, 2H). ¹³C NMR (100 MHz, CDCl₃): δ 153.9, 148.1, 144.9, 142.3, 141.7, 133.1, 132.1, 131.8, 130.3, 128.9, 89.6, 76.5. HRMS (DART): calc. for C₁₂H₇I₂N₂O [M+H]⁺: 448.8642, found: 448.8623. MP: 193-195° C. Note: This is a modified protocol from a previously published procedure.⁵³.

### General Procedure for the Monohalogenation at the 2-Position of the Phenazine Ring (5 to 10, 15, and 22)

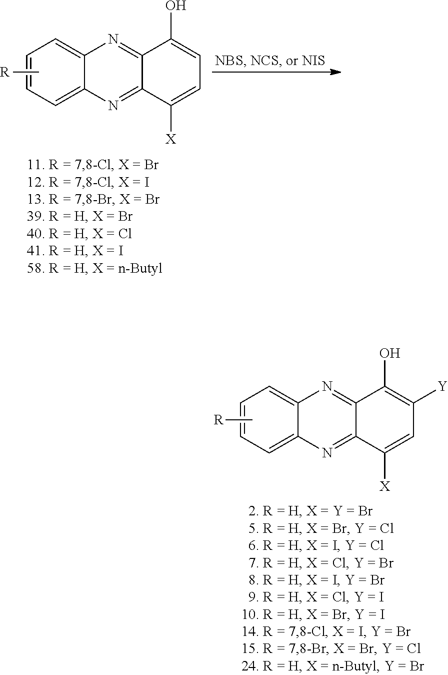

4-Chloro-1-hydroxyphenazine 40 (75.4 mg, 0.33 mmol) and N-bromosuccinimide (58.2 mg, 0.33 mmol) were dissolved in 15 mL dichloromethane and allowed to stir at room temperature for 1 hour. The reaction contents were then concentrated, adsorbed onto silica gel, and purified via column chromatography using dichloromethane to elute 2-bromo-4-chloro-1-hydroxyphenazine 7, which was isolated as a yellow solid (91%, 91.9 mg). Note: Analogous procedures were used for chlorination and iodination using N-chlorosuccinimide and N-iodosuccinimide, respectively.

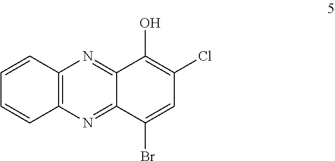 [SMILES: Oc1c(Cl)cc(Br)c2nc3ccccc3nc12]

### 4-Bromo-2-chloro-1-hydroxyphenazine (5)

34% yield; 39.4 mg of 5 was isolated as a yellow solid. ¹H NMR (400 MHz, CDCl₃): δ 8.44-8.35 (m, 2H), 8.26 (m, 1H), 8.13 (s, 1H), 7.96-7.89 (m, 2H). ¹³C NMR (100 MHz, CDCl₃): δ 147.6, 144.2, 141.6, 139.7, 135.4, 134.6, 132.3, 131.8, 130.4, 128.9, 115.2, 112.9. HRMS (DART): calc. for C₁₂H₇BrClN₂O [M+H]⁺: 308.9425, found: 308.9437. MP: 229-231° C.

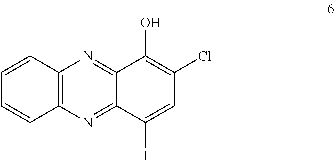 [SMILES: Oc1c(Cl)cc(I)c2nc3ccccc3nc12]

### 2-Chloro-4-iodo-1-hydroxyphenazine (6)

42% yield; 17.3 mg of 6 was isolated as a yellow solid. ¹H NMR (400 MHz, CDCl₃): δ 8.43 (br. s, 1H), 8.39 (m, 1H), 8.38 (s, 1H), 8.26 (m, 1H), 7.96-7.89 (m, 2H). ¹³C NMR (100 MHz, CDCl₃): δ 148.5, 144.6, 141.9, 141.7, 141.3, 134.0, 132.2, 131.7, 130.3, 128.8, 116.0, 88.9. HRMS (DART): calc. for C₁₂H₇ClIN₂O [M+H]⁺: 356.9286, found: 356.9281. MP: 183-185° C.

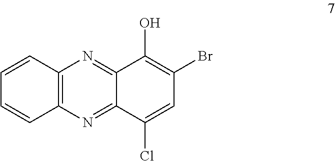 [SMILES: Oc1c(Br)cc(Cl)c2nc3ccccc3nc12]

### 2-Bromo-4-chloro-1-hydroxyphenazine (7)

91% yield; 91.9 mg of 7 was isolated as a yellow solid. ¹H NMR (400 MHz, CDCl₃): δ 8.47 (br. s, 1H), 8.39 (m, 1H), 8.25 (m, 1H), 8.03 (s, 1H), 7.99-7.86 (m, 2H). ¹³C NMR (100 MHz, CDCl₃): δ 148.6, 144.0, 141.6, 139.4, 134.4, 134.0, 132.3, 131.9, 130.5, 129.1, 123.4, 102.6. HRMS (DART): calc. for C₁₂H₇BrClN₂O [M+H]⁺: 308.9425, found: 308.9434. MP: 212-214° C.

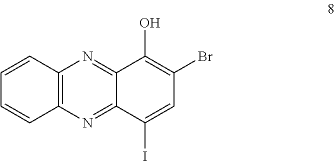 [SMILES: Oc1c(Br)cc(I)c2nc3ccccc3nc12]

### 2-Bromo-4-iodo-1-hydroxyphenazine (8)

65% yield; 88.1 mg of 8 was isolated as a yellow solid. ¹H NMR (400 MHz, CDCl₃): δ 8.57 (br. s, 1H), 8.51 (s, 1H), 8.40 (m, 1H), 8.27 (m, 1H), 7.97-7.89 (m, 2H). ¹³C NMR (100 MHz, CDCl₃): δ 150.2, 144.7, 143.8, 141.7, 141.7, 134.0, 132.2, 131.8, 130.3, 128.8, 104.2, 89.2. HRMS (DART): calc. for C₁₂H₇BrIN₂O [M+H]⁺: 400.8781, found: 400.8785. MP: 186-188° C.

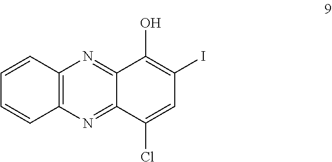 [SMILES: Oc1c(I)cc(Cl)c2nc3ccccc3nc12]

### 4-Chloro-2-iodo-1-hydroxyphenazine (9)

82% yield; 75.4 mg of 9 was isolated as a yellow solid. ¹H NMR (400 MHz, CDCl₃): δ 8.67 (br. s, 1H), 8.36 (m, 1H), 8.21 (m, 1H), 8.14 (s, 1H), 7.98-7.85 (m, 2H). ¹³C NMR (100 MHz, CDCl₃): δ 152.2, 144.1, 141.4, 139.8, 138.4, 133.3, 132.2, 131.9, 130.4, 129.1, 123.4, 74.4. HRMS (DART): calc. for C₁₂H₇ClIN₂O [M+H]⁺: 356.9286, found: 356.9299. MP: 175-177° C.

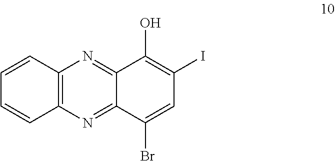 [SMILES: Oc1c(I)cc(Br)c2nc3ccccc3nc12]

### 4-Bromo-2-iodo-1-hydroxyphenazine (10)

48% yield; 20.7 mg of 10 was isolated as a yellow solid. H NMR (400 MHz, CDCl₃): δ 8.71 (br. s, 1H), 8.36 (m, 1H), 8.35 (s, 1H), 8.22 (m, 1H), 7.95-7.87 (m, 2H). ¹³C NMR (100 MHz, CDCl₃): δ 152.8, 144.4, 141.7, 141.4, 140.6, 133.4, 132.2, 131.9, 130.3, 129.0, 113.3, 75.2. HRMS (DART): calc. for C₁₂H₇BrIN₂O [M+H]⁺: 400.8781, found: 400.8770. MP: 188-190° C.

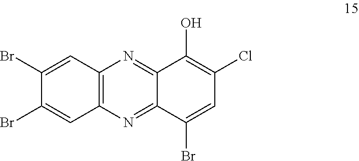 [SMILES: Oc1c(Cl)cc(Br)c2nc3cc(Br)c(Br)cc3nc12]

### 4,7,8-Tribromo-2-chloro-1-hydroxyphenazine (15)

65% yield; 15.8 mg of 15 was isolated as an orange solid. ¹H NMR (400 MHz, d₆-DMSO): δ 8.78 (s, 1H), 8.68 (s, 1H), 8.41 (s, 1H). ¹³C NMR (100 MHz, d₆-DMSO): δ 149.5, 141.7, 140.4, 139.7, 136.3, 135.9, 133.3, 132.6, 128.2, 128.1, 116.5, 111.3. HRMS (DART): calc. for C₁₂H₅Br₃ClN₂O [M+H]⁺: 464.7635, found: 464.7614. MP: 242-244° C.

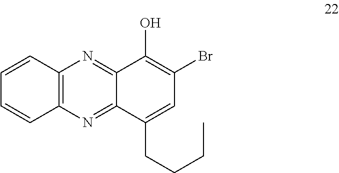 [SMILES: CCCCc1cc(Br)c(O)c2nc3ccccc3nc12]

### 2-Bromo-4-butyl-1-hydroxyphenazine (22)

53% yield; 12.9 mg of 22 was isolated as a yellow solid. ¹H NMR (400 MHz, CDCl₃): δ 8.38 (br. s, 1H), 8.28 (m, 1H), 8.21 (m, 1H), 7.90-7.80 (m, 2H), 7.67 (t, J=0.8 Hz 1H), 3.26 (dt, J=7.8, 0.9 Hz, 2H), 1.83-1.75 (m, 2H), 1.47 (tq, J=7.4, 7.4 Hz, 2H), 0.99 (t, J=7.4 Hz, 3H). ¹³C NMR (100 MHz, CDCl₃): δ 147.2, 143.4, 142.0, 141.0, 134.7, 134.0, 132.9, 131.2, 130.6, 130.5, 129.0, 103.5, 32.9, 30.1, 22.9, 14.3. HRMS (ESI): calc. for C₁₆H₁₆BrN₂O [M+H]⁺: 331.0441, found: 331.0462. MP: 135-137° C.

### General Procedure for Boron Tribromide Demethylation (11, 13, 21, 40, 41, 43, 57, 58, and 59)

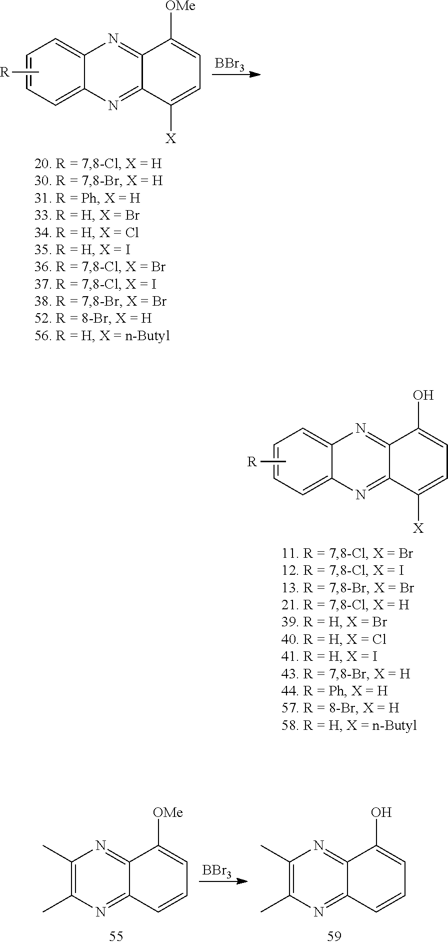

To a round bottom flask was added 7,8-dichloro-1-methoxyphenazine 20 (218 mg, 0.78 mmol) dissolved in anhydrous dichloromethane (18 mL). The mixture was brought to −78° C. in a dry ice bath before dropwise addition of 1 M boron tribromide solution in dichloromethane (5.5 mL, 5.5 mmol). The reaction was left to stir at −78° C. for 1 hour, and then allowed to reach ambient temperature for reaction overnight. The reaction was heated to reflux for 8 hours until complete (monitored by TLC). The solution was transferred to a separatory funnel containing an aqueous solution of saturated sodium bicarbonate, and then extracted with dichloromethane. Organic solvents were dried with sodium sulfate, filtered through cotton, and removed in vacuo. The resulting solid was purified via column chromatography using dichloromethane to elute compound 21 as an orange solid (>99%, 209 mg). Note: Analogous procedures were used for all demethylation reactions using BBr₃.

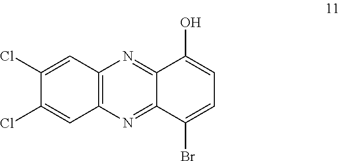 [SMILES: Oc1ccc(Br)c2nc3cc(Cl)c(Cl)cc3nc12]

Yield:

91% yield; 145 mg of 11 was isolated as an orange solid. ¹H NMR (400 MHz, CDCl₃): δ 8.58 (s, 1H), 8.40 (s, 1H), 8.12 (d, J=8.1 Hz, 1H), 8.04 (s, 1H), 7.17 (d, J=8.1 Hz, 1H). ¹³C NMR (100 MHz, CDCl₃): δ 151.8, 142.9, 141.4, 139.9, 136.9, 135.6, 135.6, 130.5, 129.2, 112.5, 110.6. Note: Absence of one ¹³C signal likely due to peak overlap. HRMS (DART): calc. for C₁₂H₆BrCl₂N₂O [M+H]⁺: 342.9035, found: 342.9047. MP: 245-247° C.

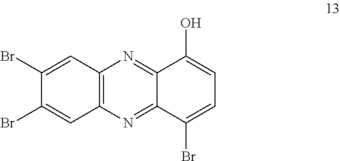 [SMILES: Oc1ccc(Br)c2nc3cc(Br)c(Br)cc3nc12]

Yield:

96% yield; 227 mg of 13 was isolated as an orange solid. ¹H NMR (400 MHz, CDCl₃): δ 8.80 (s, 1H), 8.62 (s, 1H), 8.12 (d, J=8.1 Hz, 1H), 8.04 (s, 1H), 7.18 (d, J=8.1 Hz, 1H). ¹³C NMR (100 MHz, CDCl₃): δ 151.8, 143.2, 141.4, 140.2, 135.8, 135.6, 134.0, 132.7, 129.7, 129.1, 112.5, 110.7. Note: Poor resolution due to insolubility in all solvents used (CDCl₃, d₆-DMSO, d₆-Benzene, and d₄-MeOD). HRMS (DART): calc. for C₁₂H₆Br₃N₂O [M+H]⁺: 430.8025, found: 430.8017. MP: >260° C.

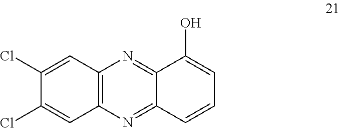 [SMILES: Oc1cccc2nc3cc(Cl)c(Cl)cc3nc12]

Yield:

>99% yield; 209 mg of 21 was isolated as an orange solid. ¹H NMR (400 MHz, d₆-DMSO): δ 8.57 (s, 1H), 8.55 (s, 1H), 7.84 (dd, J=8.8, 7.5 Hz, 1H), 7.68 (d, J=8.8 Hz, 1H), 7.24 (d, J=7.5 Hz, 1H). MP: >260° C., lit. 245-247° C. Note: ¹H NMR and melting point match those previously reported.¹

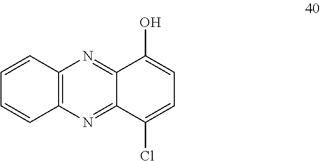 [SMILES: Oc1ccc(Cl)c2nc3ccccc3nc12]

Yield:

95% yield; 248 mg of 40 was isolated as a yellow solid. ¹H NMR (400 MHz, CDCl₃): δ 8.40 (m, 1H), 8.23 (m, 1H), 8.17 (s, 1H), 7.97-7.86 (m, 2H), 7.87 (d, J=8.2 Hz, 1H), 7.17 (d, J=8.2 Hz, 1H). ¹³C NMR (100 MHz, CDCl₃): δ 151.1, 144.2, 141.4, 140.3, 135.1, 131.6, 131.1, 130.4, 129.1, 122.5, 108.8. Note: One ¹³C signal missing, likely due to overlap. HRMS (DART): calc. for C₁₂H₈ClN₂O [M+H]⁺: 231.0320, found: 231.0330. MP: 196-198° C.

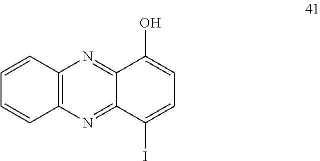 [SMILES: Oc1ccc(I)c2nc3ccccc3nc12]

Yield:

86% yield; 299 mg of 41 was isolated as a yellow solid. ¹H NMR (400 MHz, CDCl₃): δ 8.41 (m, 1H), 8.36 (d, J=8.0 Hz, 1H), 8.30-8.20 (m, 2H), 7.97-7.82 (m, 2H), 7.06 (d, J=8.0 Hz, 1H). ¹³C NMR (100 MHz, CDCl₃): δ 152.9, 144.9, 142.6, 141.6, 141.5, 134.9, 131.5, 131.5, 130.2, 128.9, 110.8, 88.4. HRMS (DART): calc. for C₁₂H₈IN₂O [M+H]⁺: 322.9676, found: 322.9689. MP: 193-195° C.

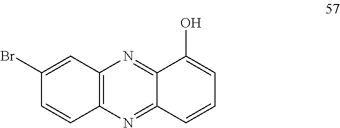 [SMILES: Oc1cccc2nc3ccc(Br)cc3nc12]

Yield:

>99% yield; 83.5 mg of 57 was isolated as a yellow solid. ¹H NMR (400 MHz, CDCl₃): δ 8.42 (dd, J=2.2, 0.5 Hz, 1H), 8.12 (dd, J=9.2, 0.5 Hz, 1H), 8.09 (s, 1H), 7.90 (dd, J=9.3, 2.2 Hz, 1H), 7.82-7.66 (m, 2H), 7.26 (dd, J=6.9, 1.7 Hz, 1H). ¹³C NMR (100 MHz, CDCl₃): δ 151.8, 144.0, 142.9, 141.5, 135.0, 134.7, 132.5, 131.3, 131.1, 125.2, 120.2, 109.9. HRMS (ESI): calc. for C₁₂H₈BrN₂O [M+H]⁺: 274.9815, found: 274.9826. MP: 189-191° C., lit. 176-177° C.².

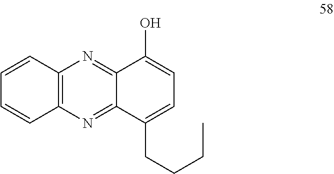 [SMILES: CCCCc1ccc(O)c2nc3ccccc3nc12]

Yield:

90% yield; 33.2 mg of 58 was isolated as a yellow solid. ¹H NMR (400 MHz, CDCl₃): δ 8.28 (m, 1H), 8.17 (m, 1H), 8.10 (br. s, 1H), 7.84-7.76 (m, 2H), 7.53 (dt, J=7.5, 0.9 Hz, 1H), 7.15 (d, J=7.5 Hz, 1H), 3.28 (td, J=7.4 Hz, 0.9 Hz, 2H), 1.84-1.75 (m, 2H), 1.47 (tq, J=7.4, 7.4 Hz, 2H), 0.99 (t, J=7.4 Hz, 3H). ¹³C NMR (100 MHz, CDCl₃): δ 149.7, 143.5, 142.9, 140.8, 135.0, 132.6, 130.4, 130.4, 130.2, 129.6, 129.1, 108.7, 33.0, 30.3, 22.9, 14.3. HRMS (ESI): calc. for C₁₆H₁₇N₂O [M+H]⁺: 253.1335, found: 253.1325. MP: 138-140° C.

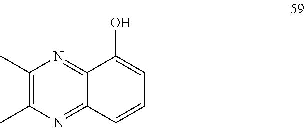

Yield:

86% yield; 149 mg of 59 was isolated as an off-white solid. ¹H NMR (400 MHz, CDCl₃): δ 7.85 (br. s, 1H), 7.55 (dd, J=8.5, 7.5 Hz, 1H), 7.49 (dd, J=8.5, 1.4 Hz, 1H), 7.11 (dd, J=7.5, 1.4 Hz, 1H), 2.72 (s, 3H), 2.69 (s, 3H). ¹³C NMR (100 MHz, CDCl₃): δ 154.5, 151.6, 151.4, 141.6, 131.0, 129.9, 118.9, 110.1, 23.3, 23.1. Note: NMR spectra match those previously reported.¹

### General Procedure for the Halogenation of the 4-Position to Synthesize 23 and 24

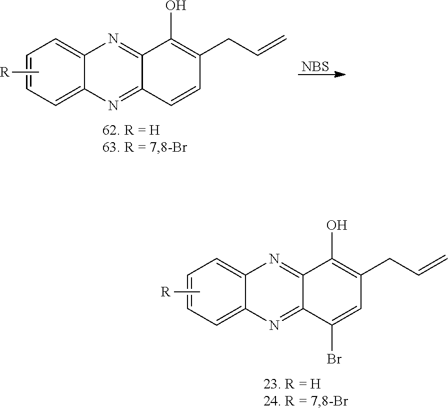

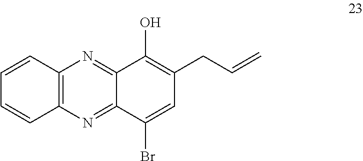 [SMILES: C=CCc1cc(Br)c2nc3ccccc3nc2c1O]

2-Allyl-1-hydroxyphenazine 62 (46.5 mg, 0.20 mmol) and N-bromosuccinimide (35.1 mg, 0.20 mmol) were dissolved in 10 mL dichloromethane and allowed to stir at room temperature for 1 hour. The reaction contents were then concentrated, adsorbed onto silica gel, and purified via column chromatography using dichloromethane to elute 2-allyl-4-bromo-1-hydroxyphenazine 23, which was isolated as a yellow solid (93%, 58.8 mg). Note: An analogous procedure was used to synthesize 24. 2-Allyl-4-bromo-1-hydroxyphenazine (23): 93%% yield; 58.8 mg of 23 was isolated as a yellow solid. ¹H NMR (400 MHz, CDCl₃): δ 8.35 (m, 1H), 8.22 (s, 1H), 8.16 (m, 1H), 7.98 (s, 1H), 7.92-7.76 (m, 2H), 6.06 (ddt, J=16.7, 10.1, 6.6 Hz, 1H), 5.21 (ddt, J=16.7, 1.7, 1.6 Hz, 1H), 5.17 (ddt, J=10.1, 1.7, 1.5 Hz, 1H) 3.66 (ddd, J=6.6, 1.6, 1.5 Hz, 2H). ¹³C NMR (100 MHz, CDCl₃): δ 148.2, 144.0, 141.4, 140.0, 136.9, 135.4, 134.8, 131.3, 131.0, 130.3, 128.9, 121.8, 117.1, 112.0, 33.8. HRMS (ESI): calc. for C₁₅H₁₂BrN₂O [M+H]⁺: 315.0128, found: 315.0127. MP: 149-151° C.

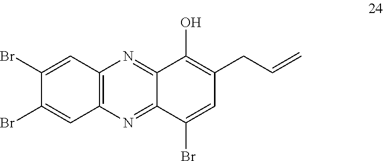 [SMILES: C=CCc1cc(Br)c2nc3cc(Br)c(Br)cc3nc2c1O]

### 2-Allyl-4, 7,8-tribromo-1-hydroxyphenazine (24)

39% yield; 17.7 mg of 24 was isolated as a yellow solid. ¹H NMR (400 MHz, CDCl₃): δ 8.74 (s, 1H), 8.57 (s, 1H), 8.07 (br. s, 1H), 8.03 (s, 1H), 6.04 (ddt, J=16.8, 10.1, 6.6 Hz, 1H), 5.21 (dd, 16.8, 1.6 Hz, 1H), 5.19 (dd, 10.1, 1.6 Hz, 1H), 3.66 (d, J=6.6 Hz, 2H). ¹³C NMR (100 MHz, CDCl₃): δ 148.2, 142.7, 140.4, 140.2, 138.1, 135.3, 135.0, 134.0, 132.6, 128.9, 128.5, 123.1, 117.5, 112.2, 33.8. HRMS (ESI): calc. for C₁₅H₁₄Br₃N₂O [M+H]⁺: 472.8318, found: 472.8331. MP: 187-189° C.

### General Procedure for Monohalogenation at the 4-Position of the Phenazine (33, 36, and 38)

7,8-Dichloro-1-methoxyphenazine 20 (151 mg, 0.54 mmol) was dissolved in dichloromethane (15 mL) before N-bromosuccinimide (106 mg, 0.60 mmol) was added, and the reaction was brought to reflux. The mixture was left to stir overnight until complete (monitored by TLC with dichloromethane). At this time, the reaction was concentrated and adsorbed onto silica gel (via dissolving the crude reaction contents and silica gel in dichloromethane, then concentrating via ROTAVAP) and purified via column chromatography using dichloromethane to elute pure 4-bromo-7,8-dichloro-1-methoxyphenazine 36, which was isolated as a dark yellow solid (96%, 185.2 mg). Notes: Analogous procedures were used for the chlorination and iodination of 1-methoxyphenazines using N-chlorosuccinimide or N-iodosuccinimide.

### 4-Chloro-1-methoxyphenazine (33)

76% yield; yellow solid. ¹H NMR (400 MHz, CDCl₃): δ 8.19-8.08 (m, 2H), 7.71-7.61 (m, 2H), 7.53 (d, J=8.2 Hz, 1H), 6.66 (d, J=8.2 Hz, 1H), 3.90 (s, 3H). ¹³C NMR (100 MHz, CDCl₃): δ 154.0, 142.9, 141.8, 139.9, 136.6, 131.1, 130.8, 129.6, 129.5, 129.2, 123.5, 105.7, 56.3. HRMS (DART): calc. for C₁₃H₁₀ClN₂O [M+H]⁺: 245.0476, found: 245.0484. MP: 149-151° C.

### 4-Bromo-7,8-dichloro-1-methoxyphenazine (36)

96% yield; yellow solid.

¹H NMR (400 MHz, CDCl₃): δ 8.56 (s, 1H), 8.54 (s, 1H), 8.11 (d, J=8.3 Hz, 1H), 6.99 (d, J=8.3 Hz, 1H), 4.17 (s, 3H). ¹³C NMR (100 MHz, CDCl₃): δ 155.3, 142.2, 141.6, 140.9, 137.8, 136.8, 136.4, 134.4, 130.2, 130.2, 114.3, 107.9, 57.0. HRMS (DART): calc. for C₁₃H₈BrCl₂N₂O [M+H]⁺: 356.9192, found: 356.9194. MP: 199-201° C.

### 4,7,8-Tribromo-1-methoxyphenazine (38)

86% yield; yellow solid. ¹H NMR (400 MHz, CDCl₃): δ 8.75 (s, 1H), 8.72 (s, 1H), 8.10 (d, J=8.3 Hz, 1H), 6.98 (d, J=8.3 Hz, 1H), 4.16 (s, 3H). ¹³C NMR (100 MHz, CDCl₃): δ 155.3, 142.5, 141.6, 141.2, 137.8, 134.5, 133.7, 133.6, 129.1, 128.6, 114.4, 107.9, 57.0. HRMS (DART): calc. for C₁₃H₈Br₃N₂O [M+H]⁺: 444.8181, found: 444.8181. MP: 241-243° C.

### Synthesis of 4-iodo-1-methoxyphenazine 35 (Potassium Iodide and Sodium Periodate)

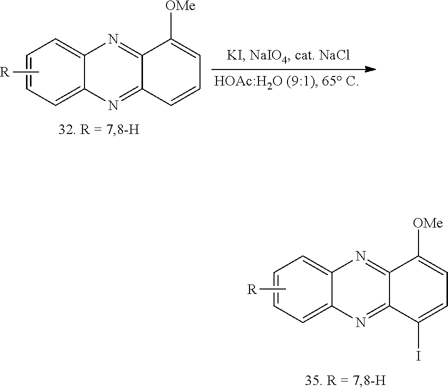

To a mixture of 1-methoxyphenazine 32 (356 mg, 1.69 mmol) in 9:1 acetic acid:water (30 mL) was added sodium chloride (435 mg, 7.45 mmol), sodium periodate (869 mg, 4.06 mmol), and then potassium iodide (675 mg, 4.06 mmol). The reaction was heated to 65° C. and allowed to stir for 21 hours. After completion of this reaction as determined by TLC analysis (with dichloromethane), the reaction mixture was washed with a saturated solution of sodium bicarbonate and then partitioned with dichloromethane. The organic layers were then collected, dried with anhydrous sodium sulfate, and concentrated in vacuo. The resulting solid was then dry loaded onto silica gel (using dichloromethane) and purified via column chromatography using first hexanes to elute purple side product, followed by dichloromethane to elute 35 as a yellow solid (68%, 388.0 mg). Note: This is a modified procedure from a previously published procedure.³

¹H NMR (400 MHz, CDCl₃): δ 8.37 (m, 1H), 8.32 (m, 1H), 8.30 (d, J=8.2 Hz, 1H), 7.89-7.79 (m, 2H), 6.81 (d, J=8.2 Hz, 1H), 4.12 (s, 3H). ¹³C NMR (100 MHz, CDCl₃): δ 156.2, 144.1, 142.6, 140.2, 136.9, 131.4, 131.1, 129.8, 129.7, 108.2, 91.0, 56.8. Note: One signal obscured likely due to overlap. HRMS (DART): calc. for C₁₃H₁₀IN₂O [M+H]⁺: 336.9832, found: 336.9829. MP: 169-171° C.

### Synthesis of 7-bromo-1-methoxyphenazine (52)

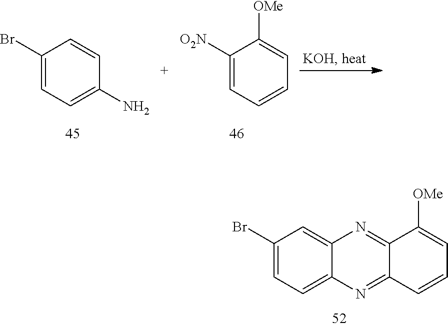 [SMILES: Nc1ccc(Br)cc1 ; COc1ccccc1[N+](=O)[O-] ; COc1cccc2nc3ccc(Br)cc3nc12]

To a 100 mL round-bottom flask was added 4-bromoaniline 45 (1.74 mL, 14.2 mmol), 2-nitroanisole 46 (2.45 g, 14.2 mmol), and potassium hydroxide (3.99 g, 71.1 mmol) in toluene (20 mL). The reaction was then allowed to reflux for 24 hours. After the reaction was complete, the reaction contents were allowed to cool to room temperature. The reaction mixture was transferred to a separatory funnel, partitioned between ethyl acetate, and then washed with water and brine. The organic layers were collected, dried with anhydrous sodium sulfate, filtered, and then concentrated in vacuo. The resulting crude solid was purified via column chromatography using 99:1 to 85:15 hexanes:ethyl acetate to afford 52, which was isolated as a yellow solid (2%, 87.7 mg).

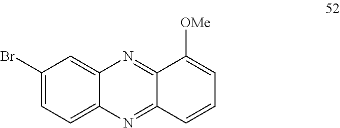 [SMILES: COc1cccc2nc3ccc(Br)cc3nc12]

¹H NMR (400 MHz, CDCl₃): δ 8.58 (d, J 2.2 Hz, 1H), 8.05 (d, J=9.2 Hz, 1H), 7.86 (dd, J 9.2, 2.2 Hz, 1H), 7.80-7.70 (m, 2H), 7.05 (dd, J=7.1, 1.5 Hz, 1H), 4.15 (s, 3H). ¹³C NMR (100 MHz, CDCl₃): δ 155.2, 144.4, 142.5, 142.2, 137.2, 134.7, 132.3, 131.1, 130.8, 124.7, 121.6, 107.3, 56.7. HRMS (ESI): calc. for C₁₃H₁₀BrN₂O [M+H]⁺: 288.9971, found: 288.9960. MP: 194-196° C., lit. 187-188° C.⁵¹.

### Synthesis of 4-butyl-1-methoxyphenazine (56)

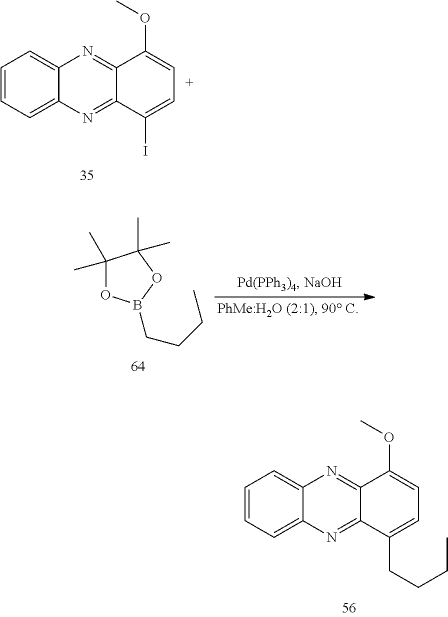 [SMILES: COc1ccc(I)c2nc3ccccc3nc12 ; CCCCB1OC(C)(C)C(C)(C)O1 ; CCCCc1ccc(OC)c2nc3ccccc3nc12]

To a 50 mL round-bottom flask was added 4-iodomethoxyphenazine 35 (146 mg, 0.43 mmol), tetrakis(triphenylphosphine)palladium(0) (100 mg, 0.1 mmol), sodium hydroxide (347 mg, 8.67 mmol), and n-butylboronic acid pinacol ester 64 (1.43 mL, 6.72 mmol) in a 2:1 solution of toluene:water (6 mL). The mixture was heated to 90° C. in an oil bath and left to stir for 48 hours. After cooling to room temperature, the reaction mixture was transferred to a separatory funnel, and partitioned between an aqueous solution of saturated sodium bicarbonate and ethyl acetate. The resulting organic layer was washed with a 1 N solution of hydrochloric acid, and then brine. The organic layers were combined and dried with anhydrous sodium sulfate and concentrated in vacuo. The resulting crude material was purified via column chromatography using 99:1 to 85:15 hexanes:ethyl acetate to afford 56 as a yellow solid (34% yield, 38.9 mg).

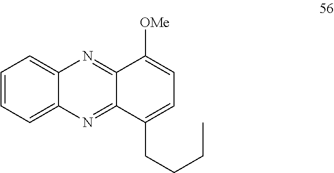 [SMILES: CCCCc1ccc(OC)c2nc3ccccc3nc12]

¹H NMR (400 MHz, CDCl₃): δ 8.38 (m, 1H), 8.26 (m, 1H), 7.85-7.78 (m, 2H), 7.54 (dt, J=7.8, 0.9 Hz, 1H), 7.00 (d, J=7.8 Hz, 1H), 4.15 (s, 3H), 3.30 (td, J=7.6, 0.9 Hz, 2H), 1.87-1.72 (m, 2H), 1.47 (tq, J=7.4, 7.4 Hz, 2H), 0.99 (t, J=7.4 Hz, 3H). ¹³C NMR (100 MHz, CDCl₃): δ 153.4, 143.3, 143.0, 141.9, 137.2, 133.9, 130.4, 130.2, 130.1, 130.1, 128.4, 106.5, 56.5, 32.9, 30.6, 22.9, 14.3. HRMS (ESI): calc. for C₁₇H₁₉N₂O [M+H]⁺: 267.1492, found: 267.1497. MP: 77-79° C.

### Synthesis of 5-methoxy-2,3,-dimethylquinoxaline (55)

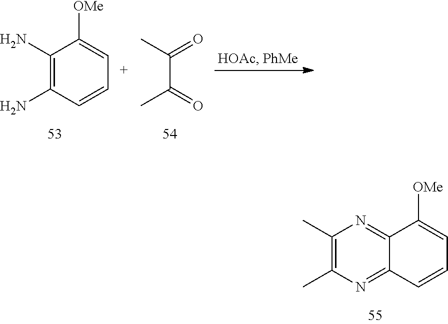

2,3-Diaminoanisole 53 (300 mg, 2.17 mmol) was added to a 100 mL round-bottom flask containing glacial acetic acid (18 mL), toluene (22 mL), and 2,3-butanedione 54 (190 μL, 2.17 mmol). The reaction was then left to stir for 21 hours at room temperature. Upon completion of the reaction, the resulting mixture was neutralized with an aqueous solution of saturated sodium bicarbonate, washed with brine, and extracted with dichloromethane. The resulting organic layers were dried with sodium sulfate, filtered, and concentrated in vacuo. The resulting crude material was purified via column chromatography using 99:1 to 97:3 dichloromethane:methanol to afford 55 in 90% yield (367 mg) as an off-white solid. Note: 2,3-Diaminoanisole was purchased as the dihydrochloride salt, which was treated with a solution of saturated aqueous sodium bicarbonate in a separatory funnel, and then extracted with ethyl acetate to afford the free-base 53, which was used in the reaction. This procedure was modified from a published reaction.²⁵

¹H NMR (400 MHz, CDCl₃): δ 7.45-7.39 (m, 2H), 6.86 (dd, J=5.8, 3.2 Hz, 1H), 3.94 (s, 3H), 2.63 (s, 3H), 2.58 (s, 3H). ¹³C NMR (100 MHz, CDCl₃): δ 154.5, 153.7, 152.1, 141.9, 132.9, 128.6, 120.1, 107.1, 56.1, 23.3, 23.1. Note: NMR spectra match those previously reported.²⁵

### Synthesis of 1-methoxyphenazine (32)

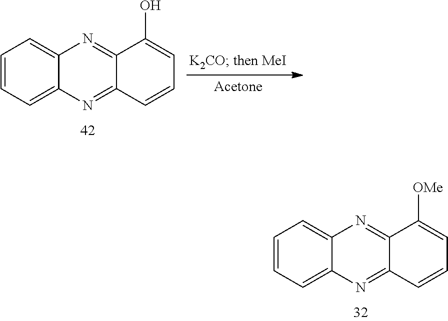 [SMILES: Oc1cccc2nc3ccccc3nc12 ; COc1cccc2nc3ccccc3nc12]

Potassium carbonate (2.26 g, 16.3 mmol) was added to a stirring solution of 1-hydroxyphenazine 42 (643 mg, 3.26 mmol) in 20 mL acetone. The resulting mixture was allowed to stir at room temperature for 30 minutes before iodomethane (2.03 mL, 32.6 mmol) was added to the reaction. The resulting reaction mixture was allowed to stir for an additional 17 hours. The reaction contents were transferred to a separatory funnel and extracted with ethyl acetate. The organic contents were combined, dried with sodium sulfate, and then removed in vacuo to afford 32 in >99% yield (683 mg) as a yellow solid.

¹H NMR (400 MHz, CDCl₃): δ 8.40 (m, 1H), 8.22 (m, 1H), 7.87-7.79 (m, 2H), 7.83 (d, J=8.9 Hz, 1H), 7.75 (dd, J=8.9, 7.5 Hz, 1H), 7.07 (dd, J=7.5, 1.4 Hz, 1H), 4.17 (s, 3H).

MP: 166-168° C., lit. 166-168° C.²⁵

Note: ¹H NMR spectrum and melting point match those previously reported.²⁵

### General Procedure for Phenolic Allylation (60 and 61)

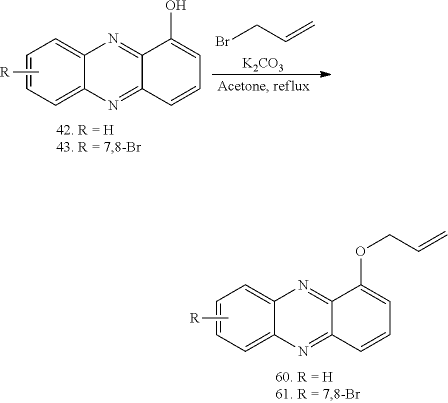

Potassium carbonate (866 mg, 6.27 mmol) was added to a stirring solution of 1-hydroxyphenazine 42 (247 mg, 1.25 mmol) in 15 mL acetone. The resulting mixture was allowed to stir at room temperature for 30 minutes before allyl bromide (0.13 mL, 1.51 mmol) was added to the reaction. The resulting reaction mixture was allowed to stir for an additional 17 hours at reflux. The reaction contents were then transferred to a separatory funnel and extracted with ethyl acetate. The organic contents were then dried with anhydrous sodium sulfate and then removed in vacuo. The resulting crude solid was purified via column chromatography using 99:1 to 90:10 hexanes:ethyl acetate to afford 1-allyloxyphenazines 60, which was isolated as a yellow solid (85%, 251.0 mg). Note: An analogous procedure was used to synthesize 61.

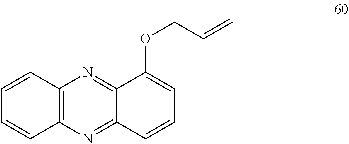 [SMILES: C=CCOc1cccc2nc3ccccc3nc12]

### 1-(Allyloxy)phenazine (60)

85% yield; 251 mg of 60 was isolated as a yellow solid. ¹H NMR (100 MHz, CDCl₃): δ 8.33 (m, 1H), 8.15 (m, 1H), 7.79-7.70 (m, 3H), 7.63 (dd, J=8.9, 7.6 Hz, 1H), 6.99 (d, J=7.6 Hz, 1H), 6.17 (ddt, J=17.3, 10.6, 5.5 Hz, 1H), 5.47 (ddt, J=17.3, 1.6, 1.6 Hz, 1H), 5.32 (ddt, J=10.6, 1.6, 1.6 Hz, 1H), 4.88 (ddd, J=5.5, 1.6, 1.6 Hz, 2H). ¹³C NMR (100 MHz, CDCl₃): δ 154.0, 144.3, 143.5, 142.3, 137.1, 132.7, 130.8, 130.4, 130.4, 130.1, 129.3, 121.6, 118.6, 108.2, 70.2. HRMS (ESI): calc. for C₁₅H₁₃N₂O [M+H]⁺: 237.1022, found: 237.1026. MP: 117-119° C.

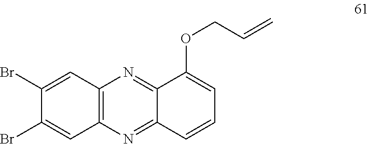 [SMILES: C=CCOc1cccc2nc3cc(Br)c(Br)cc3nc12]

### 7,8-Dibromo-1-allyloxyphenazine (61)

88% yield; 109 mg of 61 was isolated as an orange solid. ¹H NMR (400 MHz, CDCl₃): δ 8.70 (s, 1H), 8.51 (s, 1H), 7.76-7.69 (m, 2H), 7.07 (dd, J=5.9, 2.8 Hz, 1H), 6.21 (ddt, J=17.4, 10.6, 5.4 Hz, 1H), 5.53 (ddt, J=17.4, 1.5, 1.5 Hz, 1H), 5.39 (ddt, J=10.6, 1.5, 1.3 Hz, 1H), 4.92 (ddd, J=5.4, 1.5, 1.3 Hz, 2H). ¹³C NMR (100 MHz, CDCl₃): δ 154.2, 144.8, 142.4, 141.2, 137.6, 134.1, 133.2, 132.6, 131.7, 128.0, 127.2, 121.7, 119.0, 109.1, 70.4. HRMS (ESI): calc. for C₁₅H₁₀Br₂N₂O [M+H]⁺: 394.9213, found: 394.9211. MP: 170-172° C.

### General Procedure for Claisen Rearrangement (62 and 63)

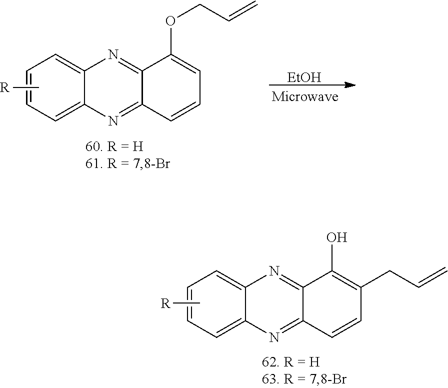

To an 8 mL sealed microwave vial was added 1-(allyloxy)phenazine 60 (19.4 mg, 0.08 mmol) in ethanol (4 mL). The resulting mixture was then heated at 150° C. in the microwave reactor for 90 minutes. The solvent was removed in vacuo and the resulting solid was purified via column chromatography using dichloromethane to elute, affording 2-allylphenazine 62, which was isolated as a yellow solid (>99%, 19.3 mg). Note: Analogous reaction conditions were used to synthesize 63 from 61. Attempts were made to conduct this reaction under traditional thermal heating; however, reaction times exceeded 5 days and resulted in poor yields.

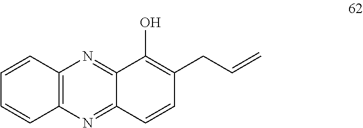 [SMILES: C=CCc1ccc2nc3ccccc3nc2c1O]

### 2-Allyl-1-hydroxyphenazine (62)

>99% yield; 19.3 mg of 62 was isolated as a yellow solid. ¹H NMR (100 MHz, CDCl₃): δ 8.28 (br. s, 1H), 8.21 (m, 1H), 8.13 (m, 1H), 7.81-7.74 (m, 2H), 7.72 (d, J=9.1 Hz, 1H), 7.64 (d, J=9.1 Hz, 1H), 6.09 (ddt, J=16.7, 10.1, 6.6 Hz, 1H), 5.18 (ddt, J=16.7, 1.7, 1.7 Hz, 1H), 5.14 (ddt, J=10.1, 1.7 Hz, 1.6 Hz, 1H) 3.68 (ddd, J=6.7, 1.7, 1.6 Hz, 2H). ¹³C NMR (100 MHz, CDCl₃): δ 148.2, 143.8, 143.0, 141.3, 136.0, 134.6, 134.5, 130.4, 130.4, 129.9, 129.2, 120.8, 119.6, 116.5, 34.0. HRMS (ESI): calc. for C₁₅H₁₃N₂O [M+H]⁺: 237.1022, found: 237.1017. MP: 112-114° C.

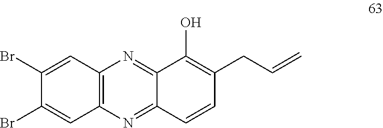 [SMILES: C=CCc1ccc2nc3cc(Br)c(Br)cc3nc2c1O]

### 2-Allyl-7,8-dibromo-1-OH hydroxyphenazine (63)

>99% yield; 39.5 mg of 63 was isolated as an orange solid. ¹H NMR (400 MHz, CDCl₃): δ 8.59 (d, J=0.4 Hz, 1H), 8.56 (d, J=0.4 Hz, 1H), 8.07 (s, 1H), 7.74-7.69 (m, 2H), 6.07 (ddt, J=17.1, 10.1, 6.6 Hz, 1H), 5.19 (ddt, J=17.1, 1.6, 1.6 Hz, 1H), 5.16 (ddt, J=10.1, 1.6, 1.5 Hz, 1H) 3.69 (ddd, J=6.6, 1.6, 1.5 Hz, 2H). ¹³C NMR (100 MHz, CDCl₃): δ 148.3, 143.5, 142.7, 140.2, 135.7, 135.6, 135.0, 133.7, 133.0, 127.7, 127.6, 122.2, 119.9, 116.8, 34.1. HRMS (ESI): calc. for C₁₅H₁₀Br₂N₂O [M+H]⁺: 394.9213, found: 394.9216. MP: 199-201° C.

### Biology

### Microdilution Minimum Inhibitory Concentration (MIC) Assay

The minimum inhibitory concentration (MIC) for each compound was determined by the broth microdilution method as recommended by the Clinical and Laboratory Standards Institute (CLSI).⁵² In a 96-well plate, eleven two-fold serial dilutions of each compound were made in a final volume of 100 μL Luria Broth. Each well was inoculated with ˜10⁵ bacterial cells at the initial time of incubation, prepared from a fresh log phase culture (OD₆₀₀ of 0.5 to 1.0 depending on bacterial strain). The MIC was defined as the lowest concentration of a compound that prevented bacterial growth after incubating 16 to 18 hours at 37° C. (MIC values were supported by spectrophotometric readings at OD₆₀₀). The concentration range tested for each phenazine analogue/antibacterial during this study was 0.10 to 100 μM. DMSO served as our vehicle and negative control in each microdilution MIC assay. DMSO was serially diluted with a top concentration of 1% v/v. All compounds were tested in two independent experiments, active compounds were tested in a third independent experiment (lead compounds were tested in more assays as positive controls during these studies).

Microdilution MIC Assay for Mycobacterium tuberculosis

M. tuberculosis H37Ra (ATCC 25177) was inoculated in 10 mL Middlebrook 7H9 medium and allowed to grow for two weeks. The culture was then diluted with fresh medium until an OD₆₀₀ of 0.01 was reached. Aliquots of 200 μL were then added to each well of a 96-well plate starting from the second column. Test compounds were dissolved in DMSO at final concentration of 10 mM. 7.5 μL of each compound along with DMSO (negative control) and streptomycin (positive control-40 mg/ml stock solution) were added to 1.5 mL of the Mycobacterium diluted cultures, resulting in 50 μM final concentration of each halogenated phenazine analogues and 340 μM for streptomycin. The final DMSO concentration was maintained at 0.5%. Aliquots of 400 μL were added to wells of the first column of the 96-well plate and serially diluted two-fold (200 μL) per well across the plate to obtain final concentrations that ranges from 0.024 to 50 μM for the test compounds and 0.16 to 340 μM for streptomycin. Three rows were reserved for each compound. The plates were then incubated at 37° C. for seven days. Minimum inhibitory concentrations are reported as the lowest concentration at which no bacterial growth was observed. OD₆₀₀ absorbance was recorded using SPECTRAMAX M5 (Molecular Devices). Data obtained from three independent experiments were analyzed using EXCEL.

### Calgary Biofilm Device (CBD) Experiments

### Minimum Bactericidal Concentrations (MBC) and Minimum Biofilm Eradication Concentrations (MBEC) Determination

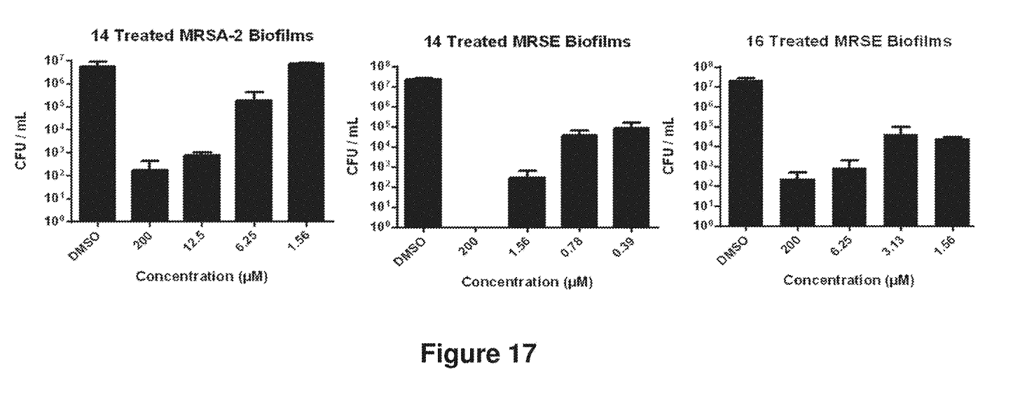

Biofilm eradication experiments were performed using the Calgary Biofilm Device to determine MBC/MBEC values for various compounds of interest (Innovotech, product code: 19111)²⁸. The Calgary device (96-well plate with lid containing pegs to establish biofilms on) was inoculated with 125 μL of a mid-log phase culture diluted 1,000-fold in tryptic soy broth with 0.5% glucose (TSBG) to establish bacterial biofilms after incubation at 37° C. for 24 hours. The lid of the Calgary device was then removed, washed and transferred to another 96-well plate containing 2-fold serial dilutions of the test compounds (the “challenge plate”). The total volume of media with compound in each well in the challenge plate is 150 μL. The Calgary device was then incubated at 37° C. for 24 hours. The lid was then removed from the challenge plate and MBC/MBEC values were determined using different final assays. To determine MBC values, 20 μL of the challenge plate was transferred into a fresh 96-well plate containing 180 μL TSBG and incubated overnight at 37° C. The MBC values were determined as the concentration giving a lack of visible bacterial growth (e.g., turbidity). For the determination of MBEC values, the Calgary device lid (with attached pegs/treated biofilms) was transferred to a new 96-well plate containing 150 μL of fresh TSBG media in each well and incubated for 24 hours at 37° C. to allow viable biofilms to grow and disperse resulting in turbidity after the incubation period. MBEC values were determined as the lowest test concentration that resulted in eradicated biofilm (e.g., wells that had no turbidity after final incubation period). In select experiments, pegs from the Calgary device were removed from lead biofilm eradicators after final incubation, sonicated for 30 minutes in PBS and plated out to determine biofilm cell killing of lead biofilm-eradicating agents (e.g., colony forming unit per milliliter, CFU/mL; FIGS. 10 and 17). All compounds were tested in two independent experiments, active compounds were tested in a third independent experiment (lead compounds were tested in more assays as positive controls during these studies). Antibiotic controls have been tested in a minimum of three independent experiments. Note: MRSA-2, S. aureus (ATCC strains: BAA-1707, BAA-44), S. epidermidis (ATCC 35984), and VRE (ATCC 700221) were tested using these assay parameters.

### Live/Dead Staining (Fluorescence Microscopy) of MRSE 35984 Biofilms

A mid-log culture of MRSE 35894 was diluted 1:1,000-fold and 500 μL was transferred to each compartment of a 4 compartment CELLview dish (Greiner Bio-One 627871). The dish was then incubated for 24 hours at 37° C. After this time, the cultures were removed and the plate was washed with 0.9% saline. The dish was then treated with the compounds in fresh media at various concentrations. DMSO was used as our negative control in this assay. The dish was incubated with the compound for 24 hours at 37° C. After this time, the cultures were removed and the dish was washed with 0.9% saline for 2 minutes. Saline was then removed and 500 μL of the stain (Live/Dead BacLight Viability Kit, Invitrogen) were added for 15 minutes and left in the dark. After this time, the stain was removed and the dish was washed twice with 0.9% saline. Then the dish was fixed with 500 μL 4% paraformaldehyde in PBS for 30 minutes. Images of remaining MRSE biofilms were then taken with a fluorescence microscope. All data were analyzed using IMAGE J software.

Hemolysis Assay with Red Blood Cells

Freshly drawn human red blood cells (hRBC with ethylenediaminetetraacetic acid (EDTA) as an anticoagulant) were washed with Tris-buffered saline (0.01M Tris-base, 0.155 M sodium chloride (NaCl), pH 7.2) and centrifuged for 5 minutes at 3,500 rpm. The washing was repeated three times with the buffer. In 96-well plate, test compounds were added to the buffer from DMSO stocks. Then 2% hRBCs (50 μL) in buffer were added to the test compounds to give a final concentration of 200 μM. The plate was then incubated for 1 hour at 37° C. After incubation, the plate was centrifuged for 5 minutes at 3,500 rpm. Then 80 μL of the supernatant was transferred to another 96-well plate and the optical density (OD) was read at 405 nm. DMSO served as our negative control (0% hemolysis) while Triton X served as our positive control (100% hemolysis). The percent of hemolysis was calculated as (OD₄₀₅ of the test compound−OD₄₀₅ DMSO)/(OD₄₀₅ Triton X−OD₄₀₅ buffer) from three independent experiments.

### LDH Release Assay for HeLa Cytotoxicity Assessment

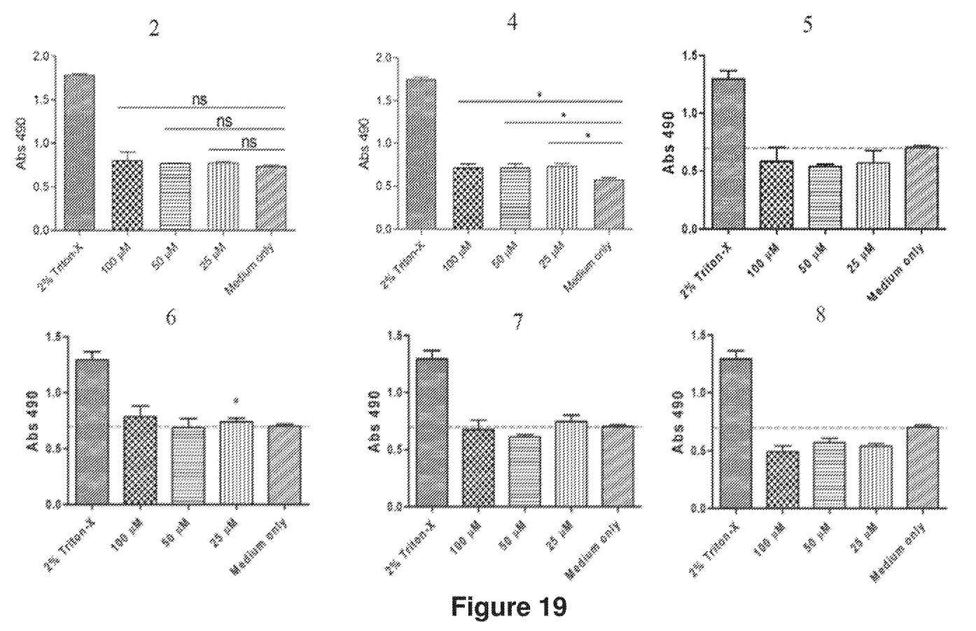

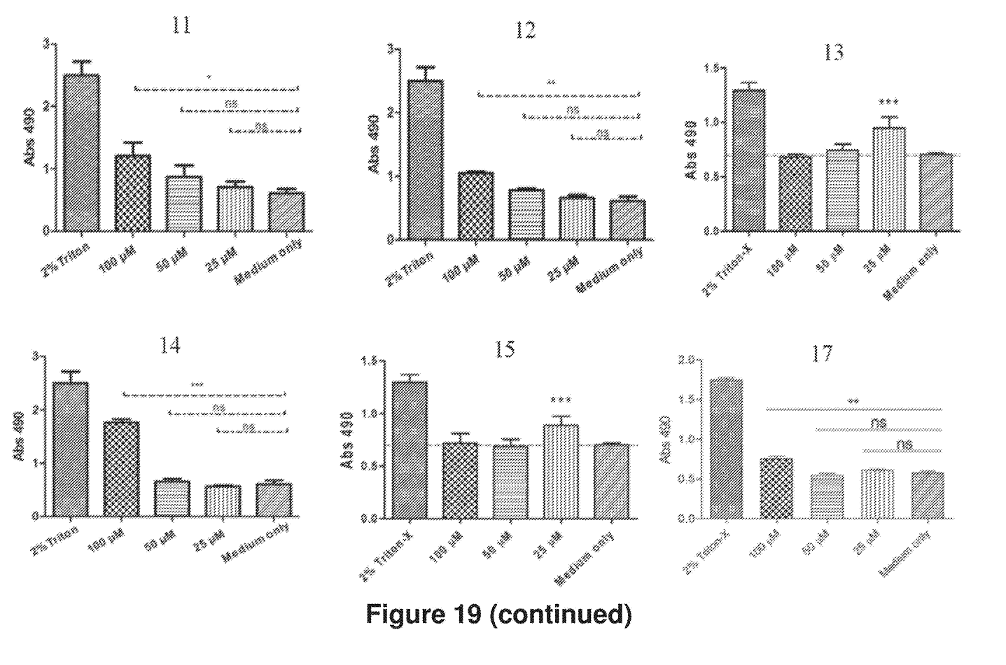

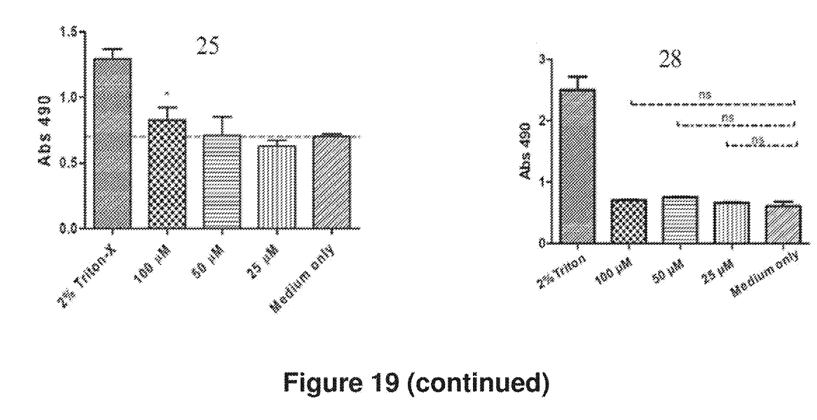

HeLa cytotoxicity was assessed using the LDH release assay described by CYTOTOX96 (Promega G1780). HeLa cells were grown in Dulbecco's Modified Eagle Medium (DMEM; Gibco) supplemented with 10% Fetal Bovine Serum (FBS) at 37° C. with 5% CO₂. When the HeLa cultures exhibited 70-80% confluence, halogenated phenazines were then diluted by DMEM (10% FBS) at concentrations of 25, 50, and 100 μM and added to HeLa cells. Triton X-100 (at 2% v/v) was used as the positive control for maximum lactate dehydrogenate (LDH) activity in this assay (e.g., complete cell death), while “medium only” lanes served as negative control lanes (e.g., no cell death). DMSO was used as our vehicle control. HeLa cells were treated with compounds for 24 hours, and then 50 μL of the supernatant was transferred into a fresh 96-well plate where 50 μL of the reaction mixture was added to the 96-well plate and incubated at room temperature for 30 minutes. Finally, Stop Solution (50 μL) was added to the incubating plates and the absorbance was measured at 490 nm. Results are from three independent experiments (FIG. 19)

HPs 2 and 29 Complex Formation with Cu(II) and Fe(II)

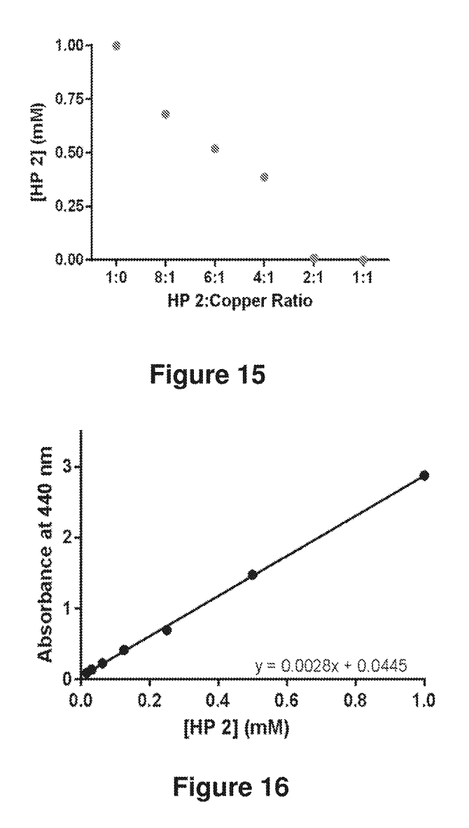

The rates of phenazine-metal(II) and quinoline-metal(II) complex formation were independently evaluated via UV-vis spectrometry following addition of 0.5 equivalents CuSO₄ or ammonium iron(II) sulfate hexahydrate to stirring solutions of HP 2 or 29 (10 mM, 20 mL) in dimethyl sulfoxide. Spectral scanning was performed from 200 to 900 nm in 2 nm increments. The λₘₐₓ values were determined to be 440 nm and 314 nm for 2 and 29 respectively. The disappearance of each complex was observed over the indicated time points. Although the copper(II) complex of 29 yielded an easily discernable UV-vis spectrum (λₘₐₓ=394 nm), the phenazine-copper complex formation yielded a loss in absorbance due to precipitation. Due to the insolubility of the phenazine:copper complex, the stoichiometry was determined by spectrophotometrically quantifying the concentration of free phenazine in solution following incubation with varying equivalents of CuSO₄ (FIG. 15). Each data point was evaluated in independent experiments wherein 10 mM solutions of HP 2 in methanol (15 mL) were stirred with CuSO₄ for 2 hours at room temperature. After this time, the mixtures were filtered and the solvent was removed in vacuo to afford free HP 2 as a solid. HP 2 solid was then dissolved in 15 mL of dimethyl sulfoxide, and the absorbance at 440 nm was evaluated after a single ten-fold dilution was made. The concentrations of free HP 2 in solution could thus be determined from a calibration curve of serial dilutions of HP 2 in dimethyl sulfoxide (FIG. 16). This data demonstrates >99% of HP 2 (ligand) is bound to copper(II) at a minimum of 2:1 ligand:copper ratio, suggesting the stoichiometry of the ligand:metal complex is 2:1.

### Kill Kinetics of Exponential Growth Cultures (Rapidly-Dividing Bacteria)

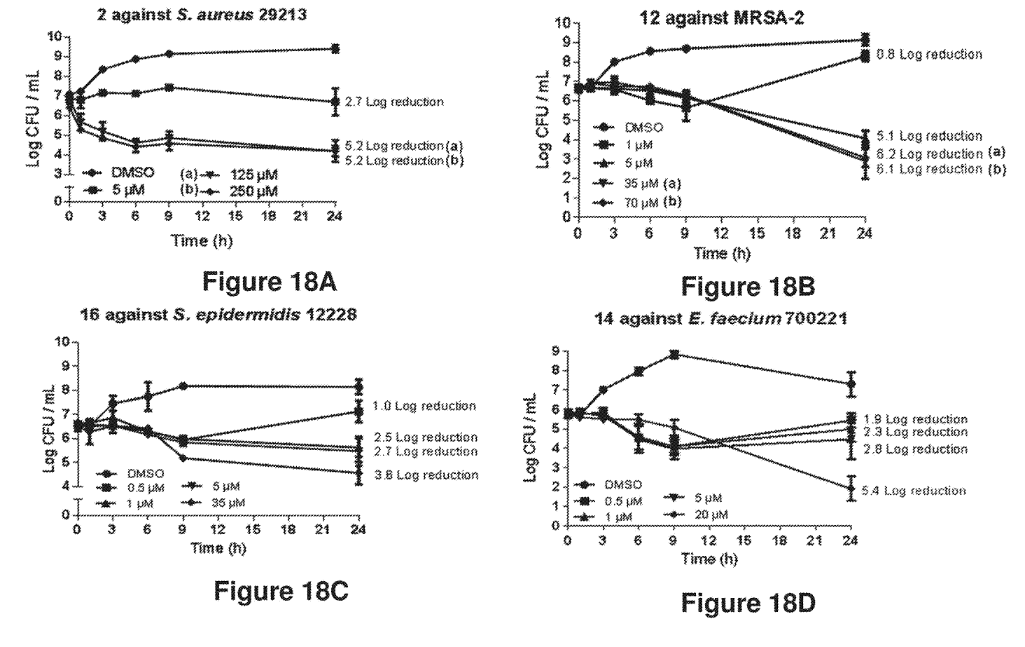

An overnight culture of MRSA-2, S. epidermidis (ATCC 12228) or E. faecium (VRE 700221) was diluted 1:1,000 in 10 mL of LB media (Brain heart infusion media was used for E. faecium) in test tubes containing test compounds. These test tubes were incubated at 37° C. with shaking at 250 rpm. At 0, 1, 3, 6, 9, and 24 hours, 100 μL aliquots were removed from each test tubes, serial diluted and spread on LB 1% agar plates. The resulting plates were then incubated at 37° C. overnight to allow viable colonies to grow. Following this incubation period, viable bacterial colonies were counted on each agar plate to determine colony forming units CFU/mL. Plates containing between 30 and 300 colonies were used to determine CFU/mL. All data was derived from three independent experiments. Compounds were considered to be bactericidal at ≥3-log₁₀ reduction in CFU/mL compared to the vehicle (DMSO) control or bacteriostatic at ≤3-log₁₀ CFU/mL reduction compared to the vehicle (DMSO) control after 24 hours. Kill curves were plotted using GRAPHPAD PRISM 6.0 and were generated from three independent experiments. From these experiments, we concluded that our HP analogues may operate through a bacteriostatic antibacterial mechanism as ≥3-log₁₀ reduction in CFU/mL required concentrations >4× MIC (FIGS. 18A to 18D).

### Spectrophotometric Determination of Dissociation Constants for Select Analogues

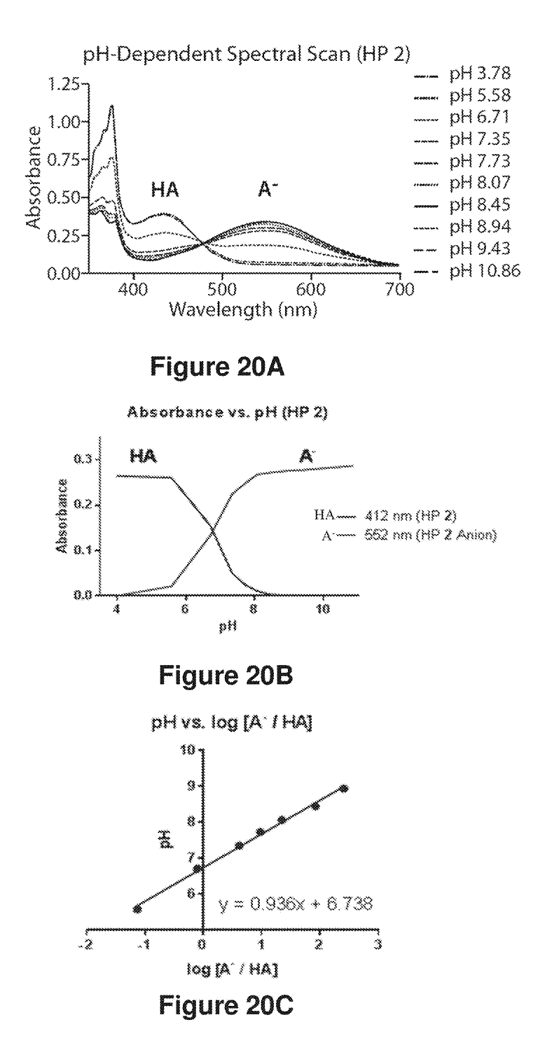

Dissociation constants (pKₐ) for select analogues were determined by implementing UV-vis spectroscopy at varying pH values. Buffers were prepared using potassium phosphate monobasic (KH₂PO₄) and sodium phosphate dibasic (Na₂HPO₄) in a 1:1 solution of water:methanol to achieve a pH range of 3.78 to 10.94. Compounds were added from 10 mM stock solutions in dimethyl sulfoxide (12.5 μL) to 987.5 μL of each buffer to yield a final compound concentration of 125 μM. Full spectral scans were performed for each evaluated analogue at pH 3.78 and pH 10.94 from 300 to 800 nm to determine λₘₐₓ values for the protonated phenol (HA; e.g., HP 2 below) and the deprotonated phenolate species (A⁻; e.g., conjugate base of HP 2 below). The change in absorption at each determined λₘₐₓ in relation to pH was then monitored in each buffer and then plotted as absorbance versus pH for each species. The pKₐ was first estimated by determining the pH of the point of intersection of the two linear curves as shown. The visual estimation was confirmed by plotting pH versus log [A⁻/HA]. The resulting plot yielded a linear regression line with a Y-intercept corresponding to a calculated pKₐ value. As a method validation, the pKₐ of 4-nitrophenol (lit. pKₐ 7.15) was determined to be 7.62 in our assay.⁵⁴ (FIGS. 20A to 20C).

### REFERENCES

[1] Donlan, R. M.; Costerton, J. W. Biofilms: survival mechanisms of clinically relevant microorganisms. Clin. Microbiol. Rev. 2002, 15, 167-193.
[2] Hall-Stoodley, L.; Costerton, J. W.; Stoodley, P. Bacterial biofilms: from the natural environment to infectious diseases. Nat. Rev. Microbiol. 2004, 2, 95-108.
[3] Lewis, K. Persister cells, dormancy and infectious disease. Nature Rev. Microbiol. 2007, 5, 48-56.
[4] Wood, T. K. Combatting bacterial persister cells. Biotechnol. Bioengineer. 2016, 113, 476-483.
[5] Young, D. B.; Perkins, M. D.; Duncan, K.; Barry, C. E. Confronting the scientific obstacles to global control of tuberculosis. J. Clin. Invest. 2008, 118, 1255-1265.
[6] Lewis, K. Persister cells. Annu. Rev. Microbiol. 2010, 64, 357-372.
[7] Conlon, B. P. Staphylococcus aureus chronic and relapsing infections: evidence of a role for persister cells. Bioessays 2014, 36, 991-996.
[8] Balaban, N. Q.; Merrin, J.; Chait, R.; Kowalik, L.; Leibler, S. Bacterial persistence as a phenotype switch. Science 2004, 305, 1622-1625.
[9] Wood, T. K.; Knabel, S. J.; Kwan, B. W. Bacterial persister cell formation and dormancy. Appl. Environ. Microbiol. 2013, 79, 7116-7121.
[10] Wolcott, R.; Dowd, S. The role of biofilms: are we hitting the right target? Plast. Reconstr. Surg. 2011, 127, Suppl. 1: 28S-35S.
[11] Worthington, R. J.: Richards, J. J.; Melander, C. Small molecule control of bacterial biofilms. Org. Biomol. Chem. 2012, 10, 7457-7474.
[12] Harbarth, S.; Theuretzbacher, U.; Hackett, J. Antibiotic research and development: business as usual? J. Antimicrob. Chemother. 2015, 70, 1604-1607.
[13] Brackman, G.; Coenye, T. Quorum sensing inhibitors as anti-biofilms agents. Curr. Pharm. Design 2015, 21, 5-11.
[14] De Zoysa, G. H.; Cameron, A. J.; Hegde, V. V.; Raghothama, S.; Sarojini, V. Antimicrobial peptides with potential for biofilm eradication: synthesis and structure activity relationship studies of battacin peptides. J. Med. Chem. 2015, 58, 625-639.
[15] Hoque, J.; Konai, M. M.; Gonuguntla, S.; Manjunath, G. B.; Samaddar, S.; Yarlagadda, V.; Haldar, J. Membrane active small molecules show selective broad spectrum antibacterial activity with no detectable resistance and eradicate biofilms. J. Med. Chem. 2015, 58, 5486-5500.
[16] Hoque, J.; Konai, M. M.; Samaddar, S.; Gonuguntla, S.; Manjunath, G. B.; Ghosh, C.; Haldar, J. Selective and broad spectrum amphiphilic small molecules to combat bacterial resistance and eradicate biofilms. Chem. Commun. 2015, 51, 13670-13673.
[17] Jennings, M. C.; Ator, L. E.; Paniak, T. J.; Minibole, K. P. C., Wuest, W. M. Biofilm eradicating properties of quaternary ammonium amphiphiles: simple mimics of antimicrobial peptides. ChemBioChem. 2014, 15, 2211-2215.
[18] Mitchell, M. A.; lannetta, A. A.; Jennings, M. C.; Fletcher, M. H.; Wuest, W. M.; Minbiole, K. P. C. Scaffold-hopping of multicationic amphiphiles yields three new classes of antimicrobials. ChemBioChem. 2015, 16, 2299-2303.
[19] Hughes, C. C.; Fenical, W. Antibacterials from the sea. Chem. Eur. J. 2010, 16, 12512-12525.
[20] Ng, W.-L.; Bassler, B. L. Bacterial quorum-sensing network architectures. Annu. Rev. Genet. 2009, 43, 197-222.
[21] Hentzer, M.; Wu, H.; Andersen, J. B.; Riedel, K.; Rasmussen, T. B.; Bagge, N.; Kumar, N.; Schembri, M. A.; Song, Z.; Kristoffersen, P.; Manefield, M.; Costerton, J. W.; Molin, S.; Eberl, L.; Steinberg, P.; Kjelleberg, S.; Høiby, N.; Givskov, M. Attenuation of Pseudomonas aeruginosa virulence by quorum sensing inhibitors. EMBO J. 2003, 22, 3803-3815.
[22] Wu, H.; Song, Z.; Hentzer, M.; Andersen, J. B.; Molin, S.; Givskov, M.; Høiby, N. Synthetic furanones inhibit quorum-sensing and enhance bacterial clearance in Pseudomonas aeruginosa lung infection in mice. J Antimicrob. Chemother. 2004, 53, 1054-1061.
[23] Kwan, J. C.; Meickle, T.; Ladwa, D.; Teplitski, M.; Paul, V. J.; Luesch, H. Lyngbyoic acid, a “tagged” fatty acid from a marine cyanobacterium, disrupts quorum sensing in Pseudomonas aeruginosa. Mol. BioSyst. 2011, 7, 1205-1216.
[24] Navarro, G.; Cheng, A. T.; Peach, K. C.; Bray, W. M.; Bernan, V. S.; Yildiz, F. H.; Linington, R. G. Image-based 384-well high-throughput screening method for the discovery of skyllamycins A to C as biofilm inhibitors and inducers of biofilm detachment in Pseudomonas aeruginosa. Antimicrob. Agents Chemother. 2014, 58, 1092-1099.
[25] Conda-Sheridan, M.; Marler, L.; Park, E. J.; Kondratyuk, T. P.; Jermihov, K.; Mesecar, A. D.; Pezzuto, J. M.; Asolkar, R. N.; Fenical, W.; Cushman, M. Potential chemopreventive agents based on the structure of the lead compound 2-bromo-1-hydroxyphenazine, isolated from Streptomyces species, strain CNS284. J. Med. Chem. 2010, 53, 8688-8699.
[26] Borrero, N. V.; Bai, F.; Perez, C.; Duong, B. Q.; Rocca, J. R.; Jin, S.; Huigens III, R. W. Phenazine antibiotic inspired discovery of potent bromophenazine antibacterial agents against Staphylococcus aureus and Staphylococcus epidermidis. Org. Biomol. Chem. 2014, 12, 881-886.
[27] Garrison, A. T.; Bai, F.; Abouelhassan, Y.; Paciaroni, N. G.; Jin, S.; Huigens III, R. W. Bromophenazine derivatives with potent inhibition, dispersion and eradication activities against Staphylococcus aureus biofilms. RSC Adv. 2015, 5, 1120-1124.
[28] Garrison, A. T.; Abouelhassan, Y.; Kallifidas, D.; Bai, F.; Ukhanova, M.; Mai, V.; Jin, S.; Luesch, H.; Huigens III, R. W. Halogenated phenazines that potently eradicate biofilms, MRSA persister cells in non-biofilm cultures, and Mycobacterium tuberculosis. Angew. Chemie., Int. Ed. 2015, 54, 14819-14823.
[29] Pachter, I. J.; Kloetzel, M. C. The Wohl-Aue reaction. I. Structure of benzo [a]phenazine oxides and syntheses of 1,6-dimethoxyphenazine and 1,6-dichlorophenazine. J. Am. Chem. Soc. 1951, 73, 4958-4961.
[30] Evangelopoulos, D.; McHugh, T. D. Improving the tuberculosis drug development pipeline. Chem. Biol. Drug Des. 2015, 86, 951-960.
[31] Pérez-Lago, L.; Navarro, Y.; Montilla, P.; Comas, I.; Herranz, M.; Rodríguez-Gallego, C.; Ruiz Serrano, M. J.; Bouza, E.; Garcia de Viedma, D. Persistent infection by a Mycobacterium tuberculosis strain that was theorized to have advantageous properties, as it was responsible for a massive outbreak. J. Clin. Microbiol. 2015, 53, 3423-3429.
[32] Weidmann, E.; Brieger, J.; Jahn, B.; Hoelzer, D.; Bergmann, L.; Mitrou, P. S. Lactate dehydrogenase-release assay: A reliable, nonradioactive technique for analysis of cytotoxic lymphocyte-mediated lytic activity against blasts from acute myelocytic leukemia. Annu. Hematol. 1995, 70, 153-158.
[33] Ceri, H.; Olson, M. E.; Stremick, C.; Read, R. R.; Morck, D.; Buret, A. The calgary biofilm device: new technology for rapid determination of antibiotic susceptibilities of bacterial biofilms. J. Clin. Microbiol. 1999, 37, 1771-1776.
[34] Harrison, J. J.; Turner, R. J.; Joo, D. A.; Stan, M. A.; Chan, C. S.; Allan, N. D.; Vrionis, H. A.; Olson, M. E.; Ceri, H. Copper and quaternary ammonium cations exert synergistic bactericidal and antibiofilm activity against Pseudomonas aeruginosa. Antimicrob. Agents Chemother. 2008, 52, 2870-2881.
[35] Harrison, J. J.; Stremick, C. A.; Turner, R. J.; Allan, N. D.; Olson, M. E.; Ceri, H. Microtiter susceptibility testing of microbes growing on peg lids: a miniaturized biofilm model for high-throughput screening. Nat. Protoc. 2010, 5, 1236-1254.
[36] Basak, A.; Abouelhassan, Y.; Huigens III, R. W. Halogenated quinolines discovered through reductive amination with potent eradication activities against MRSA, MRSE and VRE biofilms. Org. Biomol. Chem. 2015, 13, 10290-10294.
[37] Eun, Y. J.; Foss, M. H.; Kiekebusch, D.; Pauw, D. A.; Westler, W. M.; Thanbichler, M.; Weibel, D. B. DCAP: a broad-spectrum antibiotic that targets the cytoplasmic membrane of bacteria. J Am. Chem. Soc. 2012, 134, 11322-11325.
[38] Quah, S. Y.; Wu, S.; Lui, J. N.; Sum, C. P.; Tan, K. S. N-acetylcysteine inhibits growth and eradicates biofilm of Enterococcus faecalis. J. Endod. 2012, 38, 81-85.
[39] McCune, R. M.; Feldmann, F. M.; McDermott, W. Microbial persistence. II. Characteristics of the sterile state of tubercle bacilli. J Exp. Med. 1966, 123, 469-486.
[40] Shi, W.; Zhang, X.; Jiang, X.; Yuan, H.; Lee, J. S.; Barry, C. E.; Wang, H.; Zhang, W.; Zhang, Y. Pyrazinamide inhibits trans-translation in Mycobacterium tuberculosis. Science 2011, 333, 1630-1632.
[41] Abouelhassan, Y.; Garrison, A. T.; Burch, G. M.; Wong, W.; Norwood IV, V. M.; Huigens III, R. W. Discovery of quinoline small molecules with potent dispersal activity against methicillin-resistant Staphylococcus aureus and Staphylococcus epidermidis biofilms using a scaffold hopping strategy. Bioorg. Med. Chem. Lett. 2014, 24, 5076-5080.
[42] Abouelhassan, Y.; Garrison, A. T.; Bai, F.; Norwood IV, V. M.; Nguyen, M.; Jin, S.; Huigens III, R. W. A phytochemical-halogenated quinoline combination therapy strategy for the treatment of pathogenic bacteria. ChemMedChem 2015, 10, 1157-1162.
[43] Laursen, J. B.; Nielsen, J. Phenazine natural products: biosynthesis, synthetic analogues, and biological activity. Chem. Rev. 2004, 104, 1663-1685.
[44] Price-Whelan, A.; Dietrich, L. E. P.; Newman, D. K. Rethinking ‘secondary’ metabolism: physiological roles for phenazine antibiotics. Nat. Chem. Biol. 2006, 2, 71-78.
[45] Taiwo, F. A. Mechanism of tiron as scavenger of superoxide ions and free electrons. Spectroscopy 2008, 22, 491-498.
[46] Gershon, H.; Parmegiani, R. Antimicrobial activity of 8-quinolinol, its salts with salicylic acid and 3-hydroxy-2-naphthoic acid, and the respective copper (II) chelates in liquid culture. Appl. Environ. Microbiol. 1963, 11, 62-65.
[47] Deraeve, C.; Pitié, M.; Mazarguil, H.; Meunier, B. Bis-8-hydroxyquinoline ligands as potential anti-Alzheimer agents. New J. Chem. 2007, 31, 193-195.
[48] Prachayasittikul, V.; Prachayasittikul, S.; Ruchirawat, S.; Prachayasittikul, V. 8-Hydroxyquinolines: a review of their metal chelating properties and medicinal applications. Drug Des. Devel. Ther. 2013, 7, 1157-1178.
[49] Di Varia, M.; Bazzicalupi, C.; Orioli, P.; Messori, L.; Bruni, B.; Zatta, P. Clioquinol, a drug for Alzheimer's disease specifically interfering with brain metal metabolism: structural characterization of its zinc(II) and copper(II) complexes. Inorg. Chem. 2004, 43, 3795-3797.
[50] Briard, B.; Bomme, P.; Lechner, B. E.; Mislin, G. L. A.; Liar, V.; Prévost, M. C.; Latgé, J. P.; Haas, H.; Beauvais, A. Pseudomonas aeruginosa manipulates redox and iron homeostasis of its microbiota partner Aspergillus fumigatus via phenazines. Sci. Rep. 2015, 5; doi:10.1038/srep08220.
[51] Chernetskii, V. P.; Kiprianov, A. I. Synthesis of halogen derivatives of phenazine. IV. Bromophenazines. Zhurnal Obshchei Khimii 1956, 26, 3032-3036.
[52] Clinical and Laboratory Standards Institute. 2009. Methods for dilution antimicrobial susceptibility tests for bacteria that grow aerobically; approved standard, 8th edition (M7-M8), Clinical and Laboratory Standard, Wayne, Pa., 2009.
[53] Emmanuvel, L.; Shukla, R. K.; Sudalai, A.; Gurunath, S.; Sivaram, S. Tetrahedron Lett. 2006, 47, 4793-4796.
[54] Serjeant, E. P.; Dempsey, B. Ionisation Constants of Organic Acids in Aqueous Solution; IUPAC Chemical Data Series; Oxford: New York, N.Y., 1979; Vol. 23.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A compound of Formula (I′): 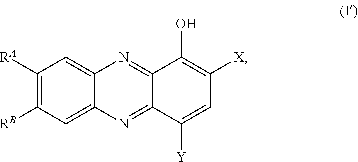
or a pharmaceutically acceptable salt thereof, wherein:
X is hydrogen, halogen, substituted or unsubstituted alkyl, substituted or unsubstituted alkenyl, or substituted or unsubstituted alkynyl;
Y is halogen, substituted or unsubstituted alkyl, substituted or unsubstituted alkenyl, or substituted or unsubstituted alkynyl;
R^{A} is hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR¹, —N(R¹)₂, —SR¹, —CN, —SCN, —C(═NR¹)R¹, —C(═NR¹)OR¹, —C(═NR¹)N(R¹)₂, —C(═O)R¹, —C(═O)OR¹, —C(═O)N(R¹)₂, —NO₂, —NR¹C(═O)R¹, —NR¹C(═O)OR¹, —NR¹C(═O)N(R¹)₂, —OC(═O)R¹, —OC(═O)OR¹, or —OC(═O)N(R¹)₂, wherein each instance of R¹ is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R¹ are joined to form a substituted or unsubstituted heterocyclic or substituted or unsubstituted heteroaryl ring; and
R^{B} is hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR², —N(R²)₂, —SR², —CN, —SCN, —C(═NR^{Z})R², —C(═NR²)OR², —C(═NR²)N(R²)₂, —C(═O)R², —C(═O)OR², —C(═O)N(R²)₂, —NO₂, —NR C(═O)R², —NR C(═O)OR², —NR C(═O)N(R²)₂, —OC(═O)R², —OC(═O)OR², or —OC(═O)N(R²)₂, wherein each instance of R² is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R² are joined to form a substituted or unsubstituted heterocyclic or substituted or unsubstituted heteroaryl ring;
or R^{A} and R^{B} are joined to form a substituted or unsubstituted phenyl ring;
provided that:
at least one of X and Y is halogen; and
the compound is not of the formula:

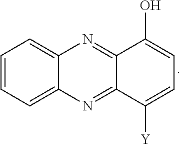

2. The compound of claim 1, wherein the compound is of Formula (I): 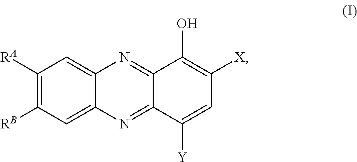
or a pharmaceutically acceptable salt thereof, wherein:
X is hydrogen or halogen;
Y is halogen;
R^{A} is hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR¹, —N(R¹)₂, —SR¹, —CN, —SCN, —C(═NR¹)R¹, —C(═NR¹)OR¹, —C(═NR¹)N(R¹)₂, —C(═O)R¹, —C(═O)OR¹, —C(═O)N(R¹)₂, —NO₂, —NR¹C(═O)R¹, —NR¹C(═O)OR¹, —NR¹C(═O)N(R¹)₂, —OC(═O)R¹, —OC(═O)OR¹, or —OC(═O)N(R¹)₂, wherein each instance of R¹ is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R¹ are joined to form a substituted or unsubstituted heterocyclic or substituted or unsubstituted heteroaryl ring; and
R^{B} is hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR², —N(R²)₂, —SR², —CN, —SCN, —C(═NR²)R², —C(═NR²)OR², —C(═NR²)N(R²)₂, —C(═O)R², —C(═O)OR², —C(═O)N(R²)₂, —NO₂, —NR C(═O)R², —NR C(═O)OR², —NR C(═O)N(R²)₂, —OC(═O)R², —OC(═O)OR², or —OC(═O)N(R²)₂, wherein each instance of R² is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R² are joined to form a substituted or unsubstituted heterocyclic or substituted or unsubstituted heteroaryl ring;
or R^{A} and R^{B} are joined to form a substituted or unsubstituted phenyl ring;
provided that the compound is not of the formula:

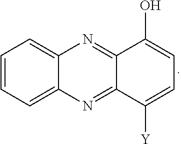

3. (canceled)

4. The compound of claim 1, wherein at least one of R^{A} and R^{B} is not hydrogen.

5-6. (canceled)

7. The compound of claim 1, wherein the compound is of the formula: 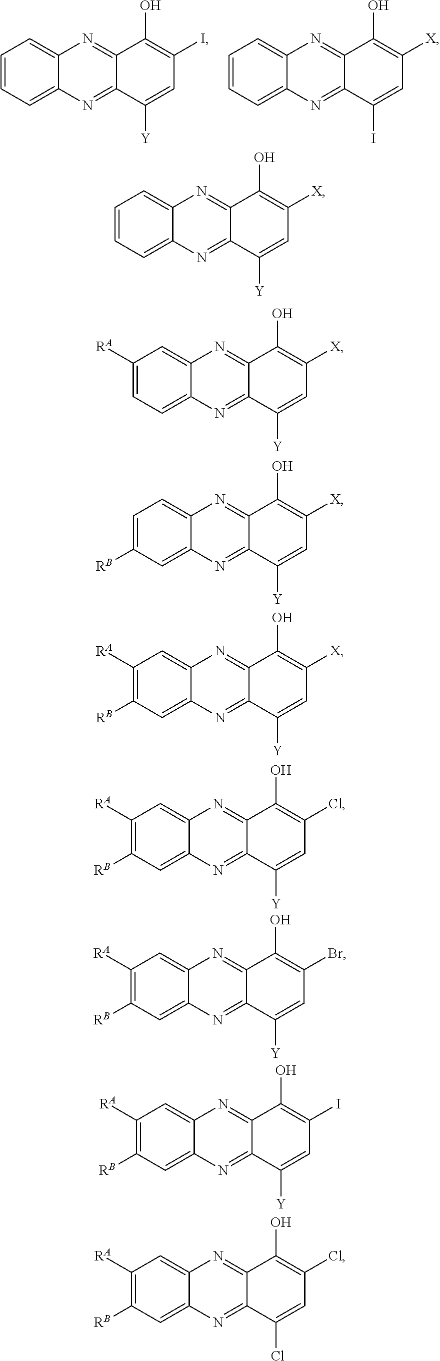 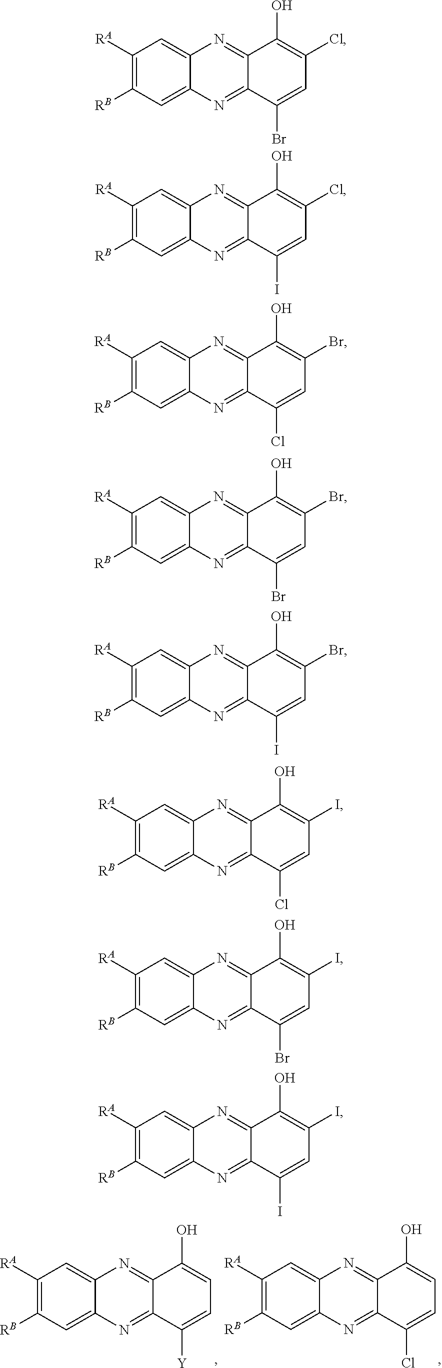 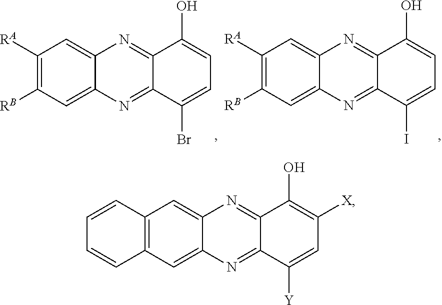
or a pharmaceutically acceptable salt thereof.

8-20. (canceled)

21. The compound of claim 1, wherein X is hydrogen.

22. The compound of claim 1, wherein X is halogen.

23-30. (canceled)

31. The compound of claim 1, wherein Y is Cl, Br, or I.

32-34. (canceled)

35. The compound of claim 1 wherein Y is substituted or unsubstituted C₁₋₆ alkyl.

36-47. (canceled)

48. The compound of claim 1, wherein R^{A} is halogen.

49. (canceled)

50. The compound of claim 1, wherein R^{A} is substituted or unsubstituted C₁₋₆ alkyl.

51. (canceled)

52. The compound of claim 1, wherein R^{A} is hydrogen.

53. The compound of claim 1, wherein R^{B} is halogen.

54. (canceled)

55. The compound of claim 1, wherein R^{B} is substituted or unsubstituted C₁₋₆ alkyl.

56. (canceled)

57. The compound of claim 1, wherein R^{B} is hydrogen.

58-59. (canceled)

60. The compound of claim 1, wherein each of R^{A} and R^{B} is independently hydrogen or halogen.

61. The compound of claim 1, wherein the compound is of the formula: 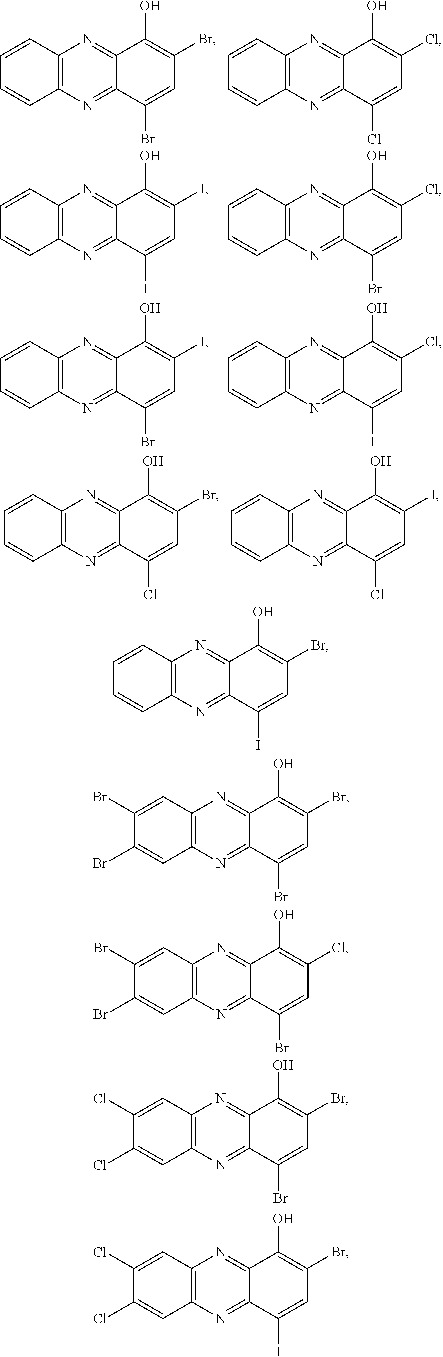 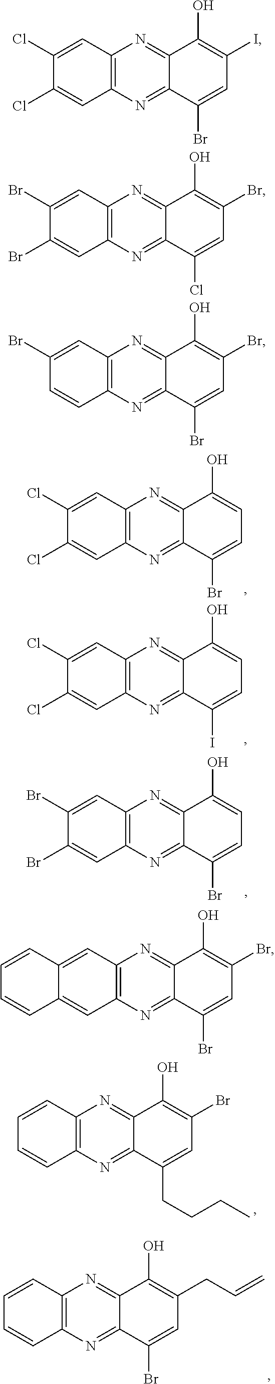 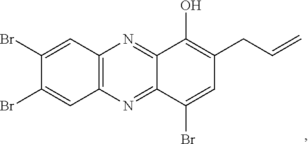 [SMILES: C=CCc1cc(Br)c2nc3cc(Br)c(Br)cc3nc2c1O]
or a pharmaceutically acceptable salt thereof.

62-70. (canceled)

71. A compound of Formula (II): 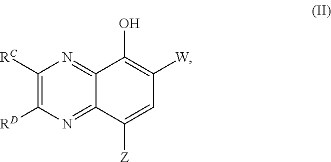
or a pharmaceutically acceptable salt thereof, wherein:
W is hydrogen or halogen;
Z is halogen;
R^{C} is hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR³, —N(R³)₂, —SR³, —CN, —SCN, —C(═NR³)R³, —C(═NR³)OR³, —C(═NR³)N(R³)₂, —C(═O)R³, —C(═O)OR³, —C(═O)N(R³)₂, —NO₂, —NR³C(═O)R³, —NR³C(═O)OR³, —NR³C(═O)N(R³)₂, —OC(═O)R³, —OC(═O)OR³, or —OC(═O)N(R³)₂, wherein each instance of R³ is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R³ are joined to form a substituted or unsubstituted heterocyclic or substituted or unsubstituted heteroaryl ring; and
R^{D} is hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR⁴, —N(R⁴)₂, —SR⁴, —CN, —SCN, —C(═NR⁴)R⁴, —C(═NR⁴)OR⁴, —C(═NR⁴)N(R⁴)₂, —C(═O)R⁴, —C(═O)OR⁴, —C(═O)N(R⁴)₂, —NO₂, —NR⁴C(═O)R⁴, —NR⁴C(═O)OR⁴, —NR⁴C(═O)N(R⁴)₂, —OC(═O)R⁴, —OC(═O)OR⁴, or —OC(═O)N(R⁴)₂, wherein each instance of R⁴ is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R⁴ are joined to form a substituted or unsubstituted heterocyclic or substituted or unsubstituted heteroaryl ring;
provided that the compound is not of the formula:

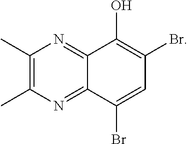

72. A compound of Formula (III): 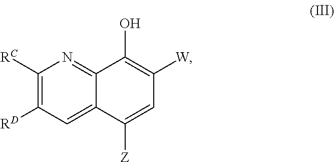
or a pharmaceutically acceptable salt thereof, wherein:
W is hydrogen or halogen;
Z is halogen;
R^{C} is hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR³, —N(R³)₂, —SR³, —CN, —SCN, —C(═NR³)R³, —C(═NR³)OR³, —C(═NR³)N(R³)₂, —C(═O)R³, —C(═O)OR³, —C(═O)N(R³)₂, —NO₂, —NR³C(═O)R³, —NR³C(═O)OR³, —NR³C(═O)N(R³)₂, —OC(═O)R³, —OC(═O)OR³, or —OC(═O)N(R³)₂, wherein each instance of R³ is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R³ are joined to form a substituted or unsubstituted heterocyclic or substituted or unsubstituted heteroaryl ring; and
R^{D} is hydrogen, halogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR⁴, —N(R⁴)₂, —SR⁴, —CN, —SCN, —C(═NR⁴)R⁴, —C(═NR⁴)OR⁴, —C(═NR⁴)N(R⁴)₂, —C(═O)R⁴, —C(═O)OR⁴, —C(═O)N(R⁴)₂, —NO₂, —NR⁴C(═O)R⁴, —NR⁴C(═O)OR⁴, —NR⁴C(═O)N(R⁴)₂, —OC(═O)R⁴, —OC(═O)OR⁴, or —OC(═O)N(R⁴)₂, wherein each instance of R⁴ is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R⁴ are joined to form a substituted or unsubstituted heterocyclic or substituted or unsubstituted heteroaryl ring.

73-89. (canceled)

90. A composition comprising a compound of claim 1, or a salt thereof, and optionally an excipient.

91. A pharmaceutical composition comprising a compound of claim 1, or a pharmaceutically acceptable salt thereof, and optionally a pharmaceutically acceptable excipient.

92-96. (canceled)

97. A method of preventing or treating a microbial infection in a subject in need thereof, the method comprising administering to the subject an effective amount of a compound of claim 1, or a pharmaceutical acceptable salt thereof.

98-113. (canceled)

114. A method of inhibiting the formation of a biofilm, growth of a biofilm, or clearing or reducing a biofilm, the method comprising contacting the biofilm with an effective amount of a compound of claim 1, or a pharmaceutical acceptable salt thereof.

115-140. (canceled)

141. A method of disinfecting a surface, the method comprising contacting the surface with an effective amount of a compound of claim 1, or a pharmaceutical acceptable salt thereof.

142. (canceled)
